(19) United States
(12) Patent Application Publication
(10) Pub. No.: US 2023/0317205 A1
(43) Pub. Date: Oct. 5, 2023

(54) SYSTEMS AND METHODS FOR PRODUCING OR IDENTIFYING NON-HUMAN ANIMALS WITH A PREDETERMINED PHENOTYPE OR GENOTYPE

(71) Applicant: Etalon Diagnostics, Menlo Park, CA (US)

(72) Inventors: Christa LAFAYETTE, Menlo Park, CA (US); Russell KERSCHMANN, Menlo Park, CA (US); Katie Anne MARTIN, Hollister, CA (US); Erica Grace WAGNER LUNDQUIST, Fountain Valley, CA (US)

(21) Appl. No.: 18/022,681

(22) PCT Filed: Aug. 31, 2021
(86) PCT No.: PCT/US21/48516

§ 371 Date: Feb. 22, 2023

Related U.S. Application Data
(60) Provisional application No. 63/072,883, filed on Aug. 31, 2020.

(51) Int. Cl. G16B 20/00; G16B 10/00

(52) U.S. Cl. CPC G16B 20/00; G16B 10/00

(57) ABSTRACT

The present disclosure provides methods and computer-implemented systems for producing, procuring, or identifying a non-human animal or offspring thereof having one or more predetermined traits. Also disclosed herein are a computer implemented graphical user interface and database system containing information associated with one or more predetermined traits for use in generating a probability matrix containing an array of probability values pertaining to the likelihood of producing a non-human animal having one or more predetermined traits.

### SEQUENCE LISTING

The instant application contains a Sequence Listing which has been submitted electronically in ASCII format and is hereby incorporated by reference in its entirety. Said ASCII copy, created on Aug. 26, 2021, is named “51370-003WO2_Sequence_Listing_8_26_21_ST25.txt” and is 2,020,983 bytes in size.

### BACKGROUND

Animal breeders have long relied on overt phenotypic proxies of animal health and performance in order to select for specific traits desired in the animal. Such proxies include personal testimony from the animal seller, morphological features, age, abilities, and pedigree of the animal. Selection strategies based on these parameters may, and often does, result in inadvertent co-selection of undesired traits (e.g., disease conditions) that are genetically linked with a desired trait and that are unrecognized by the animal enthusiast during breeding.

The technological development of clinical diagnostic testing for veterinarian use has enabled more accurate identification of genetic markers associated with specific traits. Nonetheless, existing diagnostic platforms generally focus on single gene-trait correspondences that emphasize selection of athletic traits, thus limiting the ability to provide a holistic assessment of an animal in order to predict its future health and performance. Therefore, there exists a need for an integrative diagnostic platform that evaluates a broad spectrum of animal traits based on multiple sources of information, including genetic and non-genetic information, to facilitate production or identification of animals having desirable characteristics suited for the intended use of the animal or preferred by the owner.

### SUMMARY OF THE DISCLOSURE

The present disclosure provides methods, computer-implemented systems, and databases for producing, procuring, and/or identifying a non-human animal having one or more predetermined traits (e.g., traits pertaining to physical appearance, morphology, health, and performance). The disclosure features a computer-implemented database system containing information associated with one or more predetermined traits (e.g., genetic information and non-genetic information) for use in determining a probability of producing a non-human animal having one or more predetermined. The methods and systems disclosed herein are also useful for selecting one or more predetermined traits of a non-human animal, identifying potential breeding pair matches capable of producing the animal having the traits of interest, identifying potential health risks associated with the selection of said traits, and identifying existing animals having the one or more predetermined traits. The methods and systems disclosed herein are also applicable for the production of genetically-modified (e.g., transgenic) non-human animals having one or more predetermined traits of interest. The disclosed methods may be used for assessing the health, behavior, performance, predispositions, and/or monetary value of an animal. Such information may be particularly useful in making decisions pertaining to breeding, husbandry, medical treatment, diet, exercise, environmental exposure, supplementary treatments, training, conditioning, use, limitation, and/or destiny determination (e.g., discipline) of the animal.

In a first aspect, the present disclosure provides a method of producing a non-human offspring animal having one or more (e.g., 1, 2, 3, 4, 5, 6, 7, 8, 9, 10, or more) predetermined traits. In some embodiments, the method includes selecting one more predetermined traits desired in the offspring animal using a computer-implemented database system. In some embodiments, the method includes interrogating the database system to identify one or more (e.g., 1, 2, 3, 4, 5, 6, 7, 8, 9, 10, 15, 20, or more) breeding pairs capable of producing the offspring animal. In some embodiments, the method includes generating a probability matrix for each of the one or more breeding pairs using the database system, wherein the probability matrix includes an array of probability values pertaining to the likelihood that the offspring animal produced by each said breeding pair will have the one or more predetermined traits. In some embodiments, the method includes selecting, based on the array of probability values, one or more of the breeding pairs likely to produce the offspring animal having the one or more predetermined traits. In some embodiments, the method includes breeding one or more of the selected breeding pairs to produce the offspring animal. In some embodiments, the method includes performing genetic testing of the offspring animal to detect the presence of the one or more predetermined traits. In some embodiments, the genetic testing indicates that the offspring animal has the one or more predetermined traits. In some embodiments, the genetic testing indicates that the offspring animal lacks the one or more predetermined traits. In some embodiments, the method includes genetically modifying the offspring animal lacking the one or more predetermined traits to promote or improve expression of the one or more predetermined traits. In some embodiments, the offspring animal lacking the one or more predetermined traits lacks one or more genes (e.g., any one of the genes disclosed herein) or variants thereof that promote the expression of the one or more predetermined traits. In some embodiments, the offspring animal lacking the one or more predetermined traits has one or more genes or variants thereof that that suppress the expression of the one or more predetermined traits. In still other embodiments, the offspring animal lacking the one or more predetermined traits has a variant of one or more genes (e.g., one or more SNPs or other genetic mutation(s), such as deletions, inversions, and duplications) the expression of which results in the absence of the one or more predetermined traits. The genetic variation may also occur in a non-coding, extra-coding, or other regulatory region of the gene(s).

In some embodiments, the genetic testing and/or the genetic modification is performed in the offspring animal during embryonic development. In some embodiments, the genetic testing and or the genetic modification is performed in the offspring animal 3 to 60 (e.g., 3, 4, 5, 6, 7, 8, 9, 10, 11-15, 16-20, 21-25, 26-30, 31-35, 36-40, 41-45, 46-50, 51-55, or 56-60) days after conception. In some embodiments, the method further includes birthing the offspring animal. In some embodiments, the genetic testing and/or the genetic modification is performed in the offspring animal one day to four years after birth. In some embodiments, the method further includes confirming the presence of one or more predetermined traits in the offspring animal after birth. In some embodiments, the method further includes genetically modifying the offspring animal lacking the one or more predetermined traits after birth.

In some embodiments, the genetic testing is performed in a sample obtained from the offspring animal. In some embodiments, the sample includes blood, tissue, hair, cell-free fetal DNA (e.g., cell-free fetal DNA obtained from a female parent animal during prenatal development of the offspring animal), mitochondria (e.g., mitochondria obtained from the female parent animal or the offspring animal), a gamete (e.g., a gamete of one or more parent animals of the offspring animal), or an embryo of the offspring animal implanted as part of an in vitro fertilization procedure. In some embodiments, the gamete is an oocyte. In some embodiments, the gamete is a spermatozoon. In some embodiments, the gamete includes a cytoplasm, a nucleus, and a mitochondrion.

In some embodiments, the genetic testing is performed using Sanger sequencing, Next Generation Sequencing, AmpliSeq, microarray, genomic sequencing (e.g., genome wide association study (GWAS)), or real-time quantitative polymerase chain reaction (RT-qPCR).

In some embodiments, the expression of the one or more predetermined traits results from expression of one or more (e.g., 1, 2, 3, 4, 5, 6, 7, 8, 9, 10, or more) genes. In some embodiments, genetically modifying the offspring animal includes modifying at least 1, at least 2, at least 3, at least 4, at least 5, at least 6, at least 7, at least 8, at least 9, or at least 10 genes in the offspring animal.

In some embodiments, genetically modifying the offspring animal includes editing of an endogenous gene in the offspring animal. In some embodiments, the endogenous gene includes a polymorphism (e.g., a single nucleotide polymorphism). In some embodiments, the endogenous gene includes a genetic mutation (e.g., insertion, deletion (e.g., knockout), translocation, inversion, single point mutation, or other mutation). In some embodiments, editing of the endogenous gene includes modifying, deleting, or replacing at least one nucleotide of a DNA or an RNA encoding the endogenous gene in the offspring animal. In some embodiments, the editing of an endogenous gene includes inserting a donor polynucleotide into a region of the endogenous gene in the offspring animal. In some embodiments, the donor polynucleotide includes a segment of the endogenous gene that lacks the polymorphism. In some embodiments, the donor polynucleotide includes a segment of the endogenous gene that lacks the genetic mutation. In some embodiments, the editing of an endogenous gene includes administering to the offspring animal a polynucleotide encoding one or more copies of a wild type variant of the endogenous gene. In some embodiments, the editing of an endogenous gene includes introducing a heterologous transgene into the genome of the offspring animal. In some embodiments, the editing of an endogenous gene includes modulating (e.g., increasing or decreasing) expression of an endogenous gene in the offspring animal. In some embodiments, modulating expression of an endogenous gene includes increasing expression of the endogenous gene in the offspring animal. In some embodiments, modulating expression of an endogenous gene includes decreasing expression of the endogenous gene in the offspring animal.

In some embodiments, genetically modifying the offspring animal is performed using a clustered regularly interspaced short palindromic repeats (CRISPR)-Cas system, a transcription activator-like effector nuclease (TALEN), or a zinc finger nuclease (ZFN). In some embodiments, the CRISPR-Cas system includes at least one guide RNA (gRNA) and at least one Cas9 endonuclease. In some embodiments, the Ca9 nuclease includes a nuclease-competent Cas9 endonuclease or a nuclease-inactivated Cas9 (dCas9) endonuclease. In some embodiments, a nuclease-competent Cas9 endonuclease or a nuclease-inactivated Cas9 (dCas9) endonuclease. In some embodiments, the transcriptional regulator domain is selected from a group consisting of VP16, VP64, p65, RTA, VPR, SAM, SunTag, and KRAB.

In some embodiments, the breeding pair capable of producing the offspring animal is integrated into the database system. In some embodiments, the breeding pair is integrated into the database system by providing information about one or more predetermined traits in the breeding pair. In some embodiments, the information about the one or more predetermined traits in the breeding pair includes results of previously performed genetic testing and/or non-genetic assessment of the plurality of non-human animals, wherein the results of the previously performed genetic testing or non-genetic assessment identify the breeding pair as having one or more of the predetermined traits. In some embodiments, the information about the one or more predetermined traits in the breeding pair includes results of genetic testing and/or non-genetic assessment of the breeding pair confirm the presence of one or more of the predetermined traits the breeding pair. In some embodiments, the information about the one or more predetermined traits is catalogued into the database system. In some embodiments, the database system is configured for interrogation by a user and is configured to produce the probability matrix following said interrogation.

In some embodiments, the method further includes registering the offspring animal having the one or more predetermined traits in the database system as a breeder animal.

In another aspect, the present disclosure provides a method for interrogating a computer-implemented database system to identify one or more breeding pairs capable of producing a non-human offspring animal having one or more predetermined traits. In some embodiments, the method includes selecting one or more predetermined traits desired in the offspring animal using the database system. In some embodiments, the method includes identifying the one or more breeding pairs capable of producing the offspring animal using the database system. In some embodiments, the method includes generating a probability matrix for each of the one or more breeding pairs using the database system, wherein the probability matrix includes an array of probability values pertaining to the likelihood that the offspring animal produced by each said breeding pair will have the one or more predetermined traits and displaying the probability matrix on a computer-implemented interface. In some embodiments, the method includes selecting, based on the array of probability values, one or more of the breeding pairs likely to produce the offspring animal having the one or more predetermined traits. In some embodiments, the method includes breeding the one or more breeding pairs to produce the offspring animal.

In some embodiments, the database includes a panel of at least 2 (e.g., at least 2, 4, 8, 10, 20, 30, 40, 50, 100, 200, 400, 600, 800, 1000, or more) predetermined traits. In some embodiments, the database includes a panel of 2-40 (e.g., 2, 3, 4, 5, 6, 7, 8, 9, 10, 15, 20, 25, 35, or 40) predetermined traits. In some embodiments, the database includes a panel of at least 4 predetermined traits. In some embodiments, the database includes a panel of at least 6 predetermined traits. In some embodiments, the database includes a panel of at least 8 predetermined traits. In some embodiments, the database includes a panel of at least 10 predetermined traits.

In some embodiments, the method includes selecting a panel of 2-40 predetermined traits. In some embodiments, the method includes selecting a panel of 2 or more predetermined traits. In some embodiments, the method includes selecting a panel of 4 or more predetermined traits. In some embodiments, the method includes selecting a panel of 6 or more predetermined traits. In some embodiments, the method includes selecting a panel of 8 or more predetermined traits. In some embodiments, the method includes selecting a panel of 10 or more predetermined traits.

In some embodiments, the method includes confirming the presence of one or more of the predetermined traits in the offspring animal. In some embodiments, the method includes confirming the presence of 2-40 of the predetermined traits. In some embodiments, the method includes confirming the presence of 2 or more of the predetermined traits. In some embodiments, the method includes confirming the presence of 4 or more of the predetermined traits. In some embodiments, the method includes confirming the presence of 6 or more of the predetermined traits. In some embodiments, the method includes confirming the presence of 8 or more of the predetermined traits. In some embodiments, the method includes confirming the presence of 10 or more of the predetermined traits.

In some embodiments, genetic testing was previously performed on the breeding pair.

In some embodiments, the method further includes registering the offspring animal having the one or more predetermined traits in the database system as a breeder animal.

In another aspect, the present disclosure provides a method of producing a set of guidelines for breeding of a non-human animal. In some embodiments, the method includes identifying the non-human animal in a computer-implemented database and assessing the presence of one or more predetermined traits in the non-human animal using the computer-implemented database system. In some embodiments, the identifying and assessing steps include generating a probability matrix for the non-human animal using the database system, wherein the probability matrix includes an array of probability values pertaining to the likelihood that the non-human animal has the one or more predetermined traits. In some embodiments, the identifying and assessing steps include displaying the probability matrix on a graphical user interface. In some embodiments, the method further includes determining, based on the assessment step, one or more conditions under which the non-human animal will develop or is at risk of developing a disease or non-disease condition. In some embodiments, the method further includes generating the set of guidelines for breeding of the non-human animal to avoid manifestation of the disease or non-disease condition.

In some embodiments, the non-human animal is used in a breeding pair to produce an offspring animal.

In some embodiments, the set of guidelines includes one or more (e.g., 1, 2, 3, 4, 5, 6, 7, 8, 9, 10, or more) recommendations for mitigating or avoiding the one or more conditions that would result in manifestation of one or more (e.g., 1, 2, 3, 4, 5, 6, 7, 8, 9, 10, or more) symptoms of the disease or non-disease condition and/or for reducing the likelihood of producing an effect in the offspring animal resulting from development of the disease or non-disease condition.

In some embodiments, the non-human animal is a female parent animal. In some embodiments, the non-human animal is a male parent animal.

In some embodiments, the set of guidelines provides one or more recommendations that increases the likelihood of producing a non-human offspring animal having one or more predetermined traits. In some embodiments, the one or more recommendations pertain to a diet, exercise regime, discipline, environmental exposure, medication, supplementary treatments, training, conditioning, use, and/or limitation of the non-human animal.

In another aspect, the present disclosure provides a method of generating a computer-implemented database system for producing a non-human offspring animal having one or more predetermined traits. In some embodiments, the method includes providing information about the one or more predetermined traits from a plurality of non-human animals of a same type as the offspring animal. In some embodiments, the information includes results of previously performed genetic testing and/or non-genetic assessment of the plurality of non-human animals, wherein the results of the previously performed genetic testing or non-genetic assessment identify the plurality of non-human animals as having one or more of the predetermined traits. In some embodiments, the information includes results of genetic testing and/or non-genetic assessment of the plurality of non-human animals that confirm the presence of one or more of the predetermined traits in the plurality of non-human animals. In some embodiments, the method includes cataloguing the information into the database system. In some embodiments, the database system is configured for interrogation by a user and is configured to produce a probability matrix based on the catalogued information following said interrogation.

In some embodiments, the interrogation includes selecting one or more predetermined traits desired in the offspring animal and identifying from the plurality of non-human animals a breeding pair capable of producing said offspring animal. In some embodiments, the method further includes selecting a breeding pair capable of producing the offspring having the one or more predetermined traits. In some embodiments, the probability matrix includes an array of probability values pertaining to the likelihood of the selected breeding pair producing a non-human offspring animal having the one or more predetermined traits. In some embodiments, the probability matrix includes an array of probability values pertaining to the likelihood of the plurality of non-human animals having the one or more predetermined traits.

In some embodiments, the non-genetic assessment includes obtaining or having obtained information from someone knowledgeable about the non-human animal pertaining to the one or more predetermined traits. In some embodiments, the non-genetic assessment includes performing or having performed one or more clinical assays on biological tissue obtained from the non-human animal pertaining to the one or more predetermined traits. In some embodiments, the non-genetic assessment includes performing or having performed one or more diagnostic tests on the non-human animal pertaining to the one or more predetermined traits. In some embodiments, the non-genetic assessment includes obtaining scores assigned to the non-human animal during a competition pertaining to the one or more predetermined traits.

In another aspect, the present disclosure provides a computer-implemented interface for assisting a user in producing, procuring, and/or identifying one or more non-human animals having one or more predetermined traits. In some embodiments, the interface is configured to allow a user to interrogate a computer-implemented database system. In some embodiments, the interrogation includes selecting the one or more predetermined traits of interest for the one or more non-human animals. In some embodiments, the interrogation includes identifying one or more of the non-human animals catalogued in the database system and generating a probability matrix for each of the one or more non-human animals identified using the database system. In some embodiments, the probability matrix includes an array of probability values pertaining to the likelihood that the non-human animal has the one or more predetermined traits. In some embodiments, the method includes, based on the probability matrix, displaying an overall assessment of each of the non-human animals having the one or more predetermined traits. In some embodiments, the method includes providing an option for the user to produce, purchase, or lease one or more of the non-human animals having the one or more predetermined traits. In some embodiments, the computer-implemented interface is in communication with the database system. In some embodiments, the method further includes selecting one or more predetermined traits of at least one parent of the one or more non-human animals. In some embodiments, the method further includes selecting a gene pool from which the one or more non-human animals or the at least one parent of the non-human animal are obtained. In some embodiments, the gene pool is selected from a group consisting of the user's own animals, animals owned by other users, an animal registry, and animals located within a specified geographical area.

In another aspect, the present disclosure provides a method of using a computer-implemented database system to identify a non-human animal as having one or more pre-determined traits, the method including: (a) providing information about the non-human animal; (b) cataloguing the information of step(a) into the database system; (c) comparing the information of step (b) to information about a plurality of non-human animals having the one or more predetermined traits, wherein the information about the plurality of non-human animals has been previously catalogued in the database system; (d) generating a probability matrix for the non-human animal based on the comparison of step (c) using the database system, wherein the probability matrix includes an array of probability values pertaining to the likelihood that the non-human animal has the one or more predetermined traits; (e) identifying the non-human animal as having the one or more predetermined traits based on the probability matrix of step (d); and (f) optionally, genetically modifying the non-human animal to promote or suppress an expression of the one or more predetermined traits in the non-human animal; wherein the information about the non-human animal includes results of previously performed genetic testing and/or non-genetic assessment of the non-human animal or the plurality of non-human animals.

In some embodiments of any of the foregoing aspects, the one or more predetermined traits include coat color, coat color modifier, coat texture, coat thickness, facial marking, leg marking, eye color, skin color, mane color, tail color, speed, gait, temperament, and health.

In some embodiments of any of the foregoing aspects, the coat color, mane color, and/or tail color is selected from the group consisting of amber champagne, amber champagne dun, amber cream, amber dun, amber dun pearl, apricot dun, apricot pearl, agouti, bay, bay cream pearl, bay double cream, bay pearl, black, black bay, black cream pearl, black double cream, black dun, black pearl, black/red, blanket appaloosa, blood bay, blue roan, brindle, brown, buckskin, buttermilk buckskin, champagne, champagne amber pearl, champagne classic pearl, champagne dun, champagne dun pearl, champagne gold pearl, champagne pearl, chesetnut, chestnut cream pearl, chocolate, classic champagne, classic champagne dun, classic cream, classic dun, cream, cream champagne, cream grullo, cream pearl, cremello, dapple grey, dark bay, dark brown, dark chestnut, dominant white, dun, dun bay cream, dun bay double cream, dun black cream, dun black double cream, dun chestnut cream, dun chestnut double cream, dun pearl, dun pearl bay, dun pearl black, dunalino, dunolino, dunskin, dunskin splash, flaxen chestnut, fleabitten grey, frame overo, frosted appaloosa, grey, gold champagne, gold cream, gold dun, gold dun pearl, grey, grullo, half blanket, leopard appaloosa, light grey, liver chestnut, marbled appaloosa, overo, palomino, pearl, perlino, perlino dun, pseudocream classic, red, red chestnut, red dun, red roan, rose grey, sabino, seal brown, silver, silver amber champagne, silver amber dun, silver bay cream, silver bay cream pearl, silver bay double cream, silver black, silver black cream pearl, silver black double cream, silver champagne, silver champagne dun, silver champagne pearl, silver classic champagne, silver classic champagne pearl, silver cream pearl, silver dapple, silver dapple bay, silver dapple buckskin, silver dapple cream grullo, silver dapple dun, silver dapple dunskin, silver dapple grullo, silver dapple pearl, silver dapple perlino, silver dapple perlino dun, silver dun, silver dun bay cream, silver dun bay double cream, silver dun black, silver dun black double cream, silver dun pearl, silver dun pearl bay, silver dun pearl black, silver pearl bay, silver pearl black, silver red, silver smoky cream, skewbald, smoky black, smoky cream, smoky grullo, snowflake appaloosa, sorrel, splashed white, steel grey, sun, tobiano, tovero, tri-color, white, and white spotting.

In some embodiments of any of the foregoing aspects, the coat color modifier is selected from a group consisting of apron, barring on body, barring on shoulder, belly spots (large or small), belly stripe, belted, bend or spots on body, bend or spots on head, birdcatcher spots, black spots, blagdon, blanket with roaning, blanket with spots, blaze, blaze with freckling, body spots, body white, brindle, brow spots, calico, coon/skunk tail, dapples, dilute, dorsal stripe, double dilute, ermine markings, few white hairs on body, fewspot, flaxen mane/tail, flea-bitten, fleshmark, frosted, grullo, heart marking, highlights in mane/tail, lacing pattern, leopard spotted, lightning marks, line back, frosting mane/tail, maximum tobiano, maximum overo, maximum white, maximum white sabino, medicine hat, minimal overo, minimal sabino, minimal tobiano, mixed mane/tail, mottled, no marking, overo, pangare, paw prints, pearl, piebald, pinto markings, rabicano, roan, roaning on body, rose, rump spots, sabino, skewbald, smudge marks, smutty, snip, snowcap, sooty, splash markings, star, stripe, tobiano, umbilical spots, white head, white mane/tail, white throat latch, white tipped ears, white with fading edges, white with ragged edges, white with smooth edges, wither spot, no markings, solid, white blaze, and white star.

In some embodiments of any of the foregoing aspects, the facial marking is selected from a group consisting of apron face, badger face, bald face, blaze, interrupted stripe, face, snip, star, stripe markings, both eyes amber, both eyes blue, both eyes brown, both eyes green, both eyes tiger, eyebrows, face mask, few white hairs on forehead, left eye amber, left eye blue, left eye brown, left eye green, left eye partial blue, left eye tiger, partial bald face, pigment around eye, right eye amber, right eye blue, right eye brown, right eye green, right eye partial blue, right eye tiger, white around eye, white chin, white jaw, white lip, white nose, and white sclera.

In some embodiments of any of the foregoing aspects, the leg marking is selected from a group consisting of two back white stockings, two white front socks, two white front stockings, two white hind socks, two white hind stockings, four white socks, four white stockings, back left above hock, back left cannon (¾ stocking), back left coronet, back left ermine spots, back left fetlock, back left half pastern, back left heel, back left partial heel, back left pastern, back left sock, back left stocking, back left stripes, back right above hock, back right cannon, back right coronet, back right ermine spots, back right fetlock, back right half pastern, back right heel, back right partial heel, back right pastern, back right sock, back right stocking, back right stripes, barring on legs, bend or spots on legs, front left above knee, front left cannon, front left coronet, front left ermine spots, front left fetlock, front left half pastern, front left heel, front left partial heel, front left pastern, front left sock, front left stocking, front left stripes, front right above knee, front right cannon, front right coronet, fright right ermine spots, front right fetlock, front right half pastern, front right heel, front right partial heel, front right pastern, front right sock, front right stocking, front right stripes, left front hoof stripes, left front white foot, left hind hoof stripes, left hind white foot, right front hoof stripes, right front white foot, right hind hoof stripes, right hind white foot, and roaning on white socks/stockings.

In some embodiments of any of the foregoing aspects, the coat texture includes smooth, rough, curly, straight, downy, spiky, or brindle.

In some embodiments of any of the foregoing aspects, the coat thickness includes thick, medium, or thin.

In some embodiments of any of the foregoing aspects, the eye color includes blue, amber, yellow, orange, hazel, green, or brown.

In some embodiments of any of the foregoing aspects, the skin color includes pink, black, brown, yellow, and white.

In some embodiments of any of the foregoing aspects, the speed is selected from the group consisting of endurance, mid-distance, and sprint.

In some embodiments of any of the foregoing aspects, the gait is selected from the group consisting of non-gaited, gait carrier, and gaited. In some embodiments, gaited includes walk, trot, canter, gallop, pacing, fox trot, racking, country pleasure, Indian shuffle, jogging, paso fino, paso corto, paso largo, paso llano, sobrandando, marcha picada, rack, running walk, stepping pace, singlefoot, tölt, and ravaal.

In some embodiments of any of the foregoing aspects, the temperament is selected from the group consisting of vigilant, curious/vigilant, curious, spooky, non-spooky, hot, cold, and medium.

In some embodiments of any of the foregoing aspects, the health includes variants of the one or more genes associated with one or more disease or non-disease conditions that are undesired in the non-human animal (e.g., one or more genes and associated disease or non-disease conditions recited in Table 10).

In some embodiments of any of the foregoing aspects, each of the one or more breeding pairs include at least a first non-human parent animal and a second parent animal.

In some embodiments of any of the foregoing aspects, the method further includes selecting the sex, breed, and/or discipline of the offspring animal, the first parent animal, and/or the second parent animal.

In some embodiments of any of the foregoing aspects, the breed is selected from the group consisting of Abyssianian/Ethiopian/Oromo/Gala horse, Abyssinian, Akhal Teke, Albanian, Albanian Horse, Alt-Olderburger, Altai Horse, American Cream Draft, American Cream and White, American Curly, American Gaited Mountain Horse, American Gaited Pony, American Paint Horse, American Quarter Horse, American Saddlebred, American Walking Pony, American Warmblood, Andalusian, Andravida, Andravida/Eleia Horse, Anglo European Warmblood, Anglo-Arab, Anglo-Kabarda, Appaloosa, Appendix Quarter Horse, AraAppaloosa, Arabian, Arabian Sporthorse, Ardennes, Ardennes Horse, Argentine Criollo, Asturcon, Asturian, Australian Brumby, Australian Stock Horse, Austrian Warmblood, Azteca, Baise Horse, Balearic, Baluchi, Baluchi Horse, Ban-ei, Banker, Banker Horse, Barb, Barb/Berber Horse, Bashkir, Bashkir Curly, Bashkir Curly Horse, Bashkir Horse, Basotho Pony, Basuto Pony, Belgian, Belgian Draft, Belgian Warmblood, Bhirum Pony, Bhirum/Nigerian Pony, Bhotia Pony, Bhutia Pony, Black Forest, Black Forest Pony, Blue Eyed Horse Association, Boer, Boerperd, Brandenburger, Breton, Breton Horse, British Riding Pony, British Warmblood, Buckskin, Budyonny, Budyonny Horse, Byelorussian Harness, Byelorussian Harness Horse, Camargue, Camargue Horse, Campolina, Canadian Horse, Canadian Sport Horse, Canadian Suffield Mustang, Canadian Warmblood, Carolina Marsh Tacky, Carthusian, Caspian, Caspian Horse, Cayuse, Celtic Warmblood, Cheju, Chicoteague Pony, Chilean Corralero, Chilean Corralero Horse, Chincoteague Pony, Cleveland Bay, Clydesdale, Colorado Ranger Horse, Connemara, Cream Draft, Cremello, Criollo, Crioulo, Curly Horse, Dales Pony, Danish Warmblood, Danube, Danube Delta Horse, Dartmoor Pony, Deliboz, Deliboz/Delibozskaya, Djerma, Dole, Dole Gudbrandsdal/Dolahest/Døle, Dongola, Donkey, Draft, Draft Cross, Draft Horse, Drum Horse, Dutch Draft, Dutch Harness, Dutch Harness Horse, Dutch Warmblood, East Bulgarian, East Friesian, Egyptian, Egyptian Arabian, Eriskay Pony, Estonian Horse/Estonian Native/Estonian Klepper/Eisti Hobune, Estonian Native, Exmoor Pony, Faeroe Pony, Faeroes Pony, Falabella, Falabella Miniature Horse, Fell Pony, Ferghana, Finnhorse, Finnhorse/Finnish Horse, Fjord, Fjord Horse, Fleuve, Florida Cracker, Florida Cracker Horse, Foundation Quarter Horse, Fouta, Foutanke/Fouta, Frederiksborg, Frederiksborg Horse, French Saddlebred, French Saddlebred Horse, French Trotter, Friesian, Friesian Cross, Galiceño, Galician Pony, Galician/Galician Mountain Horse, Gelderlander, Georgian Grande, German Bred, German Riding Pony, Gidran, Gidran/Hungarian Anglo-Arab, Golden American Saddlebred, Gotland, Gotland Pony, Groningen, Groningen Horse, Guangxi, Gypsy Cob, Gypsy Vanner, Hackney, Hackney Horse, Hackney Pony, Haflinger, Half-Arabian, Hanoverian, Hanoverian Warmblood, Hequ, Highland Pony, Hokkaido, Hokkaido Horse, Holsteiner, Holsteiner Warmblood, Hucul, Hucul Pony, Hungarian Warmblood, Iberian Warmblood, Icelandic, Icelandic Horse, lomud, lomud/Yomud/Yamud/Yomut, Irish Cob, Irish Draft, Irish Draught, Irish Draught Horse, Irish Sport Horse, Jeju Horse, Jinzhou, Jutland, Kabarda, Kabarda/Kabardin, Karabair, Karabakh, Kazakh, Kazakh Horse, Kentucky Mountain horse, Kentucky Mountain Saddle Horse, Kerry Bog Pony, KigeR Mustang, Kirdi Pony, Kisber Felver, Kiso, Kiso Horse, Kladbury, Kladbury Horse, Knabstrup, Knabstrupper Warmblood, Kushum, Kushum Horse, Kustanai, Kustanai Horse, Kyrgyz/Kirgiiz Horse, Latvian, Latvian Horse, Lipizzan, Lithuanian Heavy Draft, Lokai, Losino, Losino Horse, Lusitano, M Bayar, Mallorquin, Malopolski, Mangalarga, Mangalarga Marchador, Marsh Tacky, Marwari, Marwari Horse, Wrens Pony, Messara, Messara Horse, Miniature, Miniature Horse, Misaki, Misaki Horse, Missouri Fox Trotter, Missouri Fox Trotting Horse, Miyako, Miyako Pony, Mongolian, Mongolian Horse, Morab, Morgan, Mountain Pleasure Horse, Moyle, Mule, Murgese, Mustang, National Show horse, National Spotted horse, New Forest Pony, New Kirgiz, Newfoundland Pony, Nokota, Nokota Horse, Noma, Noma Horse, Nooitgedacht Pony, Nordland, Nordlandshest/Lyngshest/Nordland Pony, Norfolk Trotter, Noric, North Swedish horse, Northeastern, Ob, Oldenburg, Oldenburg Warmblood, Orlov Trotter, Paint (Overo), Paint (Solid), Paint (Tobiano), Paint (Tovero), Paints (All), Palomino, Pantaneiro, Pantaneiro horse, Paso Fino, Paulista Marchador, Peneia Pony, Percheron, Peruvian Paso, Pindos Pony, Pinia, Pintabian, Pinto, POA, Polish Horse, Ponies (All), Pony, Pony of America, Pottok, Priob/Ob, Przewalski, Przewalski Horse, Pura Raza Espanola (PRE), Pyrenean Tarpan, Qatgani, Qatgani/Qataghani Horse, Quarab, Quarter Horse, Quarter Horse Cross, Quarter Pony, Racking Horse, Rheinland Pfalz Saar, Rhenish Warmblood, Rhinelander horse, Rocky Mountain, Rocky Mountain Horse, Russian Don, Russian Heavy Draft, Russian Trakhener, Russian Trotter, Sanhe, Sanhe Horse, Schleswiger Heavy Draft, Scharzwälder Fuchs, Selle Francais, Shagya, Shagya Arabian, Shetland Pony, Shire, Single-Footing Horse, Skyros Pony, Somali Pony, Sorraia, Soviet Heavy Draft, Spanish Mustang, Spanish-Barb, Spanish-Norman, Spanish-Normal Horse, Sport Horse of Color, Spotted Draft, Spotted Draft Horse, Spotted Saddle, Spotted Saddle Horse, Standardbred, Sudan Country-Bred, Sudan Country-Bred Horse, Suffolk, Suffolk/Suffolk Punch/Suffolk Sorrel, Swedish Warmblood, Taishu/Tsushima, Taishuh, Tarpan, Tawleed, Tennessee Walker, Tennessee Walking Horse/Tennessee Walker, Tersk, Tersk Horse, Thessalian, Thessalian/Thessaly Horse, Thoroughbred, Thoroughbred Cross, Tibetan Pony, Tiger horse, Tokara, Tori, Tori Horse, Trakehner, Ukrainian Riding Horse/Ukrainian Saddle Horse, Ukrainian Saddle, Virginia Highlander, Vlaamperd, Vladimir Heavy Draft, Vyatka, Vyatka Horse, Walkaloosa, Warmblood, Warmblood Cross, Warmblood (All), Warmbloods of Color, Welara Pony, Welsh Cob, Welsh Pony, Weser-Em Pony, West African Barb, Western Sudan Pony, Westphalian, Wielkopolski, Wurttemberger Warmblood, Xilingol, Yakut, Yanqi, Yili, Yonaguni, Zangersheide, Zaniskari Pony, Zhemaichu, and Zorse.

In some embodiments of any of the foregoing aspects, the discipline is selected from the group consisting of barrel racing, beginner/family, breeding, brood mare, calf roping, companion only, competitive trail competitions, country pleasure, cowboy mounted shooting, cutting, draft, dressage, drill team, driving, endurance riding, English pleasure, equitation, eventing, field hunter, gaited, halter, harness, horsemanship, hunter, hunter under saddle, judged pleasure rides, jumper, lesson horse, longe-line, pleasure driving, pole bending, polo, racing, ranch horse, ranch sorting, reined cow horse, reining, rodeo, roping, showmanship, saddle seat, sidesaddle, steer wrestling, team penning, team roping, team sorting, trail horse, vaulting, Western pleasure, Western pleasure (show), Western riding, working cattle, youth/4-H horse, all around, 4-in hand driving, agility, breed shows, breeding stallion, broodmare, bull fighting, camping, cart, combined driving, cowboy dressage, cowboy racing, doma vaquero, field trials, gymkhana, harness racing, hazing, horse ball, marathon driving, mounted archery, mounted games, mounted patrol, mounted police, native costume, natural horsemanship, nurse mare, pacing, pack horse, parades, park, playday, polocrosse, ranch versatility, recipient broodmare, roadster, rodeo bronc, rodeo pickup horse, rodeo queen horse, roping, saddleseat, search and rescue, show hack, ski joring, steer roping, stock horse, sulky racing, team driving, team roping head horse, team roping heel horse, therapeutic riding, trial class competition, recreational trail riding, turnback horse, Western dressage, working equitation, hunt seat, show jumper, riding, pleasure, and bucking horse.

In some embodiments of any of the foregoing aspects, the likelihood that the offspring animal produced by each said breeding pair will have the one or more predetermined traits is at least 6.25%, 12.5%, 25%, 50%, 60%, 70%, 80%, 90%, 95%, 99%, or 100%.

In some embodiments of any of the foregoing aspects, the disease or non-disease condition is selected from a group consisting of anhidrosis, androgen insensitivity syndrome, cerebellar abiotrophy, curved ear tips, dwarfism, degenerative suspensory ligament disease, equine asthma, equine herpes virus risk, epistaxis risk, equine metabolic syndrome, laminitis, equine recurrent uveitis, equine viral arteritis susceptibility, foal immunodeficiency syndrome, gait transition, glycogen branching enzyme disorder, hereditary equine regional dermal asthenia, hoof wall separation disease, hydrocephalus, hyperkalemic periodic paralysis, immune-mediated myositis, impaired acrosomal reaction, junctional epidermolysis bullosa, kissing spines, lavender foal syndrome, lordosis, malignant hyperthermia, myotonia, navicular disease, osteochondritis dissecans, polysaccharide storage myopathy, recurrent laryngeal neuropathy, severe combined immunodeficiency, squamous cell carcinoma, tendinopathy, warmblood fragile foal syndrome, West Nile Virus susceptibility, wobbler syndrome, and behavioral abnormality. In some embodiments, the behavioral abnormality is selected from the group consisting of cribbing, teeth grinding, weaving, anxiety, depression, extreme aggression, extreme fearfulness, bucking, striking, biting, self-mutilating, and head shaking.

In some embodiments of any of the foregoing aspects, the one or more predetermined traits include at least 2 or more predetermined traits. In some embodiments, the one or more predetermined traits include at least 4 or more predetermined traits. In some embodiments, the one or more predetermined traits include at least 6 or more predetermined traits. In some embodiments, the one or more predetermined traits include at least 8 or more predetermined traits. In some embodiments, the one or more predetermined traits include at least 10 or more predetermined traits.

In some embodiments of any of the foregoing aspects, the computer-implemented database system includes genetic and non-genetic information about the non-human animal, non-human parent animal, and/or non-human offspring animal.

In some embodiments of any of the foregoing aspects, the genetic testing includes genotyping the non-human animal for any one of the following genetic markers: (a) agouti, black/red, brindle, champagne, cream, dominant, white, frame overo, grey, pearl, sabino, silver, splashed white, tobiano, and white spotting; (b) a curiosity gene that is a dopamine D4 receptor (DRD4; e.g., SEQ ID NO: 37) gene; (c) a speed gene that is a myostatin (MSTN; e.g., SEQ ID NO: 38) gene; (d) a gait gene that is a doublesex and mab-3-related transcription factor 3 (DMRT3; e.g., SEQ ID NO: 13) gene; and/or (e) a health gene selected from the group consisting of androgen receptor (AR; e.g., SEQ ID NO: 1), mutY DNA glycosylase (MUTYH; e.g., SEQ ID NO: 2), teashirt zinc finger homeobox 1 (TSHZ1; e.g., SEQ ID NO: 3), aggrecan (ACAN; e.g., SEQ ID NO: 4), family with sequence similarity 174 member A (FAM174A; e.g., SEQ ID NO: 5), interleukin 17A (IL17A; e.g., SEQ ID NO: 6), chemokine (C-X-C motif) ligand 16 (CXCL16; e.g., SEQ ID NOs: 7-11) interleukin 17B (IL17B; e.g., SEQ ID NO: 39), solute carrier family 5 member 3 (SLC5A3; e.g., SEQ ID NO: 12), glycogen branching enzyme (GBE1; e.g., SEQ ID NO: 14), peptidylprolyl isomerase B (PPIB; e.g., SEQ ID NO: 15), serpin family B member 11 (SERPINB11; e.g., SEQ ID NO: 16), beta-1,3-N-acetylgalactosaminyltransferase 2 (B3GALNT2; e.g., SEQ ID NO: 17), sodium voltage-gated channel alpha subunit 4 (SCN4A; e.g., SEQ ID NO: 18), myosin heavy chain 1 (MYH1; e.g., SEQ ID NO: 19), FKBP prolyl isomerase 6 (FKBP6; e.g., SEQ ID NOs: 20-22), laminin subunit gamma 2 (LAMC2; e.g., SEQ ID NO: 23), myosin 5A (MYOSA; e.g., SEQ ID NO: 24), equine chromosome 20 (ECA20; e.g., SEQ ID NOs: 25-28), ryanodine receptor 1 (RYR1; e.g., SEQ ID NO: 29), chloride voltage-gated channel 4 (CLCN4; e.g., SEQ ID NO: 30), glycogen synthase 1 (GYS1; e.g., SEQ ID NO: 31), equine chromosome 3 (ECA3; e.g., SEQ ID NO: 32), protein kinase DNA-activated catalytic subunit (DNAPK; e.g., SEQ ID NO: 33), damage specific DNA binding protein 2 (DDB2; e.g., SEQ ID NO: 34), procollagen-lysine, 2-oxoglutarate 5-dioxygenase 1 (PLOD1; e.g., SEQ ID NO: 35), 2′-5′-oligoadenylate synthetase 1 (OAS1; e.g., SEQ ID NO: 36), melanocyte inducing transcription factor (MITF; e.g., SEQ ID NO: 37), paired box (PAX), proto-oncogene receptor tyrosine kinase (KIT; e.g., SEQ ID NO: 40); high mobility group AT-hook 2 (HMGA2; e.g., SEQ ID NO: 42 and SEQ ID NO: 55); Zinc Finger And AT-Hook Domain Containing (ZFAT; e.g., SEQ ID NO: 43), ligand dependent nuclear receptor corepressor like and non-SMC condensing I complex subunit G locus (LCORL/NCAPG; SEQ ID NO: 41) and LIM and SH3 protein 1 (LASP1; e.g., SEQ ID NO: 44). In some embodiments, the genetic testing includes performing analysis of an entire genome of the non-human animal. In some embodiments, the analysis of an entire genome includes a genome-wide association study.

In some embodiments of any of the foregoing aspects, the non-human animal, non-human parent animal, or non-human offspring animal is selected from the group consisting of horse, cattle, sheep, dog, cat, camel, pig, goat, alpaca, donkey, llama, red fox, mouse, rat, ferret, non-human primate, rabbit, gerbil, hamster, chinchilla, or guinea pig.

In some embodiments of any of the foregoing aspects, the breeding pair includes at least 2 parent animals (e.g., at least 2, 3, or 4 parent animals). In some embodiments, the breeding pair includes 3 parent animals. In some embodiments, the breeding pair includes 4 parent animals. In some embodiments, the non-human offspring animal is produced using genetic material (e.g., DNA) obtained from one or more female parent animals and a male parent animal. In some embodiments, the genetic material is obtained from a mitochondria of the one or more female parent animals. In some embodiments, the genetic material from the mitochondria of the one or more female parent animals is used to produce the non-human offspring animal using mitochondrial transfer. In some embodiments, the genetic material is obtained from a nucleus of the one or more female parent animals. In some embodiments, the genetic material from the nucleus of the one or more female parent animals is used to produce the non-human offspring animal using nuclear transfer. In some embodiments, the non-human offspring animal is produced using in vitro fertilization procedure.

In some embodiments, the method further comprises generating a shared centimorgan (cM) value for the non-human animal or non-human offspring animal based on a sample obtained from the non-human animal or non-human offspring animal, respectively. In some embodiments, the method further comprises generating a kinship coefficient for the non-human animal or non-human offspring animal based on a sample obtained from the non-human animal or non-human offspring animal, respectively. In some embodiments, the method further comprises generating a heterozygosity score for the non-human animal or non-human offspring animal based on a sample obtained from the non-human animal or non-human offspring animal, respectively. In some embodiments, the method further comprises generating an inbreeding coefficient (F) for the non-human animal or non-human offspring animal based on a sample obtained from the non-human animal or non-human offspring animal, respectively. In some embodiments, the method further comprises performing a breed composition analysis on a sample from the non-human animal or the non-human offspring animal. In some embodiments, the method further comprises performing an identity-by-descent analysis on a sample from the non-human animal or the non-human offspring animal. In some embodiments, the sample is a biological sample (e.g., biopsy, such as, e.g., fresh frozen or formalin-fixed paraffin embedded tissue), blood, serum, plasma, urine, sputum, nail/hoof clippings, hair follicles, cell-free fetal DNA, mitochondria, a gamete (e.g., oocyte or spermatozoon), or an embryo of the offspring animal implanted, e.g., as part of an in vitro fertilization (IVF) procedure).

### BRIEF DESCRIPTION OF THE DRAWINGS

FIGS. 1A-1U show examples of a computer-implemented user interface of the present disclosure for use in producing, procuring, and/or identifying a non-human animal (e.g., a horse) with one or more predetermined traits. (FIG. 1A) Exemplary image of a front end of a webpage-instantiated user-interface useful for identifying or producing horses having one or more predetermined traits. The webpage presents a user with an option to identify existing horses having one or more predetermined traits that are catalogued in a computer-implemented database system of the disclosure (i.e., “Find a horse”) or to produce a horse having the one or more predetermined traits by identifying a breeding pair capable of producing the horse (i.e., “Build a horse”). (FIG. 1B) Exemplary image of a webpage-instantiated user interface designed to assist a user in finding an existing horse with a desired phenotype. This feature allows a user to select a gender of a horse (e.g., “Any,” “Gelding,” “Mare,” or “Stallion”), age, a gene pool from which the desired horse or a breeding pair originates (see FIG. 1D), specific behavioral abilities (i.e., traits) desired in the horse such as, e.g., speed, temperament, gait (see FIG. 1E) and color and/or color modifier (see FIG. 1F and FIG. 1H), features (e.g., breed and discipline; FIG. 1F and FIG. 1I), and health (e.g., disease or non-disease conditions that are undesired in the animal; see FIG. 1F and FIG. 1J). (FIG. 1C) Exemplary image of a webpage-instantiated user-interface designed to assist a user in “building” (i.e., producing) a horse having a desired phenotype. Similar to the “Find a horse” feature, the “Build a horse” feature allows a user to select the gene pool from which a horse is obtained and the option to buy or lease an animal (FIG. 1D), select a specific horse catalogued in the database system of the disclosure (FIG. 1G), select the abilities desired in the horse, (FIG. 1E), coat color or coat color modifier (FIG. 1H), features (FIG. 1I), and health (e.g., disease or non-disease conditions that are undesired in the animal; FIG. 1J). (FIG. 1K) Exemplary list of abbreviated horse profiles identified using the “Find a horse” feature by choosing a specific set of selection criteria for different traits of interest. (FIG. 1L) Example of an expanded animal profile of a horse (“Spooks Gotta Whiz”) identified using the computer-implemented user interface. The animal profile provides information pertaining to the genotype of the animal (A/A, E/E, W20/n, SW1/n, SW2/n, nd1/nd2), temperament [DNA](curious), gait/DMRT3 (non-gaited), speed (mid-distance), temperament (3/10), height (N/A; generally measured in number of hands), discipline (reining), color (Bay), markings (overo), location, owner information, and parents of the animal. (FIG. 1M). Exemplary list of potential breeding pairs and their abbreviated profiles identified using the computer-implemented user interface that are capable of producing a foal having a predetermined phenotype. In this case, a specific horse (i.e., Inferno Sixty-Six) was selected as a specific horse to be one member of a breeding pair with other horses catalogued in the database. (FIG. 1N) Example of an expanded profile of a selected breeding pair identified using the computer-implemented user interface. Information is provided pertaining to the speed, temperament, gait, genetic profile, appearance, and names of each of the mare and stallion in the selected breeding pair. (FIG. 1O) Example of a summary report of a potential foal produced by selecting one or more predetermined traits desired in the foal and selecting a breeding pair capable of producing a foal having the one or more predetermined traits. In this case, the foal was produced by selecting the breeding pair identified in FIG. 1N. A probability matrix provides a summary of the probabilities of the foal having a specific coat color, speed, temperament, gait, and possible health issues. (FIG. 1P) Example of a Trending Presets feature showing multiple categories of searches (i.e., builds) queried by users of the computer-implemented user interface system from which a particular user may select instead of creating a custom build. Exemplary Trending Presets include, but are not limited to, “Most popular searches for June 2019,” “What are the locals searching for,” “Tobiano is trending now,” “Child friendly,” “Breeding favorites,” “Buy them all,” “Champagne,” “5 Star Show Jumpers,” and “Clean Testing Sale Horses.” (FIG. 1Q) Example of a user profile generated on the computer-implemented user interface. The user interface allows the user to provide summary information about horses owned by the user, username, location, and user photo. (FIG. 1R) Example of an online portal of the user interface that allows the user to order genetic diagnostic tests for one or more horses. (FIG. 1S) Example of a summary page containing a list of genetic diagnostic tests ordered and/or performed on one or more horses by a specific user. (FIG. 1T) Example of a Sample Submission Form used to request genetic diagnostic tests for a horse using the computer-implemented user-interface. (FIG. 1U) Example of a genetic profile of a horse produced by requesting genetic testing using the database system of the disclosure.

FIGS. 2A-2C show examples of a computer-implemented user interface of the present disclosure for use in genetically modifying a horse (FIG. 2A) by selecting a trait from a trait category (e.g., coat color, health, or abilities) that is desired for modification, such as, e.g., avoiding a disease condition (e.g., Glycogen Branching Enzyme Deficiency; FIG. 2B), presenting with a specific coat color modifier (e.g., Champagne (CH/CH or CH/n); FIG. 2C).

FIGS. 3A-3D show examples of an Ancestry Report generated for a horse (“Cruising”) using the database system of the disclosure. (FIG. 3A) Geographical distribution of ancestral breeds (i.e., Thoroughbred, Carriage Horse, British Isles Native/Exmoor, European Heavy Horse, Iberian, and North Sea) identified for Cruising by generating an ancestry report using the database system of the disclosure. (FIG. 3B) Pie chart showing percentages of different ancestral breeds identified for Cruising using the database system of the disclosure. (FIG. 3C) Genetic composition key for FIG. 3A and FIG. 3B. (FIG. 3D) Graph representing the results of a principal component analysis (PCA) performed on Cruising by comparing Cruising's genealogy to a known reference population of horses. The reference horses represent the genomes of different “breeds” and regions throughout the world. Horses from each region of the world, group, or “breed” have genes in common and overlapping regions that can be identified, compared, and graphically visualized. Registries, breeds, or closed groups of horses bred through longer periods of history show more clearly defined clusters.

FIG. 4 shows an exemplary parentage report generated for a subject horse using the database system of the present disclosure.

FIG. 5 shows a schematic of an exemplary workflow utilizing the computer-implemented database system of the disclosure to produce or identify a non-human animal having one or more predetermined traits (i.e., a desired phenotype).

FIG. 6 shows an estimated inbreeding coefficient for a foal produced from a known sire (Charley Horse) and dam (Zema Gemmy). The bell curve represents a range of likely inbreeding coefficients for prospective foals that could be produced from Charley and Zema.

FIG. 7 shows an instantiation of the computer-implemented, web-based user interface that allows a user to view the profile of a specific animal (“Golden Opportunity”), including information about the animal's genetic profile, temperament, gait, and speed and to order an Ancestry Report (see top right of page) for the animal, as described herein.

FIGS. 8A-8E show examples of an Ancestry Report generated for a horse (“Spooks Gotta Whiz” or “Spooks”) using the database system of the disclosure. (FIG. 8A) Geographical distribution of ancestral breeds (i.e., Thoroughbred, Carriage Horse, European Heavy Horse, Iberian, North Sea, Exmoor, and Near East) identified for Spooks by generating an Ancestry Report using the database system of the disclosure. (FIG. 8B) Pie chart showing percentages of different ancestral breeds described in FIG. 8A identified for Spooks using the database system of the disclosure. (FIG. 8C) Genetic composition key for FIG. 8A and FIG. 8B. (FIG. 8D) Results of breed composition analysis showing the percent composition of the various ancestral breeds identified from a genetic sample obtained from Spooks Gotta Whiz. (FIG. 8E) Graph representing the results of a PCA performed on Spooks Gotta Whiz by comparing the horse's genealogy to a known reference population of horses. The reference horses represent the genomes of different “breeds” and regions throughout the world. Horses from each region of the world, group, or “breed” have genes in common and overlapping regions that can be identified, compared, and graphically visualized. Registries, breeds, or closed groups of horses bred through longer periods of history show more clearly defined clusters.

### DEFINITIONS

As used herein, the term “breeder animal” refers to a non-human animal (e.g., a horse) used for breeding. Accordingly, a breeder animal may be one that is used for breeding using conventional means, such as, e.g., mating a male breeder animal with a female breeder animal. Alternatively, a breeder animal may be one that is used as a donor of genetic material (e.g., sperm, egg, or mitochondria of the breeder animal) for the purpose of producing an offspring animal having one or more predetermined traits in the absence of physical mating with another breeder animal. In cases where an offspring animal is produced without requiring mating between two breeder animals, the genetic source material may be obtained and used from a single breeder animal or in combination with genetic material from one or more additional breeder animals. Additionally, a breeder animal may be a living animal or a deceased animal. In the case of a deceased animal, genetic material is obtained from the animal antemortem and cryopreserved for later use in producing an offspring animal having one or more predetermined traits.

As used herein, the term “breeding pair” corresponds to two or more non-human parent animals that are of the same species and that are capable of producing an offspring animal. Generally, a breeding pair includes two parent animals—a male and a female parent animal. However, recent advances in molecular genetics have enabled the creation of offspring animals having more than two parents. In such cases, the offspring animal could be produced, e.g., by using mitochondrial transfer, which involves removing the mitochondria of an oocyte from a first female parent and combining the oocyte with a spermatozoon from a male parent and mitochondria containing mitochondrial DNA from a second female parent to produce a fertilized egg that contains genetic information from three distinct parental sources. In another example, an offspring animal can be produced, e.g., by using nuclear transfer, which involves inserting the nucleus from an oocyte of a first female parent to an enucleate oocyte from a donor animal and combining the nucleated oocyte with a spermatozoon from a male parent to produce a fertilized egg containing genetic information from three distinct parental sources (the third parental source being the mitochondrial nucleic acid provided by the donor oocyte). Thus, a “breeding pair” may include, e.g., 3 parent animals. Furthermore, a fertilized egg produced using a mitochondrial or nuclear transfer method described above may be further modified by introducing one or more heterologous donor polynucleotides (e.g., a transgene) from a fourth genetic source (e.g., a fourth “parent”). In such instances, the term “breeding pair” may include, e.g., 4 parent animals. In cases where an offspring animal is produced using genetic source material from one or more parent animals without the requirement for physical mating between the parent animals, the genetic source material may be obtained from a living or deceased animal. In the case of a deceased animal, genetic material can be obtained from the animal antemortem and cryopreserved for later use in producing an offspring animal having one or more predetermined traits.

As used herein, the terms “database” and “database system” refer to an organized collection of related data stored digitally on a computer system (e.g., a non-transitory storage medium). In the context of the present disclosure, a database system may store information (e.g., genetic and non-genetic information) about a plurality of non-human animals pertaining to one or more predetermined traits. The database system may be part of a larger computer software system that allows a user to engage with one or more databases (e.g., by way of a computer-implemented graphical user interface, as is disclosed herein). Information in the database system may be queried by a user by providing input information pertaining to a specified set of selection criteria (e.g., one or more predetermined traits desired in a non-human animal) set by the user utilizing a graphical user interface. The database system may be configured for interrogation by a user to perform functions such as, e.g., adding, removing, modifying, and retrieving data (e.g., data about a non-human animal pertaining to one or more predetermined traits) within the system. Database systems may be physically instantiated on database servers that store the database information and accessed by a user by way of a graphical user interface.

As used herein, the term “donor polynucleotide” refers to a heterologous polynucleotide (e.g., a transgene or a segment thereof) that may be inserted into a targeted genomic locus as part of a gene editing effort using, e.g., transcription activator-like effector nuclease (TALEN), zinc finger nuclease (ZFN), or clustered regularly interspaced short palindromic repeats (CRISPR)-Cas technology. The donor polynucleotide may contain a segment of a gene targeted for modification. For example, a naturally-occurring variant of a gene may contain a deleterious mutation or polymorphism that would benefit from gene editing to restore the normal function of the affected gene. Thus, the donor polynucleotide may include a segment of the target gene that, when integrated into the target gene locus, restores the normal function of the gene.

As used herein, the term “embryonic development” refers to a biological process by which an embryo of a non-human animal grows and develops during prenatal (e.g., pre-birth) development. Generally, embryonic development proceeds in successive stages, wherein the process starts with the fertilization of an egg cell (i.e., ovum) with a sperm cell (i.e., spermatozoon) to produce a diploid zygote. Following a series of mitotic cell divisions, the zygote forms into a multicellular embryo. Cell division is succeeded by formation of a blastula, which then becomes a blastocyst. Further development of the blastocyst leads to the formation of the germ layers in a process known as gastrulation. Subsequent stages of embryonic development include formation of the nervous system, organs, and somites.

As used herein, the term “endogenous,” as it applies to a gene in a non-human animal, refers to the property of the gene being intrinsically expressed in one or more cell types of the animal.

As used herein, the term “expression” refers to one or more of the following events: (1) production of an RNA primary transcript from a DNA sequence by transcription; (2) processing of an RNA transcript into mature mRNA (e.g., by splicing, editing, 5′ cap formation, and/or 3′ end processing); (3) translation of an mRNA into a polypeptide or protein; (4) post-translational modification of a polypeptide or protein; and/or (5) manifestation of a particular phenotype in a non-human animal resulting from any one of events (1)-(4).

As used herein, the term “heterologous” refers to a nucleic acid sequence that is not normally contained within a specific DNA or RNA molecule, not normally expressed in a cell (e.g., a mammalian cell), and/or is not normally found occurring in nature. As used herein, a heterologous nucleic acid may, for example, include a promoter sequence, an artificial intron, a coding or non-coding exon, a transgene, or any associated regulatory sequences individually or in combination. Furthermore, the term “heterologous” may also refer to a protein that is not normally expressed in a cell (e.g., the protein is not endogenous to the cell), a protein that has been modified in its amino acid sequence relative to an endogenous protein, and/or is not normally found occurring in nature.

As used herein, the term “interrogating,” as it applies to the computer-implemented database system of the disclosure, refers to the method steps that include, but are not limited to, selecting one or more predetermined traits desired in an offspring animal, identifying one or more breeding pairs capable of producing the offspring animal, generating a probability matrix for at least one or each of the one or more breeding pairs and displaying the probability matrix on a computer-implemented user interface, selecting, based on the probability matrix, one or more of the breeding pairs likely to produce the offspring animal having the one or more predetermined traits, and/or breeding the one or more breeding pairs to produce the offspring animal. Interrogation of the computer-implemented database system can be performed according to the methods disclosed herein using, e.g., webpage-instantiated graphical user interface displayed on a computer monitor or a handheld device.

As used herein, the term “modulate” or “modulating” refers to increasing or decreasing a value of a particular variable of interest. For example, “modulating expression” of a gene may refer to increasing or decreasing the expression of a gene relative to a reference value (e.g., baseline expression level of the gene (e.g., an endogenous gene)). An increase in the value of a variable may be an increase by 1%, 5%, 10%, 20%, 30%, 40%, 50%, 100%, 200%, 300%, 400%, 500%, 1000%, or more. A decrease in the value of a variable may be a decrease by 1%, 5%, 10%, 20%, 30%, 40%, 50%, 60%, 70%, 80%, 90%, or 100%.

As used herein, the term “mutation” refers to a change in the nucleotide sequence of a gene. Mutations in a gene may occur naturally as a result of, for example, errors in DNA replication, DNA repair, irradiation, and exposure to carcinogens, or mutations may be induced as a result of administration of a transgene expressing a mutant gene. Mutations may result, e.g., from a single nucleotide substitution or deletion, inversion, duplication of a single nucleotide or of a contiguous sequence of nucleotides.

As used herein, the term “non-genetic assessment” refers to an analysis of a non-human animal based on non-genetic information about the animal. Because not all phenotypic traits can be accounted for by genes alone, and because phenotypic traits often result from interactions between genes and the environment, a holistic phenotypic analysis of an animal may also rely on non-genetic information alone or in conjunction with genetic information. Non-limiting examples of non-genetic assessment include phenotypic analysis of a non-human animal based on information gathered from someone knowledgeable about the animal (e.g., owner, breeder, caretaker, or seller) using a questionnaire, information gathered from sporting or competition events (e.g., score cards based on standardized marking systems used in formal animal sporting or competition events), information gathered from veterinary assays (e.g., blood analysis measuring, e.g., blood sugar levels, blood urea nitrogen, amylase, total calcium, X-rays, CAT scans, PET scans, and conformational and physiological parameters).

As used herein, the term “polymorphism” refers to a simultaneous occurrence in the same population of two or more alleles at a particular genetic locus, with each allele occurring at a particular frequency (usually above 1%). Non-limiting examples of a polymorphism include single nucleotide polymorphism (SNP), restriction fragment length polymorphism (RFLP), amplified fragment length polymorphism (AFLP), insertion-deletion polymorphism (INDEL), random amplified polymorphic DNA (RAPD), and a simple sequence conformation polymorphisms (SSCP), among others.

As used herein, the term “producing,” as it applies to a non-human offspring animal (e.g., a horse), refers to a method of creating the offspring animal using genetic and/or non-genetic information from one or more parent animals (e.g., a stallion and/or one or more mares). The offspring animal may be produced by breeding e.g., physically mating) a male and a female parent animal. Alternatively, the offspring animal may be produced using a combination of in vitro fertilization (IVF) and mitochondrial or nuclear transfer. In such cases, an offspring animal may be produced using genetic material (e.g., DNA) from one or more parent donor animals. For example, an offspring animal may be produced by removing the mitochondria of an oocyte from a first female parent animal, artificially fertilizing the oocyte with a spermatozoon from a male parent animal, and artificially introducing a mitochondrion (or mitochondria) from a second female parent animal into the fertilized oocyte. In another example, an offspring animal can be produced, e.g., by using nuclear transfer, which involves artificially introducing a nucleus from a female parent animal to an enucleated donor oocyte from a related female animal and fertilizing the oocyte with a spermatozoon from a male parent, animal.

As used herein, the term “trait” means a characteristic of an organism which manifests itself in a phenotype, and refers to a biological, behavioral, or any other measurable characteristic(s), which can be any variable that can be quantified in or from a biological sample or organism, which can then be used either alone or in combination with one or more other quantified variables to characterize an animal. Many traits are the result of the expression of a single gene, but some are polygenic, i.e., they result from coordinated expression of more than one gene. A “phenotype” is an outward appearance or other measurable characteristic of an organism. Many different traits can be inferred by the methods disclosed herein. As used herein, a trait may be “predetermined” if the expression of the trait was selected for by a user (e.g., animal owner, breeder, caretaker, seller, or buyer) using the methods and systems of the present disclosure. Non-limiting examples of predetermined traits include coat color, coat color modifier, coat texture, coat thickness, facial marking, leg marking, eye color, skin color, mane color, tail color, speed, gait, temperament, and health.

As used herein, the term “non-human animal” refers to any non-human mammal, including cattle, sheep, dog, cat, camel, pig, goat, alpaca, donkey, llama, red fox, mouse, rat, ferret, non-human primate, rabbit, gerbil, hamster, chinchilla, or guinea pig, among others. A non-human animal may refer to a companion animal, a farm animal, or an animal used for leisure or sporting activities. Furthermore, a non-human animal may be an offspring animal or a parent animal (e.g., male parent or female parent).

As used herein, the term “probability matrix” refers to an array of probability values pertaining to the likelihood of a non-human animal (e.g., a non-human offspring animal) having one or more predetermined traits. The array of probability values may include individual probability values corresponding to the likelihood of expression of a specific predetermined trait in the non-human animal.

As used herein, the terms “user interface” and “graphical user interface” refer to a component of a computer software program instantiated on a general-purpose computer or an electronic hand-held device that allows a user to engage with a computer program (e.g., a software program) to achieve a specific goal (e.g., producing an offspring animal having one or more predetermined traits). The user interface may be part of a larger system containing a database system and subsystems (such as, e.g., a database system disclosed herein), as well as engines for searching, comparing, saving, exporting, and transforming information using a variety of mathematical operations (e.g., statistical analysis). A user interface generally requires the user to provide one or more inputs (e.g., one or more predetermined traits desired in the offspring animal), which the interface routes to the appropriate software system (e.g., database and/or engine) or subsystem. The user interface may then receive and display output information (e.g., a probability matrix containing an array of probability values pertaining to the likelihood of the offspring animal having one or more predetermined traits) to the user based on the processing of the provided inputs by the appropriate systems. The user interface may include systems that are functionally connected to physical input hardware such as a peripheral device, such as a keyboard and a mouse, and output hardware, such as a display monitor, speakers, and printer(s).

### DETAILED DESCRIPTION

The disclosure generally relates to methods and computer-implemented systems (e.g., a database system and a graphical user interface) for producing, procuring, and/or identifying a non-human animal or offspring thereof having one or more predetermined traits (e.g., traits associated with physical appearance, morphology, health, and performance, among others). In particular, the present disclosure provides computer-implemented databases containing information associated with one or more predetermined traits (e.g., genetic and non-genetic information) for use in calculating a probability of producing a non-human animal having one or more predetermined traits. The methods and computer-implemented systems disclosed herein are also useful for selecting one or more predetermined traits of a non-human animal, identifying potential breeding pair matches capable of producing the animal having the traits of interest, identifying potential health risks associated with the selection of those traits, identifying existing animals having one or more traits of interest that are for sale or lease, and/or identifying animals that are available for stud service. The disclosed methods and systems are also applicable for the production of transgenic non-human animals having one or more predetermined traits of interest. The disclosed methods may be used for assessing the health, behavior, performance, predispositions, and/or monetary value of an animal. Such information may be particularly useful in making decisions pertaining to breeding, purchasing, medical treatment, and destiny determination (e.g., discipline) of the animal.

The sections that follow describe the methods and computer-implemented systems useful for producing a non-human animal or offspring thereof having one or more predetermined traits in detail. While the descriptions below are directed towards horses, the present disclosure can also be used with other non-human animals including, but not limited to cattle, sheep, dogs, cats, camels, pigs, goats, alpacas, donkeys, llamas, red foxes, mice, rats, ferrets, non-human primates, rabbits, gerbils, hamsters, chinchillas, or guinea pigs, among others.

### Computer-Implemented Database System

The present disclosure provides a database system for storing, analyzing, retrieving, and updating information about multiple non-human animals (e.g., horse, cattle, sheep, dog, cat, camel, pig, goat, alpaca, donkey, llama, red fox, mouse, rat, ferret, non-human primate, rabbit, gerbil, hamster, chinchilla, or guinea pig, among others) having one or more predetermined traits. The database system can include a variety of system components (e.g., subsystems). Information about the non-human animal can be stored in the various datasets of the system, categorized according to the source and/or type of information, and partitioned into unique database subsystems. An exemplary database subsystem can include a dataset of non-human animals having a specific genetic profile associated with one or more predetermined traits (e.g., a genetic profile subsystem) and characterized by, for example, expression of a specific gene(s) or lack thereof, presence or absence of a specific variant of a gene(s) (e.g., a gene having a single nucleotide polymorphism (SNP)), presence or absence of genetic mutations (e.g., indels, substitutions (transitions, transversions, silent, missense, or nonsense), duplication, repeat expansion, or frameshift). A separate database subsystem can contain information about the animal gathered from one or more secondary sources (e.g., a secondary source information subsystem).

### Use of the Database System to Produce a Non-Human Animal Having One or More Predetermined Traits

The present disclosure provides methods for using genetic and non-genetic information about one or more non-human animals that have been integrated into a database that can be interrogated in order to assist in the production of an offspring animal having one or more predetermined traits. Such methods can be used to inform breeding cross-match evaluations, in which a breeding pair is selected based on genetic and non-genetic information in order to produce an animal having one or more desired physical characteristics, behavioral characteristics, and/or predispositions. For example, horse enthusiasts have historically selected for specific traits in their foals by making breeding decisions based on information relating to the pedigree, fitness, appearance, health, and behavior of the stallion and mare. Accordingly, the breeder would optimize the odds of achieving the target phenotype (e.g., one or more desired traits) in the foal by selecting a breeding pair capable of producing the desired phenotype. This strategy is encumbered by various sources of error that introduce uncertainty of achieving the target phenotype in the foal. For example, breeding decisions based on pedigree have long been made on the basis of self-reported testimony of the horse seller and/or based on an assumption that the foal inherits 50% of its genetic information from each parent. There are at least two problems with this approach, namely that (1) pedigree assignments based on presumed genetic inheritance often underestimate the degree of genetic relatedness (i.e., inbreeding) between the stallion and the mare; and (2) small changes in genotype can produce profound changes in phenotype, thus potentially confounding the initial judgment of the breed of the stallion and/or mare.

Presently, horse breeders rely on biomechanical, geometric, and physiological criteria to evaluate young adult horses for their inherited potential to fulfill a specialized discipline (e.g., performance racing). The size and relative positions of major muscles in the fore and hind limbs can be measured to estimate stride power. Slow-motion videography can be utilized to evaluate the efficiency of a horse's gait. Blood pressure and ultrasound can be used to determine heart size, thickness, and stroke volume. Because the phenotype of an adult horse depends on the interaction of its genotype and environment, an adult phenotype does not reliably provide an accurate prediction of the horse's genetic potential. In addition, parental phenotype is a poor predictor of offspring genotype. Phenotypically superior horses often produce below-average foals, demonstrating the limitations of relying on phenotypic analysis and performance or pedigree records such as stud books or race results alone in predicting the foal's ultimate phenotype. In the absence of performance records, prospective purchasers rely largely on pedigree and physical conformation to select animals which they consider to have potential for specific disciplines. At a young age, a horse is still growing and developing, and, thus, its physical conformation may not accurately predict its adult physical capacity and its performance. Therefore, the value of breeding decisions based on parental phenotype could only be ascertained after the animal enters into the productive phase of its life and has received sufficient training. The absence of predictive information about the foal's ultimate phenotype prior to its productive phase substantially increases the risk and cost associated with a potentially unfavorable outcome.

Genetic profiling has also been employed for the purpose of selective breeding of horses that excel at specific skills. However, such efforts have been targeted almost exclusively at improving athletic performance (e.g., speed), typically by selecting for specific variants of single genes (e.g., the MSTN gene; see, e.g., Hill et al. Recent Patents on DNA & GENE Sequences 6: 203-8, 2012 and Schröder et al. Vet. J. 190: 39-48, 2011). The present disclosure improves on such approaches by providing a holistic approach for identifying or producing a non-human animal having a desired phenotype by providing an integrated database system capable of analyzing and correlating an unprecedented number of genetic and non-genetic characteristics in order to produce a desired phenotype in the animal with a high probability. Such a database system combines, stores, organizes, and correlates vast quantities of disjointed genetic and non-genetic information pertaining to one or more physical and/or behavioral traits, which a user can then leverage and interrogate using a computer-implemented user interface system to select a desired phenotype of the subject animal and produce such an animal with high certainty.

Methods disclosed herein address the problem faced by animal breeders discussed above by increasing the likelihood of producing an offspring animal (e.g., a foal) having one or more (and typically multiple) desirable characteristics by drawing from multiple sources of information integrated within a single user-friendly platform that may be instantiated, for example, as an online computer-implemented user-interface system in communication with a computer-implemented database system, as is described herein. The user platform may be part of a larger computer-implemented system that includes a remote graphical user interface, one or more digital database systems having one or more database subsystems, one or more engines for searching, comparing, storing, and manipulating information (e.g., through mathematical operations such as, e.g., statistical analysis), and computer hardware (e.g., local or remote) to implement the platform, as described in detail below. The user may be any party interested in producing, procuring, and/or identifying a non-human animal (e.g., horse, cattle, sheep, dog, cat, camel, pig, goat, alpaca, donkey, llama, red fox, mouse, rat, ferret, non-human primate, rabbit, gerbil, hamster, chinchilla, or guinea pig, among others) such as, for example, an animal owner, breeder, seller, caretaker, or first-time buyer.

Thus, the disclosed methods and systems may be applied to produce a non-human offspring animal (e.g., a foal) having one or more predetermined steps according to the following methods. First, a user (e.g., animal owner, breeder, caretaker, seller, or buyer) may use a computer-implemented user interface disclosed herein to select one or more predetermined traits desired in the offspring animal using a computer-implemented database system that includes genetic and non-genetic information about the offspring animal or one or more (e.g., 1, 2, 3, or more) parents of the offspring animal. The user may then interrogate the database system to identify one or more (e.g., 1, 2, 3, 4, 5, 6, 7, 8, 9, 10, or more) breeding pairs (e.g., a breeding pair having at least a first and a second parent animal of the same type) capable of producing the offspring animal. The user may also specify the sex, breed, and/or discipline of at least the first parent animal and/or the second parent animal of the offspring animal. The user may then use the computer-implemented database system to generate a probability matrix for each of the one or more breeding pairs, such as a probability matrix that includes an array of probability values (e.g., at least 6.25%, 12.5%, 25%, 50%, 60%, 70%, 80%, 90%, 95%, 99%, or 100%) pertaining to the likelihood that the offspring animal produced by each said breeding pair will have the one or more predetermined traits. As a subsequent step, the user may select, based on the array of probability values of the probability matrix, one or more of the breeding pairs likely to produce the offspring animal having the one or more predetermined traits. The user may then elect to breed one or more of the selected breeding pairs to produce the offspring animal (e.g., a breeding pair with a probability value above a threshold value, such as a threshold value of at least 50% (e.g., at least 60%, 70%, 80%, 90%, 95%, or 99%, or greater such as 100%)). Genetic testing may be performed on the offspring animal to detect the presence of the one or more predetermined traits. If the offspring animal is identified as lacking one or more of the predetermined traits, e.g., based on the results of the genetic testing, the offspring animal lacking the one or more predetermined traits may be genetically modified to promote or improve expression of the one or more predetermined traits, as is discussed in detail below. For example, the genetic testing in the offspring animal at an early stage, such as during embryonic development, 3-60 days after conception, or one day to four years after birth. The user may further elect to continue with the birthing the offspring animal. The user may perform further genetic or non-genetic testing on the animal one or more times to confirm the presence of the one or more predetermined traits in the offspring animal, such as after birth. Upon confirming the presence of the one or more predetermined traits in the offspring animal, the user may register the offspring animal determined to have the one or more predetermined traits in the database system, for example, as a breeder animal.

### Production of Non-Human Animals for a Specific Discipline

In the context of producing a horse, but also applicable to other non-human animals of interest, the user may desire to increase the likelihood of the offspring animal having one or more traits to achieve a phenotype suitable for one or more disciplines (e.g., such as one or more disciplines disclosed herein). In the context of horses, a discipline generally refers to one of multiple equestrian sports requiring a particular set of skills from the horse and the rider. A horse having one or more traits suitable for a particular discipline may compete in such sporting events to display the horse's and the rider's skills at that discipline. These disciplines could be used as a selection criterion in the database system, thereby allowing a user to select a desired discipline for an offspring animal. Interrogation of the database system using this selection criterion would identify possible parental animals that could be bred to produce an offspring animal that excels at the desired discipline by specifying that at least one of the parent animals in a breeding pair excel at the discipline or possesses traits that, when combined with other specifically selected traits in a second parent animal, would have a high probability of producing an offspring animal that excels at the desired discipline.

In a particular example, a user interested in producing, procuring, or identifying a non-human animal (e.g., a horse) suitable for a specific discipline, such as, e.g., roping, may select roping as a discipline at which the one or more parent animals excel at using the computer-implemented systems of the disclosure. Information pertaining to a discipline that one or more parent animals excel at may be obtained through self-reported testimony from someone familiar with the animal (e.g., an owner or a caretaker) or the information may be obtained from an ancestry report (e.g., an ancestry report described herein) that indicates a favorable likelihood that an offspring animal generated by breeding one or more parent animals that excel at the desired disciplines will also excel at that discipline. The system would then produce a list of animals exhibiting the selected discipline. The system may allow the user to select an option to buy or lease one or more of these animals, or the user could, e.g., select one or more of these animals as parent animals that could be bred to produce an offspring animal. The system may also produce a probability matrix showing the likelihood that breeding two parental animals would produce an offspring animal exhibiting the selected discipline. A detailed list of discipline selections that can be made by a user of the database system is described below.

For example, a user wishing to identify or produce a horse with a proficiency trait, such as, e.g., Show Jumper, the horse will be selected to express at least one endurance-type MSTN gene variant (“T”), at least one medium-tall height gene variant (LCORL=ATTT.C), be negative for tendinopathy, recurrent laryngeal neuropathy, negative for the DMRT3 gene, and positive for at least one vigilance trait. The user can use the above factors as selection criteria via the user interface of the disclosure to identify an existing animal having the above phenotype or to identify a breeding pair capable of producing an offspring animal having the above phenotype. The database system may then employ its search and compare engines to identify horses having the above-described genotype or to identify one or more breeding pairs capable of producing an offspring animal having said genotype.

### Information Stored in the Database

A. Genetic Information

Multiple sources of information may be used in the systems and methods of the present disclosure to produce a non-human animal having one or more predetermined traits. Among these sources, genetic information about the animal may be a critical factor in determining its ultimate phenotype. Without wishing to be bound by any theory, expression of one or more predetermined traits in the non-human animal may result from expression of one or more genes. Thus, the present disclosure provides methods for using genetic information to increase the likelihood of producing a non-human animal having one or more predetermined traits. The present disclosure also provides methods of using genetic information to produce a composition analysis for a non-human animal that assists in determining ancestral breed composition as well as parentage determination. The genetic basis for a large number of animal traits, predisposition for physical skills, and disease conditions has been established to date, as is well-known in the art, and this information can be incorporated into the system database and interrogated by a user to establish, e.g., a probability matrix, as described herein, which can be used to produce an offspring animal.

a. Genetic Markers of Physical Traits

The presence of genes or gene variants that effect coat color and markings in horses can be tested and verified using methods described herein and known in the art. This information can be included in the database system and interrogated in order to produce a probability matrix, as is described herein. Patterns and color of facial markings in horses may also be genetically determined. These patterns and markings can be reported in the database for each reference animal and can be used as a selection criterion for the desired offspring animal. Furthermore, genetic information about the color of leg markings may be employed using the methods of the disclosure. Leg markings can be reported in the database for each reference animal and used as a selection criterion for the desired offspring animal.

Conformation is yet another physical trait that may be predicted through genetic profiling of an animal in accordance with the methods disclosed herein. Conformation deals with the physical attributes of the animal (e.g., a horse) and encompasses dimensions such as bone structure, musculature, and body proportions in relation to each other and the intended use of the animal. Conformation attributes can also be reported in the database for each reference animal and used as a selection criterion for the desired offspring animal. For example, a horse that is intended for use as a draught animal would require a different conformation than one intended for use as a jumper or a cutting horse. Conformational traits may be measured in angles for shoulder, hip and neck set, as well as back length, leg length, pastern length and angle, height and weight, head and ear shape, musculature, hoof shape and hardness, and shoulder versus hip height, among others. In selecting for conformational traits, the user may need to take caution to avoid co-selecting for undesired genetic diseases that may be genetically linked. For example, Boyko et al. (Boyko et al. BMC Genomics 15:259, 2014; incorporated by reference herein in its entirety) have used genome-wide association analysis to establish an association between equine recurrent laryngeal neuropathy, also known as “roaring,” and horse height and body size, traits genetically linked to the LCORL/NCAPG locus on equine chromosome 3 (ECA3). The present disclosure addresses this problem by providing probability estimates for the likelihood of developing a genetic disease condition based upon the selection of one or more predetermined traits of the animal. For example, such estimates may be determined by performing genetic testing on the non-human animal to identify gene variants associated with an increased probability of having a particular disease or non-disease condition. In some cases, a disease or non-disease condition in a non-human animal (e.g., a horse) may be associated with multiple SNPs within a particular gene locus, such as, e.g., the disease or non-disease conditions and corresponding SNPs of Table 1, as is shown below.

TABLE 1
Disease or non-disease conditions in horses associated with multiple single nucleotide polymorphisms

| | Disease | | | | | | Publica- | | | | Alter- | | |
|---|---|---|---|---|---|---|---|---|---|---|---|---|---|
| Number | or non- | | | | | | tion | Refer- | | Alter- | native | | |
| of | disease | Trait | Relia- | Trait | | | (PubMed | ence | Reference | native | pheno- | SNP | Nucleic acid sequence |
| loci | condition | category | bility | symbol | Breeds | Gene | ID) | allele | phenotype | allele | type | Name | containing multiple SNPS |
| 2 | Suscepti- | Disease | HIGH | EAV | Multi- | CXCL16 | 27930647 | A | Resistant | T | Suscep- | CXCL16- | AGGTGAGAGGCTGCGATGGGGC |
| | bility or | Risk | | | ple | | | | | | tible | EAVRS1- | GGTGGGTTGGAGGCTAAGTAATA |
| | resistance | | | | | | | | | | | DIAG | GCTGGGGGAGCCCTTTGGCGTG |
| | to Equine | | | | | | | | | | | | GGAACCTGTGCAGACCGTCGCTG |
| | Arteritis | | | | | | | | | | | | TCTCTCTCAGGGGATGGCAACGA |
| | Virus | | | | | | | | | | | | GGGCAGCATCGCTGGAAGTTGTC |
| | | | | | | | | | | | | | GCTGTAATACAAGAT[A/T]TCCGT |
| | | | | | | | | | | | | | CCGACTCCCCTCCGGCGGTTGAC |
| | | | | | | | | | | | | | TTTATGGAACATTTCCGAAAACAA |
| | | | | | | | | | | | | | CTGAGAGGCTATGATCGCTGTGG |
| | | | | | | | | | | | | | TTCGTACGTCAGGTAATCTTAACC |
| | | | | | | | | | | | | | CCCCACACCCCCCACCCCGGGAA |
| | | | | | | | | | | | | | GACAGCGCCTCTGGGACCCTCTC |
| | | | | | | | | | | | | | TGGT |
| | | | | | | | | | | | | | (SEQ ID NO: 8) |
| | | | | | | | | G | Resistant | C | Suscep- | CXCL16- | CGACCAGACCCTGGTCTAGGTGA |
| | | | | | | | | | | | tible | EAVRS2- | GAGGCTGCGATGGGGCGGTGGG |
| | | | | | | | | | | | | DIAG | TTGGAGGCTAAGTAATAGCTGGG |
| | | | | | | | | | | | | | GGAGCCCTTTGGCGTGGGAACCT |
| | | | | | | | | | | | | | GTGCAGACCGTCGCTGTCTCTCT |
| | | | | | | | | | | | | | CAGGGGATGGCAACGAGGGCAG |
| | | | | | | | | | | | | | CATCGCTGGAAGTTGTCGCTGTA |
| | | | | | | | | | | | | | ATACAAGATATCCGTCCGACTCCC |
| | | | | | | | | | | | | | CTCCGGCGGTT[G/C]AC[T/G or |
| | | | | | | | | | | | | | T]TTATG[G/A]AACATTTCCGAAAA |
| | | | | | | | | | | | | | CAACTGAGAGGCTATGATCGCTG |
| | | | | | | | | | | | | | TGGTTCGTACGTCAGGTAATCTTA |
| | | | | | | | | | | | | | ACCCCCCACACCCCCCACCCCGG |
| | | | | | | | | | | | | | GAAGACAGCGCCTCTGGGACCCT |
| | | | | | | | | | | | | | CTCTGGTCAAACTTGAACTCCTTC |
| | | | | | | | | | | | | | TGGGAGGACCTAGCCCCCTCTCC |
| | | | | | | | | | | | | | GGCTTATAATTGCCCTGATTCTCC |
| | | | | | | | | | | | | | CAGTTCACCT |
| | | | | | | | | | | | | | (SEQ ID NO: 9) |
| | | | | | | | | T | Resistant | A or G | Suscep- | CXCL16- | CGACCAGACCCTGGTCTAGGTGA |
| | | | | | | | | | | | tible | EAVRS3- | GAGGCTGCGATGGGGCGGTGGG |
| | | | | | | | | | | | | DIAG | TTGGAGGCTAAGTAATAGCTGGG |
| | | | | | | | | | | | | | GGAGCCCTTTGGCGTGGGAACCT |
| | | | | | | | | | | | | | GTGCAGACCGTCGCTGTCTCTCT |
| | | | | | | | | | | | | | CAGGGGATGGCAACGAGGGCAG |
| | | | | | | | | | | | | | CATCGCTGGAAGTTGTCGCTGTA |
| | | | | | | | | | | | | | ATACAAGATATCCGTCCGACTCCC |
| | | | | | | | | | | | | | CTCCGGCGGTT[G/C]AC[T/G or |
| | | | | | | | | | | | | | T]TTATG[G/A]AACATTTCCGAAAA |
| | | | | | | | | | | | | | CAACTGAGAGGCTATGATCGCTG |
| | | | | | | | | | | | | | TGGTTCGTACGTCAGGTAATCTTA |
| | | | | | | | | | | | | | ACCCCCCACACCCCCCACCCCGG |
| | | | | | | | | | | | | | GAAGACAGCGCCTCTGGGACCCT |
| | | | | | | | | | | | | | CTCTGGTCAAACTTGAACTCCTTC |
| | | | | | | | | | | | | | TGGGAGGACCTAGCCCCCTCTCC |
| | | | | | | | | | | | | | GGCTTATAATTGCCCTGATTCTCC |
| | | | | | | | | | | | | | CAGTTCACCT |
| | | | | | | | | | | | | | (SEQ ID NO: 10) |
| | | | | | | | | G | Resistant | A | Suscep- | CXCL16- | CGACCAGACCCTGGTCTAGGTGA |
| | | | | | | | | | | | tible | EAVRS4- | GAGGCTGCGATGGGGCGGTGGG |
| | | | | | | | | | | | | DIAG | TTGGAGGCTAAGTAATAGCTGGG |
| | | | | | | | | | | | | | GGAGCCCTTTGGCGTGGGAACCT |
| | | | | | | | | | | | | | GTGCAGACCGTCGCTGTCTCTCT |
| | | | | | | | | | | | | | CAGGGGATGGCAACGAGGGCAG |
| | | | | | | | | | | | | | CATCGCTGGAAGTTGTCGCTGTA |
| | | | | | | | | | | | | | ATACAAGATATCCGTCCGACTCCC |
| | | | | | | | | | | | | | CTCCGGCGGTT[G/C]AC[T/G or |
| | | | | | | | | | | | | | T]TTATG[G/A]AACATTTCCGAAAA |
| | | | | | | | | | | | | | CAACTGAGAGGCTATGATCGCTG |
| | | | | | | | | | | | | | TGGTTCGTACGTCAGGTAATCTTA |
| | | | | | | | | | | | | | ACCCCCCACACCCCCCACCCCGG |
| | | | | | | | | | | | | | GAAGACAGCGCCTCTGGGACCCT |
| | | | | | | | | | | | | | CTCTGGTCAAACTTGAACTCCTTC |
| | | | | | | | | | | | | | TGGGAGGACCTAGCCCCCTCTCC |
| | | | | | | | | | | | | | GGCTTATAATTGCCCTGATTCTCC |
| | | | | | | | | | | | | | CAGTTCACCT |
| | | | | | | | | | | | | | (SEQ ID NO: 11) |
| 1 | Lordosis | Trait | High | L | Saddle | ECA2 | 21070278 | C | Non-risk | T | Risk | BIEC2- | GCTCCTAAAAGAGGCCCTTCCAC |
| | (congenial | Genetics | | | bred | 0 | | | | | haplo- | 632523- | ACCATCACACCAGGGTTCCTATGT |
| | swayback) | | | | | | | | | | type | L1 | GGGACTCCATTCACAGCCCACAC |
| | | | | | | | | | | | | | ATAGGAAACTGCCAGGACCATCT |
| | | | | | | | | | | | | | GTTTTTTTGTGATGATGATTCAAT |
| | | | | | | | | | | | | | GACTATTTGTTTTTCATTTTTTTCA |
| | | | | | | | | | | | | | CAACTGGTCTGTGCCTGGCAGTT |
| | | | | | | | | | | | | | GGCTGGATAACCCACCAACCACT |
| | | | | | | | | | | | | | CCTAACCCCTCT[C/T]GCTCACCT |
| | | | | | | | | | | | | | CCACTGCAGAGGCTGGAAAAGCT |
| | | | | | | | | | | | | | AATCGCTTCACCAGCCTCCCTTGC |
| | | | | | | | | | | | | | AGCTAGGGTTGAGCACATCGTATT |
| | | | | | | | | | | | | | CCCGGAACtgaggagacacaaagcac |
| | | | | | | | | | | | | | aaggacaaaaagccgaaatgctgagc |
| | | | | | | | | | | | | | agggcaaagcagaaagactgaggaca |
| | | | | | | | | | | | | | ttcccatcctcggtgtcaccgctgag |
| | | | | | | | | | | | | | ctgccagatcaacacca |
| | | | | | | | | | | | | | (SEQ ID NO: 25) |
| | | | | | | | | A | Non-risk | G | Risk | BIEC2- | TGCAGCAACAATATTTTAAGTTCC |
| | | | | | | | | | | | haplo- | 532534- | CTGATGGCTGGCATCCACCAATC |
| | | | | | | | | | | | type | L2 | TGGAGAATTTTCTATATTTGTGGC |
| | | | | | | | | | | | | | CTAAAGTTCCATAAAACATGGATA |
| | | | | | | | | | | | | | ATGACAAGTCCTCCGTGAAGATTA |
| | | | | | | | | | | | | | GGCCATTGGCTGTGGAAGGAAAT |
| | | | | | | | | | | | | | AAGCCTGAGGACTTGCCCTGCTG |
| | | | | | | | | | | | | | GGAGTGAACACTCCTGTCACAGT |
| | | | | | | | | | | | | | GCTGACTAAGCA[A/G]TACGCGAG |
| | | | | | | | | | | | | | GAACAAGAATTAATCTGTCCTCCA |
| | | | | | | | | | | | | | ACGGTGGGTaagggactcccacaaga |
| | | | | | | | | | | | | | ctacacagtgagttttagagctgggg |
| | | | | | | | | | | | | | gcagaaatcaggccctgcaactccca |
| | | | | | | | | | | | | | ggctgaggttctttcaaccaaatcac |
| | | | | | | | | | | | | | ATTCCCCTCCCCTCTACTTTGGAATT |
| | | | | | | | | | | | | | CCCCTATGAAATTAGAATTAAAAAGT |
| | | | | | | | | | | | | | TTCCCACTTCTG |
| | | | | | | | | | | | | | (SEQ ID NO: 26) |
| | | | | | | | | C | Non-risk | T | Risk | BIEC2- | agttgttaccctgaaaaacatttttg |
| | | | | | | | | | | | haplo- | 632578- | gtcttatcagtcctctatttaaaaac |
| | | | | | | | | | | | type | L3 | ctctgttggcatttcattgcttacaa |
| | | | | | | | | | | | | | gttaaaagccgaattcacactctcgg |
| | | | | | | | | | | | | | cccctctcaccccactcttctagccc |
| | | | | | | | | | | | | | ctatcctggtttccttgccccccagg |
| | | | | | | | | | | | | | atttcccatcttctgatgtgacaagc |
| | | | | | | | | | | | | | ctttttatgactcagggcc[c/t]gt |
| | | | | | | | | | | | | | tctgatttgatttctttggcccggac |
| | | | | | | | | | | | | | tgctttatttctctcctttctttctt |
| | | | | | | | | | | | | | ttgccaactaaactcacgttcttcct |
| | | | | | | | | | | | | | ttaggagccatatcaaaagtcacctc |
| | | | | | | | | | | | | | ttcagggacgtcctccctaattcttc |
| | | | | | | | | | | | | | aggcagagtgtgcagctcagccctct |
| | | | | | | | | | | | | | gtgttcccttatcctttgtggcccat |
| | | | | | | | | | | | | | ggagcttttgcatttc |
| | | | | | | | | | | | | | (SEQ ID NO: 27) |
| | | | | | | | | T | Non-risk | G | Risk | BIEC2- | ATTATGTCTAACTGTAATTTTCGAA |
| | | | | | | | | | | | haplo- | 632658- | AGCTGCTAAAATAAACATGTAGTG |
| | | | | | | | | | | | type | L4 | CTTTTGCAATAAAAAATAAACAATA |
| | | | | | | | | | | | | | AAAAGAGAGAACGAGAACGTCTA |
| | | | | | | | | | | | | | AAGAAAACAGTGAGGCAATTCGA |
| | | | | | | | | | | | | | ACCCAACCAGAGGCCCACGGCAG |
| | | | | | | | | | | | | | CATCTGGGATTACTGAGCTCTGTG |
| | | | | | | | | | | | | | ACCGACACCTAATTGGACACAGA |
| | | | | | | | | | | | | | CTGCCTAGAA[T/G]TGTCCCTCAT |
| | | | | | | | | | | | | | TTGTTTTCTGGGCAAGGCCATTCA |
| | | | | | | | | | | | | | TAAAGACTCATTACTCCAAAACAA |
| | | | | | | | | | | | | | CCAGCCCAAGAGCCCCTCAATGT |
| | | | | | | | | | | | | | CCAACTCCAGACACACGAAGGAG |
| | | | | | | | | | | | | | TAACAATGGCCAGAAAGGCCCTG |
| | | | | | | | | | | | | | AGGCTTTCTCCAGAGAACTGCAG |
| | | | | | | | | | | | | | TCACTAATCAATTCAAAGACAAAC |
| | | | | | | | | | | | | | GAGGTTTCTCCCTGTAAAGGAAAA |
| | | | | | | | | | | | | | TG |
| | | | | | | | | | | | | | (SEQ ID NO: 28) |
| 1 | Height | Trait | HIGH | H1 | Multi- | LCOR | 22806074 | T | Low risk | C | High | BIEC2- | AGTGCTAAAGAAAAGCCAAATAAC |
| | | Genetics | | | ple | L/NCA | | | | | risk | 806543- | TCATGATTGGAGTCAGTTGGGTTT |
| | | | | | | PG | | | | | | H1 | AATGCAAGTCAGTTTTGCAGAGG |
| | | | | | | | | | | | | | CAGTTTTAAAGGAGAGACATGAAT |
| | | | | | | | | | | | | | CGGCAGAAATGCCTGCCCTTCCA |
| | | | | | | | | | | | | | GTGACAATAAGTGAAAAGAGAAG |
| | | | | | | | | | | | | | CCATCTATTTGCATGTTCTTGAGA |
| | | | | | | | | | | | | | GAAACCAAATTTGCCTGGCTAGA |
| | | | | | | | | | | | | | GAAGCATTCCAG[C/T]TTATTTCT |
| | | | | | | | | | | | | | GTACCCCAAAGGCAGAATCACAGG |
| | | | | | | | | | | | | | GAACACAATAGTGCAAACTTAAAT |
| | | | | | | | | | | | | | GACAACCTCTACAAAGAATATTCC |
| | | | | | | | | | | | | | TCTCTCAACTCTCCTCTCTCTATG |
| | | | | | | | | | | | | | CTATCCGGTCAAAGCCAGAGGTG |
| | | | | | | | | | | | | | GAGAGTACCTATATGTTAATGCGT |
| | | | | | | | | | | | | | TGAATATATTAATAACAAACATTTG |
| | | | | | | | | | | | | | TTTTGAACTCCATTTTAAATTAGC |
| | | | | | | | | | | | | | (SEQ ID NO: 41) |
| | | | | H2 | | HMGA | | C | Low risk | T | High | BIEC2- | GTATTCTCGGGCAGGTTTCTTGAG |
| | | | | | | 2 | | | | | risk | 1024210- | CCATTTCTCTCAATAGCAAAACTG |
| | | | | | | | | | | | | H2 | CCTCGTGTTCCATTTTTGATTTCA |
| | | | | | | | | | | | | | GTGTTGCTTCTCTAACACATTTGA |
| | | | | | | | | | | | | | CTTTCAAGCAATATTGTTTTGCTA |
| | | | | | | | | | | | | | GCTATCATGCATCATGAccataaaat |
| | | | | | | | | | | | | | ttaaataatttttCATCTCTGAAGAC |
| | | | | | | | | | | | | | AAAAGGAAGCAATTGCTAATTTTAAG |
| | | | | | | | | | | | | | GAA[T/C]TGTATCGACTCAAGTTTG |
| | | | | | | | | | | | | | TTTARAAAATTTGAAAAAARCTTGTC |
| | | | | | | | | | | | | | AACTTGATATTTCTTAAGGAGATGCA |
| | | | | | | | | | | | | | TATTTAAGTAGCTRATTTACACAG |
| | | | | | | | | | | | | | GCAGATAACAACATAAACCTTACA |
| | | | | | | | | | | | | | TCAGTTCTTCAAGTACTTCAATCA |
| | | | | | | | | | | | | | ATGCCCATTTCTACTCAATTTTAAA |
| | | | | | | | | | | | | | TTAAATTCAACTGAAAAGATCTCT |
| | | | | | | | | | | | | | GTAAAGAGG |
| | | | | | | | | | | | | | (SEQ ID NO: 42) |
| | | | | H3 | | 2FAT | | C | Low risk | T | High | BIEC2- | tctggccccggctgggcgctggcctg |
| | | | | | | | | | | | risk | 1105596- | ggattcaccctctctggacaccagtt |
| | | | | | | | | | | | | H3 | tccacactaacgatatggagaggcca |
| | | | | | | | | | | | | | aagcagagaccctttgagaccccatc |
| | | | | | | | | | | | | | cagctgggatcctctaagatcccaAA |
| | | | | | | | | | | | | | GCAGTGACGGGAYTTGGAGCTCAACT |
| | | | | | | | | | | | | | GGTCACAGAGAAAGGAGACCTGT |
| | | | | | | | | | | | | | AATTTGGtgcctccatgcctt[c/t] |
| | | | | | | | | | | | | | gcacatctgatccctctacctggaat |
| | | | | | | | | | | | | | gtagctcttgtcatctttcacctggc |
| | | | | | | | | | | | | | caactcccactcattcattcacctaa |
| | | | | | | | | | | | | | cctgacaaatcctgagcacctactaa |
| | | | | | | | | | | | | | gtgctaggcaccaccagaMgcattga |
| | | | | | | | | | | | | | gaacacaacaatgaacaagacgagta |
| | | | | | | | | | | | | | tggcctccaagtagctcgcatgtagc |
| | | | | | | | | | | | | | ggaggagacagacatcac |
| | | | | | | | | | | | | | (SEQ ID NO: 43) |
| | | | | H4 | | LASP1 | | G | Low risk | A | High | BIEC2- | AATGGTGGAATAGTAAACGAAGT |
| | | | | | | | | | | | risk | 144169- | GATAGGAGAACCTCTCTTTTGGGA |
| | | | | | | | | | | | | H4 | AGAAGGCCAACTGGAAGTCTGGA |
| | | | | | | | | | | | | | AGCAAAAGGATGACTGGCCTAAG |
| | | | | | | | | | | | | | CTTGGCCCCCGCCCACCGGGGC |
| | | | | | | | | | | | | | CTATCCYGGAGCCCCAGCCCCAAC |
| | | | | | | | | | | | | | TCCTCCCGGTGCAACACCCCAAC |
| | | | | | | | | | | | | | ACATACAACCCACCAGAAAACTCG |
| | | | | | | | | | | | | | AACAGGGCAGTCCCC[A/G]CCCC |
| | | | | | | | | | | | | | CACAAACCCCCAAAACGACAGCC |
| | | | | | | | | | | | | | CCTCTCCCTCCCCCAATGCCTGG |
| | | | | | | | | | | | | | GAGGTGCTGGCAGAGTGCCCCAG |
| | | | | | | | | | | | | | TGCTGTCACCACCCAGTCTGCTG |
| | | | | | | | | | | | | | GCCTCTGCCTGCTTAGCTGCACA |
| | | | | | | | | | | | | | TGCCCCTGCCTGGGCCCCCGCCT |
| | | | | | | | | | | | | | CCCCTACCCCCGCAGGGGCCTGC |
| | | | | | | | | | | | | | CAGGAAAAACATTCTCTGGAATCC |
| | | | | | | | | | | | | | AGACCATGACC |
| | | | | | | | | | | | | | (SEQ ID NO: 44) |
| | | | | H5 | | HMGA | 26474182 | G | Low risk | A | High | HMGA2- | tcacccctgcctcgtcctccagccct |
| | | | | | | 2 | | | | | risk | H5 | atcgccccgtctcccgaaaggtgccg |
| | | | | | | | | | | | | | ggcggactcggggcggctgaggccag |
| | | | | | | | | | | | | | cggctgcagcggcggtagcggcggcg |
| | | | | | | | | | | | | | gcggcgggaggcaggatgagcgcacg |
| | | | | | | | | | | | | | cggtgagggcgccgggcagccgtcca |
| | | | | | | | | | | | | | cttcagcccagggacaacctgccgcc |
| | | | | | | | | | | | | | ccggcgcctcagaagagag[g/a]ac |
| | | | | | | | | | | | | | gcggccgacccaggaagcagcagcaa |
| | | | | | | | | | | | | | gtcagtacgcgggcggggtgggggca |
| | | | | | | | | | | | | | ccagcccgcctccgcgccctccgaga |
| | | | | | | | | | | | | | aggccaggccacgcgcggccccgagc |
| | | | | | | | | | | | | | gcgggagccgggtcgccgcgcgccgc |
| | | | | | | | | | | | | | ccgccggccggaggcgggggccgcgg |
| | | | | | | | | | | | | | cgtctccggcgcgagtgcgcggcgga |
| | | | | | | | | | | | | | gccccgggcgccc |
| | | | | | | | | | | | | | (SEQ ID NO: 55) |
| 1 | Recurrent | Disease | | RUN | Multi- | ECA3 | 24707981 | T | Non-risk | C | Risk | BIEC 2- | NC_009146.3:104403770- |
| | laryngeal | risk | | | ple | | | | | | | 808643 | 105830625 |
| | neuropathy | | | | | | | | | | | BIEC2_4 | (SEQ ID NO: 32 - See |
| | | | | | | | | | | | | 17210 | Sequence Listing.) |
| | | | | | | | | | | | | BIEC2_6 | |
| | | | | | | | | | | | | 24338 | |
| | | | | | | | | | | | | BIEC2_1 | |
| | | | | | | | | | | | | 125869 | |
| 1 | Osteo- | Bone | LOW | OCD | Dutch | Multi- | 23278111 | C | OCD Risk | T | OCD | BIEC2- | AGTGCTAAAGAAAAGCCAAATAAC |
| | chondresis | Disorder | | | Warm- | ple | | | | | Protect | 808543- | TCATGATTGGAGTCAGTTGGGTTT |
| | | | | | blood | | | | | | | OCD_D | AATGCAAGTCAGTTTTGCAGAGG |
| | | | | | | | | | | | | WB1 | CAGTTTTAAAGGAGAGACATGAAT |
| | | | | | | | | | | | | | CGGCAGAAATGCCTGCCCTTCCA |
| | | | | | | | | | | | | | GTGACAATAAGTGAAAAGAGAAG |
| | | | | | | | | | | | | | CCATCTATTTGCATGTTCTTGAGA |
| | | | | | | | | | | | | | GAAACCAAATTTGCCTGGCTAGA |
| | | | | | | | | | | | | | GAAGCATTCCAG[C/T]TTATTTCTG |
| | | | | | | | | | | | | | TACCCCAAAGGCAGAATCACAGG |
| | | | | | | | | | | | | | GAACACAATAGTGCAAACTTAAAT |
| | | | | | | | | | | | | | GAGAACCTCTACAAAGAATATTCC |
| | | | | | | | | | | | | | TCTCTCAACTCTCCTCTCTCTATG |
| | | | | | | | | | | | | | CTATCCGGTCAAAGCCAGAGGTG |
| | | | | | | | | | | | | | GAGAGTACCTATATGTTAATGCGT |
| | | | | | | | | | | | | | TGAATATATTAATAACAAACATTTG |
| | | | | | | | | | | | | | TTTTGAACTCCATTTTAAATTAGC |
| | | | | | | | | | | | | | (SEQ ID NO: 45) |
| | | | | | | | | G | | A | | BIEC2- | gactgagtgaccgtctgcacattcaa |
| | | | | | | | | | | | | 121320- | caggagaaactaagcccccttctcca |
| | | | | | | | | | | | | OCD_D | ccctcttaagagtgccagcagccagg |
| | | | | | | | | | | | | WB2 | catatgccagccatgcaggacgaagg |
| | | | | | | | | | | | | | gctcatcttttccagtaaccaaaaag |
| | | | | | | | | | | | | | ccctagaggaaaaaattctagatact |
| | | | | | | | | | | | | | aacatttgatgacacccagtcaccct |
| | | | | | | | | | | | | | gtacactgaagcccccat[c/a]aag |
| | | | | | | | | | | | | | cccaatccatccacacagcttctatc |
| | | | | | | | | | | | | | agcttttcagcgcctGGGAAACACAC |
| | | | | | | | | | | | | | ATGGTCAATTTCCCTAACATAGAAAG |
| | | | | | | | | | | | | | TCTACAAAGGGATTAAACAAAAGCAA |
| | | | | | | | | | | | | | TTTTGTAGTTAAACATTAAGGAAAA |
| | | | | | | | | | | | | | GAACTGGAATTTATAATATCAGGG |
| | | | | | | | | | | | | | AACATGCCATGCATGGACTTAATA |
| | | | | | | | | | | | | | ATTAATGCCTTTGCCCTGGT |
| | | | | | | | | | | | | | (SEQ ID NO: 46) |
| | | | | | | | | A | | C | | BIEC2- | AGAATAGAGTTTTATCATCCTCAA |
| | | | | | | | | | | | | 121823- | AAGCACAAAATTTAAATATGGTAA |
| | | | | | | | | | | | | OCD_D | TTTATCTAGTCACTCATTTTTAAAG |
| | | | | | | | | | | | | WB3 | TACCCCATATTTCCTCCAATCATTT |
| | | | | | | | | | | | | | ACCTACTAATCTAAAAAAACAGTG |
| | | | | | | | | | | | | | CCTCCCTTTTCATACTAATCTCTG |
| | | | | | | | | | | | | | GATCACCTTTCTGGTTCCCAGAAA |
| | | | | | | | | | | | | | CATCCCCTGGGAATCAATGCAATT |
| | | | | | | | | | | | | | TTAAAG[A/C]AAAGAAAGAAACGG |
| | | | | | | | | | | | | | GGAAAAAAAACACTTGCTTATATT |
| | | | | | | | | | | | | | TGTTTACTTTCATGCTATTCCACTA |
| | | | | | | | | | | | | | GTTTTTCTTTTTCTTTCTTTTCTTTT |
| | | | | | | | | | | | | | Atttatttatttttttgggaagatta |
| | | | | | | | | | | | | | gccctgaactagcatctgccgccaat |
| | | | | | | | | | | | | | cYtcctctttttgctgaggcagactg |
| | | | | | | | | | | | | | gccctaagctaacatccRtgcccatc |
| | | | | | | | | | | | | | ttcctct |
| | | | | | | | | | | | | | (SEQ ID NO: 47) |
| | | | | | | | | A | | G | | BIEC2- | CCTCATTAGCACACCTCTACAGCC |
| | | | | | | | | | | | | 121337- | AAGCCATCTACTGGGCACAAACC |
| | | | | | | | | | | | | OCD_D | TATTTTCCTCCTTTTCTTAACTAAG |
| | | | | | | | | | | | | WB4 | ATGGATCTGCTGCACTGATAGCCT |
| | | | | | | | | | | | | | ACAAAATTTCAATTTCATCCTGTAT |
| | | | | | | | | | | | | | CAAATACTTTAGGCGTATAAGAGT |
| | | | | | | | | | | | | | ATGAATGGCACGTTTTAAAAACAG |
| | | | | | | | | | | | | | AAATTAAGTAAAAATCGAGTTATA |
| | | | | | | | | | | | | | GTACTAT[A/G]AAAGACAAGTTCC |
| | | | | | | | | | | | | | AACCACACTTGGTTATCCAAAAGC |
| | | | | | | | | | | | | | CAACAGATTCAAAATATGACAGCT |
| | | | | | | | | | | | | | CTCTTGCCTTTTACTTCATGGAAG |
| | | | | | | | | | | | | | GAGTGAGGGAGTAATTTCATAAGT |
| | | | | | | | | | | | | | GATGAGGCCATGTGACAAATAAAT |
| | | | | | | | | | | | | | TCAGTGAGAGAACACACCAGAAA |
| | | | | | | | | | | | | | TGGATTTTGGAGAGAATCCAAGAA |
| | | | | | | | | | | | | | ATGTTGGTATGGAAAACATG |
| | | | | | | | | | | | | | (SEQ ID NO: 48) |
| 1 | | | | | Tho- | | 22952004 | C | | T | | BIEC2- | CTGAAATCTGTCCCCTAAATAAAG |
| | | | | | rough- | | | | | | | 793865- | GTATGTGTGCTTCACAACTATTTA |
| | | | | | bred | | | | | | | TB-OCD | TATTTACAAATAGTGTCGCTCAGG |
| | | | | | | | | | | | | | TCTTAAATCGAATGAATCTACTAA |
| | | | | | | | | | | | | | AGCATTAGCAGATGCTTATTTATG |
| | | | | | | | | | | | | | ACATATATGTGTTGTGAACACAAA |
| | | | | | | | | | | | | | TGACAGCCAATTTGGCACCTTACA |
| | | | | | | | | | | | | | GCTCACACAGAAGAGTTTCTGTAT |
| | | | | | | | | | | | | | GAAAGGTA[T/C]GATTAATTTGCTA |
| | | | | | | | | | | | | | TGGGAATACAATTACCACTCAACA |
| | | | | | | | | | | | | | TTTCCCTATTTGCAAAAAAAGTAG |
| | | | | | | | | | | | | | GTTGGGTAAAAGAAAGGCCATCC |
| | | | | | | | | | | | | | ATATTCTTACCTAGAAGATTTTTTT |
| | | | | | | | | | | | | | ACCATTACTAAGCAAATATTTTTGT |
| | | | | | | | | | | | | | GGCATCCCTACTATCAACGCTTGA |
| | | | | | | | | | | | | | TGACTGACATTAAGGACATTGTAG |
| | | | | | | | | | | | | | GAATGGAGATAGGTGCGT |
| | | | | | | | | | | | | | (SEQ ID NO: 49) |
| 4 | | | | | Fierch | | 21841084 | A | | G | | BIEC2- | CTAATCTAGGATAACCAGATTTTT |
| | | | | | Trotter | | | | | | | 808442- | GGATGTCCTTAAACTCTTCTCAAT |
| | | | | | | | | | | | | OCD_FT_{—} | ATTAAAATTTGATGAAAACATTCCA |
| | | | | | | | | | | | | Hock | TTTATCTAGCAGGCACAGATTAAC |
| | | | | | | | | | | | | | AGAAATGTAAGTCTCTTAGGATTT |
| | | | | | | | | | | | | | TCACTTGAATTCCCCTAAgttctttc |
| | | | | | | | | | | | | | ttYtgaaatattttttccagcctctt |
| | | | | | | | | | | | | | tttcctagttaattactattgacccc |
| | | | | | | | | | | | | | tca[a/g]tgtagcttaaaactcaga |
| | | | | | | | | | | | | | tctttatgaagcctttcctgataccc |
| | | | | | | | | | | | | | catgctagatcaacccctcACATGAT |
| | | | | | | | | | | | | | ATACACTGTTTCTCTTTCATGTCACT |
| | | | | | | | | | | | | | TATCACAGCTGACAAGCAACTtagag |
| | | | | | | | | | | | | | tagttgttagtatctcagactctaaa |
| | | | | | | | | | | | | | gccagactacccaggattaaatctta |
| | | | | | | | | | | | | | gctctcaatttattagctctgcagtc |
| | | | | | | | | | | | | | (SEQ ID NO: 50) |
| 1 | Impaired | Repro- | HIGH | IAR | Tho- | FKBP6 | 23284302 | G | Normal | A | Reduced | FKBP6- | AACAGGGGCAGTAACGACACCGA |
| | acrosome | ductive | | | rough | | | | | | fertil- | IAR1 | CCTGCTGTCATGGAGATGACCTC |
| | reaction | Disorder | | | bred | | | | | | ity | | TGCAGAGGCACCTGGCGCACACA |
| | (in^sub- | | | | | | | | | | | | CGGCAGTAGACAGAAGCCCACAA |
| | ferility) | | | | | | | | | | | | TCTGCAGGAGGACTAGAGCTGTG |
| | | | | | | | | | | | | | GTCACGCTTGCATCGTGGTGTTG |
| | | | | | | | | | | | | | TGGCTTTGTTTCCTCACTGGTGTC |
| | | | | | | | | | | | | | CTTGTCTTTGGTCCTCAGGAGCAA |
| | | | | | | | | | | | | | CAAGATCAATTTCC[G/A]CTTCAG |
| | | | | | | | | | | | | | AAGGTCCTGAAAGTGGCAGCTAC |
| | | | | | | | | | | | | | TGAACGGGAGTTTGGCAACTACC |
| | | | | | | | | | | | | | TTTTCCGCCAANATCGTTTCTATG |
| | | | | | | | | | | | | | ACGCCAAAGTGAGGTATAAACGG |
| | | | | | | | | | | | | | GTAAGACTTTCAAAGGATAGGTTT |
| | | | | | | | | | | | | | AGACCCTGAAACTTCCTTGTTTGA |
| | | | | | | | | | | | | | AAGATAGACTTTGTCTCTTTCCCT |
| | | | | | | | | | | | | | GGTCTTCCCTGGTCATATGCAGG |
| | | | | | | | | | | | | | CATTGA |
| | | | | | | | | | | | | | (SEQ ID NO: 21) |
| | | | | | | | | C | Normal | A | | FKBPS- | ACACACGGCAGTAGACAGAAGCC |
| | | | | | | | | | | | | IAE2 | CACAATCTGCAGGAGGACTAGAG |
| | | | | | | | | | | | | | CTGTGGTCACGCTTGCATCGTGG |
| | | | | | | | | | | | | | TGTTGTGGCTTTGTTTCCTCACTG |
| | | | | | | | | | | | | | GTGTCCTTGTCTTTGGTCCTCAGG |
| | | | | | | | | | | | | | AGCAACAAGATCAATTTCCNCTTC |
| | | | | | | | | | | | | | AGAAGGTCCTGAAAGTGGCAGCT |
| | | | | | | | | | | | | | ACTGAACGGGAGTTTGGCAACTA |
| | | | | | | | | | | | | | CCTTTTCCGCCAA[C/A]ATCGTTTC |
| | | | | | | | | | | | | | TATGACGCCAAAGTGAGGTATAAA |
| | | | | | | | | | | | | | CGGGTAAGACTTTCAAAGGATAG |
| | | | | | | | | | | | | | GTTTAGACCCTGAAACTTCCTTGT |
| | | | | | | | | | | | | | TTGAAAGATAGACTTTGTCTCTTT |
| | | | | | | | | | | | | | CCCTGGTCTTCCCTGGTCATATGC |
| | | | | | | | | | | | | | AGCCATTGAGAAAAATCTCATTAA |
| | | | | | | | | | | | | | AAAatccttgtgccagagagctaatg |
| | | | | | | | | | | | | | actaagagaggggacccaggatt |
| | | | | | | | | | | | | | (SEQ ID NO: 22) |
| | Fracture | Disease | HIGH | N/A | Tho- | | N/A | C | Risk | A | Normal | BIEC2- | atgagaattcaatgggaagaagaatc |
| | Risk - | Risk | | | rough- | | | | | | | 6883- | aggagtggaaatctagtacatttcag |
| | Thorough- | | | | bred | | | | | | | Frac1 | ttacacagcattcccgataaatattc |
| | bred | | | | | | | | | | | | gttgcatgaatgaatcgacctctgga |
| | | | | | | | | | | | | | aatattgggatagaaatgcttgactt |
| | | | | | | | | | | | | | ccaagagcctgcagtattagaggtgt |
| | | | | | | | | | | | | | gtcaagtcattttatttagacattaa |
| | | | | | | | | | | | | | taaaaagaatgctctcag[a/c]agc |
| | | | | | | | | | | | | | ttgtaggagttggggacatccaggCT |
| | | | | | | | | | | | | | CAAATTGTTGAGACCTAGATATTGGA |
| | | | | | | | | | | | | | AAATGATCCATTTGAAGAGGATGCTT |
| | | | | | | | | | | | | | TAAGGTTGAGGATGAGAAGTTGAGGC |
| | | | | | | | | | | | | | TGCTGATTCTAGAGAACTGTGTTT |
| | | | | | | | | | | | | | TGTATTTCTGCATCTTGCTTGTGT |
| | | | | | | | | | | | | | TTCTGGGAGGGAGATATGGGAAA |
| | | | | | | | | | | | | | ATGTTCTTAGATGTTTCTTTAA |
| | | | | | | | | | | | | | (SEQ ID NO: 51) |
| | | | | | | | | C | Risk | T | Normal | BIEC2- | AGTGGGGCTGTCAGAGGAACACC |
| | | | | | | | | | | | | 416680- | AATCAAGCTCATATTAATGGCAGC |
| | | | | | | | | | | | | Frac2 | AAGAGTCAGACCAATGTAACAGTC |
| | | | | | | | | | | | | | ATTCTCTGGAAGTATGTTTGGGCA |
| | | | | | | | | | | | | | CTGGCTGATGGCTCATCCGTAAAT |
| | | | | | | | | | | | | | CCTAGAGCTGAAATCCCAGTCCTA |
| | | | | | | | | | | | | | TCCAGGTGTAAATTATTGTACATC |
| | | | | | | | | | | | | | CTATATAATTGGGAAATTTAAATG |
| | | | | | | | | | | | | | ATCCTAACA[C/T]TATCTCTTACCT |
| | | | | | | | | | | | | | ATCATCCTTAGTCTGCTAGTTACA |
| | | | | | | | | | | | | | GGCATTTCTATAGCATTTGTTATT |
| | | | | | | | | | | | | | GTAAAGATGAATGAATTGGAATAT |
| | | | | | | | | | | | | | AGAATAAAACCTGCACATGGAAAG |
| | | | | | | | | | | | | | TAACAAGTAACCGCAACCAAGCAA |
| | | | | | | | | | | | | | CAAAAGGATTCACATTGGATGATA |
| | | | | | | | | | | | | | GGAAGGAAACTCATCCACAGCTT |
| | | | | | | | | | | | | | CAACTAGCATGGAACACCCCC |
| | | | | | | | | | | | | | (SEQ ID NO: 52) |
| | | | | | | | | A | Risk | G | Normal | BIEC2- | ACTGGTTTATGCATTTTGACTGGA |
| | | | | | | | | | | | | 416681- | ATGAGAATTTACTAATGGTAAAGA |
| | | | | | | | | | | | | Frac3 | TGTCTAAAAAACTGGCTATGAATA |
| | | | | | | | | | | | | | CAGAGTTTTAGAAGGACTATCAAA |
| | | | | | | | | | | | | | ATAAAACTCTCTCTACATGAAAAA |
| | | | | | | | | | | | | | ATAATACATTATAGTATTTCTATAC |
| | | | | | | | | | | | | | ACAAATCTAAATTGAATCCACCTC |
| | | | | | | | | | | | | | TAAACTGTGAGCTCAGAGCTTAGA |
| | | | | | | | | | | | | | GAGGGCA[A/G]GTGGATTTTTACT |
| | | | | | | | | | | | | | CTCATAAATTGTTACTTGGAAGCT |
| | | | | | | | | | | | | | TAGAAACTGCTTTATAGTCCTAAA |
| | | | | | | | | | | | | | TGCTTTCTCCCTTCCCTCCCAGGG |
| | | | | | | | | | | | | | ACCAGTGCTCATTGTACTGCATGC |
| | | | | | | | | | | | | | TATGACTGCCTGGAACTCCTGGA |
| | | | | | | | | | | | | | CATTGTCCCTTTGACAATGCTTCT |
| | | | | | | | | | | | | | CTCTCTCTTTACACTCATGCTCAA |
| | | | | | | | | | | | | | GAGGTTGCACGGAGCAGCCC |
| | | | | | | | | | | | | | (SEQ ID NO: 53) |
| | | | | | | | | C | Risk | T | Normal | BIEC2- | TTGCCATCAATTCTAACCTGAAAT |
| | | | | | | | | | | | | 416704- | ATGAATTGAGCATTTAAAGGATGA |
| | | | | | | | | | | | | Frac4 | TTGTATGTAAATAATATAAAAGGG |
| | | | | | | | | | | | | | GTAATAGTCATACATTGGCAATGT |
| | | | | | | | | | | | | | TGAGTTTCAAAGTTAGTTTTAAAG |
| | | | | | | | | | | | | | GAGATGCTTTATGTGTAATAGTGA |
| | | | | | | | | | | | | | AAAGTAAAGATAAAAAAATAAAATT |
| | | | | | | | | | | | | | GAGAAGAATATATCTATATCTGTA |
| | | | | | | | | | | | | | GCAATAC[C/T]TATGCCTATAACCA |
| | | | | | | | | | | | | | TATCAGAGTAGAAGCGGGACAAA |
| | | | | | | | | | | | | | ATGATTGATAAGAGCAAAGTAATT |
| | | | | | | | | | | | | | AGCAGTGAAGTTGTACAGAGGGG |
| | | | | | | | | | | | | | TATGCAGAAATGACACGTGGGCG |
| | | | | | | | | | | | | | ATGCAGAATGAATGTAATTTTGAA |
| | | | | | | | | | | | | | TGCtaaatataaaaattaataaaata |
| | | | | | | | | | | | | | ttaatttttCAATGATAACTTATCAC |
| | | | | | | | | | | | | | AACGTTATACATATATT |
| | | | | | | | | | | | | | (SEQ ID NO: 54) |

Nucleotides in bold brackets represent SNPs of the corresponding genetic locus.

The present disclosure is also useful for identifying potential health risks and harmful predispositions based on one or more genetic biomarkers obtained from a tissue sample of the animal. Mutations in several genes in horses are known risk or causative factors for development of various disease and non-disease conditions (e.g., the disease and non-disease conditions listed in Table 10, among others).

b. Genetic Markers of Behavioral Traits

The methods and systems disclosed herein encompass genetic profiling of non-human animals for use in producing an animal having an increased probability of exhibiting one or more desired physical and/or behavioral traits or predispositions. In the context of horses, but also applicable to other non-human animals, such traits and predispositions include, but are not limited to temperament, speed, and gait. Information about these traits can be added to the database for each reference animal and can be used as a selection criterion for the desired offspring animal.

i. Temperament

Animal temperament generally refers to stable individual patterns in behavior that are largely genetically determined and independent of learning. Non-limiting examples of behavioral traits pertaining to temperament in horses may include, for example, curiosity, dominance/submissiveness, passivity, aggression, anxiety, impulsiveness, plasticity, work ethic, sloth, sociability, neuroticism, playfulness, and territoriality, among others. Prior studies in horses have demonstrated a genetic link between genes and behavior, such as, e.g., polymorphisms in the dopamine receptor D4 (DRD4) gene and curiosity and vigilance traits (Momozawa et al. Mamm. Gen. 16:538-44, 2005; incorporated by reference herein). Variants of the same gene also show an association with behavioral displays of frustration in stabled horses. Temperament information (genetic or phenotypic) can be reported in the database for each reference (e.g., parental) animal and used as a selection criterion for the desired offspring animal. For example, genetic correlations between the DRD4 gene and temperament can be employed according to the methods described herein to produce an animal having a predetermined temperament (e.g., a curious, vigilant, curious/vigilant, spooky, non-spooky, hot, cold, or medium). Additional gene-behavior correlations may be employed using the methods and systems disclosed herein to produce an animal having a desired temperament.

According to the present disclosure, temperament may be assessed in a non-human animal using genetic testing by, e.g., determining the presence of one or more polymorphisms in the DRD4 gene in the animal, as is discussed above. Additionally, or alternatively, the temperament of the non-human animal may be self-reported by a person familiar with the animal, such as an owner, breeder, seller, or caretaker. Such subjective assessments may be made using conventional methods, such as, e.g., rating the animal's temperament on a scale from 1 to 10. A non-human animal assessed as having a self-reported temperament of 1 will generally be non-reactive, calm, and easy (i.e., “bombproof”). An animal assessed as having a self-reported temperament of 10 is generally high-energy, extremely spooky, and difficult to manage (i.e., “hot”). The present disclosure also allows for verification of self-reported assessments of animal temperament by means of genetic testing.

ii. Speed and Endurance

Additional behavioral traits that may be desirable in an animal, such as a horse, include traits related to athletic performance such as agility/endurance, coordination, jumping skills, gait, and ability to perform in a given discipline, among others. Polymorphisms in the myostatin (MSTN) gene have been shown to strongly associate with running speed and endurance in horses. Therefore, information pertaining to presence of particular variants of the MSTN gene in the animal may be used according to the methods described herein to produce an animal having desired agility and endurance characteristics. Horses may be produced that exhibit aptitudes for fast, short distance sprints (e.g., 7 furlongs or less [1 furlong=⅛^{th} of a mile or 220 yards]), middle-distance racing (e.g., 8-10 furlongs), or long-distance racing (e.g., >10 furlongs). It is important to note that aptitudes for sprints of a particular distance may depend on the age of the animal. Detailed characterizations of MSTN gene polymorphisms in horses as they pertain to speed and endurance aptitude in horses are described in, for example, Guilotto et al. BMC Genet. 16:126 (2015); Binns et al. Anim. Genet. 41(Suppl. 2): 154-8 (2010); Hill et al. PLoS One 5:e8645 (2010); Hill et al. Appl. Physiol. 112:86-90 (2012), and Bower et al., Nat. Comun. 3:643 (2012); the disclosures of which are incorporated herein in their entirety. Speed and endurance information can be reported in the database for each reference (e.g., parental) animal and used as a selection criterion for the desired offspring animal.

iii. Gait

Locomotion in certain breeds of horses (e.g., gaited breeds) is characterized by the ability to perform multiple unique gaits, typically corresponding to different speeds and footfall patterns. Some members of non-gaited (e.g., trotting) breeds are able to perform lateral movements. Furthermore, most horses transition into a three-beat canter at high speeds, however, certain individuals (e.g., harness racing breeds) can preserve intermediate gaits without breaking into a canter even at high speeds. Locomotion traits can be reported in the database for each reference (e.g., parental) animal and used as a selection criterion for the desired offspring animal.

Genetic determinants of gaiting in horses include the DMRT3 gene, a transcription factor expressed within spinal cord locomotor circuits. Some polymorphic variants of this gene in Icelandic Horses are associated with varying degrees of synchronization of diagonal legs and higher ratings of the trot and gallop, while other variants are associated with increased speed and coordination at the tölt. Horses having one or more copies of DMRT3 are often unable to canter or change leads. This can be problematic for Western Reining horses and a competitive advantage for Western Pleasure horses. Thus, the methods described herein encompass the incorporation of information relating to the DMRT3 gene (e.g., presence of a specific DMRT3 genotype in the animal) into the database system for the purpose of producing an animal having desired a gait characteristic.

While a large number of genes have been linked to physical and behavioral traits in horses, many remain to be discovered. Newly discovered associations may be incorporated into database and used in the methods of the present disclosure as they become available to refine the selection process for a desired phenotype in a non-human animal, to expand the breadth of genetic diagnoses of disease and non-disease conditions, and to open avenues for artificially manipulating these genes for the purpose of improving the health, performance, husbandry, and discipline-specific traits of the animal. The present disclosure allows for the discovery of new correlations between genetic markers and physical or behavioral attributes by correlating genetic information obtained from the animal (e.g., by way of genetic testing) with information gathered from secondary sources, such as behavioral data and show scores obtained from owners, breeders, and judges of various sporting events/competitions. Furthermore, the present disclosure allows for the correction of self-reported information about the animal using the animal's genetic information gathered according to the methods described herein if the genetic information is contradictory to the self-reported information.

c. Genetic Diagnostic Testing

As is described above, systems and methods of the disclosure can be used to identify a non-human animal (e.g., horse, cattle, sheep, dog, cat, camel, pig, goat, alpaca, donkey, llama, red fox, mouse, rat, ferret, non-human primate, rabbit, gerbil, hamster, chinchilla, or guinea pig, among others) as having one or more predetermined traits based on the expression levels (e.g., mRNA expression level or protein expression level) of one or more biomarkers in a biological sample obtained from the animal. The biological sample can include, for example, cells, tissue (e.g., a tissue sample obtained by hair sample or biopsy, such as, e.g., fresh frozen or formalin-fixed paraffin embedded tissue), blood, serum, plasma, urine, sputum, nail/hoof clippings, hair follicles, cell-free fetal DNA, mitochondria, a gamete (e.g., oocyte or spermatozoon), or an embryo of the offspring animal implanted as part of an in vitro fertilization (IVF) procedure. Numerous methods of determining biomarker expression levels, or expression profiling, are known in the art, including, but not limited to, microarrays, polymerase chain reaction (PCR), reverse transcriptase PCR (RT-PCR), quantitative real-time PCR (qPCR), Northern blots, Western blots, Southern blots, NanoString nCounter technologies (e.g., those described in U.S. Patent Application Nos. US 2011/0201515, US 2011/0229888, and US 2013/0017971, each of which is incorporated by reference in its entirety), next generation sequencing (e.g., RNA-Seq techniques), and proteomic techniques (e.g., mass spectrometry or protein arrays).

Accordingly, the one or more biomarkers may be a nucleotide sequence (e.g., DNA or RNA) encoding a gene of interest. The nucleotide sequence may be embodied in a single- or double-stranded polynucleotide. The presence or absence of expression of a particular gene marker (e.g., gene markers of Table 10) can be assessed using any of the methods known in the art for determining the nucleotide composition of a polynucleotide sequence. For example, the nucleotide sequence of a gene of interest can be assessed using nucleic acid sequencing technologies including but not limited to Sanger sequencing methods, Next Generation Sequencing (NGS; e.g., pyrosequencing, sequencing by reversible terminator chemistry, sequencing by ligation, and real-time sequencing) such as those offered on commercially available platforms (e.g., Illumina (e.g., AmpliSeq), Qiagen, Pacific Biosciences, Thermo Fisher, Roche, and Oxford Nanopore Technologies). Clonal amplification of target sequences for NGS may be performed using real-time polymerase chain reaction (also known as RT-qPCR) on commercially available platforms from Applied Biosystems, Roche, Stratagene, Cepheid, Eppendorf, or Bio-Rad Laboratories. Additionally, emulsion PCR methods can be used for amplification of target sequences using commercially available platforms such as Droplet Digital PCR by Bio-Rad Laboratories. Genetic analysis may be performed with respect to any one of the genes disclosed in Table 10 of the present disclosure, other genes or genetic loci associated with one or more of the additional traits and phenotypes described herein, as well as any one or more of the genes disclosed in Guerin et al., Anim. Genet. 34(3): 161-8 (2003), the disclosure of which is herein incorporated by reference in its entirety. Genomic sequencing may also be performed over the entire genome of a cell obtained from a non-human animal in order to detect a biomarker of interest. Exemplary analyses of an entire genome include but are not limited to a genome wide association study (GWAS) analysis. Other genes not described in the present disclosure may also be assayed according to known methods and information about those genes can also be incorporated into the database system for interrogation by a user according to the methods described herein. The results of a genetic analysis of an animal can also be incorporated into the database of the present disclosure and interrogated according to the systems and methods described herein.

The genetic markers for use in conjunction with the disclosure described herein may be single nucleotide polymorphism (SNP), tag SNP, microsatellite, short tandem repeat (STR), simple sequence repeat (SSR), restriction fragment length polymorphism (RFLP), variable number tandem repeats (VNTRs), amplified fragment length polymorphism (AFLP), insertion-deletion polymorphism (INDEL), random amplified polymorphic DNA (RAPD), ligase chain reaction, or a simple sequence conformation polymorphisms (SSCP).

In instances in which the genetic marker is an SNP, the disclosure provides methods for using SNP information gathered from a biological sample of the animal for genetic analysis. SNPs are particularly useful for analyzing the genome of mammals. First, SNPs occur with greater frequency (approximately 10 to 100 times larger), and with greater uniformity than other polymorphic markers such as RFLPs and VNTRs. The greater frequency of SNPs means that they can be more readily identified than the other classes of polymorphisms. The greater uniformity of their distribution permits the identification of SNPs that are “closer” to a particular attribute of interest. For example, if a particular trait (e.g., speed) reflects a mutation at a particular location (e.g., a myostatin gene locus), then any polymorphism that is linked to the specific gene locus, can be used to predict the likelihood that an individual will show that trait. SNPs are also more stable than other classes of polymorphisms. Their spontaneous mutation rate is approximately 10⁻⁹, approximately 1,000 times less frequently than VNTRs, which exhibit significantly higher mutation rates. Furthermore, SNPs have the additional advantage that their allele frequency can be determined from a smaller sample size as compared to other polymorphisms such as RFLPs or VNTRs, thereby offering a higher degree of genetic resolution for determining individual identity, pedigree, and susceptibility of an animal to a particular genetic trait. As an additional benefit, SNPs cover the whole genome, which may facilitate identification of potential interactions of gene products expressed from anywhere on the genome, without requiring prior knowledge about a potential interaction between genes.

Accordingly, SNPs (e.g., any one of the SNPs described in Table 1, among others) can be used in conjunction with the methods and systems of the present disclosure for genetic diagnostic testing and ancestry and parentage determination of a non-human animal (e.g., a horse). For example, the method of the present disclosure can be used to determine the probability that an animal, such as a horse, has a particular disease or non-disease condition (see, e.g., Table 1), is a member of a particular breed, or is an offspring of a specific animal or breeding pair. Detection of disease or non-disease conditions can be performed in the animal by detecting the presence of one or more disease- or non-disease condition-associated SNPs. Ancestry determination using SNP analysis can be carried out by analyzing genetic markers (e.g., SNPs) across the entire genome of the animal to identify the predominant overlapping markers present in the animal of interest and its ancestors. For the uses described above, lineage determination for a particular animal may be aided using the disclosure in conjunction with art-recognized methods.

d. Ancestry Determination

Certain groups of horses have been closely bred and tracked for centuries (e.g., “closed stud book”), thereby rendering their gene pool limited and easily tracked. Other breeds have rapidly or more recently developed for a variety of reasons. Many newer breeds were selectively bred to produce a specific appearance, size, conformation, or ability for different disciplines and emerging equine trends. While some sports or purposes remain specific with a stable focus (e.g., racing or carriage), others are constantly evolving with breeding trends, competition demands, and styles (e.g., hunter/jumper, color breeds, and reining). Therefore, a particular horse breed, such as, e.g., a Reining Quarter Horse, will have a dramatically different look today than it may have 40 years ago, whereas other breeds such as, e.g., Racing Harness horses, may appear largely similar with minor changes in step with each leading sire. In other examples, although most white markings may be nouveau, they are not new. While new genetic variants for white markings are constantly being discovered, some of the most popular variants have been around for a long time. For example, Tobiano horses, which are famous for making a Pinto spotting pattern on the coat) can be found in ancient horses from as far back as 3000-5000 years ago.

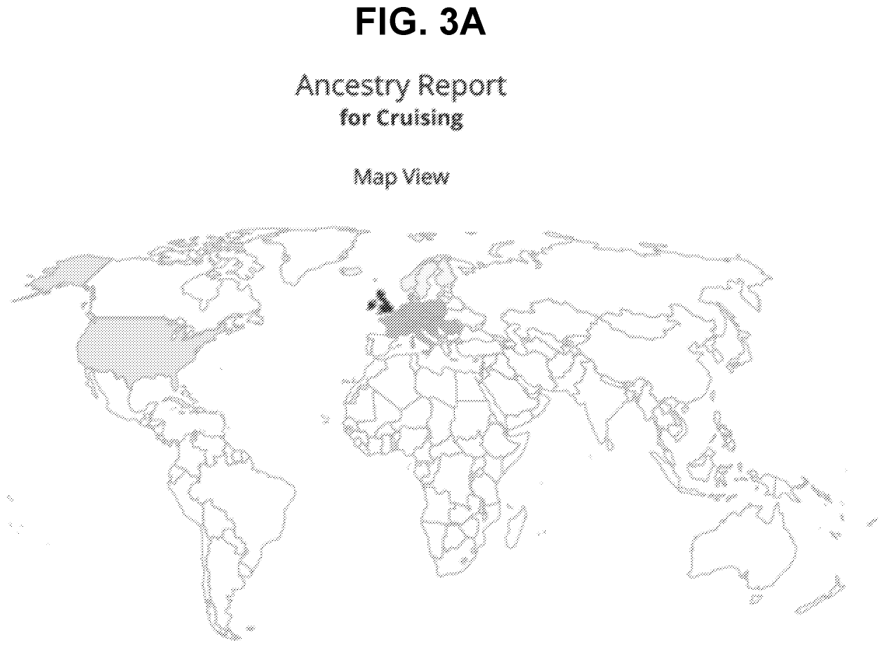

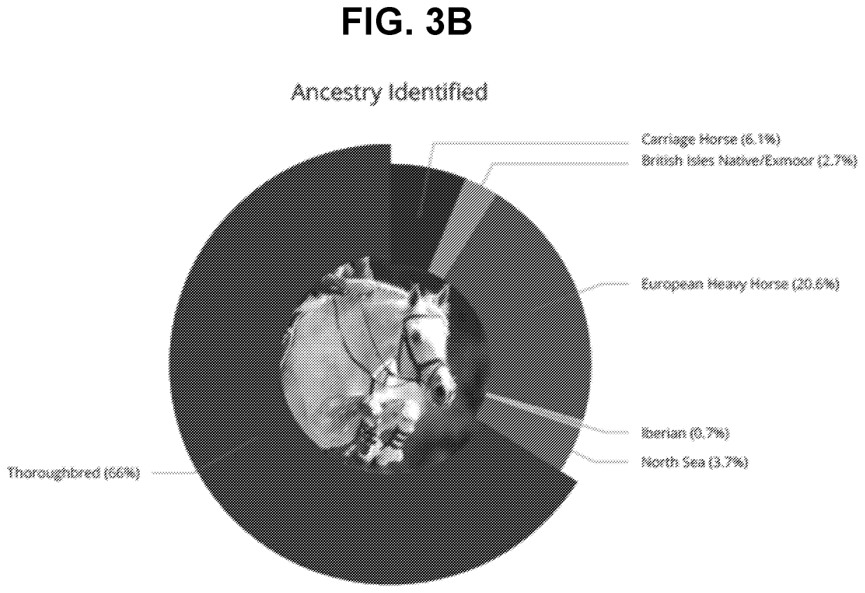

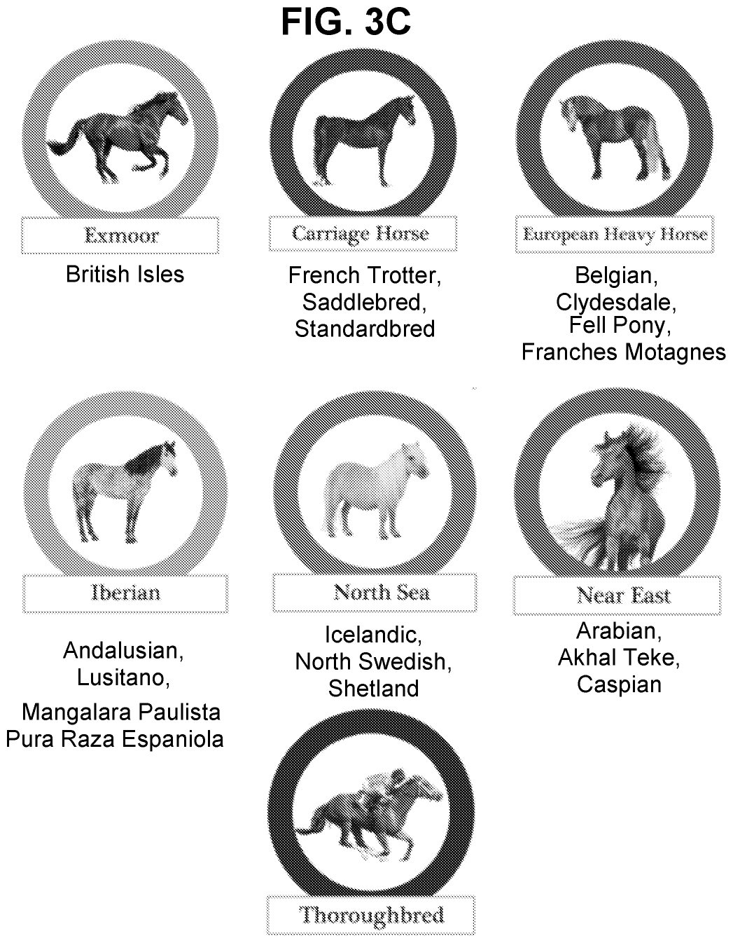

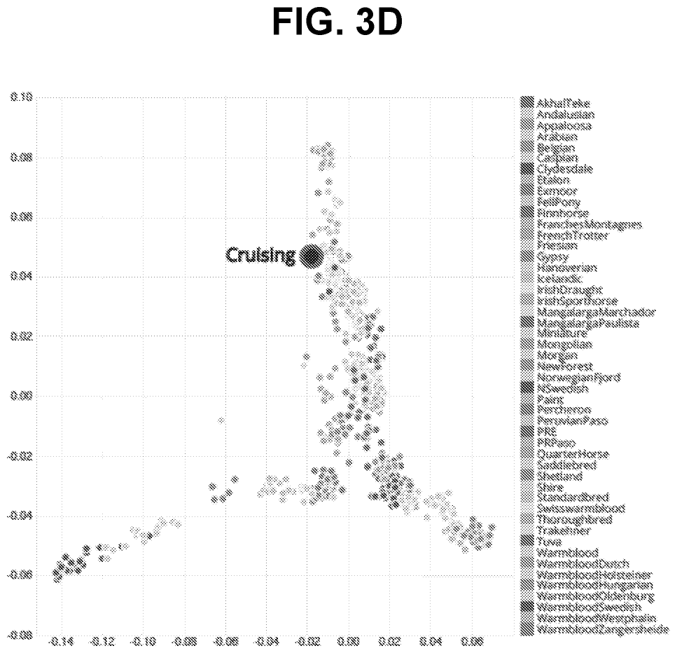

Thus, the methods and systems of the present disclosure may also be employed to perform a composition analysis that assesses non-human animal (e.g., equine) ancestry based on regional breeding practices and evolutionary drift. Horses from each region of the world (see FIGS. 3A-3C), group, or breed will have overlapping genotypes, which can be identified, compared, and graphically visualized in an Ancestry Report using the methods and systems disclosed herein (see FIG. 3D). Where horses have been closely bred (e.g., due to close proximity) or purposely bred to suit a specific discipline or need, the genetic content of these horses will cluster together and may correlate with breed designations. Dimensionality reduction methods such as, e.g., principal components analysis (PCA) can be applied to high-dimensional data sets containing genetic information about a plurality of horses to identify groups (or “clusters”) of genetically related horses. Ancestry analysis can be performed over large reference regions of the genome (e.g., using GWAS-based analysis, e.g., using SNPs as markers of genetic variance), although it can also be tailored to analyze specific genes or genetic loci. Registries, breeds, or closed groups of horses bred through longer periods of history may have more clearly defined gene clusters. In some cases, these clusters may be entirely separated from most horse samples (see FIG. 3D).

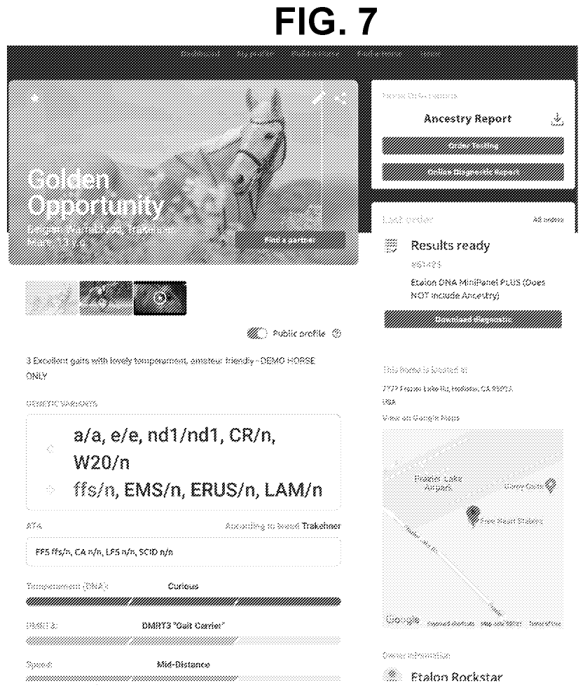

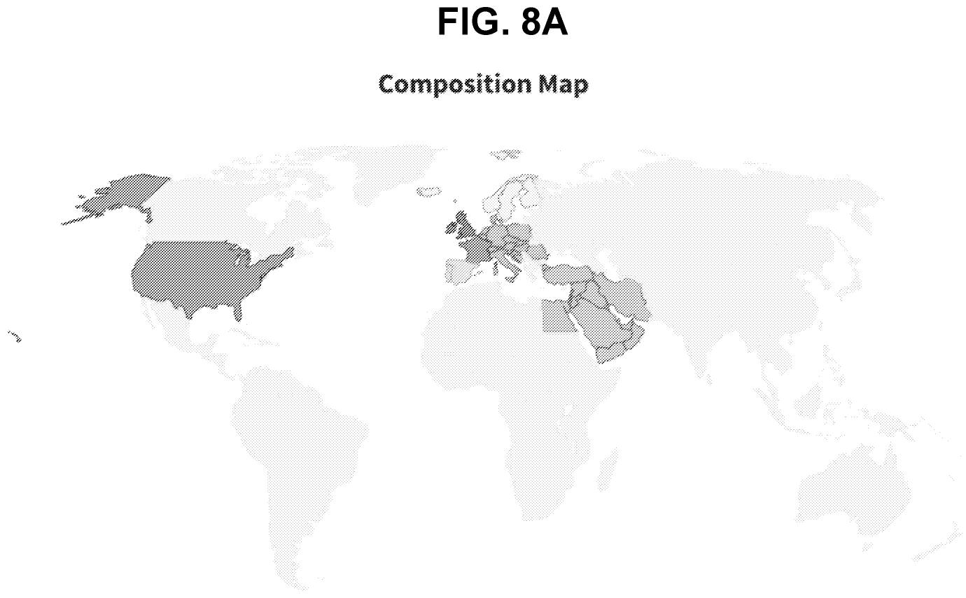

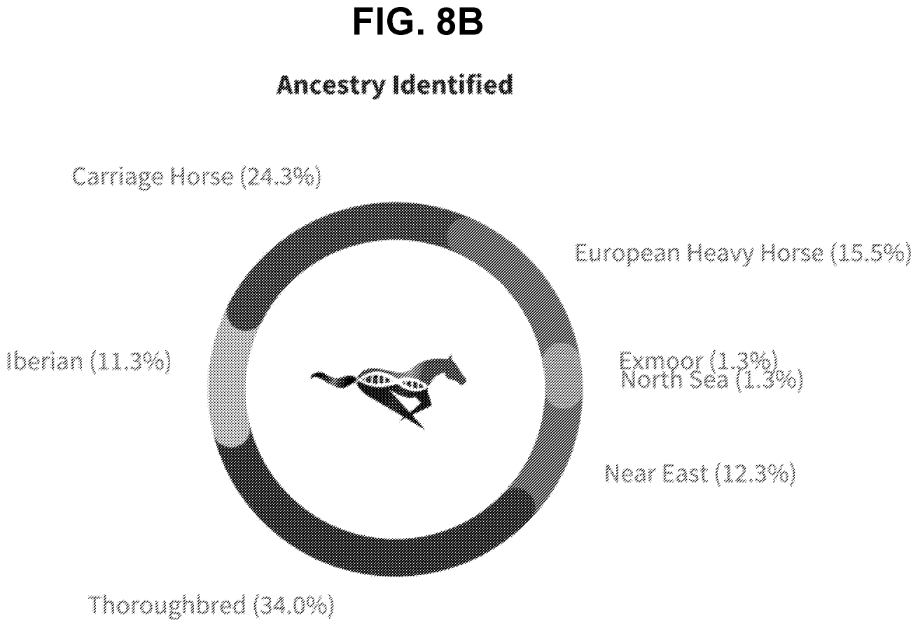

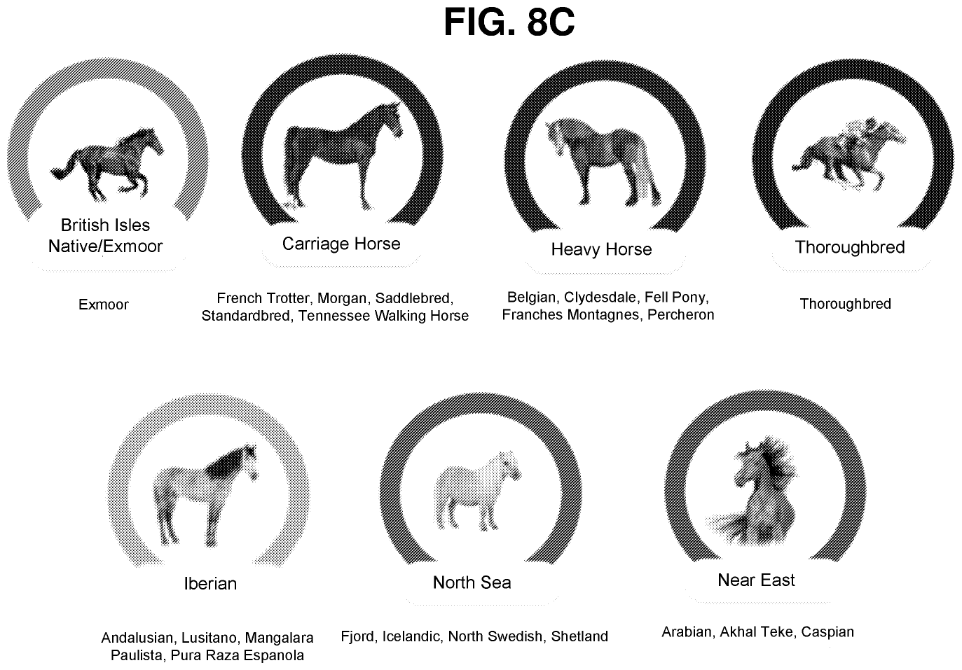

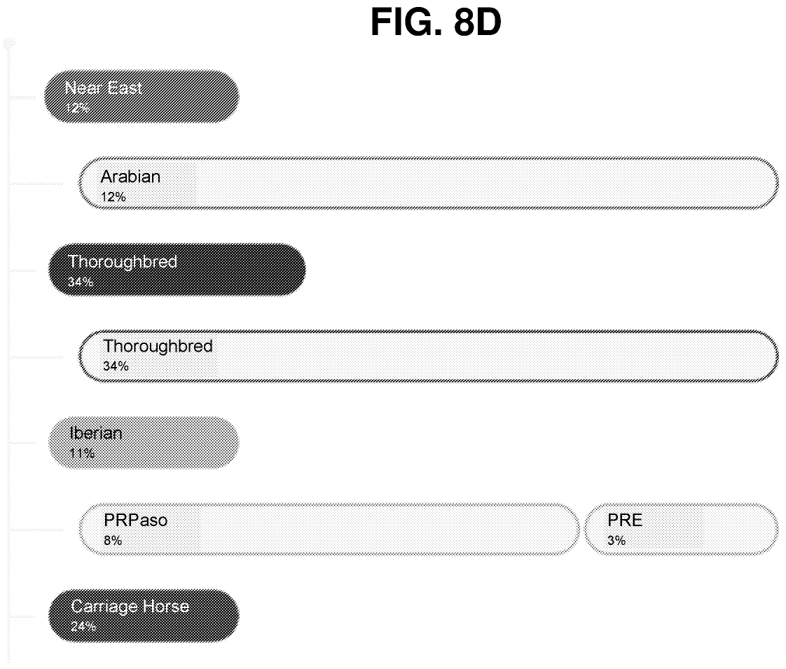

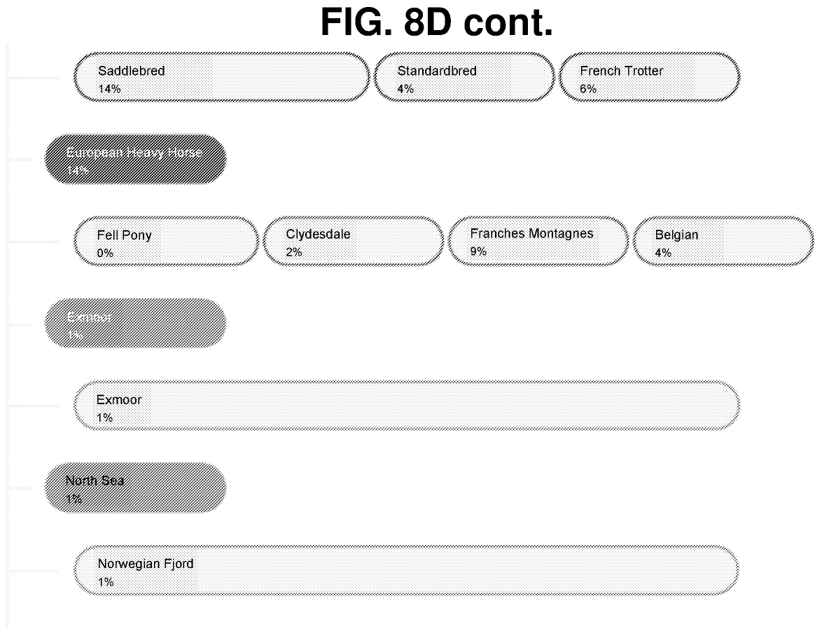

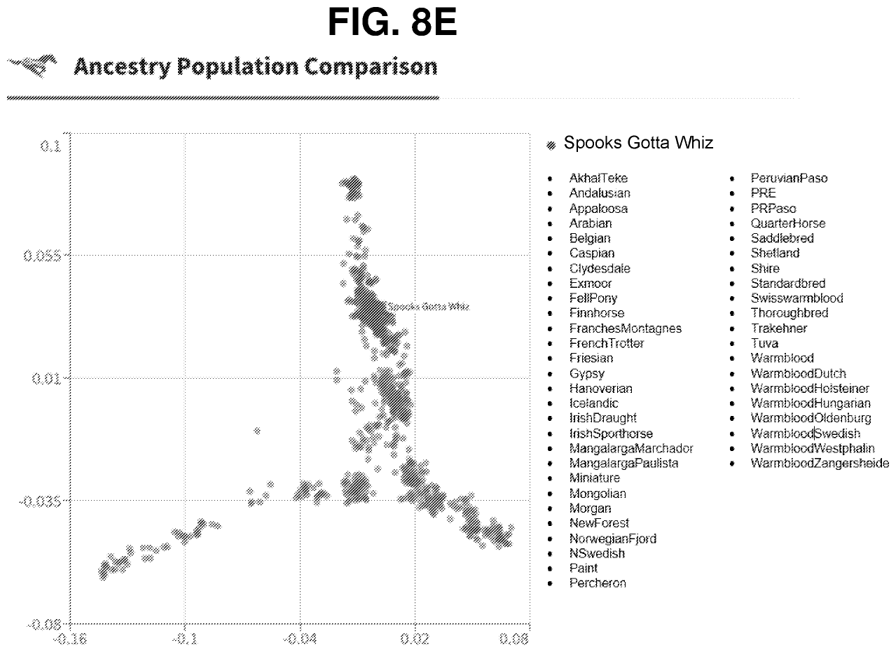

In some cases, a horse breeder may wish to determine the breed composition of a particular horse, or the potential breed composition of foal produced from the breeding of two or more horses. To this end, the user may order an Ancestry Report for an animal of interest using the database or system of the present disclosure (see FIG. 7 as an example of such a report generated using the computer-implemented user interface of the disclosure). Using genetic information obtained from a biological sample obtained from the animal of interest (e.g., by way of genetic diagnostic testing), the present disclosure provides a quantitative method for deconstructing the breed composition of the target animal based on an ADMIXTURE method described previously by Alexander et al. (Genome Res. 19:1655-64, 2009; incorporated herein by reference in its entirety). In general, the method first involves constructing a reference population of animals curated for specific individuals that fit the need and described breed or type (e.g., information, such as genetic information, of a reference population of animals of a specific type). Within the context of the present disclosure, purebred samples with known pedigrees across a large number of known breeds can be obtained and an optimal number of founder breeds (e.g., k=17) can be determined for ancestry analysis. Because each allele has a frequency that differs between breeds, comparing the breed probabilities with the actual alleles in a given horse across multiple loci allows for the estimation of breed composition (see FIG. 8D). Thus, the results of genetic diagnostic tests performed on an animal using the database system of the disclosure or the results of the genetic tests incorporated into the database system of the disclosure can be used to understand the breed composition of the animal. The same results can then be used as an exclusion or inclusion criteria to identify or produce a non-human animal having one or more desired traits using the database system of the disclosure. As an example, a horse breeder may wish to produce a racing horse that is at least, e.g., 30% Thoroughbred. The horse breeder may use the database system of the disclosure to specify “at least 30% Thoroughbred” as an inclusion criterion for selecting the desired horse phenotype.

In addition, methods for determining the ancestry or relatedness between two or more animals (e.g., two or more horses) can include using a “shared centimorgan (cM)” metric. cM is a metric commonly employed by geneticists to define the distance between chromosome positions and, therefore, can be used as a measure for assessing genetic linkage between two or more potentially related animals. While the breed-averaged recombination map spans 2.36 billion base pairs and accounts for 2,939.047 cM, one cM in a horse typically corresponds to 23.8 million base pairs (Beeson et al., Genome Res. 29:1744-52, 2019—incorporated by reference herein); however, this distance may vary between different animals, families, and especially breeds due to variations in rates of recombination across these dimensions. Within the context of the present disclosure, shared cM is useful as a metric that quantifies relatedness between two animals. As an example, an average horse has about 2,939 cM of DNA, half of which is inherited from each biological parent. Thus, any given horse inherits about 1,469.5 cM of DNA from each biological parent. In order to calculate the percentage of shared DNA between a first and a second individual, the total number of shared cM between the first and second individual can be divided by the total number of cM of the first individual. For example, a first animal having 217 shared cM with a second animal would have (217/2939=) 7.38% of shared DNA with the second animal. This measure accounts for genetic variation between two animals by comparing the differences between specific genetic regions in both animals and assessing the similarity between the regions. The similarity between the analyzed genetic loci may be useful for determining if two animals are siblings, half-siblings, cousins, or if they are more distantly related. Generally, measurement of shared cM requires performing genetic diagnostic testing on the two or more potentially related animals of interest, e.g., as is described herein, and quantifying the shared cM based on the results of the genetic tests.

In addition to shared cM, kinship analysis allows for increased accuracy in estimating relatedness between two or more animals (or two or more horses) from the same or different breeds (Manichaikul et al., Bioinformatics 26:2867-73, 2010—incorporated by reference herein). Kinship values are utilized in conjunction with shared cM to support or exclude relationships. While a negative kinship coefficient estimation indicates an unrelated relationship, close relatives can be inferred based on the estimated kinship coefficients averaged out through known related samples for each breed. As an example, an estimated kinship coefficient range >0.354, [0.177, 0.354], [0.0884, 0.177], and [0.0442, 0.0884] corresponds to duplicate/MZ twin, 1st-degree, 2nd-degree, and 3rd-degree relationships, respectively

The shared cM metric and kinship coefficients can be particularly useful to an animal breeder for producing a non-human animal having desirable characteristics using the computer-implemented database system described herein. For example, a horse breeder may desire to produce a racehorse that has a good temperament, multi-distance racing capability (e.g., CT genotype in the myostatin locus), and is slightly taller than average to achieve greater reach. In this example, the horse breeder already possesses a mare that is an Endurance-type (e.g., TT “distance” genotype in the myostatin allele) and is good for long distances but not fast enough in shorter sprints. The mare may have a relatively high F value of 15% but possesses an excellent temperament and is of medium size with good physical conformation. In order to produce a horse with characteristics desired by the breeder, the breeder may search for a sire that has: (1) “CC sprint” genotype in the myostatin allele to promote the “CT mid-distance” genotype; (2) increased height; and (3) good temperament. Upon finding one or more suitable candidate sires, the breeder may perform genetic testing or obtain prior genetic tests on the mare and sire using the database system of the disclosure to quantify how closely they are related using the shared cM and/or kinship analysis. Once the breeder has narrowed down the pool of potential sires, the shared cM and/or kinship coefficient measures can assist the breeder in determining whether the mare and the sire would share genes that are advantageous for producing a foal having the desired characteristics. For example, the breeder may use the database system of the disclosure to analyze shared cM and kinship coefficients between two horses of unknown relation on the basis of genetic diagnostic test results stored in the disclosed database system. Once the shared cM and kinship coefficient data is stored in the database system of the disclosure, the breeder may use the database system to include or exclude pairs of breeding horses on the basis of shared cM (e.g., exclude a sire and dam on the basis of a shared cM threshold) and/or kinship relatedness inferences.

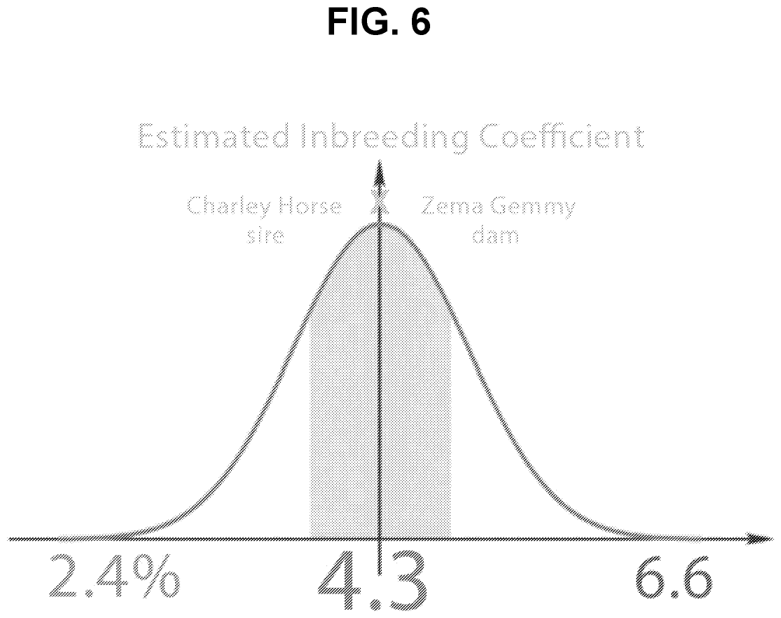

Another quantitative metric useful for assessing ancestry of an animal using the computer-implemented database system described herein is the “inbreeding coefficient” (F). This metric measures the likelihood that two alleles of any given locus in an individual are identical based on descent from the common ancestors of the two parents. F is defined by the following formula:

$$F = \frac{observed homozygosity count - expected homozygosity count}{total allele count - expected homozygosity count}$$

where the expected homozygosity count is determined using the observed allele frequencies at each genetic locus. The F value—measured as the number of genetic variants that are homozygous—can be determined over specific genes but is typically measured over larger regions of the genome and assesses homozygosity across these regions relative to a reference population (e.g., reference population information stored in the database system of the disclosure). Without wishing to be bound by any theory, a high degree of inbreeding can negatively impact athletic performance (Samsonstuen et al., Acta Agri Scand 69(3):152-6, 2020; and Todd et al., Sci Rep 8:6167, 2018) and can increase susceptibility to deleterious health conditions, thereby negatively impacting the phenotypic value of an animal. Therefore, a horse breeder may desire to minimize F in a target foal to optimize its phenotype. The F metric can be employed independently or in combination with the shared cM metric to produce a phenotypically advantageous animal. Generally, a breeder may desire to produce or purchase an animal that has an F value that is, e.g., no greater than 0.2 (20%). Thus, the F value can be a suitable exclusion criterion for identification or production of animals having advantageous phenotypes. For example, once a horse breeder has analyzed the genetic relatedness between two horses using the shared cM metric, which compares specific genetic loci/genotypes between two animals, the breeder could further limit available candidate horses for buying, leasing, or breeding by specifying a limit on the inbreeding coefficient of a specific horse (e.g., F≤15%). The same analysis can be performed for a predicted foal that would be produced from a particular mare and stallion to produce a “likely inbreeding coefficient for foal.” Such a method would allow the breeder to select a breeding pair that would produce a foal with tangible individual genetic traits (e.g., myostatin genotype, color, disease predisposition) along with the not-so-tangible overall effect of inbreeding (see FIG. 6).

Another metric useful for assessing the ancestry of an animal is the heterozygosity score, which is taken as the percentage of observed genetic loci that have two different alleles. Heterozygosity is generally linked to the health of an animal since an animal with low zygosity has a lower survival and reproductive probability. The heterozygosity score can be useful in identifying the potential bloodline (e.g., breed or type) for an animal of unknown bloodline for pedigree tracing or health assessment (Petersen et al., PLoS One 8:e54997, 2013; and Excoffier et al., Evol. Bioinform. Online 1:47-50, 2005—both incorporated by reference herein). In the context of the computer-implemented database system of the disclosure, the heterozygosity score may be employed as a filter for identifying or producing a non-human animal (e.g., a horse) having a desired phenotype. For example, a horse breeder may wish to produce an animal having a heterozygosity score above a threshold value, such as, e.g., 0.232 (23.2%). Accordingly, the results of genetic diagnostic tests performed on an animal using the database system of the disclosure or the results of the genetic tests incorporated into the database system of the disclosure can be used to assess the heterozygosity score of the animal. The same results can then be used as an exclusion or inclusion criteria to identify or produce a non-human animal having one or more desired traits using the database system of the disclosure. As an example, a horse breeder may wish to produce a horse whose overall health assessment is based on a heterozygosity score of at least, e.g., 0.31 (31%). The horse breeder may use the database system of the disclosure to specify “at least 0.29 (29%) heterozygosity score” as an inclusion criterion for selecting the desired horse phenotype.

Yet another quantitative metric useful for assessing the ancestry of pairs of seemingly unrelated individuals is the “most like me” metric, which is based on a previously developed identity-by-descent (IBD) measure (Purcell et al., AJHG 81:559-75, 2007; incorporated herein by reference) that estimates ancestries from multi-locus or whole-genome association studies (WGAS). In short, a segment of DNA can be identical by descent in two or more individuals if they have inherited the segment from a common ancestor without recombination (i.e., the segment has the same ancestral origin in both individuals). IBD can be performed between an arbitrary number of possible pairs of animals (e.g., horses) and return the highest matches to identify two animals as ancestrally related. In the context of the database system of the disclosure, the “most like me” function interrogates the database using input criteria set by the user (e.g., one or more predetermined traits) by matching the input to equivalent or similar information present in the database system for a reference population of animals (e.g., animals previously registered into the database). As an example, a horse breeder may wish to identify or produce a horse having similar characteristics to a known horse with an advantageous phenotype set. The breeder could obtain a genetic sample from the known horse and perform genetic diagnostic testing on the animal (e.g., using methods disclosed herein). The breeder could then employ the computer-implemented database system of the disclosure to find another horse similar to the known horse using the “Horse like me” function of the computer-implemented user interface or by specifying the parameters of interest (e.g., Quarter horse, higher than average percentage of Thoroughbred breed, and from the “High Brow cat” bloodlines). The database system may interrogate one or more (e.g., 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or more) genetic loci causally linked to the one or more desired parameters/traits of interest in the known horse and determine whether the same loci might also be present in another existing horse and correlate with the same desired traits. This would allow the breeder to identify two horses (a sire and a dam) that are either available for sale/lease and breeding that, when bred, produce a horse that closely resembles the target phenotype. The breeder could further employ the shared cM metric to identify a full sibling of the same gender, color, and disposition as desired to buy, lease, or breed.

Because horses are continually being purpose-bred for ever-changing disciplines and desired traits, the ancestry analysis will be updated over time to account for genetic drift in the animal populations by continually incorporating information about horses into the database system of the disclosure. The results of an ancestry analysis may include discovery or beta-stage data that may change over time as equine science evolves. The currently defined population for equine ancestry may include unintended bias towards specific breeds or regions based on sample sizes and availability of horses for genetic testing. It is assumed to have an estimated error range of 5-7% which may change with growth, sampling, and discovery in the sample population. As populations, disciplines, and breeding trends change over time, so too will the results of each horse when compared within the population at the time.

e. Parentage Determination

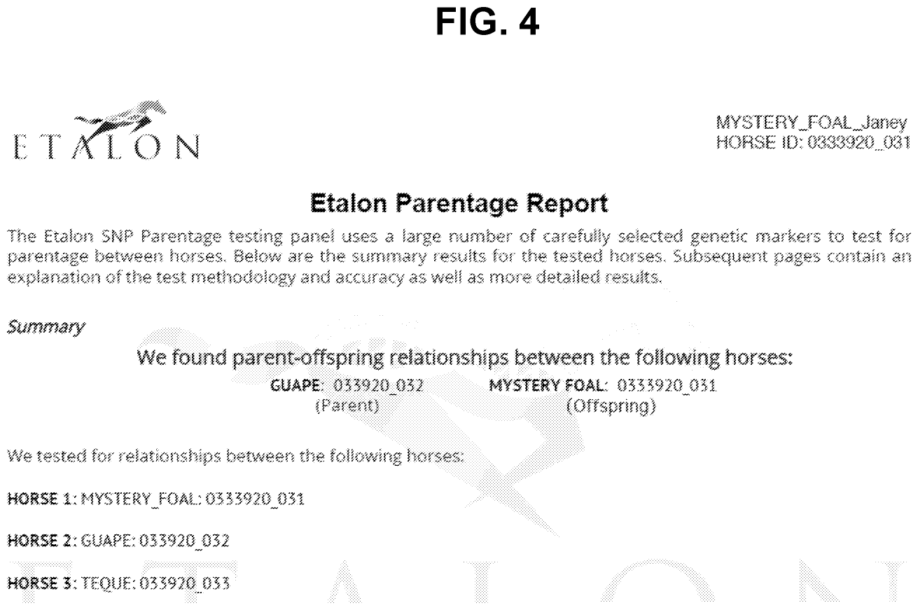

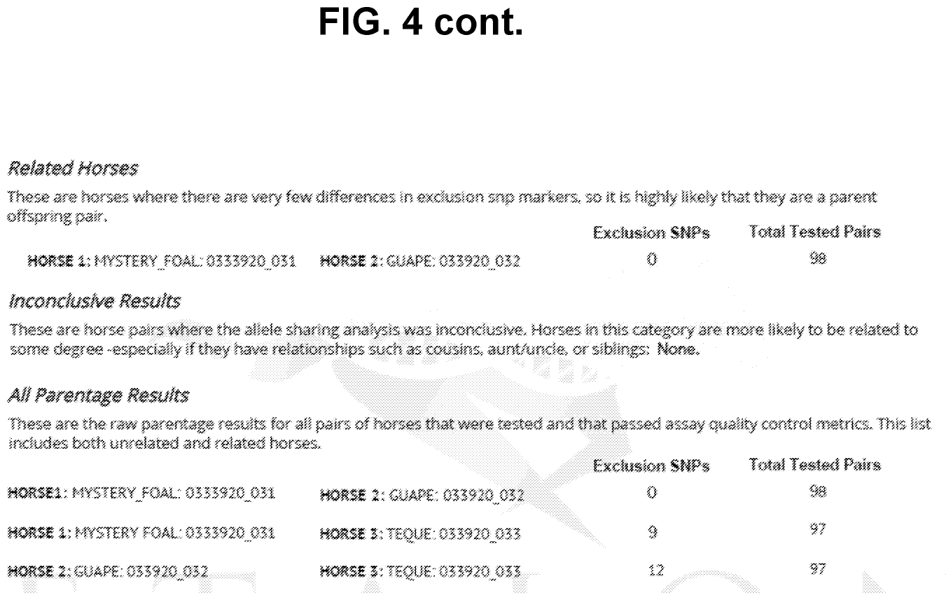

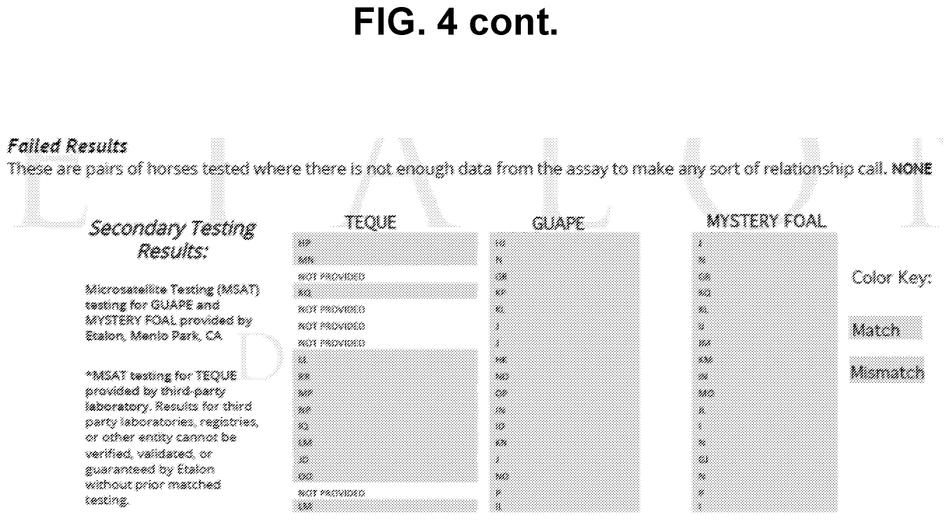

The methods and systems disclosed herein may be used to identify parentage of an offspring animal whose parentage is fully or partially unknown (i.e., both parents are unknown, or a single parent is unknown). For example, results of genetic testing of a non-human animal (e.g., horse) can be analyzed (e.g., using the shared cM metric described herein) and compared to results of genetic testing of a plurality of horses catalogued in the database system of the disclosure. A variety of genetic markers (e.g., SNPs, such as, e.g., exclusion SNPs) can be used to test across a large number of carefully selected genes to test for parentage between horses. An exclusion SNP can be used to rule out the likelihood that an offspring animal and a candidate parent animal are related. Generally, the SNPs can be used to define an exclusion probability, which is the average capability of any genetic marker to exclude a group of unrelated individuals from a family. Typically, an individual is excluded as the parent of an offspring if the offspring's genotype at a particular genetic locus cannot be produced from the genotype of the candidate parent animal based on Mendelian inheritance and genetic mutations. A detailed description of methods for calculating parentage exclusion probabilities is described in Wang, Heredity 99:205-17 (2007), the disclosure of which is incorporated by reference in its entirety herein as it relates to determination of parentage exclusion probabilities. The database system may employ a comparison engine that assesses the degree of overlap between the genotype of a subject offspring horse whose parentage is fully or partially unknown and a plurality of candidate parent animals. The database system may then produce a probability matrix containing an array of exclusion probability values for one or more candidate parent animals. A low exclusion probability value (e.g., 12% or less, such as, e.g., 12%, 11%, 10%, 9%, 8%, 7%, 6%, 5%, 4%, 3%, 2%, 1%, or less) would indicate a high likelihood that the offspring animal and the candidate parent animal are related. Similarly, the database system may produce the absolute quantities for the number of exclusion SNPs identified in the one or more candidate parent animals (e.g., see FIG. 4A). A low number (e.g., less than 5, such as, e.g., 4, 3, 2, 1, or 0) of exclusion SNPs identified between the subject offspring animal and a candidate parent animal would also indicate a high probability that the two animals are related. Methods for parentage determination using SNPs are described in detail in Holl et al. Animal Genetics 48(6):669-76 (2017), the disclosure of which is herein incorporated by reference in its entirety.

B. Non-Genetic Information Gathered from Secondary Sources

While the genetic constitution of an animal may be determinative for a variety of different physical and behavioral traits, training and environmental factors also play a critical role. Relatedly, not all traits can presently be accounted for by genetic analysis alone. Therefore, the instant disclosure provides methods for gathering information about a non-human animal (e.g., a horse) from secondary (e.g., non-genetic) sources in order to complement genetic information about the animal. Combining genetic and non-genetic information about the animal according to the methods described herein provides an holistic animal profile that can be used to predict the phenotype of an animal with high confidence and/or to inform breeding cross-match evaluations for use in producing an animal having one or more predetermined traits.

The present disclosure allows for gathering of information from a variety of secondary sources including, but not limited to someone knowledgeable about the animal such as, e.g., the owner of the animal, an animal breeder/seller, or a caretaker; scores assigned to the animal during a competition pertaining to one or more predetermined traits; clinical/veterinary diagnostic testing performed in the animal pertaining to one or more predetermined traits; and a clinical assay of biological tissue obtained from the animal pertaining to one or more predetermined traits. Other secondary sources of information about an animal pertaining to one or more predetermined traits not described herein may be catalogued and added to the database in order to provide a more comprehensive non-genetic profile of the animal. The secondary source information can be added to the database for any reference (e.g., parental) animal and can be used, if desired, as a selection criterion for the desired offspring animal

a. Information Gathered from Someone Knowledgeable about the Animal

With respect to secondary source information gathered from someone knowledgeable about the animal, such information can be obtained from the owner/breeder/seller/caretaker of the animal in the form of a questionnaire. Such questionnaires can be designed to obtain detailed information about specific behaviors and skills of the animal of interest. Questionnaires are directed to a number of traits which include, but are not limited to spooking, jumping skill, speed, dressage, temperament, and gait, among others. Additionally, questionnaires relating to the health of the animal can also be gathered. Such questionnaires can include, for example, questions about the respiratory and digestive systems, lameness, bacterial, fungal, or viral susceptibility of the animal, among others.

b. Information Gathered from Sporting or Competition Events

With respect to secondary source information about the animal gathered during a sporting event or competition, such information can readily be made available as horses often showcase their skills at various events and judged on a gamut of physical and behavioral characteristics. Judges will evaluate horses based upon standardized scoring and marking system used in official dressage, jumping events, and other officially sanctioned events. The factors that are considered during judgement include, but are not limited to jumping ability, speed, quality of walk, trot, and canter, movement to music, temperament, ability to cut cows, ability to perform specialized gaits, ability to pull carts, racing (track and cross-country), ability to change leads during canter or gallop, and the ability to maneuver around a course or objects.

c. Information Gathered from Veterinary Assays

The present disclosure provides methods for gathering non-genetic information about a non-human animal from clinical/veterinary assay obtained from the animal pertaining to one or more predetermined traits. As technology and veterinary science have advanced, clinical laboratory testing has become useful in determining the parentage, health, and any ongoing infections in the animal. Just as in humans, blood analysis can be performed to assess the health of the animal such as, for example, measuring blood sugar levels, blood urea nitrogen, amylase, total calcium, and many other clinical lab values. Equipment manufacturers have also developed new equipment which can be configured to allow X-rays, CAT scans, PET scans, and several other advanced imaging techniques with horses and other large animals. Therefore, a variety of information may be measured, especially those related to traits of interest, including those related or thought to relate to performance characteristics, physical structure or disease predisposition. These measurements may include, but are not limited to, conformational and physiological parameters such as height, weight, limb length, limb angle, muscle volume, resting heart rate, time to resting heart rate after physical exertion, blood pressure, maximum oxygen uptake (VO₂ₘₐₓ), maximum carbon dioxide production (VCO₂ₘₐₓ), blood volume at rest and exercise, rebreathing measurements of lung volumes, maximum sprint speed, heart size, complete blood counter including white blood cell count, red blood cell count, hemoglobin levels, hematocrit, platelet count, and other blood cell morphology and biomarker evaluations and measurements, metabolic assays including blood sugar, protein, cholesterol, BUN, and interleukin-6 levels, DNA-based assessment of telomere length, fibrinogen levels, fecal ulcer PCR, transthyretin levels, and health parameters such as history of joint, skin, and diseases or conditions such as cardiovascular disease, orthopedic diseases, chronic obstructive pulmonary disease, pulmonary “bleeding” during extreme exertion, muscle diseases like exertional rhabdomyolysis, immune system disorders causing sarcoid tumors, and insect bite hypersensitivity. The condition may include normal, apparently normal, pre-clinical disease, overt disease, progress and/or stage of disease, undiagnosed or unclassified conditions, presence of drugs, response to exercise, response to vaccines, therapies, nutritional states, and response to environmental conditions. The disease may include inflammation or involvement of the immune system, and conditions affecting respiratory, musculoskeletal, urinary, gastrointestinal, adnexal, cardiovascular, reticuloendothelial, nervous, special senses, reproductive, and integument systems. Such conditions in the horse include laminitis, lameness, viral or bacterial disease, colic, gastritis, gastric ulcers, respiratory ailments, epistaxis, fractures, musculoskeletal damage or disorders, and joint disease, among others (e.g., any one of the conditions disclosed in Table 10). The listed set of physical and physiological parameters that may be tested according to the methods described herein are not exhaustive and additional parameters may also be subject to clinical/veterinary assessment.

The aforementioned non-genetic information gathered from secondary sources may be used independently or in combination with one or more pieces of genetic information about a known or a desired non-human animal in order to produce a holistic assessment of the animal. Such an assessment may be useful for purchasing or breeding decisions by informing the buyer or breeder regarding any known predispositions or bases for genetically-linked traits or conditions, or by informing the buyer or breeder about known history of health conditions, past diagnostic assessments, and past performance in specific competitions or disciplines. Such information may be correlated within the database system, e.g., when an observed non-genetic source of information about an animal's health or behavior is reflected in the genetic profile of the animal. Alternatively, the non-genetic information can be independent or complementary to the genetic information.

### Methods for Interrogating the Database System

The present disclosure provides methods for interrogating a computer-implemented database system to identify one or more breeding pairs capable of producing a non-human offspring animal having one or more predetermined traits, such as a database system storing a panel of at least 2 (e.g., 2, 5, 10, 20, 40, 80, 100, 200, 300, 500, 1000, or more) predetermined traits, such as, e.g., a panel of at least 2-40 (e.g., 2, 3, 4, 5, 6, 7, 8, 9, 10, 15, 20, 25, 35, or 40) predetermined traits. A user may interrogate the database system by selecting one or more (e.g., 1, 5, 10, 20, 40, 80, 100, 200, 300, 500, 1000, or more) predetermined traits desired in the offspring animal using the database system. In particular, the selection may be made from a panel of 2-40 (e.g., 2, 3, 4, 5, 6, 7, 8, 9, 10, 15, 20, 25, 35, or 40) traits. The user may further use the database system to identify the one or more breeding pairs capable of producing the offspring animal. For each identified breeding pair, the database system can be used to generate a probability matrix that includes an array of probability values pertaining to the likelihood that the offspring animal produced by each said breeding pair will have the one or more predetermined traits and displaying the probability matrix on a computer-implemented interface (e.g., such as an interface described herein). Generally, information about the breeding pair used to produce the probability matrix was previously integrated into the database system according to the methods described herein. Such information may include, e.g., information about the results of previously performed genetic diagnostic testing and/or non-genetic assessment of each of the breeder animals in the breeding pair (e.g., genetic markers for specific physical traits (e.g., size, coloration, patterns), behavioral traits such as temperament, speed, endurance, and gait, and metrics for ancestry and inbreeding, such as, e.g., shared cM, kinship coefficient, heterozygosity score, inbreeding coefficient (F), and identity-by-descent). The user may then select, based on the array of probability values, one or more of the breeding pairs likely to produce the offspring animal having the one or more predetermined traits. If the user is satisfied with the likelihood of producing the desired animal based on the results of the probability matrix, the user may continue with breeding the one or more breeding pairs to produce the offspring animal. The user may also use the database system to confirm the presence of one or more (e.g., 1, 5, 10, 20, 40, 80, 100, 200, 300, 500, 1000, or more) predetermined traits in the offspring animal, such as, e.g., 2-40 (e.g., 2, 3, 4, 5, 6, 7, 8, 9, 10, 15, 20, 25, 35, or 40) predetermined traits. Once the user has confirmed the presence of the one or more predetermined traits in the offspring animal, the user may then register the offspring animal the database system as a breeder animal.

### Use of the Database System to Produce Guidelines for Husbandry of a Non-Human Animal

The present disclosure further provides a method of producing a set of guidelines for breeding of a non-human animal (e.g., a non-human animal that is used in a breeding pair to produce an offspring animal). For example, a user may utilize the database system of the invention to identify the non-human animal and assess the presence of one or more predetermined traits in the non-human animal. Assessment of the presence of the one or more predetermine traits may include generating a probability matrix for the non-human animal using the database system and displaying the probability matrix on a graphical user interface. The probability matrix may include an array of probability values pertaining to the likelihood that the non-human animal has the one or more predetermined traits. The user may then determine, based on the assessment of the presence of the one or more predetermined traits in the non-human animal, one or more conditions under which the non-human animal will develop or is at risk of developing a disease or non-disease condition (e.g., any one of the conditions listed in Table 10, among others). If the database system has positively identified the non-human animal as being at risk of developing a disease or non-disease condition, the database system can generate the set of guidelines for breeding of the non-human animal to avoid manifestation of the disease or non-disease condition.

The set of guidelines may include one or more (e.g., 1, 2, 3, 4, 5, 6, 7, 8, 9, 10, or more) recommendations for mitigating or avoiding the one or more conditions that would result in manifestation of, or one or more symptoms of, the disease or non-disease condition and/or for reducing the likelihood of producing an effect in the offspring animal resulting from development of the disease or non-disease condition. In instances where the non-human animal is used for breeding to produce an offspring animal, the non-human animal may be a female parent animal or a male parent animal. In such instances, the set of guidelines produced by the database system may provide one or more recommendations that increases the likelihood of producing a non-human offspring animal having one or more predetermined traits. Generally, the one or more recommendations included in the set of guidelines may pertain to a diet, exercise regime, discipline, environmental exposure, medication, supplementary treatments, training, conditioning, use, and/or limitation of the non-human animal. For example, a user may select an non-human animal (e.g., a foal) to have a specific mutation to increase the likelihood of the foal having white markings with blue eyes. This particular mutation is likely to also result in deafness in the animal. To facilitate proper care and management of the animal, the method of the disclosure would provide guidelines (e.g., recommendations or instructions) for training or acclimating an animal that is hearing impaired or potentially requiring eye protection in bright environments. As another example, a user may apply the disclosed methods and systems to create a horse that is genetically predisposed to have exceptional jumping ability at maturity. The present disclosure would then provide guidelines to feed the animal a diet that is low in protein to avoid high rate of growth or to provide injections of joint supplements (e.g., Adequan) to ensure proper joint lubrication and preparation.

### Methods of Generating the Database System

A. Integration of Non-Human Animals into the Database System

The present disclosure further provides a method of generating a computer-implemented database system for producing a non-human offspring animal having one or more predetermined traits. The database system may be generated, e.g., by having a user provide information about the one or more predetermined traits from a plurality of non-human animals of a same type as the offspring animal. The information may include results of previously performed genetic testing and/or non-genetic assessment of the plurality of non-human animals, such as, e.g., results that identify and/or confirm the plurality of non-human animals as having the one or more of the predetermined traits. Such information may then be catalogued into the database system. The database system may be configured for interrogation by the user, e.g., in order to produce a probability matrix based on the catalogued information following the interrogation, such as, e.g., a probability matrix containing an array of probability values pertaining to the likelihood of the selected breeding pair producing a non-human offspring animal having the one or more predetermined traits or a probability matrix includes an array of probability values pertaining to the likelihood of the plurality of non-human animals having the one or more predetermined traits. Interrogation of the database system may include selecting one or more predetermined traits desired in the offspring animal and identifying from the plurality of non-human animals a breeding pair capable of producing said offspring animal. The user may further interrogate the database to select a breeding pair capable of producing the offspring having the one or more predetermined traits.

Information included in the non-genetic assessment described above may include, e.g., (i) information obtained from someone knowledgeable about the non-human animal pertaining to the one or more predetermined traits; (ii) results of one or more clinical assays performed on biological tissue obtained from the non-human animal pertaining to the one or more predetermined traits; (iii) results of one or more diagnostic tests performed on the non-human animal pertaining to the one or more predetermined traits; and/or (iv) scores assigned to the non-human animal during a competition pertaining to the one or more predetermined traits.

B. Trait Discovery

The methods and systems disclosed herein may be used to facilitate the discovery of previously undetected or undiagnosed traits in a non-human animal (e.g., a horse). In particular, the database system of the present disclosure may promote the discovery of previously unrecognized traits in the non-human animal by allowing an animal owner to provide or request results of genetic diagnostic testing on the animal and to provide non-genetic information about the animal (e.g., non-genetic information described herein) using the database system. Once the genetic and/or non-genetic information is integrated into the database system, it can be cross-referenced to genetic and/or non-genetic information of one or more animals previously catalogued in the database system to determine whether the animal has the previously unrecognized trait. For example, a horse of interest may have a particular gait pattern, the genetic basis of which was previously unknown. Accordingly, the user may submit or request genetic testing of the animal and provide photos and/or videos of the animal locomoting using the database system described herein. The database system subsequently cross-references the results of the genetic testing to the genetic profiles of horses previously catalogued in the database to identify, e.g., a novel variant of the DRMT3 gene responsible for the particular gait pattern of the subject horse. Furthermore, the gait of one or more horses previously catalogued in the database system can be assessed to determine whether the novel DMRT3 gene variant can robustly predict a particular gait behavior across multiple animals. Moreover, upon the discovery of a novel genetic variant in the subject horse, genetic modification can be performed in the horse to modify the animal's gait according with the phenotype desired by the horse owner. Relatedly, the severity of a particular gait trait in a horse may be graded with the number of particular genetic mutations or SNPs in the DMRT3 gene. Thus, the novel DMRT3 variant(s) discovered using the database system of the disclosure may be used to predict (e.g., by extrapolation) the severity of a particular gait trait when two or more mutations or SNPs are present. Subsequent to the prediction step, the newly discovered relationship between the number of mutations or SNPs at a genetic site or a particular gene, e.g., the DMRT3 gene, and a particular behavior, e.g., a gait behavior, can be used to produce a genetically modified horse having a desired behavior, e.g., a gait behavior, that could not have been generated without the previously unrecognized genetic variant, such as the DMRT3 variant of this example. A similar approach can be applied across a number of different traits, such as, e.g., any one of the traits disclosed herein. Finally, once the previously unrecognized trait is identified using the database system, it can be catalogued within the system for further trait discovery, production of non-human animals, and/or genetic modification.

The “Trait Discovery” feature of the disclosed database system further allows the user to perform any one of the following tasks: (1) verify a previous non-genetic assessment of a non-human animal by performing genetic testing on the animal using the database system; (2) identify a previously unidentified susceptibility to a disease or non-disease condition and use this information to modify husbandry practice with respect to the animal in order to accommodate the susceptibility; (3) identify a previously unrecognized association between two or more genes; and (4) identify a previously unrecognized association between two or more traits.

C. Database Architecture

The database systems of the present disclosure may be queried to identify animals having one or more predetermined traits by user-provided input criteria. As described herein, a computer-implemented user interface can be used to receive user input pertaining to one or more predetermined traits of the animal. The input dataset can be converted and expanded by a conversion/formatting engine of the system into a more versatile format and stored in a separate database subsystem (e.g., a user input subsystem). A comparison engine of the database system can be used to perform a comparison between information in the user input subsystem and the genetic profile and/or secondary source information subsystems to identify candidate non-human animals having one or more predetermined traits. The comparison engine can tabulate a list of all possible combinations of traits and then perform a comparison of those combinations with traits contained within the genetic profile subsystem and/or the secondary source information subsystem. The comparison engine can store those combinations that are found to occur and meet certain selection criteria in a separate database subsystem (e.g., a match subsystem) along with a numerical frequency of occurrence obtained as a count during the comparison. A statistical computation engine of the system can perform statistical computations based on the genetic and/or non-genetic information of the animal to obtain results (e.g., numerical probability values) for the likelihood of producing an animal having the one or more predetermined traits as well as, for example, the likelihood of the animal having a lethal genotype or of the animal having or developing a genetic disorder.

According to the present disclosure a database system can be used which contains a subsystem for accessing user input information pertaining to one or more predetermined traits, a second subsystem for accessing a set of database subsystems containing genetic and non-genetic information associated with a plurality of trait-positive and trait-negative animals, a data processing subsystem for identifying combinations of genetic and non-genetic traits associated with trait-positive animals, but not with trait-negative animals, and a calculating subsystem for determining a set of statistical results that indicates a strength of association between the combinations of genetic and non-genetic traits in trait-positive animals with the user input information. The system can also include a communications subsystem for retrieving at least some of genetic and non-genetic traits from at least one external database; a ranking subsystem for ranking the co-occurring traits according to the strength of the association of each co-occurring trait with the user input information; and a storage subsystem for storing the set of statistical results indicating the strength of association between the combinations of genetic and non-genetic traits and the user input information.

In a particular example, the computer-implemented database system may include a database subsystem(s) containing genetic information pertaining to one or more predetermined traits that can be used to identify an existing non-human animal having the one or more predetermined traits or one or more members of a breeding pair that could be bred to produce an offspring animal having the one or more predetermined traits. Such database subsystems may be instantiated as, e.g., a referential table containing information about genetic determinants of a particular phenotypic trait. The referential table may include, e.g., information pertaining to expression of a specific gene or a combination of genes or the allelic variants thereof that would identify a non-human animal as having the desired trait(s) or that would indicate that a non-human animal expressing said gene or combination of genes would be capable of producing an offspring animal having the desired trait. Said referential table may also include information pertaining to a mutation(s) or polymorphism(s) (e.g., SNP) in a non-coding region (e.g., intron, 5′ untranslated region (UTR), or 3′ UTR) or regulatory region (e.g., promoter, enhancer, or silencer) of a particular gene that may affect its expression.

For example, in the case of coat color, the database system may contain a referential table containing information pertaining to the genetic determinants of coat color in the non-human animal (e.g., a horse). A user wishing to identify a horse having a particular desired coat color could use the user interface of the present disclosure to use said coat color as a selection criterion. The user interface, being operably connected to the database system of the disclosure, may query the database system to identify the genes that are expressed in a horse to produce the desired coat color. Subsequently, the database system can search across the animals already catalogued in the database system to identify one or more non-human animals expressing the gene or combination of genes that produce said coat color in horses. Table 2 below provides an exemplary referential table for coat color selection that may be used in conjunction with the database system of the disclosure.

In another example, the database system of the disclosure can similarly be used to produce an animal having a particular coat color with specific coat color modifications (i.e., markings, such as, e.g., any one of the coat color markings described herein.). Similarly, the database system may contain a referential table containing information pertaining to the genetic determinants of coat color modifications in the horse. A user wishing to identify a horse having a particular desired coat color with a desired modification(s) could use the user interface of the present disclosure to use said coat color modification as a selection criterion. The user interface can query the database system to identify the genes that are expressed in a horse to produce the desired coat color and modifications. Subsequently, the database system can search across the animals already catalogued in the database system to identify one or more non-human animals expressing the gene or combination of genes that produce said coat color and modifications in horses. Table 3 and Table 4 below provide exemplary referential tables for coat color modification selection that may be used in conjunction with the database system of the disclosure. The genetic determinants for coat color modifications can be combined with the genetic determinants of the various coat color provided in Table 2 to identify horses having complex coat colors and marking pattern genotypes (see Table 5).

In producing a horse having a desired coat color and/or coat color modification, a user may need to take caution to avoid producing a horse with a potentially deleterious or fatal genotype. Accordingly, the database system of the disclosure can be used to produce an animal having a particular coat color with or without a specific coat color modifications that avoids a deleterious or a fatal genotype. To this end, the database system of the disclosure may contain a referential table containing information pertaining to the genetic determinants of coat color and/or coat color modifications in the horse that produce a deleterious of fatal phenotype. A user wishing to identify a horse lacking the potentially deleterious or fatal genotype could use the user interface of the present disclosure to identify animals lacking this genotype. The user interface can query the database system to identify the genes that are expressed in a horse to produce the deleterious or fatal phenotype. Subsequently, the database system can search across the animals already catalogued in the database system to identify one or more non-human animals lacking the gene or combination of genes that produce the potentially deleterious or fatal phenotype. Table 6 below provides an exemplary referential table for potentially deleterious or fatal coat color/modification genotypes to be used in conjunction with the database system of the disclosure.

In another example, the database system of the disclosure can be used to produce an animal that does not express one or more disease or non-disease conditions (e.g., any one of the disease or non-disease conditions described in Table 10). To this end, the database system may contain a referential table containing information pertaining to the genetic determinants of a plurality of disease or non-disease conditions. A user wishing to identify a horse does not exhibit a particular disease or non-disease condition can use the user interface of the present disclosure to select one or more disease or non-disease conditions that are desirably avoided in the animal. The user interface can query the database system to identify the genes that are expressed in a horse to produce the disease or non-disease condition. Subsequently, the database system can search across the animals already catalogued in the database system to identify one or more non-human animals that do not express the gene(s) that produce the disease or non-disease condition in horses. Table 7 and Table 8 below provide an exemplary referential table for disease and non-disease conditions that may be used in conjunction with the database system of the disclosure.

In yet another example, the database system of the disclosure can be used to produce an animal that exhibits a particular behavioral (e.g., curiosity/vigilance) or ability (e.g., gaited/non-gaited/carrier) trait. To this end, the database system may contain a referential table containing information pertaining to the genetic determinants of a plurality behavioral/ability traits. A user wishing to identify a horse exhibiting one or more behavioral/ability traits can use the user interface of the present disclosure to select one or more behavioral/ability traits desired in the horse. The user interface can query the database system to identify the genes that are expressed in a horse to produce the one or more behavioral/ability traits. Subsequently, the database system can search across the animals already catalogued in the database system to identify one or more non-human animals that express the gene(s) that produce the behavioral/ability trait in horses. Table 9 below provides an exemplary referential table for behavioral/ability traits and their genetic determinants that may be used in conjunction with the database system of the disclosure.

TABLE 2
Referential table for coat color in horses

| ID | Coat color | Also known as | ASIPA | BIEC2816500E | TBX3nd1 | TBX3nd1alt | SLC36a1CH | PMEL17Z | SLC45A2CR | SLC45A2PRL |
|---|---|---|---|---|---|---|---|---|---|---|
| 100 | Amber Dun | Amber | X | X | X | X | X | n/n | n/n | n/n |
| | | Champagne | | | | | | | | |
| | | Dun | | | | | | | | |
| 106 | Amber Dun | Pseudocream | X | X | X | X | X | n/n | n/n | X/X |
| | Pearl | Amber | | | | | | | | |
| 2 | Bay | | X | X | n/n | n/n | n/n | n/n | n/n | n/n |
| 3 | Black | | n/n | X | n/n | n/n | n/n | n/n | n/n | n/n |
| 5 | Buckskin | Bay Cream | X | X | n/n | n/n | n/n | n/n | X/n | n/n |
| 9 | Gold | | n/n | n/n | n/n | n/n | X | n/n | n/n | n/n |
| | Champagne | | | | | | | | | |
| 8 | Classic | | n/n | X | n/n | n/n | X | n/n | n/n | n/n |
| | Champagne | | | | | | | | | |
| 7 | Amber | | X | X | n/n | n/n | X | n/n | n/n | n/n |
| | Champagne | | | | | | | | | |
| 101 | Champagne | Classic | n/n | X | X | X | X | n/n | n/n | n/n |
| | Dun | Champagne | | | | | | | | |
| | | Dun | | | | | | | | |
| 107 | Champagne | Pseudocream | n/n | X | X | X | X | n/n | n/n | X/X |
| | Dun Pearl | Classic | | | | | | | | |
| 93 | Champagne | | n/n | n/n | n/n | n/n | X | n/n | n/n | X/X |
| | Gold Pearl | | | | | | | | | |
| 91 | Champagne | | n/n | X | n/n | n/n | X | n/n | n/n | X/X |
| | Classic Pearl | | | | | | | | | |
| 89 | Champagne | | X | X | n/n | n/n | X | n/n | n/n | X/X |
| | Amber Pearl | | | | | | | | | |
| 92 | Gold Cream | | n/n | n/n | n/n | n/n | X | n/n | X | n/n |
| 90 | Classic | | n/n | X | n/n | n/n | X | n/n | X | n/n |
| | Cream | | | | | | | | | |
| 88 | Amber | | X | X | n/n | n/n | X | n/n | X | n/n |
| | Cream | | | | | | | | | |
| 99 | Cream Grullo | Dun Black | n/n | X | X | X | n/n | n/n | X/X | n/n |
| | | Double | | | | | | | | |
| | | Cream | | | | | | | | |
| 96 | Chestnut | | n/n | n/n | n/n | n/n | n/n | n/n | X/n | X/n |
| | Cream Pearl | | | | | | | | | |
| 95 | Black Cream | | n/n | X | n/n | n/n | n/n | n/n | X/n | X/n |
| | Pearl | | | | | | | | | |
| 94 | Bay Cream | | X | X | n/n | n/n | n/n | n/n | X/n | X/n |
| | Pearl | | | | | | | | | |
| 10 | Cremello | Dun Chestnut | n/n | n/n | n/n | n/n | n/n | n/n | X/X | n/n |
| | | Double | | | | | | | | |
| | | Cream | | | | | | | | |
| 18 | Red Dun | | n/n | n/n | X | X | n/n | n/n | n/n | n/n |
| 105 | Apricot Dun | | n/n | n/n | X | X | n/n | n/n | n/n | X/X |
| 102 | Gold Dun | | n/n | n/n | X | X | X | n/n | n/n | n/n |
| 17 | Classic Dun | | X | X | X | X | n/n | n/n | n/n | n/n |
| 104 | Dun Pearl | | n/n | X | X | X | n/n | n/n | n/n | X/X |
| | Black | | | | | | | | | |
| 103 | Dun Pearl | | X | X | X | X | n/n | n/n | n/n | X/X |
| | Bay | | | | | | | | | |
| 19 | Dunolino | Dun Chestnut | n/n | n/n | X | X | n/n | n/n | X/n | n/n |
| | | Cream | | | | | | | | |
| 20 | Dunskin | Dun Bay | X | X | X | X | n/n | n/n | X/n | n/n |
| | | Cream | | | | | | | | |
| 108 | Gold Dun | Pseudocream | n/n | n/n | X | X | X | n/n | n/n | X/X |
| | Pearl | Gold | | | | | | | | |
| 32 | Grullo | Black Dun | n/n | X | X | X | n/n | n/n | n/n | n/n |
| 57 | Palomino | Chestnut | | n/n | n/n | n/n | n/n | n/n | X/n | n/n |
| | | Cream | | | | | | | | |
| 58 | Apricot Pearl | | n/n | n/n | n/n | n/n | n/n | n/n | n/n | X/X |
| 62 | Black Pearl | | n/n | X | n/n | n/n | n/n | n/n | n/n | X/n |
| 60 | Bay Pearl | | X | X | n/n | n/n | n/n | n/n | n/n | X/n |
| 64 | Perlino | Bay Double | X | X | n/n | n/n | n/n | n/n | X/X | n/n |
| | | Cream | | | | | | | | |
| 97 | Perlino Dun | Dun Bay | X | X | X | X | n/n | n/n | X/X | n/n |
| | | Double | | | | | | | | |
| | | Cream | | | | | | | | |
| 67 | Red | Chestnut, | | n/n | n/n | n/n | n/n | n/n | n/n | n/n |
| | | Sorrel | | | | | | | | |
| 121 | Silver Amber | Silver Amber | X | X | X | X | X | X | n/n | n/n |
| | Dun | Champagne | | | | | | | | |
| | | Dun | | | | | | | | |
| 113 | Silver | | n/n | X | n/n | n/n | X | X | n/n | n/n |
| | Classic | | | | | | | | | |
| | Champagne | | | | | | | | | |
| 111 | Silver Amber | | X | X | n/n | n/n | X | X | n/n | n/n |
| | Champagne | | | | | | | | | |
| 122 | Silver | | n/n | X | X | X | X | X | n/n | n/n |
| | Champagne | | | | | | | | | |
| | Dun | | | | | | | | | |
| 114 | Silver | | n/n | X | n/n | n/n | X | X | n/n | X/X |
| | Classic | | | | | | | | | |
| | Champagne | | | | | | | | | |
| | Pearl | | | | | | | | | |
| 112 | Silver | | X | X | n/n | n/n | X | X | n/n | X/X |
| | Classic | | | | | | | | | |
| | Amber Pearl | | | | | | | | | |
| 116 | Silver Black | | n/n | X | n/n | n/n | n/n | X | X/n | X/n |
| | Cream Pearl | | | | | | | | | |
| 115 | Silver Bay | | X | X | n/n | n/n | n/n | X | X/n | X/n |
| | Cream Pearl | | | | | | | | | |
| 71 | Silver Dapple | Silver Black | n/n | X | n/n | n/n | n/n | X | n/n | n/n |
| 300 | Silver Red | | X or n | n/n | n/n | n/n | n/n | X | n/n | n/n |
| 70 | Silver Bay | | X | X | n/n | n/n | n/n | X | n/n | n/n |
| 72 | Silver | | X | X | n/n | n/n | n/n | X | X/n | n/n |
| | Buckskin | | | | | | | | | |
| 120 | Silver Cream | Silver Dun | n/n | X | X | X | n/n | X | X/X | n/n |
| | Grullo | Black Double | | | | | | | | |
| | | Cream | | | | | | | | |
| 73 | Silver Dun | Silver Dapple | X | X | X | X | n/n | X | n/n | n/n |
| | | Dun | | | | | | | | |
| 117 | Silver | Silver Dun | X | X | X | X | n/n | X | X/n | n/n |
| | Dunskin | Bay Cream | | | | | | | | |
| 119 | Silver Grullo | Silver Dun | n/n | X | X | X | n/n | X | n/n | n/n |
| | | Black | | | | | | | | |
| 126 | Silver Pearl | | n/n | X | n/n | n/n | n/n | X | n/n | X/X |
| | Black | | | | | | | | | |
| 125 | Silver Pearl | | X | X | n/n | n/n | n/n | X | n/n | X/X |
| | Bay | | | | | | | | | |
| 109 | Silver Perlino | Silver Bay | X | X | n/n | n/n | n/n | X | X/X | n/n |
| | | Double | | | | | | | | |
| | | Cream | | | | | | | | |
| 118 | Silver Perlino | Silver Dun | X | X | X | X | n/n | X | X/X | n/n |
| | Dun | Bay Double | | | | | | | | |
| | | Cream | | | | | | | | |
| 124 | Silver Dun | | n/n | X | X | X | n/n | X | n/n | X/X |
| | Pearl Black | | | | | | | | | |
| 123 | Silver Dun | | X | X | X | X | n/n | X | n/n | X/X |
| | Pearl Bay | | | | | | | | | |
| 110 | Silver Smoky | Silver Black | n/n | X | n/n | n/n | n/n | X | X/X | n/n |
| | Cream | Double | | | | | | | | |
| | | Cream | | | | | | | | |
| 74 | Smoky Black | Black Cream | n/n | X | n/n | n/n | n/n | n/n | X/n | n/n |
| 75 | Smoky | Black Double | n/n | X | n/n | n/n | n/n | n/n | X/X | n/n |
| | Cream | Cream | | | | | | | | |
| 98 | Smoky | Dun Black | n/n | X | X | X | n/n | n/n | X/n | n/n |
| | Grullo | Cream | | | | | | | | |
| 171 | Champagne | | | | | | X | | | X/X |
| | Pearl | | | | | | | | | |
| 172 | Cream Pearl | | | | | | | | X/n | X/n |
| 173 | Dun Pearl | | | | X | X | | | | X/X |
| 174 | Silver | | | | | | X | X | | |
| | Champagne | | | | | | | | | |
| 175 | Silver | | | | | | X | X | | X/X |
| | Champagne | | | | | | | | | |
| | Pearl | | | | | | | | | |
| 176 | Silver Cream | | | | | | | X | X/n | X/n |
| | Pearl | | | | | | | | | |
| 177 | Silver Pearl | | | X | | | | X | | X/X |
| 178 | Silver Dun | | | X | X | X | | X | | X/X |
| | Pearl | | | | | | | | | |
| 185 | Silver Bay | | X | X | | | | X | X | |
| | Cream | | | | | | | | | |
| | Chestnut/Red/ | | X or n | n/n | | | | | | |
| | Sorrel | | | | | | | | | |

TABLE 3
Referential table of coat color modifications in horses

| | Coat color | | | | | | | | |
|---|---|---|---|---|---|---|---|---|---|
| ID | modification | EDNRB_TC_to_AG | STX17A | TRPM1LP | RFWD3PATN1 | KIT1SB12 | TobianoE2 | KRT25_CurlyHipo | SP6_Curly |
| 14 | Dominant | | | | | | | | |
| | White | | | | | | | | |
| | W20/n | | | | | | | | |
| 15 | Dominant | | | | | | | | |
| | White | | | | | | | | |
| | W20/W20 | | | | | | | | |
| 16 | Dominant | | | | | | | | |
| | White | | | | | | | | |
| | W5/W20 | | | | | | | | |
| 31 | Grey | | X | | | | | | |
| 25 | Leopard | | | X/n | | | | | |
| | Spotting | | | | | | | | |
| | Complex | | | | | | | | |
| | (varnish) | | | | | | | | |
| | Leopard | | | X/X | n/n | | | | |
| | Spotting | | | | | | | | |
| | Complex | | | | | | | | |
| | (varnish) | | | | | | | | |
| | Leopard | | | X/n | X/n | | | | |
| | Spotting | | | | | | | | |
| | Complex | | | | | | | | |
| | (varnish | | | | | | | | |
| | leopard) | | | | | | | | |
| | Leopard | | | X/X | X/n | | | | |
| | Spotting | | | | | | | | |
| | Complex | | | | | | | | |
| | (fewspot) | | | | | | | | |
| | Leopard | | | X/n | X/X | | | | |
| | Spotting | | | | | | | | |
| | Complex | | | | | | | | |
| | (leopard) | | | | | | | | |
| | Leopard | | | X/X | X/X | | | | |
| | Spotting | | | | | | | | |
| | Complex | | | | | | | | |
| 42 | Pattern 1 | | | | X | | | | |
| 56 | Overo | X/n | | | | | | | |
| | (LWO/n) | | | | | | | | |
| 68 | Sabino1 | | | | | X | | | |
| 76 | Splashed | | | | | | | | |
| | White 1 | | | | | | | | |
| 77 | Splashed | | | | | | | | |
| | White 2 | | | | | | | | |
| 78 | Splashed | | | | | | | | |
| | White 3 | | | | | | | | |
| 79 | Splashed | | | | | | | | |
| | White 4 | | | | | | | | |
| 80 | Splashed | | | | | | | | |
| | White 5 | | | | | | | | |
| 84 | Tobiano | | | | | | X | | |
| 85 | Tovero | X/n | | | | | X | | |
| 184 | Curly Coat | | | | | | | X | X |

| | Coat color | | | | | | | | |
|---|---|---|---|---|---|---|---|---|---|
| ID | modification | MITFS-W1 | MITFS-W3 | MITFS-W5 | PAX3SW2 | PAX3P32R | KITW5 | KITW20 | Description |
| 14 | Dominant | | | | | | | X/n | One W20 variant; may result |
| | White | | | | | | | | in White markings. |
| | W20/n | | | | | | | | |
| 15 | Dominant | | | | | | | X/X | Two W20 variant; may result |
| | White | | | | | | | | in White markings. |
| | W20/W20 | | | | | | | | |
| 16 | Dominant | | | | | | X/n | X/n | One W20 variant and one |
| | White | | | | | | | | W5 variant; may result in |
| | W5/W20 | | | | | | | | White markings. |
| 31 | Grey | | | | | | | | Grey variant; may cause |
| | | | | | | | | | progressive greying of entire |
| | | | | | | | | | coat (possibly appearing |
| | | | | | | | | | white at maturity) - increased |
| | | | | | | | | | risk of melanoma |
| 25 | Leopard | | | | | | | | One LP variant; may result in |
| | Spotting | | | | | | | | White markings. |
| | Complex | | | | | | | | |
| | (varnish) | | | | | | | | |
| | Leopard | | | | | | | | |
| | Spotting | | | | | | | | |
| | Complex | | | | | | | | |
| | (varnish) | | | | | | | | |
| | Leopard | | | | | | | | |
| | Spotting | | | | | | | | |
| | Complex | | | | | | | | |
| | (varnish | | | | | | | | |
| | leopard) | | | | | | | | |
| | Leopard | | | | | | | | |
| | Spotting | | | | | | | | |
| | Complex | | | | | | | | |
| | (fewspot) | | | | | | | | |
| | Leopard | | | | | | | | |
| | Spotting | | | | | | | | |
| | Complex | | | | | | | | |
| | (leopard) | | | | | | | | |
| | Leopard | | | | | | | | |
| | Spotting | | | | | | | | |
| | Complex | | | | | | | | |
| 42 | Pattern 1 | | | | | | | | One or two PATN1 variant in |
| | | | | | | | | | combination; may result in a |
| | | | | | | | | | “leopard pattern with |
| | | | | | | | | | Leopard Spotting Complex |
| | | | | | | | | | (LP/n).” |
| 56 | Overo | | | | | | | | One LWO variant; may result |
| | (LWO/n) | | | | | | | | in White markings. |
| 68 | Sabino1 | | | | | | | | One SB1 variant; may result |
| | | | | | | | | | in White markings. |
| 76 | Splashed | X | | | | | | | One Splashed White One |
| | White 1 | | | | | | | | variant; may result in White |
| | | | | | | | | | markings on the face, legs |
| | | | | | | | | | and body with possible blue |
| | | | | | | | | | eye(s) |
| 77 | Splashed | | | | X/n | | | | One Splashed White Two |
| | White 2 | | | | | | | | variant; may result in White |
| | | | | | | | | | markings on face/head, legs |
| | | | | | | | | | and body with possible blue |
| | | | | | | | | | eye(s) and risk of deafness in |
| | | | | | | | | | one or both ears |
| 78 | Splashed | | X/n | | | | | | One Splashed White Three |
| | White 3 | | | | | | | | variant; may result in White |
| | | | | | | | | | markings |
| 79 | Splashed | | | | | X | | | One Splashed White Four |
| | White 4 | | | | | | | | variant, may result in White |
| | | | | | | | | | markings. |
| 80 | Splashed | | | X/n | | | | | One Splashed White Five |
| | White 5 | | | | | | | | variant; may result white/bald |
| | | | | | | | | | face marking, blue eye(s), |
| | | | | | | | | | white on legs and body with |
| | | | | | | | | | high risk for deafness in one |
| | | | | | | | | | or both ears |
| 84 | Tobiano | | | | | | | | One Tobiano variant; may |
| | | | | | | | | | result in large white markings |
| | | | | | | | | | on body, face and legs |
| | | | | | | | | | (occasionally produces |
| | | | | | | | | | “minimal” or “slipped” |
| | | | | | | | | | Tobiano horse with very little |
| | | | | | | | | | white only on face and lower |
| | | | | | | | | | legs (ongoing heritability |
| | | | | | | | | | study for minimal Tobiano) |
| 85 | Tovero | | | | | | | | Tobiano and Lethal White |
| | | | | | | | | | Overo variants may result in |
| | | | | | | | | | large white spots (to nearly |
| | | | | | | | | | white) with likely blue eye(s) |
| 184 | Curly Coat | | | | | | | | Curly variant; results in curly |
| | | | | | | | | | texture to coat, mane and tail |

TABLE 4
Referential table for coat color modifications in horses (continued)

| ID | Name | ASIPA | BIEC2816500E | TBX3nd1 | TBX3nd1alt | SLC36a1CH | PMEL17Z | SLC45A2CR | SLC45A2PRL |
|---|---|---|---|---|---|---|---|---|---|
| 394 | Agouti (A/A) | X/X | | | | | | | |
| 395 | Agouti (A/a) | X/n | | | | | | | |
| 396 | Black (E/E) | | X/X | | | | | | |
| 397 | Black (E/e) | | X/n | | | | | | |
| 398 | Red (e/e) | | n/n | | | | | | |
| 399 | Dun (D/D) | | | X/X | X/X | | | | |
| 400 | D/nd1**or D/nd2*** | | | X/n | X/n | | | | |
| 401 | Champagne (CH/CH) | | | | | X/X | | | |
| 402 | Champagne (CH/n) | | | | | X/n | | | |
| 403 | Silver (Z/Z) | | | | | | X/X | | |
| 404 | Silver (Z/n) | | | | | | X/n | | |
| 405 | Cream (CR/CR) | | | | | | | X/X | |
| 406 | Cream (CR/n) | | | | | | | X/n | |
| 407 | Pearl (prl/prl) | | | | | | | | X/X |
| 408 | Pearl (prl/n) | | | | | | | | X/n |

TABLE 5
Referential table for genotypes of coat color modifications in combination with various coat color genotypes

| | Coat | | | | | | | | | | |
|---|---|---|---|---|---|---|---|---|---|---|---|
| | color + | | | | | | | | | | EDNRB_{—} |
| Modifi- | modifi- | Also | | | TBX3- | TBX3nd1- | | | | | TC_{—} |
| cation | cation | known as | ASIPA | BIEC2816500E | nd1 | alt | SLC36a1CH | PMEL17Z | SLC45A2CR | SLC45A2PRL | to_AG |
| Overo | Amber | Amber | X | X | X | X | X | n/n | n/n | n/n | X/n |
| | Dun | Cham- | | | | | | | | | |
| | Overo | pagne | | | | | | | | | |
| | | Dun | | | | | | | | | |
| | | Overo | | | | | | | | | |
| | Amber | Pseudo- | X | X | X | X | X | n/n | n/n | X/X | X/n |
| | Dun | cream | | | | | | | | | |
| | Pearl | Amber | | | | | | | | | |
| | Overo | Overo | | | | | | | | | |
| | Bay | | X | X | n/n | n/n | n/n | n/n | n/n | n/n | X/n |
| | Overo | | | | | | | | | | |
| | Black | | n/n | X | n/n | n/n | n/n | n/n | n/n | n/n | X/n |
| | Overo | | | | | | | | | | |
| | Buck- | | X | X | n/n | n/n | n/n | n/n | X/n | n/n | X/n |
| | skin | | | | | | | | | | |
| | Overo | | | | | | | | | | |
| | | Gold | n/n | n/n | n/n | n/n | X | n/n | n/n | n/n | X/n |
| | | Cham- | | | | | | | | | |
| | | pagne | | | | | | | | | |
| | | Overo | | | | | | | | | |
| | | Classic | n/n | X | n/n | n/n | X | n/n | n/n | n/n | X/n |
| | | Cham- | | | | | | | | | |
| | | pagne | | | | | | | | | |
| | | Overo | | | | | | | | | |
| | | Amber | X | X | n/n | n/n | X | n/n | n/n | n/n | X/n |
| | | Cham- | | | | | | | | | |
| | | pagne | | | | | | | | | |
| | | Overo | | | | | | | | | |
| | Cham- | Classic | n/n | X | X | X | X | n/n | n/n | n/n | X/n |
| | pagne | Cham- | | | | | | | | | |
| | Dun | pagne | | | | | | | | | |
| | Overo | Dun | | | | | | | | | |
| | | Overo | | | | | | | | | |
| | Cham- | Pseudo- | n/n | X | X | X | X | n/n | n/n | X/X | X/n |
| | pagne | cream | | | | | | | | | |
| | Dun | Classic | | | | | | | | | |
| | Pearl | Overo | | | | | | | | | |
| | Overo | | | | | | | | | | |
| | | Cham- | n/n | n/n | n/n | n/n | X | n/n | n/n | X/X | X/n |
| | | pagne | | | | | | | | | |
| | | Gold | | | | | | | | | |
| | | Pearl | | | | | | | | | |
| | | Overo | | | | | | | | | |
| | | Cham- | n/n | X | n/n | n/n | X | n/n | n/n | X/X | X/n |
| | | pagne | | | | | | | | | |
| | | Classic | | | | | | | | | |
| | | Pearl | | | | | | | | | |
| | | Overo | | | | | | | | | |
| | | Cham- | X | X | n/n | n/n | X | n/n | n/n | X/X | X/n |
| | | pagne | | | | | | | | | |
| | | Amber | | | | | | | | | |
| | | Pearl | | | | | | | | | |
| | | Overo | | | | | | | | | |
| | | Gold | n/n | n/n | n/n | n/n | X | n/n | X | n/n | X/n |
| | | Cream | | | | | | | | | |
| | | Overo | | | | | | | | | |
| | | Classic | n/n | X | n/n | n/n | X | n/n | X | n/n | X/n |
| | | Cream | | | | | | | | | |
| | | Overo | | | | | | | | | |
| | | Amber | X | X | n/n | n/n | X | n/n | X | n/n | X/n |
| | | Cream | | | | | | | | | |
| | | Overo | | | | | | | | | |
| | Cream | | n/n | X | X | X | n/n | n/n | X/X | n/n | X/n |
| | Grullo | | | | | | | | | | |
| | Overo | | | | | | | | | | |
| | | Chest- | n/n | n/n | n/n | n/n | n/n | n/n | X/n | X/n | X/n |
| | | nut | | | | | | | | | |
| | | Cream | | | | | | | | | |
| | | Pearl | | | | | | | | | |
| | | Overo | | | | | | | | | |
| | | Black | n/n | X | n/n | n/n | n/n | n/n | X/n | X/n | X/n |
| | | Cream | | | | | | | | | |
| | | Pearl | | | | | | | | | |
| | | Overo | | | | | | | | | |
| | | Bay | X | X | n/n | n/n | n/n | n/n | X/n | X/n | X/n |
| | | Cream | | | | | | | | | |
| | | Pearl | | | | | | | | | |
| | | Overo | | | | | | | | | |
| | Cremello | | n/n | n/n | n/n | n/n | n/n | n/n | X/X | n/n | X/n |
| | Overo | | | | | | | | | | |
| | | Red Dun | n/n | n/n | X | X | n/n | n/n | n/n | n/n | X/n |
| | | Overo | | | | | | | | | |
| | | Apricot | n/n | n/n | X | X | n/n | n/n | n/n | X/X | X/n |
| | | Dun | | | | | | | | | |
| | | Overo | | | | | | | | | |
| | | Gold | n/n | n/n | X | X | X | n/n | n/n | n/n | X/n |
| | | Dun | | | | | | | | | |
| | | Overo | | | | | | | | | |
| | | Classic | X | X | X | X | n/n | n/n | n/n | n/n | X/n |
| | | Dun | | | | | | | | | |
| | | Overo | | | | | | | | | |
| | | Dun | n/n | X | X | X | n/n | n/n | n/n | X/X | X/n |
| | | Pearl | | | | | | | | | |
| | | Black | | | | | | | | | |
| | | Overo | | | | | | | | | |
| | | Dun | X | X | X | X | n/n | n/n | n/n | X/X | X/n |
| | | Pearl | | | | | | | | | |
| | | Bay | | | | | | | | | |
| | | Overo | | | | | | | | | |
| | Dunolino | | n/n | n/n | X | X | n/n | n/n | X/n | n/n | X/n |
| | Overo | | | | | | | | | | |
| | Dunskin | | X | X | X | X | n/n | n/n | X/n | n/n | X/n |
| | Overo | | | | | | | | | | |
| | Gold Dun | Pseudo- | n/n | n/n | X | X | X | n/n | n/n | X/X | X/n |
| | Pearl | cream | | | | | | | | | |
| | Overo | Gold | | | | | | | | | |
| | | Overo | | | | | | | | | |
| | Grullo | | n/n | X | X | X | n/n | n/n | n/n | n/n | X/n |
| | Overo | | | | | | | | | | |
| | Palomino | | | n/n | n/n | n/n | n/n | n/n | X/n | n/n | X/n |
| | Overo | | | | | | | | | | |
| | | Apricot | n/n | n/n | n/n | n/n | n/n | n/n | n/n | X/X | X/n |
| | | Pearl | | | | | | | | | |
| | | Overo | | | | | | | | | |
| | | Black | n/n | X | n/n | n/n | n/n | n/n | n/n | X/X | X/n |
| | | Pearl | | | | | | | | | |
| | | Overo | | | | | | | | | |
| | | Bay | X | X | n/n | n/n | n/n | n/n | n/n | X/X | X/n |
| | | Pearl | | | | | | | | | |
| | | Over | | | | | | | | | |
| | Perlino | | X | X | n/n | n/n | n/n | n/n | X/X | n/n | X/n |
| | Overo | | | | | | | | | | |
| | Perlino | | X | X | X | X | n/n | n/n | X/X | n/n | X/n |
| | Dun | | | | | | | | | | |
| | Overo | | | | | | | | | | |
| | Red | Chest- | | n/n | n/n | n/n | n/n | n/n | n/n | n/n | X/n |
| | Overo | nut, | | | | | | | | | |
| | | Sorrel | | | | | | | | | |
| | | Overo | | | | | | | | | |
| | Silver | Silver | X | X | X | X | X | X | n/n | n/n | X/n |
| | Amber | Amber | | | | | | | | | |
| | Dun | Cham- | | | | | | | | | |
| | Overo | pagne | | | | | | | | | |
| | | Dun | | | | | | | | | |
| | | Overo | | | | | | | | | |
| | | Silver | n/n | X | n/n | n/n | X | X | n/n | n/n | X/n |
| | | Classic | | | | | | | | | |
| | | Cham- | | | | | | | | | |
| | | pagne | | | | | | | | | |
| | | Overo | | | | | | | | | |
| | | Silver | X | X | n/n | n/n | X | X | n/n | n/n | X/n |
| | | Amber | | | | | | | | | |
| | | Cham- | | | | | | | | | |
| | | pagne | | | | | | | | | |
| | | Overo | | | | | | | | | |
| | Silver | | n/n | X | X | X | X | X | n/n | n/n | X/n |
| | Cham- | | | | | | | | | | |
| | pagne | | | | | | | | | | |
| | Dun | | | | | | | | | | |
| | Overo | | | | | | | | | | |
| | | Silver | n/n | X | n/n | n/n | X | X | n/n | X/X | X/n |
| | | Classic | | | | | | | | | |
| | | Cham- | | | | | | | | | |
| | | pagne | | | | | | | | | |
| | | Pearl | | | | | | | | | |
| | | Overo | | | | | | | | | |
| | | Silver | X | X | n/n | n/n | X | X | n/n | X/X | X/n |
| | | Classic | | | | | | | | | |
| | | Amber | | | | | | | | | |
| | | Pearl | | | | | | | | | |
| | | Overo | | | | | | | | | |
| | | Silver | n/n | X | n/n | n/n | n/n | X | X/n | X/n | X/n |
| | | Black | | | | | | | | | |
| | | Cream | | | | | | | | | |
| | | Pearl | | | | | | | | | |
| | | Overo | | | | | | | | | |
| | | Silver | X | X | n/n | n/n | n/n | X | X/n | X/n | X/n |
| | | Bay | | | | | | | | | |
| | | Cream | | | | | | | | | |
| | | Pearl | | | | | | | | | |
| | | Overo | | | | | | | | | |
| | Silver | Silver | n/n | X | n/n | n/n | n/n | X | n/n | n/n | X/n |
| | Dapple | Black | | | | | | | | | |
| | Overo | Overo | | | | | | | | | |
| | Silver | | X | X | n/n | n/n | n/n | X | n/n | n/n | X/n |
| | Bay | | | | | | | | | | |
| | Overo | | | | | | | | | | |
| | Silver | | X | X | n/n | n/n | n/n | X | X/n | n/n | X/n |
| | Buck- | | | | | | | | | | |
| | skin | | | | | | | | | | |
| | Overo | | | | | | | | | | |
| | Silver | | n/n | X | X | X | n/n | X | X/X | n/n | X/n |
| | Cream | | | | | | | | | | |
| | Grullo | | | | | | | | | | |
| | Overo | | | | | | | | | | |
| | Silver | Silver | | X | X | X | n/n | X | n/n | n/n | X/n |
| | Dun | Dapple | | | | | | | | | |
| | Overo | Dun | | | | | | | | | |
| | | Overo | | | | | | | | | |
| | Silver | Silver | X | X | X | X | n/n | X | X/n | n/n | X/n |
| | Dunskin | Dun Bay | | | | | | | | | |
| | Overo | Cream | | | | | | | | | |
| | | Overo | | | | | | | | | |
| | Silver | Silver | n/n | X | X | X | n/n | X | n/n | n/n | X/n |
| | Grullo | Dun | | | | | | | | | |
| | Overo | Black | | | | | | | | | |
| | | Overo | | | | | | | | | |
| | | Silver | n/n | X | n/n | n/n | n/n | X | n/n | X/X | X/n |
| | | Pearl | | | | | | | | | |
| | | Black | | | | | | | | | |
| | | Overo | | | | | | | | | |
| | | Silver | X | X | n/n | n/n | n/n | X | n/n | X/X | X/n |
| | | Pearl | | | | | | | | | |
| | | Bay | | | | | | | | | |
| | | Overo | | | | | | | | | |
| | Silver | | X | X | n/n | n/n | n/n | X | X/X | n/n | X/n |
| | Perlino | | | | | | | | | | |
| | Overo | | | | | | | | | | |
| | Silver | | X | X | X | X | n/n | X | X/X | n/n | X/n |
| | Perlino | | | | | | | | | | |
| | Dun | | | | | | | | | | |
| | Overo | | | | | | | | | | |
| | | Silver | n/n | X | X | X | n/n | X | n/n | X/X | X/n |
| | | Dun | | | | | | | | | |
| | | Pearl | | | | | | | | | |
| | | Black | | | | | | | | | |
| | | Overo | | | | | | | | | |
| | | Silver | X | X | X | X | n/n | X | n/n | X/X | X/n |
| | | Dun | | | | | | | | | |
| | | Pearl | | | | | | | | | |
| | | Bay | | | | | | | | | |
| | | Overo | | | | | | | | | |
| | Silver | | n/n | X | n/n | n/n | n/n | X | X/X | n/n | X/n |
| | Smoky | | | | | | | | | | |
| | Cream | | | | | | | | | | |
| | Overo | | | | | | | | | | |
| | Smoky | Black | n/n | X | n/n | n/n | n/n | n/n | X/n | n/n | X/n |
| | Black | Cream | | | | | | | | | |
| | Overo | Overo | | | | | | | | | |
| | Smoky | | n/n | X | n/n | n/n | n/n | n/n | X/X | n/n | X/n |
| | Cream | | | | | | | | | | |
| | Overo | | | | | | | | | | |
| | Smoky | Dun | n/n | X | X | X | n/n | n/n | X/n | n/n | X/n |
| | Grullo | Black | | | | | | | | | |
| | Overo | Cream | | | | | | | | | |
| | | Overo | | | | | | | | | |
| | Cham- | | | | | | X | | | X/X | X/n |
| | pagne | | | | | | | | | | |
| | Pearl | | | | | | | | | | |
| | Overo | | | | | | | | | | |
| | Cream | | | | | | | | X/n | X/n | X/n |
| | Pearl | | | | | | | | | | |
| | Overo | | | | | | | | | | |
| | Dun | | | | X | X | | | | X/X | X/n |
| | Pearl | | | | | | | | | | |
| | Overo | | | | | | | | | | |
| | Silver | | | | | | X | X | | | X/n |
| | Cham- | | | | | | | | | | |
| | pagne | | | | | | | | | | |
| | Overo | | | | | | | | | | |
| | Silver | | | | | | X | X | | X/X | X/n |
| | Cham- | | | | | | | | | | |
| | pagne | | | | | | | | | | |
| | Pearl | | | | | | | | | | |
| | Overo | | | | | | | | | | |
| | Silver | | | | | | | X | X/n | X/n | X/n |
| | Cream | | | | | | | | | | |
| | Pearl | | | | | | | | | | |
| | Overo | | | | | | | | | | |
| | Silver | | | X | | | | X | | X/X | X/n |
| | Pearl | | | | | | | | | | |
| | Overo | | | | | | | | | | |
| | Silver | | | X | X | X | | X | | X/X | X/n |
| | Dun | | | | | | | | | | |
| | Pearl | | | | | | | | | | |
| | Overo | | | | | | | | | | |
| | Silver | Silver | X | X | | | | X | X | | X/n |
| | Bay | Buck- | | | | | | | | | |
| | Cream | skin | | | | | | | | | |
| | Overo | Overo | | | | | | | | | |
| Tovero | Amber | Amber | X | X | X | X | X | n/n | n/n | n/n | X/n |
| | Dun | Cham- | | | | | | | | | |
| | Tovero | pagne | | | | | | | | | |
| | | Dun | | | | | | | | | |
| | | Tovero | | | | | | | | | |
| | Amber | Pseudo- | X | X | X | X | X | n/n | n/n | X/X | X/n |
| | Dun | cream | | | | | | | | | |
| | Pearl | Amber | | | | | | | | | |
| | Tovero | Tovero | | | | | | | | | |
| | Bay | | X | X | n/n | n/n | n/n | n/n | n/n | n/n | X/n |
| | Tovero | | | | | | | | | | |
| | Black | | n/n | X | n/n | n/n | n/n | n/n | n/n | n/n | X/n |
| | Tovero | | | | | | | | | | |
| | Buck- | | X | X | n/n | n/n | n/n | n/n | X/n | n/n | X/n |
| | skin | | | | | | | | | | |
| | Tovero | | | | | | | | | | |
| | | Gold | n/n | n/n | n/n | n/n | X | n/n | n/n | n/n | X/n |
| | | Cham- | | | | | | | | | |
| | | pagne | | | | | | | | | |
| | | Tovero | | | | | | | | | |
| | | Classic | n/n | X | n/n | n/n | X | n/n | n/n | n/n | X/n |
| | | Cham- | | | | | | | | | |
| | | pagne | | | | | | | | | |
| | | Tovero | | | | | | | | | |
| | | Amber | X | X | n/n | n/n | X | n/n | n/n | n/n | X/n |
| | | Cham- | | | | | | | | | |
| | | pagne | | | | | | | | | |
| | | Tovero | | | | | | | | | |
| | Cham- | Classic | n/n | X | X | X | X | n/n | n/n | n/n | X/n |
| | pagne | Cham- | | | | | | | | | |
| | Dun | pagne | | | | | | | | | |
| | Tovero | Dun | | | | | | | | | |
| | | Tovero | | | | | | | | | |
| | Cham- | Pseudo- | n/n | X | X | X | X | n/n | n/n | X/X | X/n |
| | pagne | cream | | | | | | | | | |
| | Dun | Classic | | | | | | | | | |
| | Pearl | Tovero | | | | | | | | | |
| | Tovero | | | | | | | | | | |
| | | Cham- | n/n | n/n | n/n | n/n | X | n/n | n/n | X/X | X/n |
| | | pagne | | | | | | | | | |
| | | Gold | | | | | | | | | |
| | | Pearl | | | | | | | | | |
| | | Tovero | | | | | | | | | |
| | | Cham- | n/n | X | n/n | n/n | X | n/n | n/n | X/X | X/n |
| | | pagne | | | | | | | | | |
| | | Classic | | | | | | | | | |
| | | Pearl | | | | | | | | | |
| | | Tovero | | | | | | | | | |
| | | Cham- | X | X | n/n | n/n | X | n/n | n/n | X/X | X/n |
| | | pagne | | | | | | | | | |
| | | Amber | | | | | | | | | |
| | | Pearl | | | | | | | | | |
| | | Tovero | | | | | | | | | |
| | | Gold | n/n | n/n | n/n | n/n | X | n/n | X | n/n | X/n |
| | | Cream | | | | | | | | | |
| | | Tovero | | | | | | | | | |
| | | Classic | n/n | X | n/n | n/n | X | n/n | X | n/n | X/n |
| | | Cream | | | | | | | | | |
| | | Tovero | | | | | | | | | |
| | | Amber | X | X | n/n | n/n | X | n/n | X | n/n | X/n |
| | | Cream | | | | | | | | | |
| | | Tovero | | | | | | | | | |
| | Cream | | n/n | X | X | X | n/n | n/n | X/X | n/n | X/n |
| | Grullo | | | | | | | | | | |
| | Tovero | | | | | | | | | | |
| | | Chest- | n/n | n/n | n/n | n/n | n/n | n/n | X/n | X/n | X/n |
| | | nut | | | | | | | | | |
| | | Cream | | | | | | | | | |
| | | Pearl | | | | | | | | | |
| | | Tovero | | | | | | | | | |
| | | Black | n/n | X | n/n | n/n | n/n | n/n | X/n | X/n | X/n |
| | | Cream | | | | | | | | | |
| | | Pearl | | | | | | | | | |
| | | Tovero | | | | | | | | | |
| | | Bay | X | X | n/n | n/n | n/n | n/n | X/n | X/n | X/n |
| | | Cream | | | | | | | | | |
| | | Pearl | | | | | | | | | |
| | | Tovero | | | | | | | | | |
| | Cremello | | n/n | n/n | n/n | n/n | n/n | n/n | X/X | n/n | X/n |
| | Tovero | | | | | | | | | | |
| | | Red Dun | n/n | n/n | X | X | n/n | n/n | n/n | n/n | X/n |
| | | Tovero | | | | | | | | | |
| | | Apricot | n/n | n/n | X | X | n/n | n/n | n/n | X/X | X/n |
| | | Dun | | | | | | | | | |
| | | Tovero | | | | | | | | | |
| | | Gold | n/n | n/n | X | X | X | n/n | n/n | n/n | X/n |
| | | Dun | | | | | | | | | |
| | | Tovero | | | | | | | | | |
| | | Classic | X | X | X | X | n/n | n/n | n/n | n/n | X/n |
| | | Dun | | | | | | | | | |
| | | Tovero | | | | | | | | | |
| | | Dun | n/n | X | X | X | n/n | n/n | n/n | X/X | X/n |
| | | Pearl | | | | | | | | | |
| | | Black | | | | | | | | | |
| | | Tovero | | | | | | | | | |
| | | Dun | X | X | X | X | n/n | n/n | n/n | X/X | X/n |
| | | Pearl | | | | | | | | | |
| | | Bay | | | | | | | | | |
| | | Tovero | | | | | | | | | |
| | Dunolino | | n/n | n/n | X | X | n/n | n/n | X/n | n/n | X/n |
| | Tovero | | | | | | | | | | |
| | Dunskin | | X | X | X | X | n/n | n/n | X/n | n/n | X/n |
| | Tovero | | | | | | | | | | |
| | Gold | Pseudo- | n/n | n/n | X | X | X | n/n | n/n | X/X | X/n |
| | Dun | cream | | | | | | | | | |
| | Pearl | Gold | | | | | | | | | |
| | Tovero | Tovero | | | | | | | | | |
| | Grullo | | n/n | X | X | X | n/n | n/n | n/n | n/n | X/n |
| | Tovero | | | | | | | | | | |
| | Palomino | | | n/n | n/n | n/n | n/n | n/n | X/n | n/n | X/n |
| | Tovero | | | | | | | | | | |
| | | Apricot | n/n | n/n | n/n | n/n | n/n | n/n | n/n | X/X | X/n |
| | | Pearl | | | | | | | | | |
| | | Tovero | | | | | | | | | |
| | | Black | n/n | X | n/n | n/n | n/n | n/n | n/n | X/X | X/n |
| | | Pearl | | | | | | | | | |
| | | Tovero | | | | | | | | | |
| | | Bay | X | X | n/n | n/n | n/n | n/n | n/n | X/X | X/n |
| | | Pearl | | | | | | | | | |
| | | Tovero | | | | | | | | | |
| | Perlino | | X | X | n/n | n/n | n/n | n/n | X/X | n/n | X/n |
| | Tovero | | | | | | | | | | |
| | Perlino | | X | X | X | X | n/n | n/n | X/X | n/n | X/n |
| | Dun | | | | | | | | | | |
| | Tovero | | | | | | | | | | |
| | Red | Chest- | | n/n | n/n | n/n | n/n | n/n | n/n | n/n | X/n |
| | Tovero | nut, | | | | | | | | | |
| | | Sorrel | | | | | | | | | |
| | | Tovero | | | | | | | | | |
| | Silver | Silver | X | X | X | X | X | X | n/n | n/n | X/n |
| | Amber | Amber | | | | | | | | | |
| | Dun | Cham- | | | | | | | | | |
| | Tovero | pagne | | | | | | | | | |
| | | Dun | | | | | | | | | |
| | | Tovero | | | | | | | | | |
| | | Silver | n/n | X | n/n | n/n | X | X | n/n | n/n | X/n |
| | | Classic | | | | | | | | | |
| | | Cham- | | | | | | | | | |
| | | pagne | | | | | | | | | |
| | | Tovero | | | | | | | | | |
| | | Silver | X | X | n/n | n/n | X | X | n/n | n/n | X/n |
| | | Amber | | | | | | | | | |
| | | Cham- | | | | | | | | | |
| | | pagne | | | | | | | | | |
| | | Tovero | | | | | | | | | |
| | Silver | | n/n | X | X | X | X | X | n/n | n/n | X/n |
| | Cham- | | | | | | | | | | |
| | pagne | | | | | | | | | | |
| | Dun | | | | | | | | | | |
| | Tovero | | | | | | | | | | |
| | | Silver | n/n | X | n/n | n/n | X | X | n/n | X/X | X/n |
| | | Classic | | | | | | | | | |
| | | Cham- | | | | | | | | | |
| | | pagne | | | | | | | | | |
| | | Pearl | | | | | | | | | |
| | | Tovero | | | | | | | | | |
| | | Silver | X | X | n/n | n/n | X | X | n/n | X/X | X/n |
| | | Classic | | | | | | | | | |
| | | Amber | | | | | | | | | |
| | | Pearl | | | | | | | | | |
| | | Tovero | | | | | | | | | |
| | | Silver | n/n | X | n/n | n/n | n/n | X | X/n | X/n | X/n |
| | | Black | | | | | | | | | |
| | | Cream | | | | | | | | | |
| | | Pearl | | | | | | | | | |
| | | Tovero | | | | | | | | | |
| | | Silver | X | X | n/n | n/n | n/n | X | X/n | X/n | X/n |
| | | Bay | | | | | | | | | |
| | | Cream | | | | | | | | | |
| | | Pearl | | | | | | | | | |
| | | Tovero | | | | | | | | | |
| | Silver | Silver | n/n | X | n/n | n/n | n/n | X | n/n | n/n | X/n |
| | Dapple | Black | | | | | | | | | |
| | Tovero | Tovero | | | | | | | | | |
| | Silver | | X | X | n/n | n/n | n/n | X | n/n | n/n | X/n |
| | Bay | | | | | | | | | | |
| | Tovero | | | | | | | | | | |
| | Silver | | X | X | n/n | n/n | n/n | X | X/n | n/n | X/n |
| | Buck- | | | | | | | | | | |
| | skin | | | | | | | | | | |
| | Tovero | | | | | | | | | | |
| | Silver | | n/n | X | X | X | n/n | X | X/X | n/n | X/n |
| | Cream | | | | | | | | | | |
| | Grullo | | | | | | | | | | |
| | Tovero | | | | | | | | | | |
| | Silver | Silver | | X | X | X | n/n | X | n/n | n/n | X/n |
| | Dun | Dapple | | | | | | | | | |
| | Tovero | Dun | | | | | | | | | |
| | | Tovero | | | | | | | | | |
| | Silver | Silver | X | X | X | X | n/n | X | X/n | n/n | X/n |
| | Dunskin | Dun Bay | | | | | | | | | |
| | Tovero | Cream | | | | | | | | | |
| | | Tovero | | | | | | | | | |
| | Silver | Silver | n/n | X | X | X | n/n | X | n/n | n/n | X/n |
| | Grullo | Dun | | | | | | | | | |
| | Tovero | Black | | | | | | | | | |
| | | Tovero | | | | | | | | | |
| | | Silver | n/n | X | n/n | n/n | n/n | X | n/n | X/X | X/n |
| | | Pearl | | | | | | | | | |
| | | Black | | | | | | | | | |
| | | Tovero | | | | | | | | | |
| | | Silver | X | X | n/n | n/n | n/n | X | n/n | X/X | X/n |
| | | Pearl | | | | | | | | | |
| | | Bay | | | | | | | | | |
| | | Tovero | | | | | | | | | |
| | Silver | | X | X | n/n | n/n | n/n | X | X/X | n/n | X/n |
| | Perlino | | | | | | | | | | |
| | Tovero | | | | | | | | | | |
| | Silver | | X | X | X | X | n/n | X | X/X | n/n | X/n |
| | Perlino | | | | | | | | | | |
| | Dun | | | | | | | | | | |
| | Tovero | | | | | | | | | | |
| | | Silver | n/n | X | X | X | n/n | X | n/n | X/X | X/n |
| | | Dun | | | | | | | | | |
| | | Pearl | | | | | | | | | |
| | | Black | | | | | | | | | |
| | | Tovero | | | | | | | | | |
| | | Silver | X | X | X | X | n/n | X | n/n | X/X | X/n |
| | | Dun | | | | | | | | | |
| | | Pearl | | | | | | | | | |
| | | Bay | | | | | | | | | |
| | | Tovero | | | | | | | | | |
| | Silver | | n/n | X | n/n | n/n | n/n | X | X/X | n/n | X/n |
| | Smoky | | | | | | | | | | |
| | Cream | | | | | | | | | | |
| | Tovero | | | | | | | | | | |
| | Smoky | Black | n/n | X | n/n | n/n | n/n | n/n | X/n | n/n | X/n |
| | Black | Cream | | | | | | | | | |
| | Tovero | Tovero | | | | | | | | | |
| | Smoky | | n/n | X | n/n | n/n | n/n | n/n | X/X | n/n | X/n |
| | Cream | | | | | | | | | | |
| | Tovero | | | | | | | | | | |
| | Smoky | Dun | n/n | X | X | X | n/n | n/n | X/n | n/n | X/n |
| | Grullo | Black | | | | | | | | | |
| | Tovero | Cream | | | | | | | | | |
| | | Tovero | | | | | | | | | |
| | Cham- | | | | | | X | | | X/X | X/n |
| | pagne | | | | | | | | | | |
| | Pearl | | | | | | | | | | |
| | Tovero | | | | | | | | | | |
| | Cream | | | | | | | | X/n | X/n | X/n |
| | Pearl | | | | | | | | | | |
| | Tovero | | | | | | | | | | |
| | Dun | | | | X | X | | | | X/X | X/n |
| | Pearl | | | | | | | | | | |
| | Tovero | | | | | | | | | | |
| | Silver | | | | | | X | X | | | X/n |
| | Cham- | | | | | | | | | | |
| | pagne | | | | | | | | | | |
| | Tovero | | | | | | | | | | |
| | Silver | | | | | | X | X | | X/X | X/n |
| | Cham- | | | | | | | | | | |
| | pagne | | | | | | | | | | |
| | Pearl | | | | | | | | | | |
| | Tovero | | | | | | | | | | |
| | Silver | | | | | | | X | X/n | X/n | X/n |
| | Cream | | | | | | | | | | |
| | Pearl | | | | | | | | | | |
| | Tovero | | | | | | | | | | |
| | Silver | | | X | | | | X | | X/X | X/n |
| | Pearl | | | | | | | | | | |
| | Tovero | | | | | | | | | | |
| | Silver | | | X | X | X | | X | | X/X | X/n |
| | Dun | | | | | | | | | | |
| | Pearl | | | | | | | | | | |
| | Tovero | | | | | | | | | | |
| | Silver | Silver | X | X | | | | X | X | | X/n |
| | Bay | Buck- | | | | | | | | | |
| | Cream | skin | | | | | | | | | |
| | Tovero | Tovero | | | | | | | | | |
| Splash | Amber | Amber | X | X | X | X | X | n/n | n/n | n/n | |
| | Dun | Cham- | | | | | | | | | |
| | Splash | pagne | | | | | | | | | |
| | | Dun | | | | | | | | | |
| | | Splash | | | | | | | | | |
| | Amber | Pseudo- | X | X | X | X | X | n/n | n/n | X/X | |
| | Dun | cream | | | | | | | | | |
| | Pearl | Amber | | | | | | | | | |
| | Splash | Splash | | | | | | | | | |
| | Bay | | X | X | n/n | n/n | n/n | n/n | n/n | n/n | |
| | Splash | | | | | | | | | | |
| | Black | | n/n | X | n/n | n/n | n/n | n/n | n/n | n/n | |
| | Splash | | | | | | | | | | |
| | Buck- | | X | X | n/n | n/n | n/n | n/n | X/n | n/n | |
| | skin | | | | | | | | | | |
| | Splash | | | | | | | | | | |
| | | Gold | n/n | n/n | n/n | n/n | X | n/n | n/n | n/n | |
| | | Cham- | | | | | | | | | |
| | | pagne | | | | | | | | | |
| | | Splash | | | | | | | | | |
| | | Classic | n/n | X | n/n | n/n | X | n/n | n/n | n/n | |
| | | Cham- | | | | | | | | | |
| | | pagne | | | | | | | | | |
| | | Splash | | | | | | | | | |
| | | Amber | X | X | n/n | n/n | X | n/n | n/n | n/n | |
| | | Cham- | | | | | | | | | |
| | | pagne | | | | | | | | | |
| | | Splash | | | | | | | | | |
| | Cham- | Classic | n/n | X | X | X | X | n/n | n/n | n/n | |
| | pagne | Cham- | | | | | | | | | |
| | Dun | pagne | | | | | | | | | |
| | Splash | Dun | | | | | | | | | |
| | | Splash | | | | | | | | | |
| | Cham- | Pseudo- | n/n | X | X | X | X | n/n | n/n | X/X | |
| | pagne | cream | | | | | | | | | |
| | Dun | Classic | | | | | | | | | |
| | Pearl | Splash | | | | | | | | | |
| | Splash | | | | | | | | | | |
| | | Cham- | n/n | n/n | n/n | n/n | X | n/n | n/n | X/X | |
| | | pagne | | | | | | | | | |
| | | Gold | | | | | | | | | |
| | | Pearl | | | | | | | | | |
| | | Splash | | | | | | | | | |
| | | Cham- | n/n | X | n/n | n/n | X | n/n | n/n | X/X | |
| | | pagne | | | | | | | | | |
| | | Classic | | | | | | | | | |
| | | Pearl | | | | | | | | | |
| | | Splash | | | | | | | | | |
| | | Cham- | X | X | n/n | n/n | X | n/n | n/n | X/X | |
| | | pagne | | | | | | | | | |
| | | Amber | | | | | | | | | |
| | | Pearl | | | | | | | | | |
| | | Splash | | | | | | | | | |
| | | Gold | n/n | n/n | n/n | n/n | X | n/n | X | n/n | |
| | | Cream | | | | | | | | | |
| | | Splash | | | | | | | | | |
| | | Classic | n/n | X | n/n | n/n | X | n/n | X | n/n | |
| | | Cream | | | | | | | | | |
| | | Splash | | | | | | | | | |
| | | Amber | X | X | n/n | n/n | X | n/n | X | n/n | |
| | | Cream | | | | | | | | | |
| | | Splash | | | | | | | | | |
| | Cream | | n/n | X | X | X | n/n | n/n | X/X | n/n | |
| | Grullo | | | | | | | | | | |
| | Splash | | | | | | | | | | |
| | | Chest- | n/n | n/n | n/n | n/n | n/n | n/n | X/n | X/n | |
| | | nut | | | | | | | | | |
| | | Cream | | | | | | | | | |
| | | Pearl | | | | | | | | | |
| | | Splash | | | | | | | | | |
| | | Black | n/n | X | n/n | n/n | n/n | n/n | X/n | X/n | |
| | | Cream | | | | | | | | | |
| | | Pearl | | | | | | | | | |
| | | Splash | | | | | | | | | |
| | | Bay | X | X | n/n | n/n | n/n | n/n | X/n | X/n | |
| | | Cream | | | | | | | | | |
| | | Pearl | | | | | | | | | |
| | | Splash | | | | | | | | | |
| | Cremello | | n/n | n/n | n/n | n/n | n/n | n/n | X/X | n/n | |
| | Splash | | | | | | | | | | |
| | | Red Dun | n/n | n/n | X | X | n/n | n/n | n/n | n/n | |
| | | Splash | | | | | | | | | |
| | | Apricot | n/n | n/n | X | X | n/n | n/n | n/n | X/X | |
| | | Dun | | | | | | | | | |
| | | Splash | | | | | | | | | |
| | | Gold | n/n | n/n | X | X | X | n/n | n/n | n/n | |
| | | Dun | | | | | | | | | |
| | | Splash | | | | | | | | | |
| | | Classic | X | X | X | X | n/n | n/n | n/n | n/n | |
| | | Dun | | | | | | | | | |
| | | Splash | | | | | | | | | |
| | | Dun | n/n | X | X | X | n/n | n/n | n/n | X/X | |
| | | Pearl | | | | | | | | | |
| | | Black | | | | | | | | | |
| | | Splash | | | | | | | | | |
| | | Dun | X | X | X | X | n/n | n/n | n/n | X/X | |
| | | Pearl | | | | | | | | | |
| | | Bay | | | | | | | | | |
| | | Splash | | | | | | | | | |
| | Dunolino | | n/n | n/n | X | X | n/n | n/n | X/n | n/n | |
| | Splash | | | | | | | | | | |
| | Dunskin | | X | X | X | X | n/n | n/n | X/n | n/n | |
| | Splash | | | | | | | | | | |
| | Gold | Pseudo- | n/n | n/n | X | X | X | n/n | n/n | X/X | |
| | Dun | cream | | | | | | | | | |
| | Pearl | Gold | | | | | | | | | |
| | Splash | Splash | | | | | | | | | |
| | Grullo | | n/n | X | X | X | n/n | n/n | n/n | n/n | |
| | Splash | | | | | | | | | | |
| | Palomino | | | n/n | n/n | n/n | n/n | n/n | X/n | n/n | |
| | Splash | | | | | | | | | | |
| | | Apricot | n/n | n/n | n/n | n/n | n/n | n/n | n/n | X/X | |
| | | Pearl | | | | | | | | | |
| | | Splash | | | | | | | | | |
| | | Black | n/n | X | n/n | n/n | n/n | n/n | n/n | X/X | |
| | | Pearl | | | | | | | | | |
| | | Splash | | | | | | | | | |
| | | Bay | X | X | n/n | n/n | n/n | n/n | n/n | X/X | |
| | | Pearl | | | | | | | | | |
| | | Splash | | | | | | | | | |
| | Perlino | | X | X | n/n | n/n | n/n | n/n | X/X | n/n | |
| | Splash | | | | | | | | | | |
| | Perlino | | X | X | X | X | n/n | n/n | X/X | n/n | |
| | Dun | | | | | | | | | | |
| | Splash | | | | | | | | | | |
| | Red | Chest- | | n/n | n/n | n/n | n/n | n/n | n/n | n/n | |
| | Splash | nut, | | | | | | | | | |
| | | Sorrel | | | | | | | | | |
| | | Splash | | | | | | | | | |
| | Silver | Silver | X | X | X | X | X | X | n/n | n/n | |
| | Amber | Amber | | | | | | | | | |
| | Dun | Cham- | | | | | | | | | |
| | Splash | pagne | | | | | | | | | |
| | | Dun | | | | | | | | | |
| | | Splash | | | | | | | | | |
| | | Silver | n/n | X | n/n | n/n | X | X | n/n | n/n | |
| | | Classic | | | | | | | | | |
| | | Cham- | | | | | | | | | |
| | | pagne | | | | | | | | | |
| | | Splash | | | | | | | | | |
| | | Silver | X | X | n/n | n/n | X | X | n/n | n/n | |
| | | Amber | | | | | | | | | |
| | | Cham- | | | | | | | | | |
| | | pagne | | | | | | | | | |
| | | Splash | | | | | | | | | |
| | Silver | | n/n | X | X | X | X | X | n/n | n/n | |
| | Cham- | | | | | | | | | | |
| | pagne | | | | | | | | | | |
| | Dun | | | | | | | | | | |
| | Splash | | | | | | | | | | |
| | | Silver | n/n | X | n/n | n/n | X | X | n/n | X/X | |
| | | Classic | | | | | | | | | |
| | | Cham- | | | | | | | | | |
| | | pagne | | | | | | | | | |
| | | Pearl | | | | | | | | | |
| | | Splash | | | | | | | | | |
| | | Silver | X | X | n/n | n/n | X | X | n/n | X/X | |
| | | Classic | | | | | | | | | |
| | | Amber | | | | | | | | | |
| | | Pearl | | | | | | | | | |
| | | Splash | | | | | | | | | |
| | | Silver | n/n | X | n/n | n/n | n/n | X | X/n | X/n | |
| | | Black | | | | | | | | | |
| | | Cream | | | | | | | | | |
| | | Pearl | | | | | | | | | |
| | | Splash | | | | | | | | | |
| | | Silver | X | X | n/n | n/n | n/n | X | X/n | X/n | |
| | | Bay | | | | | | | | | |
| | | Cream | | | | | | | | | |
| | | Pearl | | | | | | | | | |
| | | Splash | | | | | | | | | |
| | Silver | Silver | n/n | X | n/n | n/n | n/n | X | n/n | n/n | |
| | Dapple | Black | | | | | | | | | |
| | Splash | Splash | | | | | | | | | |
| | Silver | | X | X | n/n | n/n | n/n | X | n/n | n/n | |
| | Bay | | | | | | | | | | |
| | Splash | | | | | | | | | | |
| | Silver | | X | X | n/n | n/n | n/n | X | X/n | n/n | |
| | Buck- | | | | | | | | | | |
| | skin | | | | | | | | | | |
| | Splash | | | | | | | | | | |
| | Silver | | n/n | X | X | X | n/n | X | X/X | n/n | |
| | Cream | | | | | | | | | | |
| | Grullo | | | | | | | | | | |
| | Splash | | | | | | | | | | |
| | Silver | Silver | | X | X | X | n/n | X | n/n | n/n | |
| | Dun | Dapple | | | | | | | | | |
| | Splash | Dun | | | | | | | | | |
| | | Splash | | | | | | | | | |
| | Silver | Dun Bay | X | X | X | X | n/n | X | X/n | n/n | |
| | Dunskin | Silver | | | | | | | | | |
| | Splash | Cream | | | | | | | | | |
| | | Splash | | | | | | | | | |
| | Silver | Silver | n/n | X | X | X | n/n | X | n/n | n/n | |
| | Grullo | Dun | | | | | | | | | |
| | Splash | Black | | | | | | | | | |
| | | Splash | | | | | | | | | |
| | | Silver | n/n | X | n/n | n/n | n/n | X | n/n | X/X | |
| | | Pearl | | | | | | | | | |
| | | Black | | | | | | | | | |
| | | Splash | | | | | | | | | |
| | | Silver | X | X | n/n | n/n | n/n | X | n/n | X/X | |
| | | Pearl | | | | | | | | | |
| | | Bay | | | | | | | | | |
| | | Splash | | | | | | | | | |
| | Silver | | X | X | n/n | n/n | n/n | X | X/X | n/n | |
| | Perlino | | | | | | | | | | |
| | Splash | | | | | | | | | | |
| | Silver | | X | X | X | X | n/n | X | X/X | n/n | |
| | Perlino | | | | | | | | | | |
| | Dun | | | | | | | | | | |
| | Splash | | | | | | | | | | |
| | | Silver | n/n | X | X | X | n/n | X | n/n | X/X | |
| | | Dun | | | | | | | | | |
| | | Pearl | | | | | | | | | |
| | | Black | | | | | | | | | |
| | | Splash | | | | | | | | | |
| | | Silver | X | X | X | X | n/n | X | n/n | X/X | |
| | | Dun | | | | | | | | | |
| | | Pearl | | | | | | | | | |
| | | Bay | | | | | | | | | |
| | | Splash | | | | | | | | | |
| | Silver | | n/n | X | n/n | n/n | n/n | X | X/X | n/n | |
| | Smoky | | | | | | | | | | |
| | Cream | | | | | | | | | | |
| | Splash | | | | | | | | | | |
| | Smoky | Black | n/n | X | n/n | n/n | n/n | n/n | X/n | n/n | |
| | Black | Cream | | | | | | | | | |
| | Splash | Splash | | | | | | | | | |
| | Smoky | | n/n | X | n/n | n/n | n/n | n/n | X/X | n/n | |
| | Cream | | | | | | | | | | |
| | Splash | | | | | | | | | | |
| | Smoky | Dun | n/n | X | X | X | n/n | n/n | X/n | n/n | |
| | Grullo | Black | | | | | | | | | |
| | Splash | Cream | | | | | | | | | |
| | | Splash | | | | | | | | | |
| | Cham- | | | | | | X | | | X/X | |
| | pagne | | | | | | | | | | |
| | Pearl | | | | | | | | | | |
| | Splash | | | | | | | | | | |
| | Cream | | | | | | | | X/n | X/n | |
| | Pearl | | | | | | | | | | |
| | Splash | | | | | | | | | | |
| | Dun | | | | X | X | | | | X/X | |
| | Pearl | | | | | | | | | | |
| | Splash | | | | | | | | | | |
| | Silver | | | | | | X | X | | | |
| | Cham- | | | | | | | | | | |
| | pagne | | | | | | | | | | |
| | Splash | | | | | | | | | | |
| | Silver | | | | | | X | X | | X/X | |
| | Cham- | | | | | | | | | | |
| | pagne | | | | | | | | | | |
| | Pearl | | | | | | | | | | |
| | Splash | | | | | | | | | | |
| | Silver | | | | | | | X | X/n | X/n | |
| | Cream | | | | | | | | | | |
| | Pearl | | | | | | | | | | |
| | Splash | | | | | | | | | | |
| | Silver | | | X | | | | X | | X/X | |
| | Pearl | | | | | | | | | | |
| | Splash | | | | | | | | | | |
| | Silver | | | X | X | X | | X | | X/X | |
| | Dun | | | | | | | | | | |
| | Pearl | | | | | | | | | | |
| | Splash | | | | | | | | | | |
| | Silver | | X | X | | | | X | X | | |
| | Bay | | | | | | | | | | |
| | Cream | | | | | | | | | | |
| | Splash | | | | | | | | | | |
| Tobiano | Amber | Amber | X | X | X | X | X | n/n | n/n | n/n | |
| | Dun | Cham- | | | | | | | | | |
| | Tobiano | pagne | | | | | | | | | |
| | | Dun | | | | | | | | | |
| | | Tobiano | | | | | | | | | |
| | Amber | Pseudo- | X | X | X | X | X | n/n | n/n | X/X | |
| | Dun | cream | | | | | | | | | |
| | Pearl | Amber | | | | | | | | | |
| | Tobiano | Tobiano | | | | | | | | | |
| | Bay | | X | X | n/n | n/n | n/n | n/n | n/n | n/n | |
| | Tobiano | | | | | | | | | | |
| | Black | | n/n | X | n/n | n/n | n/n | n/n | n/n | n/n | |
| | Tobiano | | | | | | | | | | |
| | Buck- | | X | X | n/n | n/n | n/n | n/n | X/n | n/n | |
| | skin | | | | | | | | | | |
| | Tobiano | | | | | | | | | | |
| | | Gold | n/n | n/n | n/n | n/n | X | n/n | n/n | n/n | |
| | | Cham- | | | | | | | | | |
| | | pagne | | | | | | | | | |
| | | Tobiano | | | | | | | | | |
| | | Classic | n/n | X | n/n | n/n | X | n/n | n/n | n/n | |
| | | Cham- | | | | | | | | | |
| | | pagne | | | | | | | | | |
| | | Tobiano | | | | | | | | | |
| | | Amber | X | X | n/n | n/n | X | n/n | n/n | n/n | |
| | | Cham- | | | | | | | | | |
| | | pagne | | | | | | | | | |
| | | Tobiano | | | | | | | | | |
| | Cham- | Classic | n/n | X | X | X | X | n/n | n/n | n/n | |
| | pagne | Cham- | | | | | | | | | |
| | Dun | pagne | | | | | | | | | |
| | Tobiano | Dun | | | | | | | | | |
| | | Tobiano | | | | | | | | | |
| | Cham- | Pseudo- | n/n | X | X | X | X | n/n | n/n | X/X | |
| | pagne | cream | | | | | | | | | |
| | Dun | Classic | | | | | | | | | |
| | Pearl | Tobiano | | | | | | | | | |
| | Tobiano | | | | | | | | | | |
| | | Cham- | n/n | n/n | n/n | n/n | X | n/n | n/n | X/X | |
| | | pagne | | | | | | | | | |
| | | Gold | | | | | | | | | |
| | | Pearl | | | | | | | | | |
| | | Tobiano | | | | | | | | | |
| | | Cham- | n/n | X | n/n | n/n | X | n/n | n/n | X/X | |
| | | pagne | | | | | | | | | |
| | | Classic | | | | | | | | | |
| | | Pearl | | | | | | | | | |
| | | Tobiano | | | | | | | | | |
| | | Cham- | X | X | n/n | n/n | X | n/n | n/n | X/X | |
| | | pagne | | | | | | | | | |
| | | Amber | | | | | | | | | |
| | | Pearl | | | | | | | | | |
| | | Tobiano | | | | | | | | | |
| | | Gold | n/n | n/n | n/n | n/n | X | n/n | X | n/n | |
| | | Cream | | | | | | | | | |
| | | Tobiano | | | | | | | | | |
| | | Classic | n/n | X | n/n | n/n | X | n/n | X | n/n | |
| | | Cream | | | | | | | | | |
| | | Tobiano | | | | | | | | | |
| | | Amber | X | X | n/n | n/n | X | n/n | X | n/n | |
| | | Cream | | | | | | | | | |
| | | Tobiano | | | | | | | | | |
| | Cream | | n/n | X | X | X | n/n | n/n | X/X | n/n | |
| | Grullo | | | | | | | | | | |
| | Tobiano | | | | | | | | | | |
| | | Chest- | n/n | n/n | n/n | n/n | n/n | n/n | X/n | X/n | |
| | | nut | | | | | | | | | |
| | | Cream | | | | | | | | | |
| | | Pearl | | | | | | | | | |
| | | Tobiano | | | | | | | | | |
| | | Black | n/n | X | n/n | n/n | n/n | n/n | X/n | X/n | |
| | | Cream | | | | | | | | | |
| | | Pearl | | | | | | | | | |
| | | Tobiano | | | | | | | | | |
| | | Bay | X | X | n/n | n/n | n/n | n/n | X/n | X/n | |
| | | Cream | | | | | | | | | |
| | | Pearl | | | | | | | | | |
| | | Tobiano | | | | | | | | | |
| | Cremello | | n/n | n/n | n/n | n/n | n/n | n/n | X/X | n/n | |
| | Tobiano | | | | | | | | | | |
| | | Red Dun | n/n | n/n | X | X | n/n | n/n | n/n | n/n | |
| | | Tobiano | | | | | | | | | |
| | | Apricot | n/n | n/n | X | X | n/n | n/n | n/n | X/X | |
| | | Dun | | | | | | | | | |
| | | Tobiano | | | | | | | | | |
| | | Gold | n/n | n/n | X | X | X | n/n | n/n | n/n | |
| | | Dun | | | | | | | | | |
| | | Tobiano | | | | | | | | | |
| | | Classic | X | X | X | X | n/n | n/n | n/n | n/n | |
| | | Dun | | | | | | | | | |
| | | Tobiano | | | | | | | | | |
| | | Dun | n/n | X | X | X | n/n | n/n | n/n | X/X | |
| | | Pearl | | | | | | | | | |
| | | Black | | | | | | | | | |
| | | Tobiano | | | | | | | | | |
| | | Dun | X | X | X | X | n/n | n/n | n/n | X/X | |
| | | Pearl | | | | | | | | | |
| | | Bay | | | | | | | | | |
| | | Tobiano | | | | | | | | | |
| | Dunolino | | n/n | n/n | X | X | n/n | n/n | X/n | n/n | |
| | Tobiano | | | | | | | | | | |
| | Dunskin | | X | X | X | X | n/n | n/n | X/n | n/n | |
| | Tobiano | | | | | | | | | | |
| | Gold Dun | Pseudo- | n/n | n/n | X | X | X | n/n | n/n | X/X | |
| | Pearl | cream | | | | | | | | | |
| | Tobiano | Gold | | | | | | | | | |
| | | Tobiano | | | | | | | | | |
| | Grullo | | n/n | X | X | X | n/n | n/n | n/n | n/n | |
| | Tobiano | | | | | | | | | | |
| | Palomino | | | n/n | n/n | n/n | n/n | n/n | X/n | n/n | |
| | Tobiano | | | | | | | | | | |
| | | Apricot | n/n | n/n | n/n | n/n | n/n | n/n | n/n | X/X | |
| | | Pearl | | | | | | | | | |
| | | Tobiano | | | | | | | | | |
| | | Black | n/n | X | n/n | n/n | n/n | n/n | n/n | X/X | |
| | | Pearl | | | | | | | | | |
| | | Tobiano | | | | | | | | | |
| | | Bay | X | X | n/n | n/n | n/n | n/n | n/n | X/X | |
| | | Pearl | | | | | | | | | |
| | | Tobiano | | | | | | | | | |
| | Perlino | | X | X | n/n | n/n | n/n | n/n | X/X | n/n | |
| | Tobiano | | | | | | | | | | |
| | Perlino | | X | X | X | X | n/n | n/n | X/X | n/n | |
| | Dun | | | | | | | | | | |
| | Tobiano | | | | | | | | | | |
| | Red | Chest- | | n/n | n/n | n/n | n/n | n/n | n/n | n/n | |
| | Tobiano | nut, | | | | | | | | | |
| | | Sorrel | | | | | | | | | |
| | Silver | Silver | X | X | X | X | X | X | n/n | n/n | |
| | Amber | Amber | | | | | | | | | |
| | Dun | Cham- | | | | | | | | | |
| | Tobiano | pagne | | | | | | | | | |
| | | Dun | | | | | | | | | |
| | | Tobiano | | | | | | | | | |
| | | Silver | n/n | X | n/n | n/n | X | X | n/n | n/n | |
| | | Classic | | | | | | | | | |
| | | Cham- | | | | | | | | | |
| | | pagne | | | | | | | | | |
| | | Tobiano | | | | | | | | | |
| | | Silver | X | X | n/n | n/n | X | X | n/n | n/n | |
| | | Amber | | | | | | | | | |
| | | Cham- | | | | | | | | | |
| | | pagne | | | | | | | | | |
| | | Tobiano | | | | | | | | | |
| | Silver | | n/n | X | X | X | X | X | n/n | n/n | |
| | Cham- | | | | | | | | | | |
| | pagne | | | | | | | | | | |
| | Dun | | | | | | | | | | |
| | Tobiano | | | | | | | | | | |
| | | Silver | n/n | X | n/n | n/n | X | X | n/n | X/X | |
| | | Classic | | | | | | | | | |
| | | Cham- | | | | | | | | | |
| | | pagne | | | | | | | | | |
| | | Pearl | | | | | | | | | |
| | | Tobiano | | | | | | | | | |
| | | Silver | X | X | n/n | n/n | X | X | n/n | X/X | |
| | | Classic | | | | | | | | | |
| | | Amber | | | | | | | | | |
| | | Pearl | | | | | | | | | |
| | | Tobiano | | | | | | | | | |
| | | Silver | n/n | X | n/n | n/n | n/n | X | X/n | X/n | |
| | | Black | | | | | | | | | |
| | | Cream | | | | | | | | | |
| | | Pearl | | | | | | | | | |
| | | Tobiano | | | | | | | | | |
| | | Silver | X | X | n/n | n/n | n/n | X | X/n | X/n | |
| | | Bay | | | | | | | | | |
| | | Cream | | | | | | | | | |
| | | Pearl | | | | | | | | | |
| | | Tobiano | | | | | | | | | |
| | Silver | Silver | n/n | X | n/n | n/n | n/n | X | n/n | n/n | |
| | Dapple | Black | | | | | | | | | |
| | Tobiano | Tobiano | | | | | | | | | |
| | Silver | | X | X | n/n | n/n | n/n | X | n/n | n/n | |
| | Bay | | | | | | | | | | |
| | Tobiano | | | | | | | | | | |
| | Silver | | X | X | n/n | n/n | n/n | X | X/n | n/n | |
| | Buck- | | | | | | | | | | |
| | skin | | | | | | | | | | |
| | Tobiano | | | | | | | | | | |
| | Silver | | n/n | X | X | X | n/n | X | X/X | n/n | |
| | Cream | | | | | | | | | | |
| | Grullo | | | | | | | | | | |
| | Tobiano | | | | | | | | | | |
| | Silver | Silver | | X | X | X | n/n | X | n/n | n/n | |
| | Dun | Dapple | | | | | | | | | |
| | Tobiano | Dun | | | | | | | | | |
| | | Tobiano | | | | | | | | | |
| | Silver | Silver | X | X | X | X | n/n | X | X/n | n/n | |
| | Dunskin | Dun Bay | | | | | | | | | |
| | Tobiano | Cream | | | | | | | | | |
| | | Tobiano | | | | | | | | | |
| | Silver | Silver | n/n | X | X | X | n/n | X | n/n | n/n | |
| | Grullo | Dun | | | | | | | | | |
| | Tobiano | Black | | | | | | | | | |
| | | Tobiano | | | | | | | | | |
| | | Silver | n/n | X | n/n | n/n | n/n | X | n/n | X/X | |
| | | Pearl | | | | | | | | | |
| | | Black | | | | | | | | | |
| | | Tobiano | | | | | | | | | |
| | | Silver | X | X | n/n | n/n | n/n | X | n/n | X/X | |
| | | Pearl | | | | | | | | | |
| | | Bay | | | | | | | | | |
| | | Tobiano | | | | | | | | | |
| | Silver | | X | X | n/n | n/n | n/n | X | X/X | n/n | |
| | Perlino | | | | | | | | | | |
| | Tobiano | | | | | | | | | | |
| | Silver | | X | X | X | X | n/n | X | X/X | n/n | |
| | Perlino | | | | | | | | | | |
| | Dun | | | | | | | | | | |
| | Tobiano | | | | | | | | | | |
| | | Silver | n/n | X | X | X | n/n | X | n/n | X/X | |
| | | Dun | | | | | | | | | |
| | | Pearl | | | | | | | | | |
| | | Black | | | | | | | | | |
| | | Tobiano | | | | | | | | | |
| | | Silver | X | X | X | X | n/n | X | n/n | X/X | |
| | | Dun | | | | | | | | | |
| | | Pearl | | | | | | | | | |
| | | Bay | | | | | | | | | |
| | | Tobiano | | | | | | | | | |
| | Silver | | n/n | X | n/n | n/n | n/n | X | X/X | n/n | |
| | Smoky | | | | | | | | | | |
| | Cream | | | | | | | | | | |
| | Tobiano | | | | | | | | | | |
| | Smoky | Black | n/n | X | n/n | n/n | n/n | n/n | X/n | n/n | |
| | Black | Cream | | | | | | | | | |
| | Tobiano | Tobiano | | | | | | | | | |
| | Smoky | | n/n | X | n/n | n/n | n/n | n/n | X/X | n/n | |
| | Cream | | | | | | | | | | |
| | Tobiano | | | | | | | | | | |
| | Smoky | Dun | n/n | X | X | X | n/n | n/n | X/n | n/n | |
| | Grullo | Black | | | | | | | | | |
| | Tobiano | Cream | | | | | | | | | |
| | | Tobiano | | | | | | | | | |
| | Cham- | | | | | | X | | | X/X | |
| | pagne | | | | | | | | | | |
| | Pearl | | | | | | | | | | |
| | Tobiano | | | | | | | | | | |
| | Cream | | | | | | | | X/n | X/n | |
| | Pearl | | | | | | | | | | |
| | Tobiano | | | | | | | | | | |
| | Dun | | | | X | X | | | | X/X | |
| | Pearl | | | | | | | | | | |
| | Tobiano | | | | | | | | | | |
| | Silver | | | | | | X | X | | | |
| | Cham- | | | | | | | | | | |
| | pagne | | | | | | | | | | |
| | Tobiano | | | | | | | | | | |
| | Silver | | | | | | X | X | | X/X | |
| | Cham- | | | | | | | | | | |
| | pagne | | | | | | | | | | |
| | Pearl | | | | | | | | | | |
| | Tobiano | | | | | | | | | | |
| | Silver | | | | | | | X | X/n | X/n | |
| | Cream | | | | | | | | | | |
| | Pearl | | | | | | | | | | |
| | Tobiano | | | | | | | | | | |
| | Silver | | | X | | | | X | | X/X | |
| | Pearl | | | | | | | | | | |
| | Tobiano | | | | | | | | | | |
| | Silver | | | X | X | X | | X | | X/X | |
| | Dun | | | | | | | | | | |
| | Pearl | | | | | | | | | | |
| | Tobiano | | | | | | | | | | |
| | Silver | | X | X | | | | X | X | | |
| | Bay | | | | | | | | | | |
| | Cream | | | | | | | | | | |
| | Tobiano | | | | | | | | | | |
| Sabino | Amber | Amber | X | X | X | X | X | n/n | n/n | n/n | |
| | Dun | Cham- | | | | | | | | | |
| | Sabino | pagne | | | | | | | | | |
| | | Dun | | | | | | | | | |
| | | Sabino | | | | | | | | | |
| | Amber | Pseudo- | X | X | X | X | X | n/n | n/n | X/X | |
| | Dun | cream | | | | | | | | | |
| | Pearl | Amber | | | | | | | | | |
| | Sabino | Sabino | | | | | | | | | |
| | Bay | | X | X | n/n | n/n | n/n | n/n | n/n | n/n | |
| | Sabino | | | | | | | | | | |
| | Black | | n/n | X | n/n | n/n | n/n | n/n | n/n | n/n | |
| | Sabino | | | | | | | | | | |
| | Buck- | | X | X | n/n | n/n | n/n | n/n | X/n | n/n | |
| | skin | | | | | | | | | | |
| | Sabino | | | | | | | | | | |
| | | Gold | n/n | n/n | n/n | n/n | X | n/n | n/n | n/n | |
| | | Cham- | | | | | | | | | |
| | | pagne | | | | | | | | | |
| | | Sabino | | | | | | | | | |
| | | Classic | n/n | X | n/n | n/n | X | n/n | n/n | n/n | |
| | | Cham- | | | | | | | | | |
| | | pagne | | | | | | | | | |
| | | Sabino | | | | | | | | | |
| | | Amber | X | X | n/n | n/n | X | n/n | n/n | n/n | |
| | | Cham- | | | | | | | | | |
| | | pagne | | | | | | | | | |
| | | Sabino | | | | | | | | | |
| | Cham- | Classic | n/n | X | X | X | X | n/n | n/n | n/n | |
| | pagne | Cham- | | | | | | | | | |
| | Dun | pagne | | | | | | | | | |
| | Sabino | Dun | | | | | | | | | |
| | | Sabino | | | | | | | | | |
| | Cham- | Pseudo- | n/n | X | X | X | X | n/n | n/n | X/X | |
| | pagne | cream | | | | | | | | | |
| | Dun | Classic | | | | | | | | | |
| | Pearl | Sabino | | | | | | | | | |
| | Sabino | | | | | | | | | | |
| | | Cham- | n/n | n/n | n/n | n/n | X | n/n | n/n | X/X | |
| | | pagne | | | | | | | | | |
| | | Gold | | | | | | | | | |
| | | Pearl | | | | | | | | | |
| | | Sabino | | | | | | | | | |
| | | Cham- | n/n | X | n/n | n/n | X | n/n | n/n | X/X | |
| | | pagne | | | | | | | | | |
| | | Classic | | | | | | | | | |
| | | Pearl | | | | | | | | | |
| | | Sabino | | | | | | | | | |
| | | Cham- | X | X | n/n | n/n | X | n/n | n/n | X/X | |
| | | pagne | | | | | | | | | |
| | | Amber | | | | | | | | | |
| | | Pearl | | | | | | | | | |
| | | Sabino | | | | | | | | | |
| | | Gold | n/n | n/n | n/n | n/n | X | n/n | X | n/n | |
| | | Cream | | | | | | | | | |
| | | Sabino | | | | | | | | | |
| | | Classic | n/n | X | n/n | n/n | X | n/n | X | n/n | |
| | | Cream | | | | | | | | | |
| | | Sabino | | | | | | | | | |
| | | Amber | X | X | n/n | n/n | X | n/n | X | n/n | |
| | | Cream | | | | | | | | | |
| | | Sabino | | | | | | | | | |
| | Cream | | n/n | X | X | X | n/n | n/n | X/X | n/n | |
| | Grullo | | | | | | | | | | |
| | Sabino | | | | | | | | | | |
| | | Chest- | n/n | n/n | n/n | n/n | n/n | n/n | X/n | X/n | |
| | | nut | | | | | | | | | |
| | | Cream | | | | | | | | | |
| | | Pearl | | | | | | | | | |
| | | Sabino | | | | | | | | | |
| | | Black | n/n | X | n/n | n/n | n/n | n/n | X/n | X/n | |
| | | Cream | | | | | | | | | |
| | | Pearl | | | | | | | | | |
| | | Sabino | | | | | | | | | |
| | | Bay | X | X | n/n | n/n | n/n | n/n | X/n | X/n | |
| | | Cream | | | | | | | | | |
| | | Pearl | | | | | | | | | |
| | | Sabino | | | | | | | | | |
| | Cremello | | n/n | n/n | n/n | n/n | n/n | n/n | X/X | n/n | |
| | Sabino | | | | | | | | | | |
| | | Red Dun | n/n | n/n | X | X | n/n | n/n | n/n | n/n | |
| | | Sabino | | | | | | | | | |
| | | Apricot | n/n | n/n | X | X | n/n | n/n | n/n | X/X | |
| | | Dun | | | | | | | | | |
| | | Sabino | | | | | | | | | |
| | | Gold | n/n | n/n | X | X | X | n/n | n/n | n/n | |
| | | Dun | | | | | | | | | |
| | | Sabino | | | | | | | | | |
| | | Classic | X | X | X | X | n/n | n/n | n/n | n/n | |
| | | Dun | | | | | | | | | |
| | | Sabino | | | | | | | | | |
| | | Dun | n/n | X | X | X | n/n | n/n | n/n | X/X | |
| | | Pearl | | | | | | | | | |
| | | Black | | | | | | | | | |
| | | Sabino | | | | | | | | | |
| | | Dun | X | X | X | X | n/n | n/n | n/n | X/X | |
| | | Pearl | | | | | | | | | |
| | | Bay | | | | | | | | | |
| | | Sabino | | | | | | | | | |
| | Dunolino | | n/n | n/n | X | X | n/n | n/n | X/n | n/n | |
| | Sabino | | | | | | | | | | |
| | Dunskin | | X | X | X | X | n/n | n/n | X/n | n/n | |
| | Sabino | | | | | | | | | | |
| | Gold Dun | Pseudo- | n/n | n/n | X | X | X | n/n | n/n | X/X | |
| | Pearl | cream | | | | | | | | | |
| | Sabino | Gold | | | | | | | | | |
| | | Sabino | | | | | | | | | |
| | Grullo | | n/n | X | X | X | n/n | n/n | n/n | n/n | |
| | Sabino | | | | | | | | | | |
| | Palomino | | | n/n | n/n | n/n | n/n | n/n | X/n | n/n | |
| | Sabino | | | | | | | | | | |
| | | Apricot | n/n | n/n | n/n | n/n | n/n | n/n | n/n | X/X | |
| | | Pearl | | | | | | | | | |
| | | Sabino | | | | | | | | | |
| | | Black | n/n | X | n/n | n/n | n/n | n/n | n/n | X/X | |
| | | Pearl | | | | | | | | | |
| | | Sabino | | | | | | | | | |
| | | Bay | X | X | n/n | n/n | n/n | n/n | n/n | X/X | |
| | | Pearl | | | | | | | | | |
| | | Sabino | | | | | | | | | |
| | Perlino | | X | X | n/n | n/n | n/n | n/n | X/X | n/n | |
| | Sabino | | | | | | | | | | |
| | Perlino | | X | X | X | X | n/n | n/n | X/X | n/n | |
| | Dun | | | | | | | | | | |
| | Sabino | | | | | | | | | | |
| | Red | Chest- | | n/n | n/n | n/n | n/n | n/n | n/n | n/n | |
| | Sabino | nut | | | | | | | | | |
| | | Sorrel | | | | | | | | | |
| | | Sabino | | | | | | | | | |
| | Silver | Silver | X | X | X | X | X | X | n/n | n/n | |
| | Amber | Amber | | | | | | | | | |
| | Dun | Cham- | | | | | | | | | |
| | Sabino | pagne | | | | | | | | | |
| | | Dun | | | | | | | | | |
| | | Sabino | | | | | | | | | |
| | | Silver | n/n | X | n/n | n/n | X | X | n/n | n/n | |
| | | Classic | | | | | | | | | |
| | | Cham- | | | | | | | | | |
| | | pagne | | | | | | | | | |
| | | Sabino | | | | | | | | | |
| | | Silver | X | X | n/n | n/n | X | X | n/n | n/n | |
| | | Amber | | | | | | | | | |
| | | Cham- | | | | | | | | | |
| | | pagne | | | | | | | | | |
| | | Sabino | | | | | | | | | |
| | Silver | | n/n | X | X | X | X | X | n/n | n/n | |
| | Cham- | | | | | | | | | | |
| | pagne | | | | | | | | | | |
| | Dun | | | | | | | | | | |
| | Sabino | | | | | | | | | | |
| | | Silver | n/n | X | n/n | n/n | X | X | n/n | X/X | |
| | | Classic | | | | | | | | | |
| | | Cham- | | | | | | | | | |
| | | pagne | | | | | | | | | |
| | | Pearl | | | | | | | | | |
| | | Sabino | | | | | | | | | |
| | | Silver | X | X | n/n | n/n | X | X | n/n | X/X | |
| | | Classic | | | | | | | | | |
| | | Amber | | | | | | | | | |
| | | Pearl | | | | | | | | | |
| | | Sabino | | | | | | | | | |
| | | Silver | n/n | X | n/n | n/n | n/n | X | X/n | X/n | |
| | | Black | | | | | | | | | |
| | | Cream | | | | | | | | | |
| | | Pearl | | | | | | | | | |
| | | Sabino | | | | | | | | | |
| | | Silver | X | X | n/n | n/n | n/n | X | X/n | X/n | |
| | | Bay | | | | | | | | | |
| | | Cream | | | | | | | | | |
| | | Pearl | | | | | | | | | |
| | | Sabino | | | | | | | | | |
| | Silver | Silver | n/n | X | n/n | n/n | n/n | X | n/n | n/n | |
| | Dapple | Black | | | | | | | | | |
| | Sabino | Sabino | | | | | | | | | |
| | Silver | | X | X | n/n | n/n | n/n | X | n/n | n/n | |
| | Bay | | | | | | | | | | |
| | Sabino | | | | | | | | | | |
| | Silver | | X | X | n/n | n/n | n/n | X | X/n | n/n | |
| | Buck- | | | | | | | | | | |
| | skin | | | | | | | | | | |
| | Sabino | | | | | | | | | | |
| | Silver | | n/n | X | X | X | n/n | X | X/X | n/n | |
| | Cream | | | | | | | | | | |
| | Grullo | | | | | | | | | | |
| | Sabino | | | | | | | | | | |
| | Silver | Silver | | X | X | X | n/n | X | n/n | n/n | |
| | Dun | Dapple | | | | | | | | | |
| | Sabino | Dun | | | | | | | | | |
| | | Sabino | | | | | | | | | |
| | Silver | Silver | X | X | X | X | n/n | X | X/n | n/n | |
| | Dunskin | Dun Bay | | | | | | | | | |
| | Sabino | Cream | | | | | | | | | |
| | | Sabino | | | | | | | | | |
| | Silver | Silver | n/n | X | X | X | n/n | X | n/n | n/n | |
| | Grullo | Dun | | | | | | | | | |
| | Sabino | Black | | | | | | | | | |
| | | Sabino | | | | | | | | | |
| | | Silver | n/n | X | n/n | n/n | n/n | X | n/n | X/X | |
| | | Pearl | | | | | | | | | |
| | | Black | | | | | | | | | |
| | | Sabino | | | | | | | | | |
| | | Silver | X | X | n/n | n/n | n/n | X | n/n | X/X | |
| | | Pearl | | | | | | | | | |
| | | Bay | | | | | | | | | |
| | | Sabino | | | | | | | | | |
| | Silver | | X | X | n/n | n/n | n/n | X | X/X | n/n | |
| | Perlino | | | | | | | | | | |
| | Sabino | | | | | | | | | | |
| | Silver | | X | X | X | X | n/n | X | X/X | n/n | |
| | Perlino | | | | | | | | | | |
| | Dun | | | | | | | | | | |
| | Sabino | | | | | | | | | | |
| | | Silver | n/n | X | X | X | n/n | X | n/n | X/X | |
| | | Dun | | | | | | | | | |
| | | Pearl | | | | | | | | | |
| | | Black | | | | | | | | | |
| | | Sabino | | | | | | | | | |
| | | Silver | X | X | X | X | n/n | X | n/n | X/X | |
| | | Dun | | | | | | | | | |
| | | Pearl | | | | | | | | | |
| | | Bay | | | | | | | | | |
| | | Sabino | | | | | | | | | |
| | Silver | | n/n | X | n/n | n/n | n/n | X | X/X | n/n | |
| | Smoky | | | | | | | | | | |
| | Cream | | | | | | | | | | |
| | Sabino | | | | | | | | | | |
| | Smoky | Black | n/n | X | n/n | n/n | n/n | n/n | X/n | n/n | |
| | Black | Cream | | | | | | | | | |
| | Sabino | Sabino | | | | | | | | | |
| | Smoky | | n/n | X | n/n | n/n | n/n | n/n | X/X | n/n | |
| | Cream | | | | | | | | | | |
| | Sabino | | | | | | | | | | |
| | Smoky | Dun | n/n | X | X | X | n/n | n/n | X/n | n/n | |
| | Grullo | Black | | | | | | | | | |
| | Sabino | Cream | | | | | | | | | |
| | | Sabino | | | | | | | | | |
| | Cham- | | | | | | X | | | X/X | |
| | pagne | | | | | | | | | | |
| | Pearl | | | | | | | | | | |
| | Sabino | | | | | | | | | | |
| | Cream | | | | | | | | X/n | X/n | |
| | Pearl | | | | | | | | | | |
| | Sabino | | | | | | | | | | |
| | Dun | | | | X | X | | | | X/X | |
| | Pearl | | | | | | | | | | |
| | Sabino | | | | | | | | | | |
| | Silver | | | | | | X | X | | | |
| | Cham- | | | | | | | | | | |
| | pagne | | | | | | | | | | |
| | Sabino | | | | | | | | | | |
| | Silver | | | | | | X | X | | X/X | |
| | Cham- | | | | | | | | | | |
| | pagne | | | | | | | | | | |
| | Pearl | | | | | | | | | | |
| | Sabino | | | | | | | | | | |
| | Silver | | | | | | | X | X/n | X/n | |
| | Cream | | | | | | | | | | |
| | Pearl | | | | | | | | | | |
| | Sabino | | | | | | | | | | |
| | Silver | | | X | | | | X | | X/X | |
| | Pearl | | | | | | | | | | |
| | Sabino | | | | | | | | | | |
| | Silver | | | X | X | X | | X | | X/X | |
| | Dun | | | | | | | | | | |
| | Pearl | | | | | | | | | | |
| | Sabino | | | | | | | | | | |
| | Silver | | X | X | | | | X | X | | |
| | Bay | | | | | | | | | | |
| | Cream | | | | | | | | | | |
| | Tobiano | | | | | | | | | | |

| | Coat | | | | | | | | | | |
|---|---|---|---|---|---|---|---|---|---|---|---|
| | color + | | | | | | | | | | |
| Modifi- | modifi- | Also | | Tobiano- | | | | | | | |
| cation | cation | known as | KIT1SB12 | E2 | MITFSW1 | MITFSW3 | MITFSW5 | PAX3SW2 | PAX3P32R | KITW5 | KITW20 |
| Overo | Amber | Amber | | | | | | | | | X/n |
| | Dun | Cham- | | | | | | | | | |
| | Overo | pagne | | | | | | | | | |
| | | Dun | | | | | | | | | |
| | | Overo | | | | | | | | | |
| | Amber | Pseudo- | | | | | | | | | X/X |
| | Dun | cream | | | | | | | | | |
| | Pearl | Amber | | | | | | | | | |
| | Overo | Overo | | | | | | | | | |
| | Bay | | | | | | | | | | X/n |
| | Overo | | | | | | | | | | |
| | Black | | | | | | | | | | |
| | Overo | | | | | | | | | | |
| | Buck- | | | | | | | | | | |
| | skin | | | | | | | | | | |
| | Overo | | | | | | | | | | |
| | | Gold | | | | | | | | | |
| | | Cham- | | | | | | | | | |
| | | pagne | | | | | | | | | |
| | | Overo | | | | | | | | | |
| | | Classic | | | | | | | | | |
| | | Cham- | | | | | | | | | |
| | | pagne | | | | | | | | | |
| | | Overo | | | | | | | | | |
| | | Amber | | | | | | | | | |
| | | Cham- | | | | | | | | | |
| | | pagne | | | | | | | | | |
| | | Overo | | | | | | | | | |
| | Cham- | Classic | | | X | | | | | | |
| | pagne | Cham- | | | | | | | | | |
| | Dun | pagne | | | | | | | | | |
| | Overo | Dun | | | | | | | | | |
| | | Overo | | | | | | | | | |
| | Cham- | Pseudo- | | | | | | X/n | | | |
| | pagne | cream | | | | | | | | | |
| | Dun | Classic | | | | | | | | | |
| | Pearl | Overo | | | | | | | | | |
| | Overo | | | | | | | | | | |
| | | Cham- | | | | X/n | | | | | |
| | | pagne | | | | | | | | | |
| | | Gold | | | | | | | | | |
| | | Pearl | | | | | | | | | |
| | | Overo | | | | | | | | | |
| | | Cham- | | | | | | | X | | |
| | | pagne | | | | | | | | | |
| | | Classic | | | | | | | | | |
| | | Pearl | | | | | | | | | |
| | | Overo | | | | | | | | | |
| | | Cham- | | | | | X/n | | | | |
| | | pagne | | | | | | | | | |
| | | Amber | | | | | | | | | |
| | | Pearl | | | | | | | | | |
| | | Overo | | | | | | | | | |
| | | Gold | | | | | | | | | |
| | | Cream | | | | | | | | | |
| | | Overo | | | | | | | | | |
| | | Classic | | | | | | | | | |
| | | Cream | | | | | | | | | |
| | | Overo | | | | | | | | | |
| | | Amber | | | | | | | | | |
| | | Cream | | | | | | | | | |
| | | Overo | | | | | | | | | |
| | Cream | | | | | | | | | | |
| | Grullo | | | | | | | | | | |
| | Overo | | | | | | | | | | |
| | | Chest- | | | | | | | | | |
| | | nut | | | | | | | | | |
| | | Cream | | | | | | | | | |
| | | Pearl | | | | | | | | | |
| | | Overo | | | | | | | | | |
| | | Black | | | | | | | | | |
| | | Cream | | | | | | | | | |
| | | Pearl | | | | | | | | | |
| | | Overo | | | | | | | | | |
| | | Bay | | | | | | | | | |
| | | Cream | | | | | | | | | |
| | | Pearl | | | | | | | | | |
| | | Overo | | | | | | | | | |
| | Cremello | | | | | | | | | | |
| | Overo | | | | | | | | | | |
| | | Red Dun | | | | | | | | | |
| | | Overo | | | | | | | | | |
| | | Apricot | | | | | | | | | |
| | | Dun | | | | | | | | | |
| | | Overo | | | | | | | | | |
| | | Gold | | | | | | | | | |
| | | Dun | | | | | | | | | |
| | | Overo | | | | | | | | | |
| | | Classic | | | | | | | | | |
| | | Dun | | | | | | | | | |
| | | Overo | | | | | | | | | |
| | | Dun | | | | | | | | | |
| | | Pearl | | | | | | | | | |
| | | Black | | | | | | | | | |
| | | Overo | | | | | | | | | |
| | | Dun | | | | | | | | | |
| | | Pearl | | | | | | | | | |
| | | Bay | | | | | | | | | |
| | | Overo | | | | | | | | | |
| | Dunolino | | | | | | | | | | |
| | Overo | | | | | | | | | | |
| | Dunskin | | | | | | | | | | |
| | Overo | | | | | | | | | | |
| | Gold Dun | Pseudo- | | | | | | | | | |
| | Pearl | cream | | | | | | | | | |
| | Overo | Gold | | | | | | | | | |
| | | Overo | | | | | | | | | |
| | Grullo | | | | | | | | | | |
| | Overo | | | | | | | | | | |
| | Palomino | | | | | | | | | | |
| | Overo | | | | | | | | | | |
| | | Apricot | | | | | | | | | |
| | | Pearl | | | | | | | | | |
| | | Overo | | | | | | | | | |
| | | Black | | | | | | | | | |
| | | Pearl | | | | | | | | | |
| | | Overo | | | | | | | | | |
| | | Bay | | | | | | | | | |
| | | Pearl | | | | | | | | | |
| | | Over | | | | | | | | | |
| | Perlino | | | | | | | | | | |
| | Overo | | | | | | | | | | |
| | Perlino | | | | | | | | | | |
| | Dun | | | | | | | | | | |
| | Overo | | | | | | | | | | |
| | Red | Chest- | | | | | | | | | |
| | Overo | nut, | | | | | | | | | |
| | | Sorrel | | | | | | | | | |
| | | Overo | | | | | | | | | |
| | Silver | Silver | | | | | | | | | |
| | Amber | Amber | | | | | | | | | |
| | Dun | Cham- | | | | | | | | | |
| | Overo | pagne | | | | | | | | | |
| | | Dun | | | | | | | | | |
| | | Overo | | | | | | | | | |
| | | Silver | | | | | | | | | |
| | | Classic | | | | | | | | | |
| | | Cham- | | | | | | | | | |
| | | pagne | | | | | | | | | |
| | | Overo | | | | | | | | | |
| | | Silver | | | | | | | | | |
| | | Amber | | | | | | | | | |
| | | Cham- | | | | | | | | | |
| | | pagne | | | | | | | | | |
| | | Overo | | | | | | | | | |
| | Silver | | | | | | | | | | |
| | Cham- | | | | | | | | | | |
| | pagne | | | | | | | | | | |
| | Dun | | | | | | | | | | |
| | Overo | | | | | | | | | | |
| | | Silver | | | | | | | | | |
| | | Classic | | | | | | | | | |
| | | Cham- | | | | | | | | | |
| | | pagne | | | | | | | | | |
| | | Pearl | | | | | | | | | |
| | | Overo | | | | | | | | | |
| | | Silver | | | | | | | | | |
| | | Classic | | | | | | | | | |
| | | Amber | | | | | | | | | |
| | | Pearl | | | | | | | | | |
| | | Overo | | | | | | | | | |
| | | Silver | | | | | | | | | |
| | | Black | | | | | | | | | |
| | | Cream | | | | | | | | | |
| | | Pearl | | | | | | | | | |
| | | Overo | | | | | | | | | |
| | | Silver | | | | | | | | | |
| | | Bay | | | | | | | | | |
| | | Cream | | | | | | | | | |
| | | Pearl | | | | | | | | | |
| | | Overo | | | | | | | | | |
| | Silver | Silver | | | | | | | | | |
| | Dapple | Black | | | | | | | | | |
| | Overo | Overo | | | | | | | | | |
| | Silver | | | | | | | | | | |
| | Bay | | | | | | | | | | |
| | Overo | | | | | | | | | | |
| | Silver | | | | | | | | | | |
| | Buck- | | | | | | | | | | |
| | skin | | | | | | | | | | |
| | Overo | | | | | | | | | | |
| | Silver | | | | | | | | | | |
| | Cream | | | | | | | | | | |
| | Grullo | | | | | | | | | | |
| | Overo | | | | | | | | | | |
| | Silver | Silver | | | | | | | | | |
| | Dun | Dapple | | | | | | | | | |
| | Overo | Dun | | | | | | | | | |
| | | Overo | | | | | | | | | |
| | Silver | Silver | | | | | | | | | |
| | Dunskin | Dun Bay | | | | | | | | | |
| | Overo | Cream | | | | | | | | | |
| | | Overo | | | | | | | | | |
| | Silver | Silver | | | | | | | | | |
| | Grullo | Dun | | | | | | | | | |
| | Overo | Black | | | | | | | | | |
| | | Overo | | | | | | | | | |
| | | Silver | | | | | | | | | |
| | | Pearl | | | | | | | | | |
| | | Black | | | | | | | | | |
| | | Overo | | | | | | | | | |
| | | Silver | | | | | | | | | |
| | | Pearl | | | | | | | | | |
| | | Bay | | | | | | | | | |
| | | Overo | | | | | | | | | |
| | Silver | | | | | | | | | | |
| | Perlino | | | | | | | | | | |
| | Overo | | | | | | | | | | |
| | Silver | | | | | | | | | | |
| | Perlino | | | | | | | | | | |
| | Dun | | | | | | | | | | |
| | Overo | | | | | | | | | | |
| | | Silver | | | | | | | | | |
| | | Dun | | | | | | | | | |
| | | Pearl | | | | | | | | | |
| | | Black | | | | | | | | | |
| | | Overo | | | | | | | | | |
| | | Silver | | | | | | | | | |
| | | Dun | | | | | | | | | |
| | | Pearl | | | | | | | | | |
| | | Bay | | | | | | | | | |
| | | Overo | | | | | | | | | |
| | Silver | | | | | | | | | | |
| | Smoky | | | | | | | | | | |
| | Cream | | | | | | | | | | |
| | Overo | | | | | | | | | | |
| | Smoky | Black | | | | | | | | | |
| | Black | Cream | | | | | | | | | |
| | Overo | Overo | | | | | | | | | |
| | Smoky | | | | | | | | | | |
| | Cream | | | | | | | | | | |
| | Overo | | | | | | | | | | |
| | Smoky | Dun | | | | | | | | | |
| | Grullo | Black | | | | | | | | | |
| | Overo | Cream | | | | | | | | | |
| | | Overo | | | | | | | | | |
| | Cham- | | | | | | | | | | |
| | pagne | | | | | | | | | | |
| | Pearl | | | | | | | | | | |
| | Overo | | | | | | | | | | |
| | Cream | | | | | | | | | | |
| | Pearl | | | | | | | | | | |
| | Overo | | | | | | | | | | |
| | Dun | | | | | | | | | | |
| | Pearl | | | | | | | | | | |
| | Overo | | | | | | | | | | |
| | Silver | | | | | | | | | | |
| | Cham- | | | | | | | | | | |
| | pagne | | | | | | | | | | |
| | Overo | | | | | | | | | | |
| | Silver | | | | | | | | | | |
| | Cham- | | | | | | | | | | |
| | pagne | | | | | | | | | | |
| | Pearl | | | | | | | | | | |
| | Overo | | | | | | | | | | |
| | Silver | | | | | | | | | | |
| | Cream | | | | | | | | | | |
| | Pearl | | | | | | | | | | |
| | Overo | | | | | | | | | | |
| | Silver | | | | | | | | | | |
| | Pearl | | | | | | | | | | |
| | Overo | | | | | | | | | | |
| | Silver | | | | | | | | | | |
| | Dun | | | | | | | | | | |
| | Pearl | | | | | | | | | | |
| | Overo | | | | | | | | | | |
| | Silver | Silver | | | | | | | | | |
| | Bay | Buck- | | | | | | | | | |
| | Cream | skin | | | | | | | | | |
| | Overo | Overo | | | | | | | | | |
| Tovero | Amber | Amber | | X | | | | | | | X/n |
| | Dun | Cham- | | | | | | | | | |
| | Tovero | pagne | | | | | | | | | |
| | | Dun | | | | | | | | | |
| | | Tovero | | | | | | | | | |
| | Amber | Pseudo- | | X | | | | | | | X/X |
| | Dun | cream | | | | | | | | | |
| | Pearl | Amber | | | | | | | | | |
| | Tovero | Tovero | | | | | | | | | |
| | Bay | | | X | | | | | | X/n | X/n |
| | Tovero | | | | | | | | | | |
| | Black | | | X | | | | | | | |
| | Tovero | | | | | | | | | | |
| | Buck- | | | X | | | | | | | |
| | skin | | | | | | | | | | |
| | Tovero | | | | | | | | | | |
| | | Gold | | X | | | | | | | |
| | | Cham- | | | | | | | | | |
| | | pagne | | | | | | | | | |
| | | Tovero | | | | | | | | | |
| | | Classic | | X | | | | | | | |
| | | Cham- | | | | | | | | | |
| | | pagne | | | | | | | | | |
| | | Tovero | | | | | | | | | |
| | | Amber | | X | | | | | | | |
| | | Cham- | | | | | | | | | |
| | | pagne | | | | | | | | | |
| | | Tovero | | | | | | | | | |
| | Cham- | Classic | | X | X | | | | | | |
| | pagne | Cham- | | | | | | | | | |
| | Dun | pagne | | | | | | | | | |
| | Tovero | Dun | | | | | | | | | |
| | | Tovero | | | | | | | | | |
| | Cham- | Pseudo- | | X | | | | X/n | | | |
| | pagne | cream | | | | | | | | | |
| | Dun | Classic | | | | | | | | | |
| | Pearl | Tovero | | | | | | | | | |
| | Tovero | | | | | | | | | | |
| | | Cham- | | X | | X/n | | | | | |
| | | pagne | | | | | | | | | |
| | | Gold | | | | | | | | | |
| | | Pearl | | | | | | | | | |
| | | Tovero | | | | | | | | | |
| | | Cham- | | X | | | | | X | | |
| | | pagne | | | | | | | | | |
| | | Classic | | | | | | | | | |
| | | Pearl | | | | | | | | | |
| | | Tovero | | | | | | | | | |
| | | Cham- | | X | | | X/n | | | | |
| | | pagne | | | | | | | | | |
| | | Amber | | | | | | | | | |
| | | Pearl | | | | | | | | | |
| | | Tovero | | | | | | | | | |
| | | Gold | | X | | | | | | | |
| | | Cream | | | | | | | | | |
| | | Tovero | | | | | | | | | |
| | | Classic | | X | | | | | | | |
| | | Cream | | | | | | | | | |
| | | Tovero | | | | | | | | | |
| | | Amber | | X | | | | | | | |
| | | Cream | | | | | | | | | |
| | | Tovero | | | | | | | | | |
| | Cream | | | X | | | | | | | |
| | Grullo | | | | | | | | | | |
| | Tovero | | | | | | | | | | |
| | | Chest- | | X | | | | | | | |
| | | nut | | | | | | | | | |
| | | Cream | | | | | | | | | |
| | | Pearl | | | | | | | | | |
| | | Tovero | | | | | | | | | |
| | | Black | | X | | | | | | | |
| | | Cream | | | | | | | | | |
| | | Pearl | | | | | | | | | |
| | | Tovero | | | | | | | | | |
| | | Bay | | X | | | | | | | |
| | | Cream | | | | | | | | | |
| | | Pearl | | | | | | | | | |
| | | Tovero | | | | | | | | | |
| | Cremello | | | X | | | | | | | |
| | Tovero | | | | | | | | | | |
| | | Red Dun | | X | | | | | | | |
| | | Tovero | | | | | | | | | |
| | | Apricot | | X | | | | | | | |
| | | Dun | | | | | | | | | |
| | | Tovero | | | | | | | | | |
| | | Gold | | X | | | | | | | |
| | | Dun | | | | | | | | | |
| | | Tovero | | | | | | | | | |
| | | Classic | | X | | | | | | | |
| | | Dun | | | | | | | | | |
| | | Tovero | | | | | | | | | |
| | | Dun | | X | | | | | | | |
| | | Pearl | | | | | | | | | |
| | | Black | | | | | | | | | |
| | | Tovero | | | | | | | | | |
| | | Dun | | X | | | | | | | |
| | | Pearl | | | | | | | | | |
| | | Bay | | | | | | | | | |
| | | Tovero | | | | | | | | | |
| | Dunolino | | | X | | | | | | | |
| | Tovero | | | | | | | | | | |
| | Dunskin | | | X | | | | | | | |
| | Tovero | | | | | | | | | | |
| | Gold | Pseudo- | | X | | | | | | | |
| | Dun | cream | | | | | | | | | |
| | Pearl | Gold | | | | | | | | | |
| | Tovero | Tovero | | | | | | | | | |
| | Grullo | | | X | | | | | | | |
| | Tovero | | | | | | | | | | |
| | Palomino | | | X | | | | | | | |
| | Tovero | | | | | | | | | | |
| | | Apricot | | X | | | | | | | |
| | | Pearl | | | | | | | | | |
| | | Tovero | | | | | | | | | |
| | | Black | | X | | | | | | | |
| | | Pearl | | | | | | | | | |
| | | Tovero | | | | | | | | | |
| | | Bay | | X | | | | | | | |
| | | Pearl | | | | | | | | | |
| | | Tovero | | | | | | | | | |
| | Perlino | | | X | | | | | | | |
| | Tovero | | | | | | | | | | |
| | Perlino | | | X | | | | | | | |
| | Dun | | | | | | | | | | |
| | Tovero | | | | | | | | | | |
| | Red | Chest- | | X | | | | | | | |
| | Tovero | nut, | | | | | | | | | |
| | | Sorrel | | | | | | | | | |
| | | Tovero | | | | | | | | | |
| | Silver | Silver | | X | | | | | | | |
| | Amber | Amber | | | | | | | | | |
| | Dun | Cham- | | | | | | | | | |
| | Tovero | pagne | | | | | | | | | |
| | | Dun | | | | | | | | | |
| | | Tovero | | | | | | | | | |
| | | Silver | | X | | | | | | | |
| | | Classic | | | | | | | | | |
| | | Cham- | | | | | | | | | |
| | | pagne | | | | | | | | | |
| | | Tovero | | | | | | | | | |
| | | Silver | | X | | | | | | | |
| | | Amber | | | | | | | | | |
| | | Cham- | | | | | | | | | |
| | | pagne | | | | | | | | | |
| | | Tovero | | | | | | | | | |
| | Silver | | | X | | | | | | | |
| | Cham- | | | | | | | | | | |
| | pagne | | | | | | | | | | |
| | Dun | | | | | | | | | | |
| | Tovero | | | | | | | | | | |
| | | Silver | | X | | | | | | | |
| | | Classic | | | | | | | | | |
| | | Cham- | | | | | | | | | |
| | | pagne | | | | | | | | | |
| | | Pearl | | | | | | | | | |
| | | Tovero | | | | | | | | | |
| | | Silver | | X | | | | | | | |
| | | Classic | | | | | | | | | |
| | | Amber | | | | | | | | | |
| | | Pearl | | | | | | | | | |
| | | Tovero | | | | | | | | | |
| | | Silver | | X | | | | | | | |
| | | Black | | | | | | | | | |
| | | Cream | | | | | | | | | |
| | | Pearl | | | | | | | | | |
| | | Tovero | | | | | | | | | |
| | | Silver | | X | | | | | | | |
| | | Bay | | | | | | | | | |
| | | Cream | | | | | | | | | |
| | | Pearl | | | | | | | | | |
| | | Tovero | | | | | | | | | |
| | Silver | Silver | | X | | | | | | | |
| | Dapple | Black | | | | | | | | | |
| | Tovero | Tovero | | | | | | | | | |
| | Silver | | | X | | | | | | | |
| | Bay | | | | | | | | | | |
| | Tovero | | | | | | | | | | |
| | Silver | | | X | | | | | | | |
| | Buck- | | | | | | | | | | |
| | skin | | | | | | | | | | |
| | Tovero | | | | | | | | | | |
| | Silver | | | X | | | | | | | |
| | Cream | | | | | | | | | | |
| | Grullo | | | | | | | | | | |
| | Tovero | | | | | | | | | | |
| | Silver | Silver | | X | | | | | | | |
| | Dun | Dapple | | | | | | | | | |
| | Tovero | Dun | | | | | | | | | |
| | | Tovero | | | | | | | | | |
| | Silver | Silver | | X | | | | | | | |
| | Dunskin | Dun Bay | | | | | | | | | |
| | Tovero | Cream | | | | | | | | | |
| | | Tovero | | | | | | | | | |
| | Silver | Silver | | X | | | | | | | |
| | Grullo | Dun | | | | | | | | | |
| | Tovero | Black | | | | | | | | | |
| | | Tovero | | | | | | | | | |
| | | Silver | | X | | | | | | | |
| | | Pearl | | | | | | | | | |
| | | Black | | | | | | | | | |
| | | Tovero | | | | | | | | | |
| | | Silver | | X | | | | | | | |
| | | Pearl | | | | | | | | | |
| | | Bay | | | | | | | | | |
| | | Tovero | | | | | | | | | |
| | Silver | | | X | | | | | | | |
| | Perlino | | | | | | | | | | |
| | Tovero | | | | | | | | | | |
| | Silver | | | X | | | | | | | |
| | Perlino | | | | | | | | | | |
| | Dun | | | | | | | | | | |
| | Tovero | | | | | | | | | | |
| | | Silver | | X | | | | | | | |
| | | Dun | | | | | | | | | |
| | | Pearl | | | | | | | | | |
| | | Black | | | | | | | | | |
| | | Tovero | | | | | | | | | |
| | | Silver | | X | | | | | | | |
| | | Dun | | | | | | | | | |
| | | Pearl | | | | | | | | | |
| | | Bay | | | | | | | | | |
| | | Tovero | | | | | | | | | |
| | Silver | | | X | | | | | | | |
| | Smoky | | | | | | | | | | |
| | Cream | | | | | | | | | | |
| | Tovero | | | | | | | | | | |
| | Smoky | Black | | X | | | | | | | |
| | Black | Cream | | | | | | | | | |
| | Tovero | Tovero | | | | | | | | | |
| | Smoky | | | X | | | | | | | |
| | Cream | | | | | | | | | | |
| | Tovero | | | | | | | | | | |
| | Smoky | Dun | | X | | | | | | | |
| | Grullo | Black | | | | | | | | | |
| | Tovero | Cream | | | | | | | | | |
| | | Tovero | | | | | | | | | |
| | Cham- | | | X | | | | | | | |
| | pagne | | | | | | | | | | |
| | Pearl | | | | | | | | | | |
| | Tovero | | | | | | | | | | |
| | Cream | | | X | | | | | | | |
| | Pearl | | | | | | | | | | |
| | Tovero | | | | | | | | | | |
| | Dun | | | X | | | | | | | |
| | Pearl | | | | | | | | | | |
| | Tovero | | | | | | | | | | |
| | Silver | | | X | | | | | | | |
| | Cham- | | | | | | | | | | |
| | pagne | | | | | | | | | | |
| | Tovero | | | | | | | | | | |
| | Silver | | | X | | | | | | | |
| | Cham- | | | | | | | | | | |
| | pagne | | | | | | | | | | |
| | Pearl | | | | | | | | | | |
| | Tovero | | | | | | | | | | |
| | Silver | | | X | | | | | | | |
| | Cream | | | | | | | | | | |
| | Pearl | | | | | | | | | | |
| | Tovero | | | | | | | | | | |
| | Silver | | | X | | | | | | | |
| | Pearl | | | | | | | | | | |
| | Tovero | | | | | | | | | | |
| | Silver | | | X | | | | | | | |
| | Dun | | | | | | | | | | |
| | Pearl | | | | | | | | | | |
| | Tovero | | | | | | | | | | |
| | Silver | Silver | | X | | | | | | | |
| | Bay | Buck- | | | | | | | | | |
| | Cream | skin | | | | | | | | | |
| | Tovero | Tovero | | | | | | | | | |
| Splash | Amber | Amber | | | X | X/n | X/n | X/n | X/n | | X/n |
| | Dun | Cham- | | | | | | | | | |
| | Splash | pagne | | | | | | | | | |
| | | Dun | | | | | | | | | |
| | | Splash | | | | | | | | | |
| | Amber | Pseudo- | | | X | X/n | X/n | X/n | X/n | | |
| | Dun | cream | | | | | | | | | |
| | Pearl | Amber | | | | | | | | | |
| | Splash | Splash | | | | | | | | | |
| | Bay | | | | X | X/n | X/n | X/n | X/n | | |
| | Splash | | | | | | | | | | |
| | Black | | | | X | X/n | X/n | X/n | X/n | | |
| | Splash | | | | | | | | | | |
| | Buck- | | | | X | X/n | X/n | X/n | X/n | | |
| | skin | | | | | | | | | | |
| | Splash | | | | | | | | | | |
| | | Gold | | | X | X/n | X/n | X/n | X/n | | |
| | | Cham- | | | | | | | | | |
| | | pagne | | | | | | | | | |
| | | Splash | | | | | | | | | |
| | | Classic | | | X | X/n | X/n | X/n | X/n | | |
| | | Cham- | | | | | | | | | |
| | | pagne | | | | | | | | | |
| | | Splash | | | | | | | | | |
| | | Amber | | | X | X/n | X/n | X/n | X/n | | |
| | | Cham- | | | | | | | | | |
| | | pagne | | | | | | | | | |
| | | Splash | | | | | | | | | |
| | Cham- | Classic | | | X | X/n | X/n | X/n | X/n | | |
| | pagne | Cham- | | | | | | | | | |
| | Dun | pagne | | | | | | | | | |
| | Splash | Dun | | | | | | | | | |
| | | Splash | | | | | | | | | |
| | Cham- | Pseudo- | | | X | X/n | X/n | X/n | X/n | | |
| | pagne | cream | | | | | | | | | |
| | Dun | Classic | | | | | | | | | |
| | Pearl | Splash | | | | | | | | | |
| | Splash | | | | | | | | | | |
| | | Cham- | | | X | X/n | X/n | X/n | X/n | | |
| | | pagne | | | | | | | | | |
| | | Gold | | | | | | | | | |
| | | Pearl | | | | | | | | | |
| | | Splash | | | | | | | | | |
| | | Cham- | | | X | X/n | X/n | X/n | X/n | | |
| | | pagne | | | | | | | | | |
| | | Classic | | | | | | | | | |
| | | Pearl | | | | | | | | | |
| | | Splash | | | | | | | | | |
| | | Cham- | | | X | X/n | X/n | X/n | X/n | | |
| | | pagne | | | | | | | | | |
| | | Amber | | | | | | | | | |
| | | Pearl | | | | | | | | | |
| | | Splash | | | | | | | | | |
| | | Gold | | | X | X/n | X/n | X/n | X/n | | |
| | | Cream | | | | | | | | | |
| | | Splash | | | | | | | | | |
| | | Classic | | | X | X/n | X/n | X/n | X/n | | |
| | | Cream | | | | | | | | | |
| | | Splash | | | | | | | | | |
| | | Amber | | | X | X/n | X/n | X/n | X/n | | |
| | | Cream | | | | | | | | | |
| | | Splash | | | | | | | | | |
| | Cream | | | | X | X/n | X/n | X/n | X/n | | |
| | Grullo | | | | | | | | | | |
| | Splash | | | | | | | | | | |
| | | Chest- | | | X | X/n | X/n | X/n | X/n | | |
| | | nut | | | | | | | | | |
| | | Cream | | | | | | | | | |
| | | Pearl | | | | | | | | | |
| | | Splash | | | | | | | | | |
| | | Black | | | X | X/n | X/n | X/n | X/n | | |
| | | Cream | | | | | | | | | |
| | | Pearl | | | | | | | | | |
| | | Splash | | | | | | | | | |
| | | Bay | | | X | X/n | X/n | X/n | X/n | | |
| | | Cream | | | | | | | | | |
| | | Pearl | | | | | | | | | |
| | | Splash | | | | | | | | | |
| | Cremello | | | | X | X/n | X/n | X/n | X/n | | |
| | Splash | | | | | | | | | | |
| | | Red Dun | | | X | X/n | X/n | X/n | X/n | | |
| | | Splash | | | | | | | | | |
| | | Apricot | | | X | X/n | X/n | X/n | X/n | | |
| | | Dun | | | | | | | | | |
| | | Splash | | | | | | | | | |
| | | Gold | | | X | X/n | X/n | X/n | X/n | | |
| | | Dun | | | | | | | | | |
| | | Splash | | | | | | | | | |
| | | Classic | | | X | X/n | X/n | X/n | X/n | | |
| | | Dun | | | | | | | | | |
| | | Splash | | | | | | | | | |
| | | Dun | | | X | X/n | X/n | X/n | X/n | | |
| | | Pearl | | | | | | | | | |
| | | Black | | | | | | | | | |
| | | Splash | | | | | | | | | |
| | | Dun | | | X | X/n | X/n | X/n | X/n | | |
| | | Pearl | | | | | | | | | |
| | | Bay | | | | | | | | | |
| | | Splash | | | | | | | | | |
| | Dunolino | | | | X | X/n | X/n | X/n | X/n | | |
| | Splash | | | | | | | | | | |
| | Dunskin | | | | X | X/n | X/n | X/n | X/n | | |
| | Splash | | | | | | | | | | |
| | Gold | Pseudo- | | | X | X/n | X/n | X/n | X/n | | |
| | Dun | cream | | | | | | | | | |
| | Pearl | Gold | | | | | | | | | |
| | Splash | Splash | | | | | | | | | |
| | Grullo | | | | X | X/n | X/n | X/n | X/n | | |
| | Splash | | | | | | | | | | |
| | Palomino | | | | X | X/n | X/n | X/n | X/n | | |
| | Splash | | | | | | | | | | |
| | | Apricot | | | X | X/n | X/n | X/n | X/n | | |
| | | Pearl | | | | | | | | | |
| | | Splash | | | | | | | | | |
| | | Black | | | X | X/n | X/n | X/n | X/n | | |
| | | Pearl | | | | | | | | | |
| | | Splash | | | | | | | | | |
| | | Bay | | | X | X/n | X/n | X/n | X/n | | |
| | | Pearl | | | | | | | | | |
| | | Splash | | | | | | | | | |
| | Perlino | | | | X | X/n | X/n | X/n | X/n | | |
| | Splash | | | | | | | | | | |
| | Perlino | | | | X | X/n | X/n | X/n | X/n | | |
| | Dun | | | | | | | | | | |
| | Splash | | | | | | | | | | |
| | Red | Chest- | | | X | X/n | X/n | X/n | X/n | | |
| | Splash | nut, | | | | | | | | | |
| | | Sorrel | | | | | | | | | |
| | | Splash | | | | | | | | | |
| | Silver | Silver | | | X | X/n | X/n | X/n | X/n | | |
| | Amber | Amber | | | | | | | | | |
| | Dun | Cham- | | | | | | | | | |
| | Splash | pagne | | | | | | | | | |
| | | Dun | | | | | | | | | |
| | | Splash | | | | | | | | | |
| | | Silver | | | X | X/n | X/n | X/n | X/n | | |
| | | Classic | | | | | | | | | |
| | | Cham- | | | | | | | | | |
| | | pagne | | | | | | | | | |
| | | Splash | | | | | | | | | |
| | | Silver | | | X | X/n | X/n | X/n | X/n | | |
| | | Amber | | | | | | | | | |
| | | Cham- | | | | | | | | | |
| | | pagne | | | | | | | | | |
| | | Splash | | | | | | | | | |
| | Silver | | | | X | X/n | X/n | X/n | X/n | | |
| | Cham- | | | | | | | | | | |
| | pagne | | | | | | | | | | |
| | Dun | | | | | | | | | | |
| | Splash | | | | | | | | | | |
| | | Silver | | | X | X/n | X/n | X/n | X/n | | |
| | | Classic | | | | | | | | | |
| | | Cham- | | | | | | | | | |
| | | pagne | | | | | | | | | |
| | | Pearl | | | | | | | | | |
| | | Splash | | | | | | | | | |
| | | Silver | | | X | X/n | X/n | X/n | X/n | | |
| | | Classic | | | | | | | | | |
| | | Amber | | | | | | | | | |
| | | Pearl | | | | | | | | | |
| | | Splash | | | | | | | | | |
| | | Silver | | | X | X/n | X/n | X/n | X/n | | |
| | | Black | | | | | | | | | |
| | | Cream | | | | | | | | | |
| | | Pearl | | | | | | | | | |
| | | Splash | | | | | | | | | |
| | | Silver | | | X | X/n | X/n | X/n | X/n | | |
| | | Bay | | | | | | | | | |
| | | Cream | | | | | | | | | |
| | | Pearl | | | | | | | | | |
| | | Splash | | | | | | | | | |
| | Silver | Silver | | | X | X/n | X/n | X/n | X/n | | |
| | Dapple | Black | | | | | | | | | |
| | Splash | Splash | | | | | | | | | |
| | Silver | | | | X | X/n | X/n | X/n | X/n | | |
| | Bay | | | | | | | | | | |
| | Splash | | | | | | | | | | |
| | Silver | | | | X | X/n | X/n | X/n | X/n | | |
| | Buck- | | | | | | | | | | |
| | skin | | | | | | | | | | |
| | Splash | | | | | | | | | | |
| | Silver | | | | X | X/n | X/n | X/n | X/n | | |
| | Cream | | | | | | | | | | |
| | Grullo | | | | | | | | | | |
| | Splash | | | | | | | | | | |
| | Silver | Silver | | | X | X/n | X/n | X/n | X/n | | |
| | Dun | Dapple | | | | | | | | | |
| | Splash | Dun | | | | | | | | | |
| | | Splash | | | | | | | | | |
| | Silver | Silver | | | X | X/n | X/n | X/n | X/n | | |
| | Dunskin | Dun Bay | | | | | | | | | |
| | Splash | Cream | | | | | | | | | |
| | | Splash | | | | | | | | | |
| | Silver | Silver | | | X | X/n | X/n | X/n | X/n | | |
| | Grullo | Dun | | | | | | | | | |
| | Splash | Black | | | | | | | | | |
| | | Splash | | | | | | | | | |
| | | Silver | | | X | X/n | X/n | X/n | X/n | | |
| | | Pearl | | | | | | | | | |
| | | Black | | | | | | | | | |
| | | Splash | | | | | | | | | |
| | | Silver | | | X | X/n | X/n | X/n | X/n | | |
| | | Pearl | | | | | | | | | |
| | | Bay | | | | | | | | | |
| | | Splash | | | | | | | | | |
| | Silver | | | | X | X/n | X/n | X/n | X/n | | |
| | Perlino | | | | | | | | | | |
| | Splash | | | | | | | | | | |
| | Silver | | | | X | X/n | X/n | X/n | X/n | | |
| | Perlino | | | | | | | | | | |
| | Dun | | | | | | | | | | |
| | Splash | | | | | | | | | | |
| | | Silver | | | X | X/n | X/n | X/n | X/n | | |
| | | Dun | | | | | | | | | |
| | | Pearl | | | | | | | | | |
| | | Black | | | | | | | | | |
| | | Splash | | | | | | | | | |
| | | Silver | | | X | X/n | X/n | X/n | X/n | | |
| | | Dun | | | | | | | | | |
| | | Pearl | | | | | | | | | |
| | | Bay | | | | | | | | | |
| | | Splash | | | | | | | | | |
| | Silver | | | | X | X/n | X/n | X/n | X/n | | |
| | Smoky | | | | | | | | | | |
| | Cream | | | | | | | | | | |
| | Splash | | | | | | | | | | |
| | Smoky | Black | | | X | X/n | X/n | X/n | X/n | | |
| | Black | Cream | | | | | | | | | |
| | Splash | Splash | | | | | | | | | |
| | Smoky | | | | X | X/n | X/n | X/n | X/n | | |
| | Cream | | | | | | | | | | |
| | Splash | | | | | | | | | | |
| | Smoky | Dun | | | X | X/n | X/n | X/n | X/n | | |
| | Grullo | Black | | | | | | | | | |
| | Splash | Cream | | | | | | | | | |
| | | Splash | | | | | | | | | |
| | Cham- | | | | X | X/n | X/n | X/n | X/n | | |
| | pagne | | | | | | | | | | |
| | Pearl | | | | | | | | | | |
| | Splash | | | | | | | | | | |
| | Cream | | | | X | X/n | X/n | X/n | X/n | | |
| | Pearl | | | | | | | | | | |
| | Splash | | | | | | | | | | |
| | Dun | | | | X | X/n | X/n | X/n | X/n | | |
| | Pearl | | | | | | | | | | |
| | Splash | | | | | | | | | | |
| | Silver | | | | X | X/n | X/n | X/n | X/n | | |
| | Cham- | | | | | | | | | | |
| | pagne | | | | | | | | | | |
| | Splash | | | | | | | | | | |
| | Silver | | | | X | X/n | X/n | X/n | X/n | | |
| | Cham- | | | | | | | | | | |
| | pagne | | | | | | | | | | |
| | Pearl | | | | | | | | | | |
| | Splash | | | | | | | | | | |
| | Silver | | | | X | X/n | X/n | X/n | X/n | | |
| | Cream | | | | | | | | | | |
| | Pearl | | | | | | | | | | |
| | Splash | | | | | | | | | | |
| | Silver | | | | X | X/n | X/n | X/n | X/n | | |
| | Pearl | | | | | | | | | | |
| | Splash | | | | | | | | | | |
| | Silver | | | | X | X/n | X/n | X/n | X/n | | |
| | Dun | | | | | | | | | | |
| | Pearl | | | | | | | | | | |
| | Splash | | | | | | | | | | |
| | Silver | | | | X | X/n | X/n | X/n | X/n | | |
| | Bay | | | | | | | | | | |
| | Cream | | | | | | | | | | |
| | Splash | | | | | | | | | | |
| Tobiano | Amber | Amber | | X | | | | | | | X/n |
| | Dun | Cham- | | | | | | | | | |
| | Tobiano | pagne | | | | | | | | | |
| | | Dun | | | | | | | | | |
| | | Tobiano | | | | | | | | | |
| | Amber | Pseudo- | | X | | | | | | | X/X |
| | Dun | cream | | | | | | | | | |
| | Pearl | Amber | | | | | | | | | |
| | Tobiano | Tobiano | | | | | | | | | |
| | Bay | | | X | | | | | | X/n | X/n |
| | Tobiano | | | | | | | | | | |
| | Black | | | X | | | | | | | |
| | Tobiano | | | | | | | | | | |
| | Buck- | | | X | | | | | | | |
| | skin | | | | | | | | | | |
| | Tobiano | | | | | | | | | | |
| | | Gold | | X | | | | | | | |
| | | Cham- | | | | | | | | | |
| | | pagne | | | | | | | | | |
| | | Tobiano | | | | | | | | | |
| | | Classic | | X | | | | | | | |
| | | Cham- | | | | | | | | | |
| | | pagne | | | | | | | | | |
| | | Tobiano | | | | | | | | | |
| | | Amber | | X | | | | | | | |
| | | Cham- | | | | | | | | | |
| | | pagne | | | | | | | | | |
| | | Tobiano | | | | | | | | | |
| | Cham- | Classic | | X | X | | | | | | |
| | pagne | Cham- | | | | | | | | | |
| | Dun | pagne | | | | | | | | | |
| | Tobiano | Dun | | | | | | | | | |
| | | Tobiano | | | | | | | | | |
| | Cham- | Pseudo- | | X | | | | X/n | | | |
| | pagne | cream | | | | | | | | | |
| | Dun | Classic | | | | | | | | | |
| | Pearl | Tobiano | | | | | | | | | |
| | Tobiano | | | | | | | | | | |
| | | Cham- | | X | | X/n | | | | | |
| | | pagne | | | | | | | | | |
| | | Gold | | | | | | | | | |
| | | Pearl | | | | | | | | | |
| | | Tobiano | | | | | | | | | |
| | | Cham- | | X | | | | | X | | |
| | | pagne | | | | | | | | | |
| | | Classic | | | | | | | | | |
| | | Pearl | | | | | | | | | |
| | | Tobiano | | | | | | | | | |
| | | Cham- | | X | | | X/n | | | | |
| | | pagne | | | | | | | | | |
| | | Amber | | | | | | | | | |
| | | Pearl | | | | | | | | | |
| | | Tobiano | | | | | | | | | |
| | | Gold | | X | | | | | | | |
| | | Cream | | | | | | | | | |
| | | Tobiano | | | | | | | | | |
| | | Classic | | X | | | | | | | |
| | | Cream | | | | | | | | | |
| | | Tobiano | | | | | | | | | |
| | | Amber | | X | | | | | | | |
| | | Cream | | | | | | | | | |
| | | Tobiano | | | | | | | | | |
| | Cream | | | X | | | | | | | |
| | Grullo | | | | | | | | | | |
| | Tobiano | | | | | | | | | | |
| | | Chest- | | X | | | | | | | |
| | | nut | | | | | | | | | |
| | | Cream | | | | | | | | | |
| | | Pearl | | | | | | | | | |
| | | Tobiano | | | | | | | | | |
| | | Black | | X | | | | | | | |
| | | Cream | | | | | | | | | |
| | | Pearl | | | | | | | | | |
| | | Tobiano | | | | | | | | | |
| | | Bay | | X | | | | | | | |
| | | Cream | | | | | | | | | |
| | | Pearl | | | | | | | | | |
| | | Tobiano | | | | | | | | | |
| | Cremello | | | X | | | | | | | |
| | Tobiano | | | | | | | | | | |
| | | Red Dun | | X | | | | | | | |
| | | Tobiano | | | | | | | | | |
| | | Apricot | | X | | | | | | | |
| | | Dun | | | | | | | | | |
| | | Tobiano | | | | | | | | | |
| | | Gold | | X | | | | | | | |
| | | Dun | | | | | | | | | |
| | | Tobiano | | | | | | | | | |
| | | Classic | | X | | | | | | | |
| | | Dun | | | | | | | | | |
| | | Tobiano | | | | | | | | | |
| | | Dun | | X | | | | | | | |
| | | Pearl | | | | | | | | | |
| | | Black | | | | | | | | | |
| | | Tobiano | | | | | | | | | |
| | | Dun | | X | | | | | | | |
| | | Pearl | | | | | | | | | |
| | | Bay | | | | | | | | | |
| | | Tobiano | | | | | | | | | |
| | Dunolino | | | X | | | | | | | |
| | Tobiano | | | | | | | | | | |
| | Dunskin | | | X | | | | | | | |
| | Tobiano | | | | | | | | | | |
| | Gold Dun | Pseudo- | | X | | | | | | | |
| | Pearl | cream | | | | | | | | | |
| | Tobiano | Gold | | | | | | | | | |
| | | Tobiano | | | | | | | | | |
| | Grullo | | | X | | | | | | | |
| | Tobiano | | | | | | | | | | |
| | Palomino | | | X | | | | | | | |
| | Tobiano | | | | | | | | | | |
| | | Apricot | | X | | | | | | | |
| | | Pearl | | | | | | | | | |
| | | Tobiano | | | | | | | | | |
| | | Black | | X | | | | | | | |
| | | Pearl | | | | | | | | | |
| | | Tobiano | | | | | | | | | |
| | | Bay | | X | | | | | | | |
| | | Pearl | | | | | | | | | |
| | | Tobiano | | | | | | | | | |
| | Perlino | | | X | | | | | | | |
| | Tobiano | | | | | | | | | | |
| | Perlino | | | X | | | | | | | |
| | Dun | | | | | | | | | | |
| | Tobiano | | | | | | | | | | |
| | Red | Chest- | | X | | | | | | | |
| | Tobiano | nut, | | | | | | | | | |
| | | Sorrel | | | | | | | | | |
| | Silver | Silver | | X | | | | | | | |
| | Amber | Amber | | | | | | | | | |
| | Dun | Cham- | | | | | | | | | |
| | Tobiano | pagne | | | | | | | | | |
| | | Dun | | | | | | | | | |
| | | Tobiano | | | | | | | | | |
| | | Silver | | X | | | | | | | |
| | | Classic | | | | | | | | | |
| | | Cham- | | | | | | | | | |
| | | pagne | | | | | | | | | |
| | | Tobiano | | | | | | | | | |
| | | Silver | | X | | | | | | | |
| | | Amber | | | | | | | | | |
| | | Cham- | | | | | | | | | |
| | | pagne | | | | | | | | | |
| | | Tobiano | | | | | | | | | |
| | Silver | | | X | | | | | | | |
| | Cham- | | | | | | | | | | |
| | pagne | | | | | | | | | | |
| | Dun | | | | | | | | | | |
| | Tobiano | | | | | | | | | | |
| | | Silver | | X | | | | | | | |
| | | Classic | | | | | | | | | |
| | | Cham- | | | | | | | | | |
| | | pagne | | | | | | | | | |
| | | Pearl | | | | | | | | | |
| | | Tobiano | | | | | | | | | |
| | | Silver | | X | | | | | | | |
| | | Classic | | | | | | | | | |
| | | Amber | | | | | | | | | |
| | | Pearl | | | | | | | | | |
| | | Tobiano | | | | | | | | | |
| | | Silver | | X | | | | | | | |
| | | Black | | | | | | | | | |
| | | Cream | | | | | | | | | |
| | | Pearl | | | | | | | | | |
| | | Tobiano | | | | | | | | | |
| | | Silver | | X | | | | | | | |
| | | Bay | | | | | | | | | |
| | | Cream | | | | | | | | | |
| | | Pearl | | | | | | | | | |
| | | Tobiano | | | | | | | | | |
| | Silver | Silver | | X | | | | | | | |
| | Dapple | Black | | | | | | | | | |
| | Tobiano | Tobiano | | | | | | | | | |
| | Silver | | | X | | | | | | | |
| | Bay | | | | | | | | | | |
| | Tobiano | | | | | | | | | | |
| | Silver | | | X | | | | | | | |
| | Buck- | | | | | | | | | | |
| | skin | | | | | | | | | | |
| | Tobiano | | | | | | | | | | |
| | Silver | | | X | | | | | | | |
| | Cream | | | | | | | | | | |
| | Grullo | | | | | | | | | | |
| | Tobiano | | | | | | | | | | |
| | Silver | Silver | | X | | | | | | | |
| | Dun | Dapple | | | | | | | | | |
| | Tobiano | Dun | | | | | | | | | |
| | | Tobiano | | | | | | | | | |
| | Silver | Silver | | X | | | | | | | |
| | Dunskin | Dun Bay | | | | | | | | | |
| | Tobiano | Cream | | | | | | | | | |
| | | Tobiano | | | | | | | | | |
| | Silver | Silver | | X | | | | | | | |
| | Grullo | Dun | | | | | | | | | |
| | Tobiano | Black | | | | | | | | | |
| | | Tobiano | | | | | | | | | |
| | | Silver | | X | | | | | | | |
| | | Pearl | | | | | | | | | |
| | | Black | | | | | | | | | |
| | | Tobiano | | | | | | | | | |
| | | Silver | | X | | | | | | | |
| | | Pearl | | | | | | | | | |
| | | Bay | | | | | | | | | |
| | | Tobiano | | | | | | | | | |
| | Silver | | | X | | | | | | | |
| | Perlino | | | | | | | | | | |
| | Tobiano | | | | | | | | | | |
| | Silver | | | X | | | | | | | |
| | Perlino | | | | | | | | | | |
| | Dun | | | | | | | | | | |
| | Tobiano | | | | | | | | | | |
| | | Silver | | X | | | | | | | |
| | | Dun | | | | | | | | | |
| | | Pearl | | | | | | | | | |
| | | Black | | | | | | | | | |
| | | Tobiano | | | | | | | | | |
| | | Silver | | X | | | | | | | |
| | | Dun | | | | | | | | | |
| | | Pearl | | | | | | | | | |
| | | Bay | | | | | | | | | |
| | | Tobiano | | | | | | | | | |
| | Silver | | | X | | | | | | | |
| | Smoky | | | | | | | | | | |
| | Cream | | | | | | | | | | |
| | Tobiano | | | | | | | | | | |
| | Smoky | Black | | X | | | | | | | |
| | Black | Cream | | | | | | | | | |
| | Tobiano | Tobiano | | | | | | | | | |
| | Smoky | | | X | | | | | | | |
| | Cream | | | | | | | | | | |
| | Tobiano | | | | | | | | | | |
| | Smoky | Dun | | X | | | | | | | |
| | Grullo | Black | | | | | | | | | |
| | Tobiano | Cream | | | | | | | | | |
| | | Tobiano | | | | | | | | | |
| | Cham- | | | X | | | | | | | |
| | pagne | | | | | | | | | | |
| | Pearl | | | | | | | | | | |
| | Tobiano | | | | | | | | | | |
| | Cream | | | X | | | | | | | |
| | Pearl | | | | | | | | | | |
| | Tobiano | | | | | | | | | | |
| | Dun | | | X | | | | | | | |
| | Pearl | | | | | | | | | | |
| | Tobiano | | | | | | | | | | |
| | Silver | | | X | | | | | | | |
| | Cham- | | | | | | | | | | |
| | pagne | | | | | | | | | | |
| | Tobiano | | | | | | | | | | |
| | Silver | | | X | | | | | | | |
| | Cham- | | | | | | | | | | |
| | pagne | | | | | | | | | | |
| | Pearl | | | | | | | | | | |
| | Tobiano | | | | | | | | | | |
| | Silver | | | X | | | | | | | |
| | Cream | | | | | | | | | | |
| | Pearl | | | | | | | | | | |
| | Tobiano | | | | | | | | | | |
| | Silver | | | X | | | | | | | |
| | Pearl | | | | | | | | | | |
| | Tobiano | | | | | | | | | | |
| | Silver | | | X | | | | | | | |
| | Dun | | | | | | | | | | |
| | Pearl | | | | | | | | | | |
| | Tobiano | | | | | | | | | | |
| | Silver | | | | | | | | | | |
| | Bay | | | | | | | | | | |
| | Cream | | | | | | | | | | |
| | Tobiano | | | | | | | | | | |
| Sabino | Amber | Amber | | | | | | | | | X/n |
| | Dun | Cham- | | | | | | | | | |
| | Sabino | pagne | | | | | | | | | |
| | | Dun | | | | | | | | | |
| | | Sabino | | | | | | | | | |
| | Amber | Pseudo- | | | | | | | | | X/X |
| | Dun | cream | | | | | | | | | |
| | Pearl | Amber | | | | | | | | | |
| | Sabino | Sabino | | | | | | | | | |
| | Bay | | X | | | | | | | X/n | X/n |
| | Sabino | | | | | | | | | | |
| | Black | | X | | | | | | | | |
| | Sabino | | | | | | | | | | |
| | Buck- | | X | | | | | | | | |
| | skin | | | | | | | | | | |
| | Sabino | | | | | | | | | | |
| | | Gold | X | | | | | | | | |
| | | Cham- | | | | | | | | | |
| | | pagne | | | | | | | | | |
| | | Sabino | | | | | | | | | |
| | | Classic | X | | | | | | | | |
| | | Cham- | | | | | | | | | |
| | | pagne | | | | | | | | | |
| | | Sabino | | | | | | | | | |
| | | Amber | X | | | | | | | | |
| | | Cham- | | | | | | | | | |
| | | pagne | | | | | | | | | |
| | | Sabino | | | | | | | | | |
| | Cham- | Classic | X | | X | | | | | | |
| | pagne | Cham- | | | | | | | | | |
| | Dun | pagne | | | | | | | | | |
| | Sabino | Dun | | | | | | | | | |
| | | Sabino | | | | | | | | | |
| | Cham- | Pseudo- | X | | | | | X/n | | | |
| | pagne | cream | | | | | | | | | |
| | Dun | Classic | | | | | | | | | |
| | Pearl | Sabino | | | | | | | | | |
| | Sabino | | | | | | | | | | |
| | | Cham- | X | | | X/n | | | | | |
| | | pagne | | | | | | | | | |
| | | Gold | | | | | | | | | |
| | | Pearl | | | | | | | | | |
| | | Sabino | | | | | | | | | |
| | | Cham- | X | | | | | | X | | |
| | | pagne | | | | | | | | | |
| | | Classic | | | | | | | | | |
| | | Pearl | | | | | | | | | |
| | | Sabino | | | | | | | | | |
| | | Cham- | X | | | | X/n | | | | |
| | | pagne | | | | | | | | | |
| | | Amber | | | | | | | | | |
| | | Pearl | | | | | | | | | |
| | | Sabino | | | | | | | | | |
| | | Gold | X | | | | | | | | |
| | | Cream | | | | | | | | | |
| | | Sabino | | | | | | | | | |
| | | Classic | X | | | | | | | | |
| | | Cream | | | | | | | | | |
| | | Sabino | | | | | | | | | |
| | | Amber | X | | | | | | | | |
| | | Cream | | | | | | | | | |
| | | Sabino | | | | | | | | | |
| | Cream | | X | | | | | | | | |
| | Grullo | | | | | | | | | | |
| | Sabino | | | | | | | | | | |
| | | Chest- | X | | | | | | | | |
| | | nut | | | | | | | | | |
| | | Cream | | | | | | | | | |
| | | Pearl | | | | | | | | | |
| | | Sabino | | | | | | | | | |
| | | Black | X | | | | | | | | |
| | | Cream | | | | | | | | | |
| | | Pearl | | | | | | | | | |
| | | Sabino | | | | | | | | | |
| | | Bay | X | | | | | | | | |
| | | Cream | | | | | | | | | |
| | | Pearl | | | | | | | | | |
| | | Sabino | | | | | | | | | |
| | Cremello | | X | | | | | | | | |
| | Sabino | | | | | | | | | | |
| | | Red Dun | X | | | | | | | | |
| | | Sabino | | | | | | | | | |
| | | Apricot | X | | | | | | | | |
| | | Dun | | | | | | | | | |
| | | Sabino | | | | | | | | | |
| | | Gold | X | | | | | | | | |
| | | Dun | | | | | | | | | |
| | | Sabino | | | | | | | | | |
| | | Classic | X | | | | | | | | |
| | | Dun | | | | | | | | | |
| | | Sabino | | | | | | | | | |
| | | Dun | X | | | | | | | | |
| | | Pearl | | | | | | | | | |
| | | Black | | | | | | | | | |
| | | Sabino | | | | | | | | | |
| | | Dun | X | | | | | | | | |
| | | Pearl | | | | | | | | | |
| | | Bay | | | | | | | | | |
| | | Sabino | | | | | | | | | |
| | Dunolino | | X | | | | | | | | |
| | Sabino | | | | | | | | | | |
| | Dunskin | | X | | | | | | | | |
| | Sabino | | | | | | | | | | |
| | Gold Dun | Pseudo- | X | | | | | | | | |
| | Pearl | cream | | | | | | | | | |
| | Sabino | Gold | | | | | | | | | |
| | | Sabino | | | | | | | | | |
| | Grullo | | X | | | | | | | | |
| | Sabino | | | | | | | | | | |
| | Palomino | | X | | | | | | | | |
| | Sabino | | | | | | | | | | |
| | | Apricot | X | | | | | | | | |
| | | Pearl | | | | | | | | | |
| | | Sabino | | | | | | | | | |
| | | Black | X | | | | | | | | |
| | | Pearl | | | | | | | | | |
| | | Sabino | | | | | | | | | |
| | | Bay | X | | | | | | | | |
| | | Pearl | | | | | | | | | |
| | | Sabino | | | | | | | | | |
| | Perlino | | X | | | | | | | | |
| | Sabino | | | | | | | | | | |
| | Perlino | | X | | | | | | | | |
| | Dun | | | | | | | | | | |
| | Sabino | | | | | | | | | | |
| | Red | Chest- | X | | | | | | | | |
| | Sabino | nut | | | | | | | | | |
| | | Sorrel | | | | | | | | | |
| | | Sabino | | | | | | | | | |
| | Silver | Silver | X | | | | | | | | |
| | Amber | Amber | | | | | | | | | |
| | Dun | Cham- | | | | | | | | | |
| | Sabino | pagne | | | | | | | | | |
| | | Dun | | | | | | | | | |
| | | Sabino | | | | | | | | | |
| | | Silver | X | | | | | | | | |
| | | Classic | | | | | | | | | |
| | | Cham- | | | | | | | | | |
| | | pagne | | | | | | | | | |
| | | Sabino | | | | | | | | | |
| | | Silver | X | | | | | | | | |
| | | Amber | | | | | | | | | |
| | | Cham- | | | | | | | | | |
| | | pagne | | | | | | | | | |
| | | Sabino | | | | | | | | | |
| | Silver | | X | | | | | | | | |
| | Cham- | | | | | | | | | | |
| | pagne | | | | | | | | | | |
| | Dun | | | | | | | | | | |
| | Sabino | | | | | | | | | | |
| | | Silver | X | | | | | | | | |
| | | Classic | | | | | | | | | |
| | | Cham- | | | | | | | | | |
| | | pagne | | | | | | | | | |
| | | Pearl | | | | | | | | | |
| | | Sabino | | | | | | | | | |
| | | Silver | X | | | | | | | | |
| | | Classic | | | | | | | | | |
| | | Amber | | | | | | | | | |
| | | Pearl | | | | | | | | | |
| | | Sabino | | | | | | | | | |
| | | Silver | X | | | | | | | | |
| | | Black | | | | | | | | | |
| | | Cream | | | | | | | | | |
| | | Pearl | | | | | | | | | |
| | | Sabino | | | | | | | | | |
| | | Silver | X | | | | | | | | |
| | | Bay | | | | | | | | | |
| | | Cream | | | | | | | | | |
| | | Pearl | | | | | | | | | |
| | | Sabino | | | | | | | | | |
| | Silver | Silver | X | | | | | | | | |
| | Dapple | Black | | | | | | | | | |
| | Sabino | Sabino | | | | | | | | | |
| | Silver | | X | | | | | | | | |
| | Bay | | | | | | | | | | |
| | Sabino | | | | | | | | | | |
| | Silver | | X | | | | | | | | |
| | Buck- | | | | | | | | | | |
| | skin | | | | | | | | | | |
| | Sabino | | | | | | | | | | |
| | Silver | | X | | | | | | | | |
| | Cream | | | | | | | | | | |
| | Grullo | | | | | | | | | | |
| | Sabino | | | | | | | | | | |
| | Silver | Silver | X | | | | | | | | |
| | Dun | Dapple | | | | | | | | | |
| | Sabino | Dun | | | | | | | | | |
| | | Sabino | | | | | | | | | |
| | Silver | Silver | X | | | | | | | | |
| | Dunskin | Dun Bay | | | | | | | | | |
| | Sabino | Cream | | | | | | | | | |
| | | Sabino | | | | | | | | | |
| | Silver | Silver | X | | | | | | | | |
| | Grullo | Dun | | | | | | | | | |
| | Sabino | Black | | | | | | | | | |
| | | Sabino | | | | | | | | | |
| | | Silver | X | | | | | | | | |
| | | Pearl | | | | | | | | | |
| | | Black | | | | | | | | | |
| | | Sabino | | | | | | | | | |
| | | Silver | X | | | | | | | | |
| | | Pearl | | | | | | | | | |
| | | Bay | | | | | | | | | |
| | | Sabino | | | | | | | | | |
| | Silver | | X | | | | | | | | |
| | Perlino | | | | | | | | | | |
| | Sabino | | | | | | | | | | |
| | Silver | | X | | | | | | | | |
| | Perlino | | | | | | | | | | |
| | Dun | | | | | | | | | | |
| | Sabino | | | | | | | | | | |
| | | Silver | X | | | | | | | | |
| | | Dun | | | | | | | | | |
| | | Pearl | | | | | | | | | |
| | | Black | | | | | | | | | |
| | | Sabino | | | | | | | | | |
| | | Silver | X | | | | | | | | |
| | | Dun | | | | | | | | | |
| | | Pearl | | | | | | | | | |
| | | Bay | | | | | | | | | |
| | | Sabino | | | | | | | | | |
| | Silver | | X | | | | | | | | |
| | Smoky | | | | | | | | | | |
| | Cream | | | | | | | | | | |
| | Sabino | | | | | | | | | | |
| | Smoky | Black | X | | | | | | | | |
| | Black | Cream | | | | | | | | | |
| | Sabino | Sabino | | | | | | | | | |
| | Smoky | | X | | | | | | | | |
| | Cream | | | | | | | | | | |
| | Sabino | | | | | | | | | | |
| | Smoky | Dun | X | | | | | | | | |
| | Grullo | Black | | | | | | | | | |
| | Sabino | Cream | | | | | | | | | |
| | | Sabino | | | | | | | | | |
| | Cham- | | X | | | | | | | | |
| | pagne | | | | | | | | | | |
| | Pearl | | | | | | | | | | |
| | Sabino | | | | | | | | | | |
| | Cream | | X | | | | | | | | |
| | Pearl | | | | | | | | | | |
| | Sabino | | | | | | | | | | |
| | Dun | | X | | | | | | | | |
| | Pearl | | | | | | | | | | |
| | Sabino | | | | | | | | | | |
| | Silver | | X | | | | | | | | |
| | Cham- | | | | | | | | | | |
| | pagne | | | | | | | | | | |
| | Sabino | | | | | | | | | | |
| | Silver | | X | | | | | | | | |
| | Cham- | | | | | | | | | | |
| | pagne | | | | | | | | | | |
| | Pearl | | | | | | | | | | |
| | Sabino | | | | | | | | | | |
| | Silver | | X | | | | | | | | |
| | Cream | | | | | | | | | | |
| | Pearl | | | | | | | | | | |
| | Sabino | | | | | | | | | | |
| | Silver | | X | | | | | | | | |
| | Pearl | | | | | | | | | | |
| | Sabino | | | | | | | | | | |
| | Silver | | X | | | | | | | | |
| | Dun | | | | | | | | | | |
| | Pearl | | | | | | | | | | |
| | Sabino | | | | | | | | | | |
| | Silver | | | | | | | | | | |
| | Bay | | | | | | | | | | |
| | Cream | | | | | | | | | | |
| | Tobiano | | | | | | | | | | |

TABLE 6
Referential table for potentially deleterious or fatal coat color/coat color modification genotypes

| Coat | | | | | | | |
|---|---|---|---|---|---|---|---|
| color/modification | EDNRB_TC_to_AG | KITW2 | KITW5 | KITW6 | KITW7 | KITW10 | KITW12 |
| Dominant White | | | | | | X/X | |
| W10/W10 | | | | | | | |
| Dominant White | | | | | | | X/X |
| W12/W12 | | | | | | | |
| Dominant White | | | | | | | |
| W13/W13 | | | | | | | |
| Dominant White | | | | | | | |
| W14/W14 | | | | | | | |
| Dominant White | | | | | | | |
| W19/W19 | | | | | | | |
| Dominant White | | X/X | | | | | |
| W2/W2 | | | | | | | |
| Dominant White | | | X/X | | | | |
| W5/W5 | | | | | | | |
| Dominant White | | | | X/X | | | |
| W6/W6 | | | | | | | |
| Dominant White | | | | | X/X | | |
| W7/W7 | | | | | | | |
| Lethal Frame | X/X | | | | | | |
| Overo (LWO) | | | | | | | |

| Coat | | | | |
|---|---|---|---|---|
| color/modification | KITW13 | KITW14 | KITW19 | Description |
| Dominant White | | | | Two W10 variants may be incompatible with |
| W10/W10 | | | | life - estimated 25% chance of no foal. |
| Dominant White | | | | Two W12 variants may be incompatible with |
| W12/W12 | | | | life - estimated 25% chance of no foal. |
| Dominant White | X/X | | | Two W13 variants may be incompatible with |
| W13/W13 | | | | life - estimated 25% chance of no foal. |
| Dominant White | | X/X | | Two W14 variants may be incompatible with |
| W14/W14 | | | | life - estimated 25% chance of no foal. |
| Dominant White | | | X/X | Two W19 variants may result in all white coat |
| W19/W19 | | | | and possible health issues, growth issues, or |
| | | | | foal death. |
| Dominant White | | | | Two W2 variants may be incompatible with |
| W2/W2 | | | | life - estimated 25% chance of no foal. |
| Dominant White | | | | Two W5 variants may be incompatible with |
| W5/W5 | | | | life - estimated 25% chance of no foal. |
| Dominant White | | | | Two W6 variants may be incompatible with |
| W6/W6 | | | | life - estimated 25% chance of no foal. |
| Dominant White | | | | Two W7 variants may be incompatible with |
| W7/W7 | | | | life - estimated 25% chance of no foal. |
| Lethal Frame | | | | Two LWO variants are known to lead to |
| Overo (LWO) | | | | Lethal White Syndrome; results in failed |
| | | | | pregnancy or foal death. |

TABLE 7
Referential table for disease and non-disease conditions in horses

| Disease or | | | | | | | |
|---|---|---|---|---|---|---|---|
| non-disease | | | | | | | |
| condition | MUTYHCA | SLC5A3FIS | GBE1GBED | PPIBHERDA | LAMC2JEB1 | LAMA3JEB2 | MYO5ALFS |
| Cerebellar | X | | | | | | |
| Abiotrophy (CA) | | | | | | | |
| Foal | | X | | | | | |
| Immunodeficiency | | | | | | | |
| Syndrome (FIS) | | | | | | | |
| Glycogen | | | X | | | | |
| Branching Enzyme | | | | | | | |
| Deficiency (GBED) | | | | | | | |
| Hereditary Equine | | | | X | | | |
| Regional Dermal | | | | | | | |
| Asthenia | | | | | | | |
| (HERDA) | | | | | | | |
| Junctional | | | | | X | | |
| Epidermolysis | | | | | | | |
| Bullosis | | | | | | | |
| (type 1; JEB1) | | | | | | | |
| Junctional | | | | | | X | |
| Epidermolysis | | | | | | | |
| Bullosis | | | | | | | |
| (type 2; JEB2) | | | | | | | |
| Lavender Foal | | | | | | | X |
| Syndrome (LFS) | | | | | | | |
| Malignant | | | | | | | |
| Hyperthermia | | | | | | | |
| (MH) | | | | | | | |
| Myotonia | | | | | | | |
| Polysaccharide | | | | | | | |
| Storage Myopathy | | | | | | | |
| (type 1; PSSM1) | | | | | | | |
| Severe Combined | | | | | | | |
| Immunodeficiency | | | | | | | |
| Warmblood | | | | | | | |
| Fragile Foal | | | | | | | |
| Syndrome (FFS) | | | | | | | |
| Hoof Wall | | | | | | | |
| Separation Disease | | | | | | | |
| (HWSD) | | | | | | | |
| Hydrocephalu [symbol] | | | | | | | |
| (HYD) | | | | | | | |
| Hyperkalemic | | | | | | | |
| Partial Paralysis | | | | | | | |
| (HYPP) | | | | | | | |

| Disease or | | | | | | |
|---|---|---|---|---|---|---|
| non-disease | | | | | | |
| condition | RYR1MH | CLCN4MYT | GYS1PSSM1 | DNAPKSCID | PLOD1WFFS | SERPINB11HWSD |
| Cerebellar | | | | | | |
| Abiotrophy (CA) | | | | | | |
| Foal | | | | | | |
| Immunodeficiency | | | | | | |
| Syndrome (FIS) | | | | | | |
| Glycogen | | | | | | |
| Branching Enzyme | | | | | | |
| Deficiency (GBED) | | | | | | |
| Hereditary Equine | | | | | | |
| Regional Dermal | | | | | | |
| Asthenia | | | | | | |
| (HERDA) | | | | | | |
| Junctional | | | | | | |
| Epidermolysis | | | | | | |
| Bullosis | | | | | | |
| (type 1; JEB1) | | | | | | |
| Junctional | | | | | | |
| Epidermolysis | | | | | | |
| Bullosis | | | | | | |
| (type 2; JEB2) | | | | | | |
| Lavender Foal | | | | | | |
| Syndrome (LFS) | | | | | | |
| Malignant | X/X | | | | | |
| Hyperthermia | | | | | | |
| (MH) | | | | | | |
| Myotonia | | X/X | | | | |
| Polysaccharide | | | X/X | | | |
| Storage Myopathy | | | | | | |
| (type 1; PSSM1) | | | | | | |
| Severe Combined | | | | X | | |
| Immunodeficiency | | | | | | |
| Warmblood | | | | | X | |
| Fragile Foal | | | | | | |
| Syndrome (FFS) | | | | | | |
| Hoof Wall | | | | | | X |
| Separation Disease | | | | | | |
| (HWSD) | | | | | | |
| Hydrocephalu [symbol] | | | | | | |
| (HYD) | | | | | | |
| Hyperkalemic | | | | | | |
| Partial Paralysis | | | | | | |
| (HYPP) | | | | | | |

| Disease or | | | |
|---|---|---|---|
| non-disease | | | |
| condition | B3GALNT2hydroceph | SCN4AHYPP | Description |
| Cerebellar | | | Two CA variants - may result in mild to severe |
| Abiotrophy (CA) | | | neurological symptoms. One CA variant - may |
| | | | result in mild to severe neurological symptoms. |
| Foal | | | Two FIS variants - may exhibit signs of anemia, |
| Immunodeficiency | | | diarrhea, and pneumonia due to a failing immune |
| Syndrome (FIS) | | | system which can lead to foal death. One FIS |
| | | | variant - may exhibit signs of anemia, diarrhea, |
| | | | and pneumonia due to a failing immune system |
| | | | which can lead to foal death. |
| Glycogen | | | Two GBED variants - may lead to failed |
| Branching Enzyme | | | pregnancy and/or severely compromised/dead foal. |
| Deficiency (GBED) | | | One GBED variant - may lead to failed pregnancy |
| | | | and/or severely compromised/dead foal. |
| Hereditary Equine | | | Two HERDA variants - may result in serious |
| Regional Dermal | | | skin-related damage resulting in severe/fatal |
| Asthenia | | | health issues. One HERDA variant - may result |
| (HERDA) | | | in serious skin-related damage resulting in |
| | | | severe/fatal health issues. |
| Junctional | | | Two JEB1 variants - may exhibit serious health |
| Epidermolysis | | | issues, such as blisters at pressure points, hoof |
| Bullosis | | | detachment, and oral ulcer, resulting foal death. |
| (type 1; JEB1) | | | One JEB1 variant - may exhibit serious health |
| | | | issues, such as blisters at pressure points, hoof |
| | | | detachment, and oral ulcer, resulting foal death. |
| Junctional | | | Two JEB2 variants - may exhibit serious health |
| Epidermolysis | | | issues, such as blisters at pressure points, hoof |
| Bullosis | | | detachment, and oral ulcer, resulting foal death. |
| (type 2; JEB2) | | | One JEB2 variant - may exhibit serious health |
| | | | issues, such as blisters at pressure points, hoof |
| | | | detachment, and oral ulcer, resulting foal death. |
| Lavender Foal | | | Two LFS variants - may lead to failed pregnancy |
| Syndrome (LFS) | | | and/or severely compromised/dead foal. One |
| | | | LFS variant - may lead to failed pregnancy and/or |
| | | | severely compromised/dead foal. |
| Malignant | | | Two MH variants - may result in severe health |
| Hyperthermia | | | issues, such as fever, increased heart rate, high |
| (MH) | | | blood pressure, seating, acidosis, and muscle |
| | | | rigidity. |
| Myotonia | | | Two Myotonia variants - may result in health |
| | | | issues due to problems with muscle stiffness. |
| Polysaccharide | | | Two PSSM1 variants - may result in severe |
| Storage Myopathy | | | chronic exertional rhabdomyolysis (“tying-up”) |
| (type 1; PSSM1) | | | episodes which may lead to health issues. |
| Severe Combined | | | Two SCI variants; may exhibit signs of fever, |
| Immunodeficiency | | | diarrhea, and respiratory distress due to a failing |
| | | | immune system which can lead to foal death. |
| | | | One FIS variant - may exhibit signs of fever, |
| | | | diarrhea, and respiratory distress due to a failing |
| | | | immune system which can lead to foal death. |
| Warmblood | | | Two FFS variants - may lead to failed pregnancy |
| Fragile Foal | | | and/or severely compromised/dead foal. |
| Syndrome (FFS) | | | One FFS variant - may lead to failed pregnancy |
| | | | and/or severely compromised/dead foal |
| Hoof Wall | | | Two HWSD variants - may result in serious hoof |
| Separation Disease | | | wall damage or failure which may lead to severe |
| (HWSD) | | | mobility and health issues. One HWSD |
| | | | variant - may result in serious hoof wall damage |
| | | | or failure which may lead to severe mobility and |
| | | | health issues. |
| Hydrocephalu [symbol] | X | | Two HYD variants - may lead to failed pregnancy |
| (HYD) | | | and/or severely compromised/dead foal. |
| | | | One HYD variant - may lead to failed pregnancy |
| | | | and/or severely compromised/dead foal |
| Hyperkalemic | | X/X | Two HYPP variants - may result in tremors and |
| Partial Paralysis | | | muscles weakness which may lead to severe |
| (HYPP) | | | paralysis and/or death. |

[symbol]

indicates data missing or illegible when filed

TABLE 8
Referential table for disease and non-disease conditions in horses (continued)

| Disease or | | | | | | |
|---|---|---|---|---|---|---|
| non-disease | | | | | | |
| condition | ARAS | BIEC2263524EMS | BIEC2536712WB_ERU_Risk | SCN4AHYPP | FKBP6IAR1 | FKBP6IAR2 |
| Androgen | X | | | | | |
| Insensitivity (AI) | | | | | | |
| Equine Metabolic | | X | | | | |
| Syndrome - Risk | | | | | | |
| (EMS) | | | | | | |
| Equine Recurrent | | | X | | | |
| Uveitis - Risk | | | | | | |
| (ERU) | | | | | | |
| Hyperkalemic | | | | X/n | | |
| Partial Paralysis | | | | | | |
| (HYPP) | | | | | | |
| Impaired | | | | | X/X | X/X |
| Acrosomal | | | | | | |
| Reaction (IAR) | | | | | | |
| Laminitis Risk | | | | | | |
| (LAM) | | | | | | |
| Lordosis (affected) | | | | | | |
| Lordosis (carrier) | | | | | | |
| Malignant | | | | | | |
| Hyper-thermia | | | | | | |
| (MH) | | | | | | |
| Myotonia (MYT) | | | | | | |
| Polysaccharide | | | | | | |
| Storage Myopathy | | | | | | |
| (type 1; PSSM) | | | | | | |
| Squamous Cell | | | | | | |
| Carcinoma | | | | | | |
| (SCC) - Risk | | | | | | |
| West Nile | | | | | | |
| Virus (WNV) | | | | | | |
| Susceptibility | | | | | | |
| Naked Foal | | | | | | |
| Syndrome (NFS) | | | | | | |
| Dwarfism (affected) | | | | | | |
| Dwarfism (carrier) | | | | | | |

| Disease or | | | | | | |
|---|---|---|---|---|---|---|
| non-disease | | | | | | |
| condition | BIEC2263524_LAM | BIEC2532523L1 | BiEC2532534L2 | BIEC2532578L3 | BIEC2532658L4 | RYR1MH |
| Androgen | | | | | | |
| Insensitivity (AI) | | | | | | |
| Equine Metabolic | | | | | | |
| Syndrome - Risk | | | | | | |
| (EMS) | | | | | | |
| Equine Recurrent | | | | | | |
| Uveitis - Risk | | | | | | |
| (ERU) | | | | | | |
| Hyperkalemic | | | | | | |
| Partial Paralysis | | | | | | |
| (HYPP) | | | | | | |
| Impaired | | | | | | |
| Acrosomal | | | | | | |
| Reaction (IAR) | | | | | | |
| Laminitis Risk | X | | | | | |
| (LAM) | | | | | | |
| Lordosis (affected) | | X/X | X/X | X/X | X/X | |
| Lordosis (carrier) | | X/n | X/n | X/n | X/n | |
| Malignant | | | | | | X/n |
| Hyper-thermia | | | | | | |
| (MH) | | | | | | |
| Myotonia (MYT) | | | | | | |
| Polysaccharide | | | | | | |
| Storage Myopathy | | | | | | |
| (type 1; PSSM) | | | | | | |
| Squamous Cell | | | | | | |
| Carcinoma | | | | | | |
| (SCC) - Risk | | | | | | |
| West Nile | | | | | | |
| Virus (WNV) | | | | | | |
| Susceptibility | | | | | | |
| Naked Foal | | | | | | |
| Syndrome (NFS) | | | | | | |
| Dwarfism (affected) | | | | | | |
| Dwarfism (carrier) | | | | | | |

| Disease or | | | | | | | |
|---|---|---|---|---|---|---|---|
| non-disease | | | | | | | |
| condition | CLCN4MYT | GYS1PSSM1 | DDB2LSCC | OAS1WNVR | ST14_NFSYN | ST14NFS | ACAN_D1 |
| Androgen | | | | | | | |
| Insensitivity (AI) | | | | | | | |
| Equine Metabolic | | | | | | | |
| Syndrome - Risk | | | | | | | |
| (EMS) | | | | | | | |
| Equine Recurrent | | | | | | | |
| Uveitis - Risk | | | | | | | |
| (ERU) | | | | | | | |
| Hyperkalemic | | | | | | | |
| Partial Paralysis | | | | | | | |
| (HYPP) | | | | | | | |
| Impaired | | | | | | | |
| Acrosomal | | | | | | | |
| Reaction (IAR) | | | | | | | |
| Laminitis Risk | | | | | | | |
| (LAM) | | | | | | | |
| Lordosis (affected) | | | | | | | |
| Lordosis (carrier) | | | | | | | |
| Malignant | | | | | | | |
| Hyper-thermia | | | | | | | |
| (MH) | | | | | | | |
| Myotonia (MYT) | X/n | | | | | | |
| Polysaccharide | | X/n | | | | | |
| Storage Myopathy | | | | | | | |
| (type 1; PSSM) | | | | | | | |
| Squamous Cell | | | X | | | | |
| Carcinoma | | | | | | | |
| (SCC) - Risk | | | | | | | |
| West Nile | | | | X | | | |
| Virus (WNV) | | | | | | | |
| Susceptibility | | | | | | | |
| Naked Foal | | | | | X/X | X/X | |
| Syndrome (NFS) | | | | | | | |
| Dwarfism (affected) | | | | | | | X/X |
| Dwarfism (carrier) | | | | | | | X/n |

| Disease or | | | | | |
|---|---|---|---|---|---|
| non-disease | | | | | |
| condition | ACAN_D2 | ACAN_D3 | ACAN_D4 | ACAN_D5 | Description |
| Androgen | | | | | Horses with AI may appear to be |
| Insensitivity (AI) | | | | | females however may be genetically |
| | | | | | male making them unable to reproduce. |
| Equine Metabolic | | | | | EMS variant - horse may have |
| Syndrome - Risk | | | | | increased susceptibility to EMS |
| (EMS) | | | | | over average horse. |
| Equine Recurrent | | | | | ERU risk variant - horse may have |
| Uveitis - Risk | | | | | increased susceptibility to ERU |
| (ERU) | | | | | over average horse. |
| Hyperkalemic | | | | | HYPP variant - symptoms for |
| Partial Paralysis | | | | | HYPP may be present along with |
| (HYPP) | | | | | greater than average muscle |
| | | | | | development at an early age. |
| Impaired | | | | | IAR variants - only shown to |
| Acrosomal | | | | | affect Thoroughbred stallions |
| Reaction (IAR) | | | | | (IAR or subfertility) |
| Laminitis Risk | | | | | LAM risk variant - Horse may |
| (LAM) | | | | | have increased risk of |
| | | | | | EMS-related Laminitis over |
| | | | | | average horse. |
| Lordosis (affected) | | | | | Lordosis variants - 80% of horses |
| | | | | | with this genotype will display |
| | | | | | lordosis phenotype (Swaybacked). |
| Lordosis (carrier) | | | | | Lordosis variants - carrier status |
| | | | | | for lordosis (swayback seen in |
| | | | | | North American |
| | | | | | Saddlebred) - probability of |
| | | | | | producing swaybacked foal with |
| | | | | | another carrier is approximately |
| | | | | | 3% risk. |
| Malignant | | | | | MH variant - horse may experience |
| Hyper-thermia | | | | | Malignant Hyperthermia if unduly |
| (MH) | | | | | stressed or anesthetized, please |
| | | | | | consult your veterinarian. |
| Myotonia (MYT) | | | | | MYT variant - horse may be at |
| | | | | | increased risk for MYT - increased |
| | | | | | muscle stiffness or sudden muscle |
| | | | | | relaxation/collapse (“Fainting Goat |
| | | | | | Syndrome”). |
| Polysaccharide | | | | | PSSM Type 1 variant - horse may |
| Storage Myopathy | | | | | experience symptoms of |
| (type 1; PSSM) | | | | | “tying up” due to specific diet and |
| | | | | | exercise/work regimen, please |
| | | | | | consult with your veterinarian. |
| Squamous Cell | | | | | SCC Risk variant - horse may |
| Carcinoma | | | | | have increased risk for Squamous |
| (SCC) - Risk | | | | | Cell Carcinoma of the eye over |
| | | | | | average horse. |
| West Nile | | | | | WNV Risk variant - horse may |
| Virus (WNV) | | | | | suffer greater, longer or higher risk |
| Susceptibility | | | | | symptoms if he/she contracts West |
| | | | | | Nile Virus (*NOT a test for the |
| | | | | | presence of WNV). |
| Naked Foal | | | | | NFS variant - horse may be born |
| Syndrome (NFS) | | | | | without coat, mane, tail, dry skin. |
| | | | | | NFS/NFS foals are at high risk for |
| | | | | | death. |
| Dwarfism (affected) | X/X | X/X | X/X | X/X | Dwarfism variants - may result in |
| | | | | | stunted growth, abnormalities in |
| | | | | | bone and tissue, health risks, |
| | | | | | failed pregnancies and possible |
| | | | | | foal death |
| Dwarfism (carrier) | X/n | X/n | X/n | X/n | Dwarfism variant - carrier for |
| | | | | | Dwarfism, do not breed to another |
| | | | | | carrier to avoid affected foal |

TABLE 9
Referential table for behavioral/ability genotypes in horses

| Behavioral | | DMRT3 | MSTN | Myo- | |
|---|---|---|---|---|---|
| trait/ability | DRD4CV | Gait | DIST | statin | Description |
| Curious & Vigilant | X/n | | | | One Curiosity and one Vigilance variant; horse is likely both curious and vigilant. |
| Endurance | | | n/n | n/n | Endurance type myostatin; horse may excel at longer distance travel versus sprint |
| | | | | | type activity. |
| DMRT3 “Gait” Carrier | | X/n | | | One DMRT3 or “gaited” variant; may display a wide variation in locomotion, partial |
| (One Variant) | | | | | “gaiting” and possible difficulty with lead change, tempi changes, canter, or gait |
| | | | | | transitions. |
| DMRT3 “Gaited” | | X/X | | | Two DMRT3 or “gaited” variants; may display “gaiting”, 4 beat or possible loss of |
| (Two Variants) | | | | | canter). |
| Mid-Distance | | | X/n | X/n | Mid-distance type MSTN; horse may have multi-distance capabilities including sprint |
| | | | | | and endurance. |
| More Curious | X/X | | | | Two Curiosity variants; horse may be more curious than vigilant. |
| More Vigilant | n/n | | | | Two Vigilance variants; horse may be more vigilant than curious. |
| Non-Gaited | | n/n | | | No DMRT3 variants; likely non-gaited. |
| Sprint | | | X/X | X/X | Two Sprint type variants; horse may accel at short distance, quick bursts of speed |
| | | | | | over endurance type activity |

Tables 2-9 above contain the letters “X” and “n” in columns corresponding to individual variant copy number requirements to designate the genotype or phenotype of the horse. For example, “X/X” corresponds to an animal that is homozygous with respect to a particular variant. “X/n” corresponds to an animal that is heterozygous with respect to a particular variant. The label “n/n” corresponds to an animal that is either negative for the variant in question or the variant is irrelevant to the particular trait. The label “X” corresponds to a genotype that is either homozygous or heterozygous with respect to a particular variant. Thus, in a particular example, a horse having an “X/n” genotype for a MSTN gene variant would be heterozygous (i.e., MSTN/mstn) for a particular MSTN gene variant.

In a particular example, a user wishing to identify or produce a horse having a Dunskin Splash coat color/modification will require that the horse expresses a combination of at least one Black (E), Agouti (A), Cream (CR), Dun (D), and one or more of the splash genes (e.g., Splashed White 1, 2, 3, 4, 5, or 6). Therefore, the user may employ the database system of the disclosure by providing Dunkin Splash as a desired coat color/modification via the computer-implemented user interface. The user interface will then relay the preferred phenotype to the database system, which, using its search and comparison engines, will identify existing animals expressing the aforementioned genes or identify one or more breeding pairs capable of producing an offspring animal expressing the aforementioned genes to produce the Dunkin Splash coat phenotype.

In another example, a user wishing to identify or produce a horse having a Bay Overo coat color/modification will require that the horse expresses a combination of at least one copy of Black (E), Agouti (A), and one Lethal White Overo (LWO or “O”), which is occasionally not visible on the horse. However, a horse that is homozygous for LWO (i.e., LWO/LWO) is likely to die shortly after birth. Therefore, the database system of the disclosure can use this information to caution the user than the desired Bay Overo coat may produce a high probability of a fatal outcome if the identified or produced horse is an LWO homozygote. As such, the methods and systems described herein may prevent or reduce the likelihood of undesirable breeding outcomes for users wishing to produce an animal having one or more predetermined traits.

Inaccuracies can occur in the collection and reporting of information from secondary sources, sometimes due to misrepresentations of the animal's traits, habits, predisposition, or performance. For example, in order to increase the likelihood of a sale, an animal seller may misrepresent information about the animal to a buyer to inflate its value. Relatedly, an animal owner may assess the animal as being of a particular breed or as having a particular set of predispositions based on inaccurate pedigree information. To address this issue, the database can include an input for information from secondary sources, such as information obtained from, e.g., someone knowledgeable about the animal, which may be used to correct or produce more accurate determinations of relevant traits. The secondary source information can be combined with other data in the database such as a genetic profile of the animal, clinical assays of biological tissue obtained from the animal, and/or diagnostic tests performed in the animal to produce an overall characterization of the animal. In some cases, heuristic rules may be used to generate data based on measured (e.g., genetic/clinical/diagnostic data), rather than self-reported traits of the animal. Heuristic rules are defined as rules which relate measurable (or accurately measurable) traits to less measurable or less reliable traits such as those from self-reported data. As such, one or more heuristic rules, typically based on research which statistically links a variety of parameters related to one or more traits of interest, can be applied to the data representing genetic and non-genetic information about the animal to derive more accurate assessment of the probability of obtaining an animal having one or more predetermined traits.

According to the present disclosure, the database system described herein may further include unique database subsystems containing information about each of, for example, user profiles, user farm profiles, user settings, animal profiles, veterinary diagnostic tests, user payment information, and user selection parameters pertaining to one or more predetermined traits of the animal.

Although represented as a system and search engines, the system and engines can be considered subsystems of a larger database system, and as such referred to as subsystems. Such subsystems may be implemented as sections of code, objects, or classes of objects within a single system, or may be separate hardware and software platforms which are integrated with other subsystems to form the final system. The database system described herein may employ a number of database architectures including, but not limited to flat files, relational databases and object-oriented databases. The various subsystems can be discrete components, configurations of electronic circuits within other circuits, software modules running on computing platforms including classes of objects and object code, or individual commands or lines of code working in conjunction with one or more Central Processing Units (CPUs). A variety of storage units can be used including but not limited to electronic, magnetic, electromagnetic, optical, opto-magnetic and electro-optical storage (e.g., non-transitory storage media).

The database system may include database subsystems that may be combined into a single database or kept separated. Separate database subsystems may be stored on a single computing device or distributed across a plurality of devices. As such, a memory for storing such datasets, while referred to as a singular memory, may in reality be a distributed memory including a plurality of separate physical or virtual memory locations distributed over a plurality of devices such as over a computer network. Data, datasets, databases, methods and software of the present disclosure can be embodied on a computer-readable media (medium), computer-readable memory (including computer readable memory devices), and program storage devices readable by a machine.

The computer-implemented systems and databases disclosed herein can be accessed remotely as part of a web-based health analysis and diagnostics platform in which the users are able to submit biological samples of one or more non-human animals to obtain services such as pedigree analysis, health assessment, and facilitation of breeding strategies. In these scenarios, the database disclosed herein can be used to determine such parameters as the likelihood that the animal will develop or has a particular disease or have an injury related to likelihood of disease development, and or the particular breed or combination of breeds of the animal. The user may submit the genetic sample of the non-human animal(s) by, e.g., mailing the sample to an analysis center, where genetic and, optionally, epigenetic testing is performed, and the data stored in an appropriate database. A veterinarian, user, or someone familiar with the animal may report other data from which non-genetic information is obtained and stored. Using the methods and systems disclosed herein, an assessment of the animal can be developed and presented via the user interface described herein. These assessments may relate to physical and/or behavioral characteristics or predispositions of the animal.

### Use of a Database System to Produce a Genetically Modified Animal Having One or More Predetermined Traits

A non-human animal with one or more predetermined traits may be produced using the systems and methods described herein, including by genetically modifying the animal to produce a transgenic non-human animal. For example, for a variety reasons, a user may not be successful in the production of an offspring animal having one or more traits from a breeding pair identified using the database system as being capable of producing the offspring animal having one or more predetermined traits. In such a case, a user may elect to further genetically modify the offspring animal (e.g., during prenatal development or after birth) to increase the likelihood of the offspring animal having the one or more predetermined traits. Alternatively, a user, using the database system, may be unsuccessful in the identification of a breeding pair (from among the entries in the database) that is capable of producing a desired offspring animal having one or more predetermined traits (e.g., some, but not all, of the desired traits may be result by breeding the selected parental animals) In such a scenario, a user may decide to genetically modify at least one of the animals in the breeding pair so as to increase the likelihood that the breeding pair will produce an offspring animal having all (or most of) the one or more predetermined traits. In another example, a user may be successful in producing an offspring animal having some, but not all, desired traits from a breeding pair identified using the disclosed database system. To ensure the expression of all desired traits in the offspring animal, the user may genetically modify the offspring animal to increase the likelihood of the offspring animal expressing all of the predetermined traits desired by the user. In yet another example, a user may be successful in producing an offspring animal having partial, but not complete expression of a particular desired trait, which results in, e.g., a mixed phenotype. To remedy the incomplete expression of the desired trait in the offspring animal, the user may genetically modify the offspring animal to increase (e.g., by 5%, 10%, 15%, 20%, 30%, 40%, 50%, 60%, 70%, 80%, 90%, 100% or more) the expression of the incompletely expressed trait. Methods for genetic modification of non-human animals is discussed in further detail below.

Genetically modifying the offspring animal may include, e.g., modifying at least 1 (e.g., at least 1, 2, 3, 4, 5, 6, 7, 8, 9, 10, or more) genes in the offspring animal to produce a desired genotype or phenotype. Genetic modification may be achieved in the offspring animal by introduction of heterologous polynucleotides encoding a transgene of interest (e.g., wild-type variants of genes disclosed in Table 10, among others) into the cells of the non-human animal by editing one or more endogenous genes in the non-human animal, as is described in detail below. Genetic modification may be performed in an animal in which, for example, an endogenous gene corresponding to the transgene is lacking or the endogenous gene corresponding to the transgene produces an undesirable trait.

A. Delivery of a Heterologous Gene to Animal Cells

The present disclosure provides methods for producing transgenic non-human animals (e.g., horse, cattle, sheep, dog, cat, camel, pig, goat, alpaca, donkey, llama, red fox, mouse, rat, ferret, non-human primate, rabbit, gerbil, hamster, chinchilla, or guinea pig) having one or more predetermined traits by introducing a heterologous polynucleotide encoding a transgene that increases the probability of producing one or more desirable traits in the animal. Transgenic animals may be produced by artificially incorporating a heterologous transgene of interest into one or more cells (e.g., incorporation into the genome of the one or more cells), or the germline, of the animal, a parent or ancestor of the animal, the animal at an early developmental stage (e.g., embryonic stage, such as a single-cell stage), or the animal after birth. Introduction of an exogenous transgene to the animal has the intended effect of increasing the probability of producing one or more predetermined traits in the animal or offspring of the animal.

Several methods are available by which a transgene can be introduced into an animal so as to be chromosomally incorporated into the animal genome. Techniques that can be used to introduce a heterologous transgene an animal cell are well known in the art. For example, electroporation can be used to permeabilize target cells by the application of an electrostatic potential to the cell of interest. Target cells, such as mammalian or insect cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells are the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for example, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA including the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107: 1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods in Cell Biology 82:309 (2007), the disclosure of which is incorporated herein by reference.

B. Gene Editing

The present disclosure also provides methods for producing transgenic non-human animals (e.g., horse, cow, sheep, dog, camel, among others) having one or more predetermined traits by using targeted gene editing methods for modifying chromosomal DNA of the animal. For example, the disclosed methods and systems may be used for the purpose of producing a transgenic non-human animal having one or more predetermined traits by genetically modifying the non-human offspring animal. Genetic modification may be performed on one or more (e.g., 1, 2, 3, 4, 5, 6, 7, 8, 9, 10, or more) genes, given that the genes contribute to or are determinant of the phenotypic expression of the one or more predetermined traits of interest. Gene editing may be performed, e.g., to modify an endogenous gene in the non-human animal. Without wishing to be bound by theory, editing may be performed on a particular endogenous gene if the gene contains a deleterious polymorphism (e.g., SNP) or a genetic mutation (insertion, deletion (e.g., knockout), translocation, inversion, single point mutation, or other mutation) by modifying, deleting, or replacing at least one nucleotide of a DNA or an RNA encoding the endogenous gene in the non-human animal. Gene modifications may be carried out by, e.g., inserting a donor polynucleotide (such as, e.g., donor polynucleotide including a segment of the endogenous gene that lacks the polymorphism or mutation) into a region of the endogenous gene in the non-human animal. Genetic modification may also be performed by, e.g., introducing into the non-human animal (e.g., into the genome of the non-human animal) a polynucleotide encoding one or more copies of a wild-type variant of the endogenous gene. Alternatively, gene editing of an endogenous gene may include modulating (e.g., increasing or decreasing) expression of an endogenous gene in the non-human animal.

Methods for producing a transgenic animal described herein may include the use of a gene editing system. For example, the gene editing system may introduce an alteration (e.g., insertion, deletion (e.g., knockout), translocation, inversion, single point mutation, or other mutation) in a gene in the animal. Exemplary gene editing systems include the zinc finger nucleases (ZFNs), Transcription Activator-Like Effector-based Nucleases (TALEN), and the clustered regularly interspaced short palindromic repeat (CRISPR) system. ZFNs, TALENs, and CRISPR-based methods are described, e.g., in Gaj et al., Trends Biotechnol. 31(7):397-405, 2013.

In a typical CRISPR/Cas system, an endonuclease is directed to a target nucleotide sequence (e.g., a site in the genome that is to be sequence-edited) by sequence-specific, non-coding guide RNAs that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (crRNA), and a trans-activating crRNA (tracrRNA). The crRNA contains a guide RNA, i.e., typically an about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The RNAs serve as guides to direct Cas proteins to silence specific DNA/RNA sequences, depending on the spacer sequence. See, e.g., Horvath et al., Science 327:167-170, 2010; Makarova et al., Biology Direct 1:7, 2006; Pennisi, Science 341:833-836, 2013. The target DNA sequence must generally be adjacent to a protospacer adjacent motif (PAM) that is specific for a given Cas endonuclease; however, PAM sequences appear throughout a given genome. CRISPR endonucleases identified from various prokaryotic species have unique PAM sequence requirements. Some endonucleases, e.g., Cas9 endonucleases, are associated with G-rich PAM sites and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; stated differently, a Cpf1 system requires only the Cpf1 nuclease and a crRNA to cleave the target DNA sequence. Cpf1 endonucleases, are associated with T-rich PAM sites. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al., Cell 163:759-771, 2015.

For the purposes of gene editing, CRISPR arrays can be designed to contain one or multiple guide RNA sequences corresponding to a desired target DNA sequence; see, for example, Cong et al., Science 339:819-823, 2013; and Ran et al., Nature Protocols 8:2281-2308, 2013. At least about 16 or 17 nucleotides of gRNA sequence are required by Cas9 for DNA cleavage to occur; for Cpf1 at least about 16 nucleotides of gRNA sequence is needed to achieve detectable DNA cleavage. In practice, guide RNA sequences are generally designed to have a length of between 17-24 nucleotides (e.g., 19, 20, or 21 nucleotides) and complementarity to the targeted gene or nucleic acid sequence. Custom gRNA generators and algorithms are available commercially for use in the design of effective guide RNAs. Gene editing has also been achieved using a chimeric single guide RNA (sgRNA), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al., Nature Biotechnol. 985-991, 2015. In some instances, the heterologous animal-modifying agent includes a ribonucleoprotein complex (RNP) including one or more RNA molecules, e.g., a gRNA or a sgRNA, and one or more RNA-binding proteins, e.g., an endonuclease, e.g., a Cas endonuclease (e.g., a Cas9 endonuclease).

Whereas wild-type Cas9 generates double-strand breaks (DSBs) at specific DNA sequences targeted by a gRNA, a number of CRISPR endonucleases having modified functionalities are available, for example: a nickase version of Cas9 generates only a single-strand break; a catalytically inactive Cas9 (dCas9) does not cut the target DNA but interferes with transcription by steric hindrance. dCas9 can further be fused with an effector to repress (CRISPRi) or activate (CRISPRa) expression of a target gene. For example, Cas9 can be fused to a transcriptional repressor (e.g., a KRAB domain) or a transcriptional activator (e.g., a dCas9—VP64 fusion). A catalytically inactive Cas9 (dCas9) fused to FokI nuclease (dCas9-FokI) can be used to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, MA 02139; addgene.org/crispr/). A double nickase Cas9 that introduces two separate double-strand breaks, each directed by a separate guide RNA, is described as achieving more accurate genome editing by Ran et al., Cell 154:1380-1389, 2013.

CRISPR technology for editing the genes of eukaryotes is disclosed in US Patent Application Publications US 2016/0138008 A1 and US 2015/0344912 A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

The desired genome modification may require homologous recombination, wherein one or more double-stranded DNA breaks in the target nucleotide sequence is generated by the RNA-guided nuclease and guide RNA(s), followed by repair of the break(s) using a homologous recombination mechanism (homology-directed repair). In such instances, a donor template that encodes the desired nucleotide sequence to be inserted or knocked-in at the double-stranded break is provided to the cell or subject; examples of suitable templates include single-stranded DNA templates and double-stranded DNA templates (e.g., linked to the polypeptide described herein). In general, a donor template encoding a nucleotide change over a region of less than about 50 nucleotides is provided in the form of single-stranded DNA; larger donor templates (e.g., more than 100 nucleotides) are often provided as double-stranded DNA plasmids. The donor template may be provided to the cell or animal in a quantity that is sufficient to achieve the desired homology-directed repair but that does not persist in the cell or animal after a given period of time (e.g., after one or more cell division cycles). The donor template may include a core nucleotide sequence that differs from the target nucleotide sequence (e.g., a homologous endogenous genomic region) by at least 1, at least 5, at least 10, at least 20, at least 30, at least 40, at least 50, or more nucleotides. This core sequence is flanked by homology arms or regions of high sequence identity with the targeted nucleotide sequence; in some instances, the regions of high identity include at least 10, at least 50, at least 100, at least 150, at least 200, at least 300, at least 400, at least 500, at least 600, at least 750, or at least 1000 nucleotides on each side of the core sequence. In some instances where the donor template is in the form of a single-stranded DNA, the core sequence is flanked by homology arms including at least 10, at least 20, at least 30, at least 40, at least 50, at least 60, at least 70, at least 80, or at least 100 nucleotides on each side of the core sequence. In instances where the donor template is in the form of a double-stranded DNA, the core sequence is flanked by homology arms including at least 500, at least 600, at least 700, at least 800, at least 900, or at least 1000 nucleotides on each side of the core sequence. Two separate double-strand breaks may be introduced into the cell or animal's target nucleotide sequence with a double nickase Cas9 (see Ran et al., Cell 154:1380-1389, 2013), followed by delivery of the donor template.

Gene editing methods disclosed herein may involve a guide RNA (gRNA) and a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. The choice of nuclease and gRNA(s) is determined by whether the targeted mutation is a deletion, substitution, or addition of nucleotides, e.g., a deletion, substitution, or addition of nucleotides to a targeted sequence. Fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be linked to the polypeptide to guide the composition to specific DNA sites by one or more RNA sequences (sgRNA) to modulate activity and/or expression of one or more target nucleic acids sequences. In some instances, the gRNA and the targeted nuclease are provided as a ribonucleoprotein complex (RNP).

The gene editing system may include a guide RNA (gRNA) for use in a CRISPR system for gene editing. Alternatively, the gene editing system may include a zinc finger nuclease (ZFN), or an mRNA encoding a ZFN, that targets (e.g., cleaves) a nucleic acid sequence (e.g., DNA sequence) of a gene in the animal. In other alternatives, the gene editing system may include a TALEN, or an mRNA encoding a TALEN, that targets (e.g., cleaves) a nucleic acid sequence (e.g., DNA sequence) in a gene in the animal.

For example, the gRNA can be used in a CRISPR system to engineer an alteration in a gene in the animal. In other examples, the ZFN and/or TALEN can be used to engineer an alteration in a gene in the animal. Exemplary alterations include insertions, deletions (e.g., knockouts), translocations, inversions, single point mutations, or other mutations. The alteration can be introduced in the gene in a cell, e.g., in vitro, ex vivo, or in vivo. The alteration may increase or decrease (e.g., knock-down or knock-out) the level and/or activity of a gene in the animal. In yet another example, the alteration corrects a defect (e.g., a mutation causing a defect), in a gene in the animal.

In some instances, the CRISPR system is used to edit (e.g., to add or delete a base pair) a target gene in the animal. In other instances, the CRISPR system is used to introduce a premature stop codon, e.g., thereby decreasing the expression of a target gene. In yet other instances, the CRISPR system is used to turn off a target gene in a reversible manner, e.g., similarly to RNA interference. In some instances, the CRISPR system is used to direct Cas to a promoter of a gene, thereby blocking an RNA polymerase sterically.

A CRISPR system can be generated to edit a gene in the animal, using technology described in, e.g., U.S. Publication No. 20140068797, Cong, Science 339: 819-823, 2013; Tsai, Nature Biotechnol. 32:6 569-576, 2014; U.S. Pat. Nos. 8,871,445; 8,865,406; 8,795,965; 8,771,945; and 8,697,359.

The CRISPR interference (CRISPRi) technique can be used for transcriptional repression of specific genes in the animal. In CRISPRi, an engineered Cas9 protein (e.g., nuclease-null dCas9, or dCas9 fusion protein, e.g., dCas9—KRAB or dCas9—SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

Alternatively, CRISPR-mediated gene activation (CRISPRa) can be used for transcriptional activation of a gene in the animal. In the CRISPRa technique, dCas9 fusion proteins recruit transcriptional activators. For example, dCas9 can be fused to polypeptides (e.g., activation domains) such as VP16, VP64, p65 activation domain (p65D), RTA, VPR, or SunTag and used with sgRNA (e.g., a single sgRNA or multiple sgRNAs), to activate a gene or genes in the animal. Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1).

The CRISPRi and CRISPRa techniques are described in greater detail, e.g., in Dominguez et al., Nat. Rev. Mol. Cell Biol. 17:5-15, 2016, incorporated herein by reference. In addition, dCas9-mediated epigenetic modifications and simultaneous activation and repression using CRISPR systems, as described in Dominguez et al., can be used to modulate a gene in the animal.

i. Generation of a Guide RNA for Use in CRISPR-Cas-Based Genetic Modification

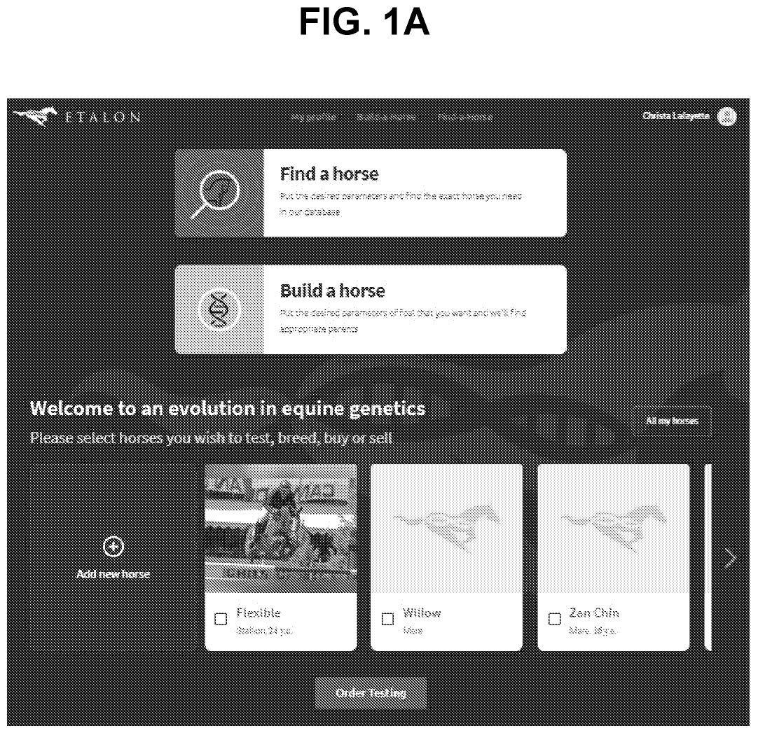

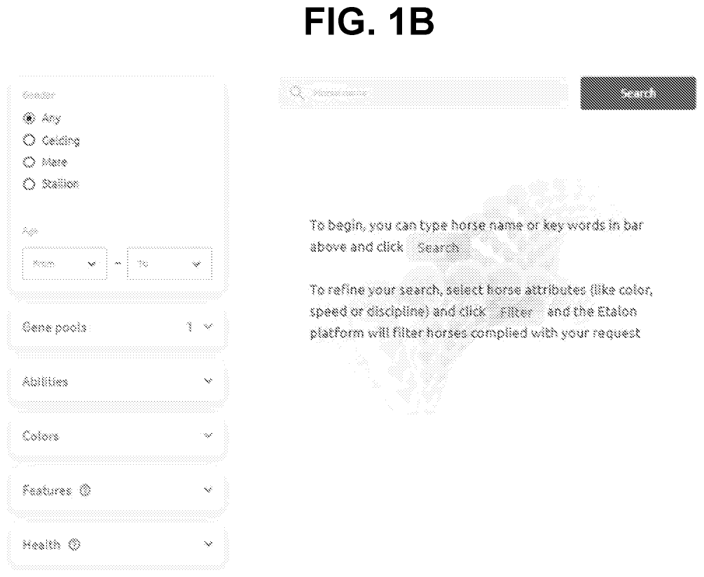

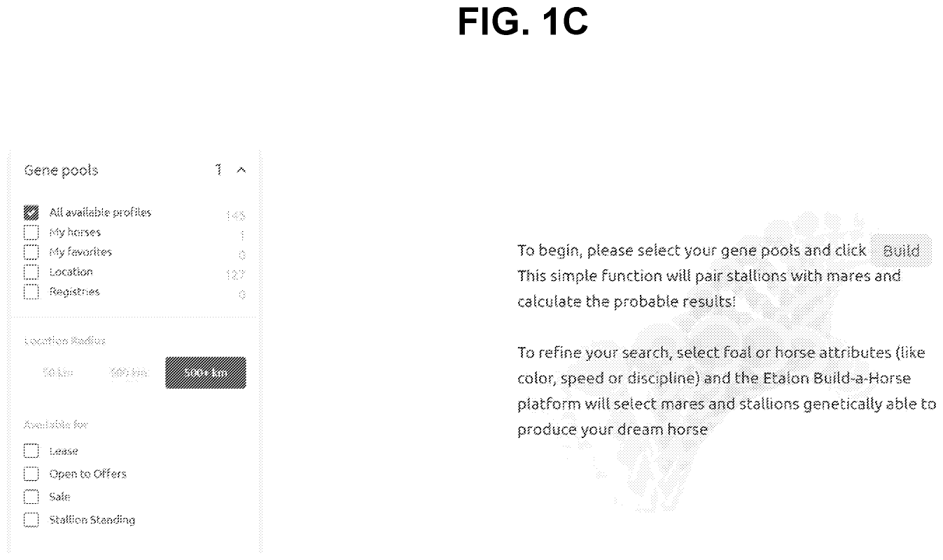

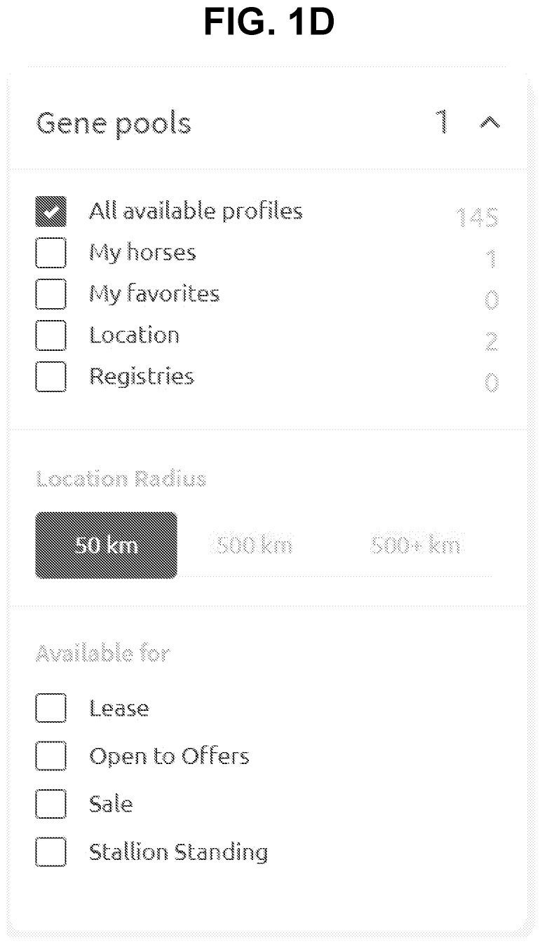

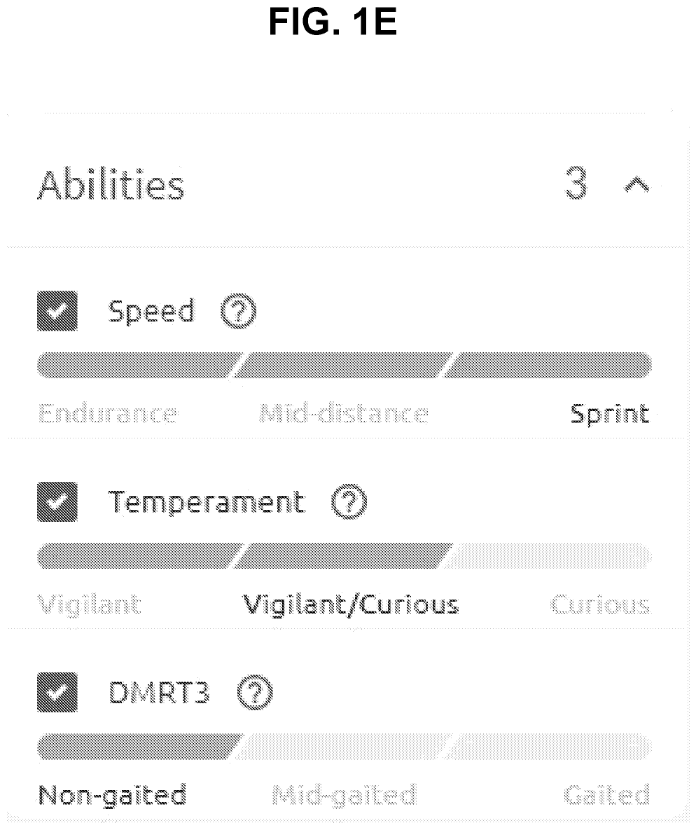

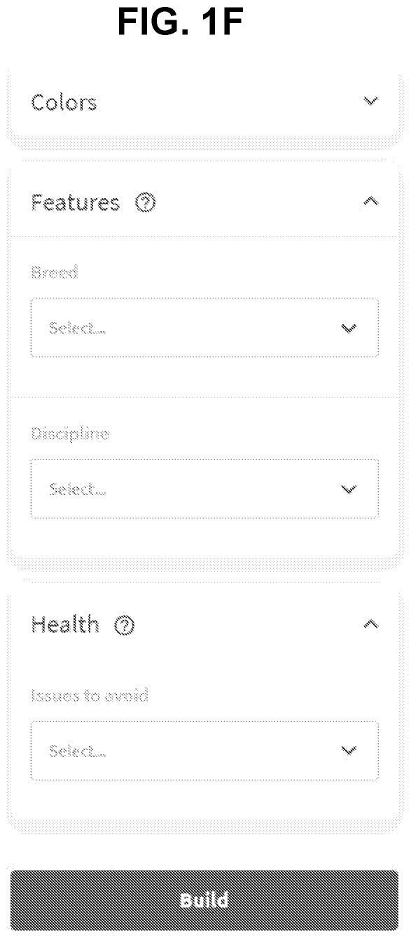

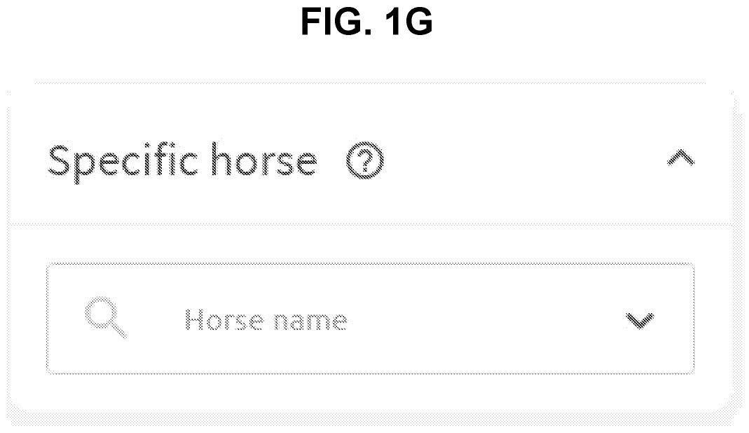

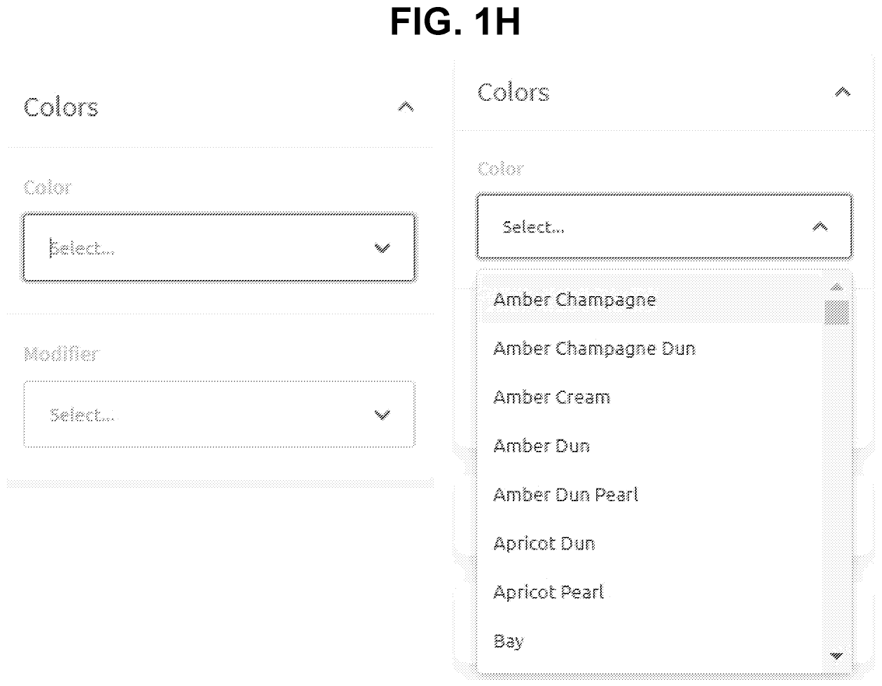

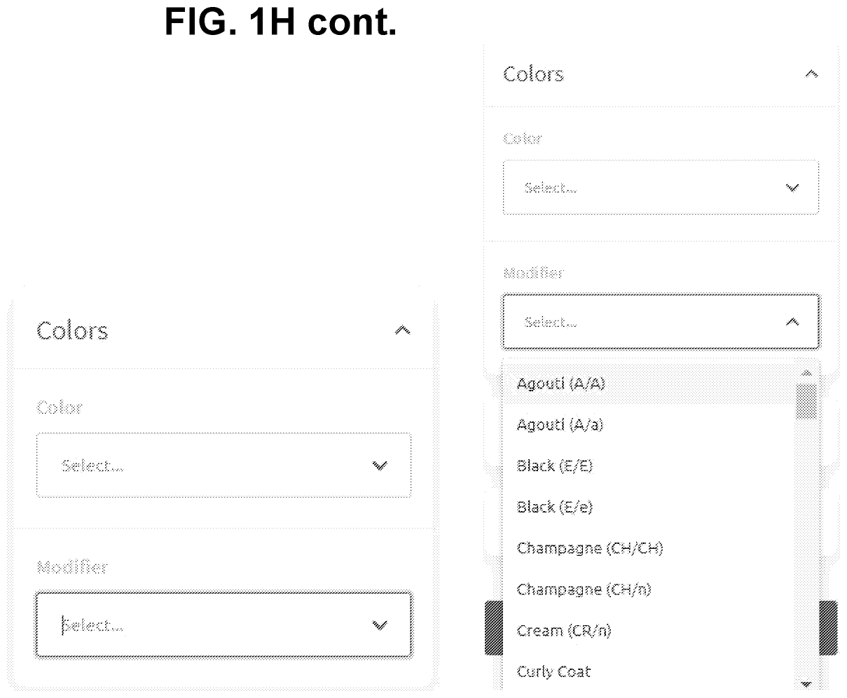

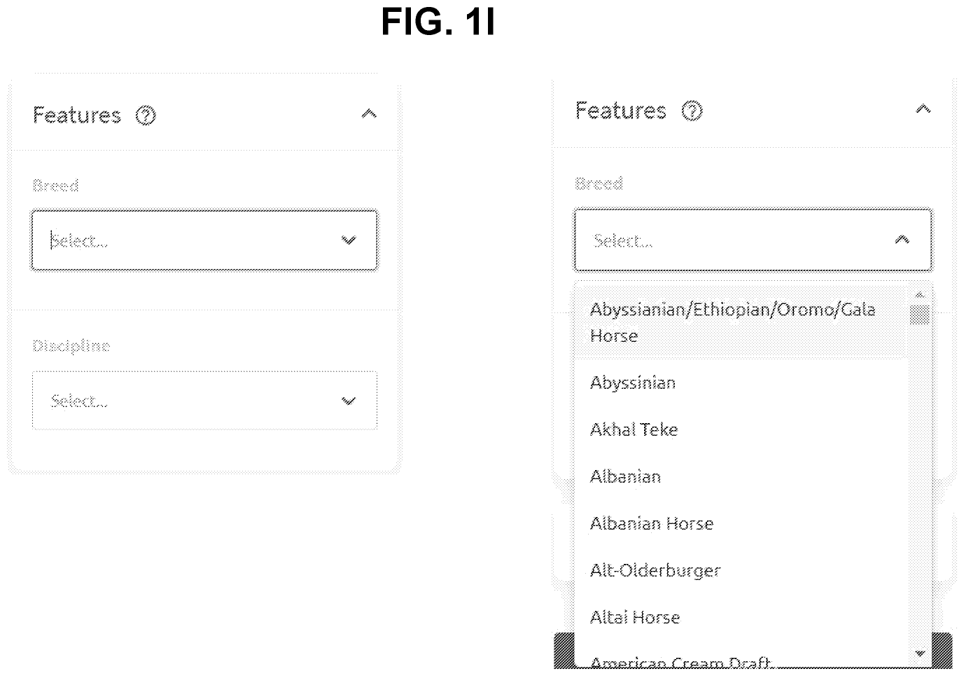

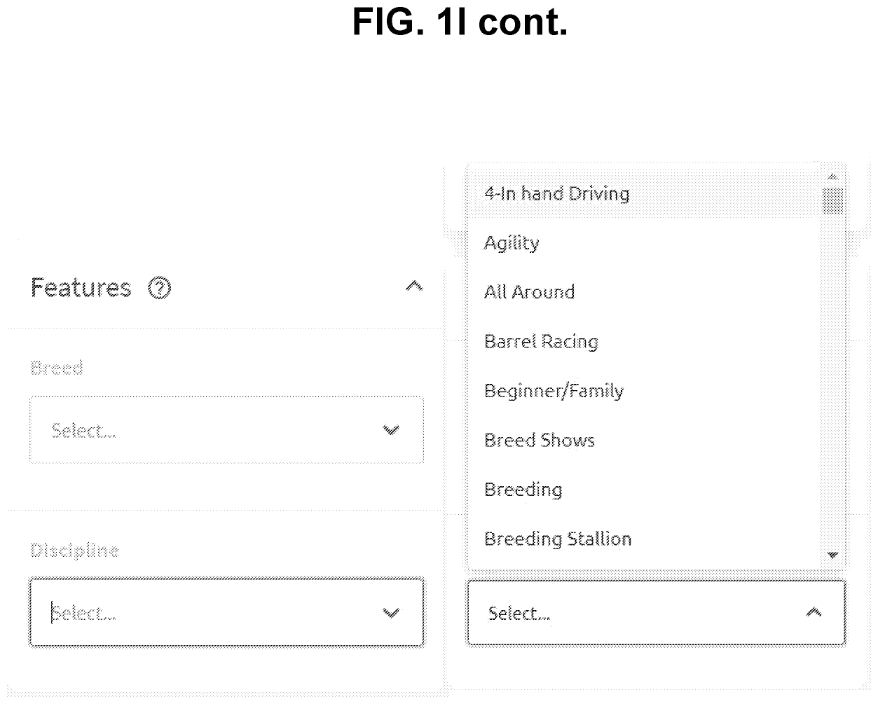

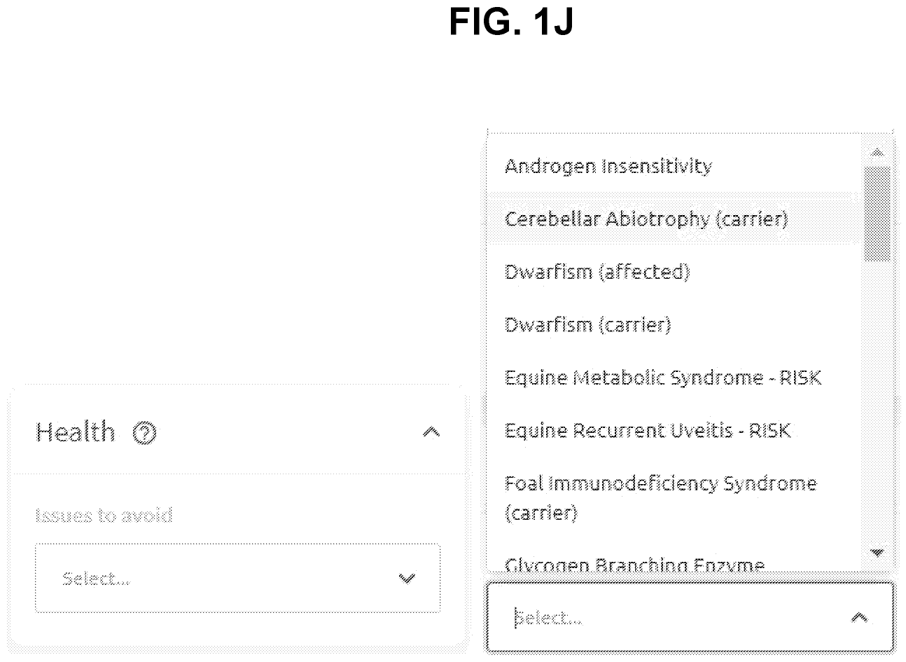

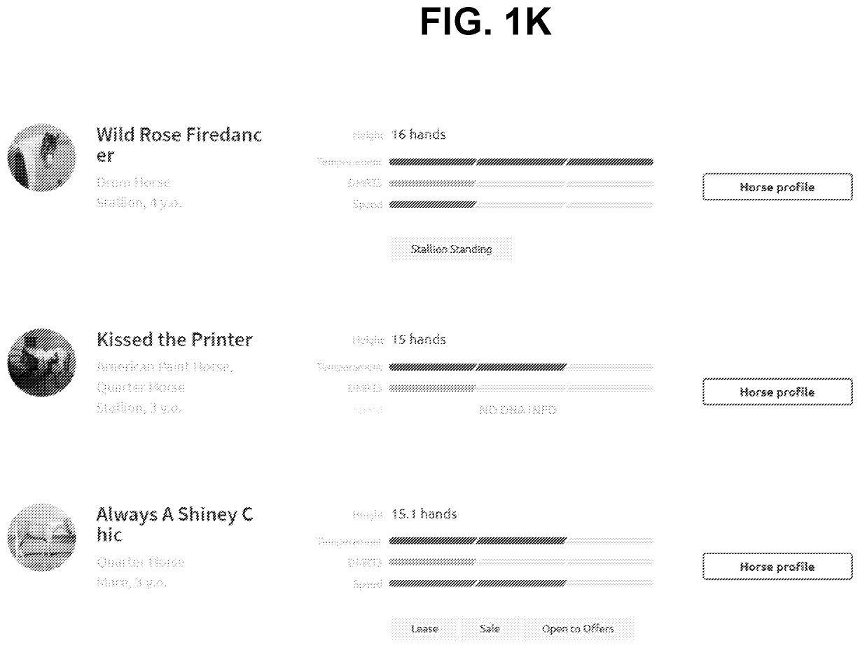

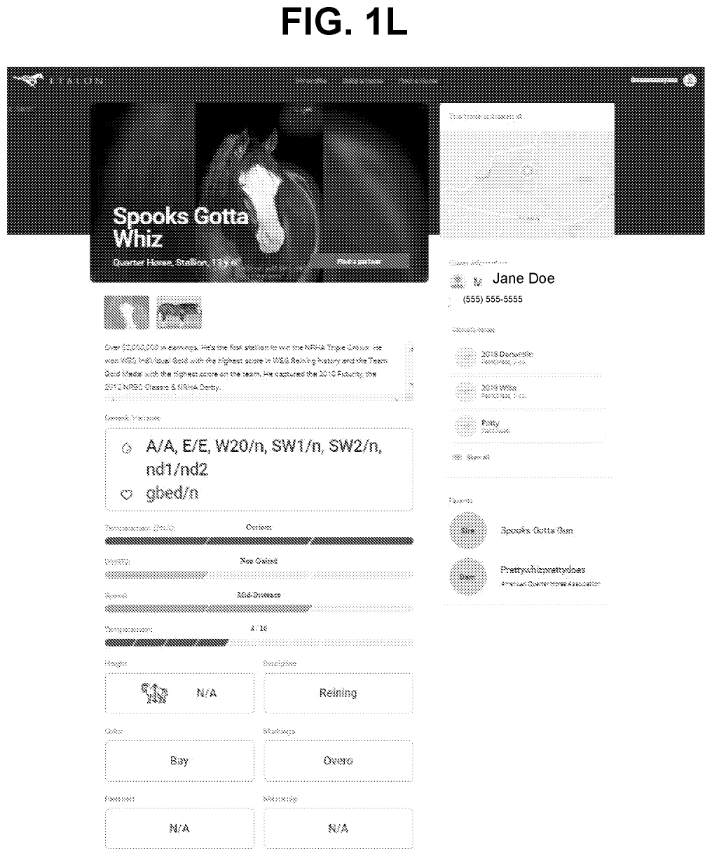

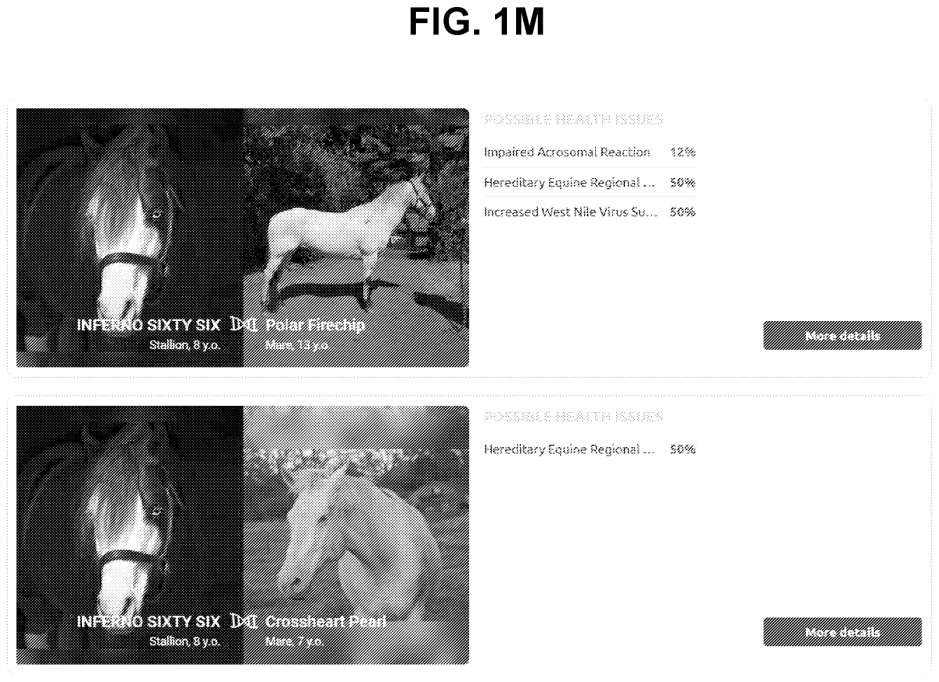

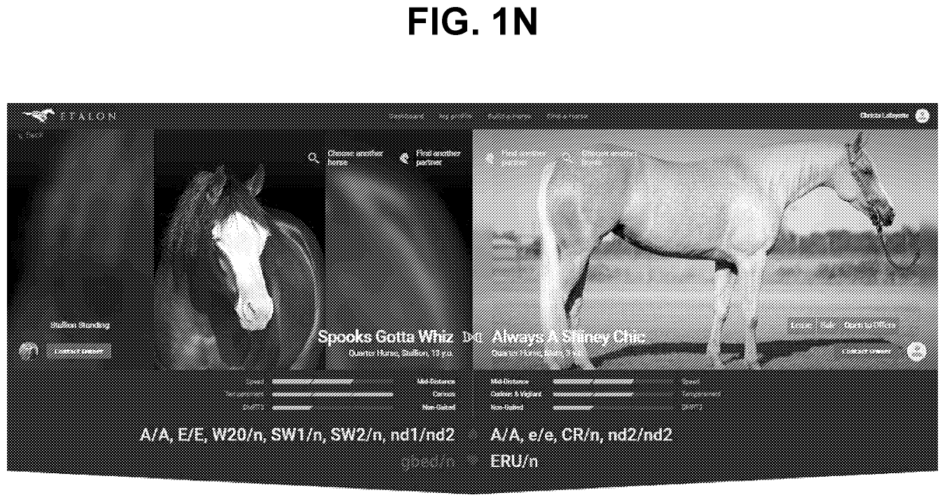

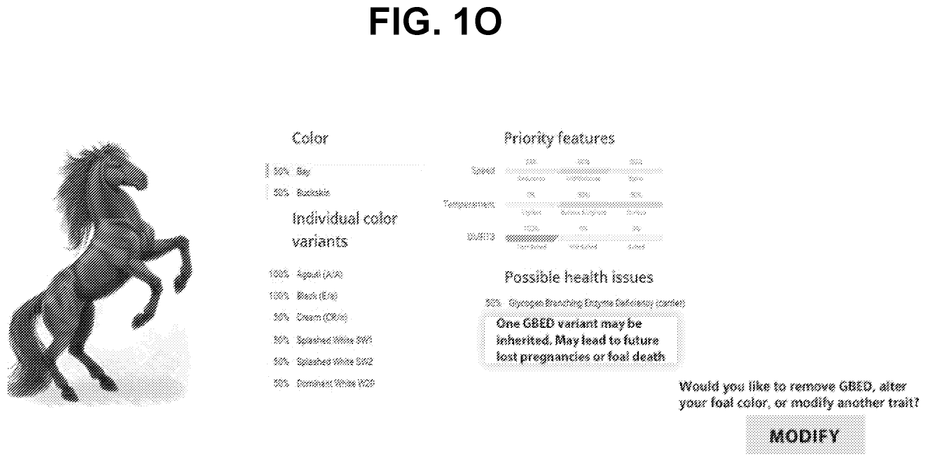

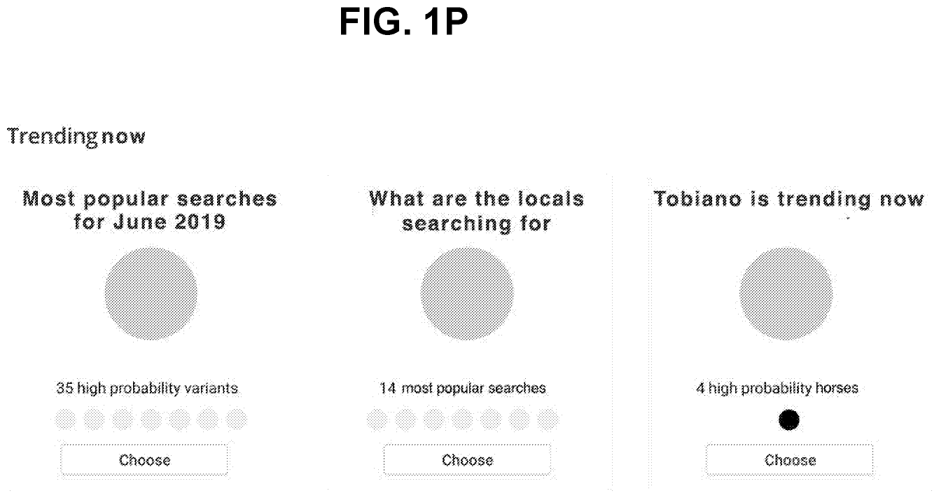

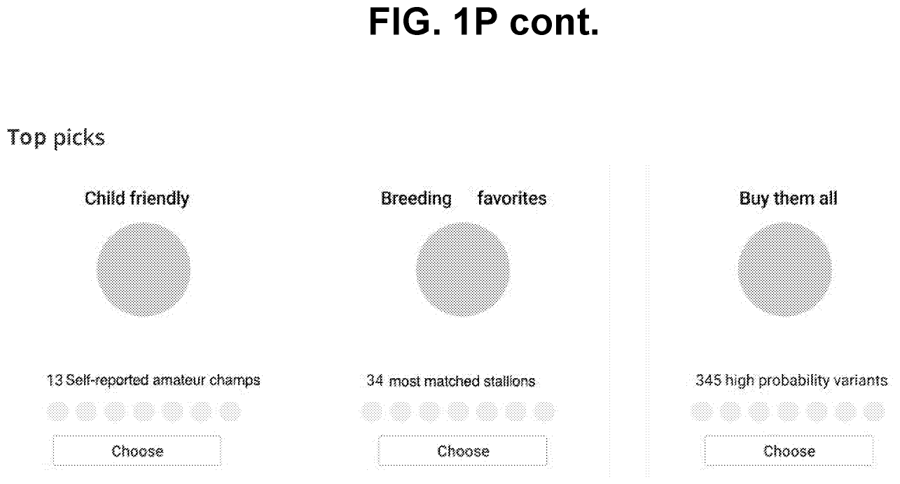

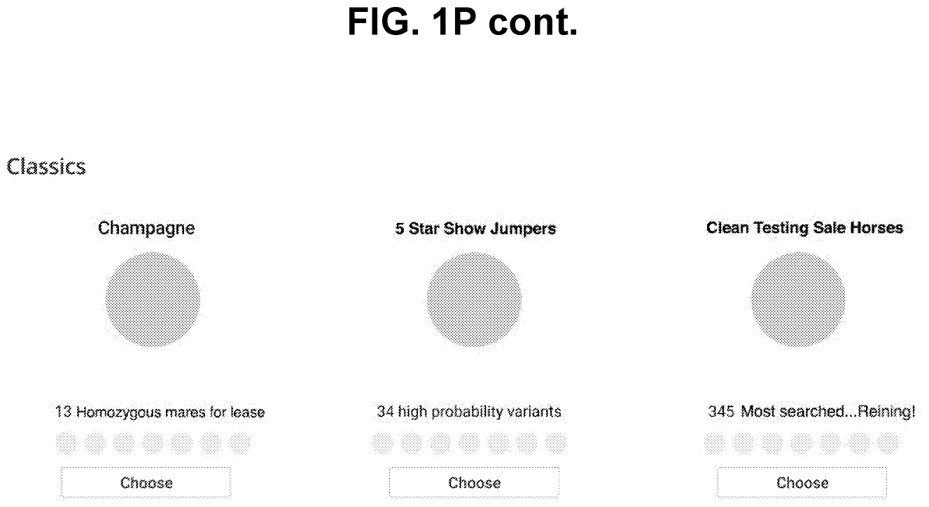

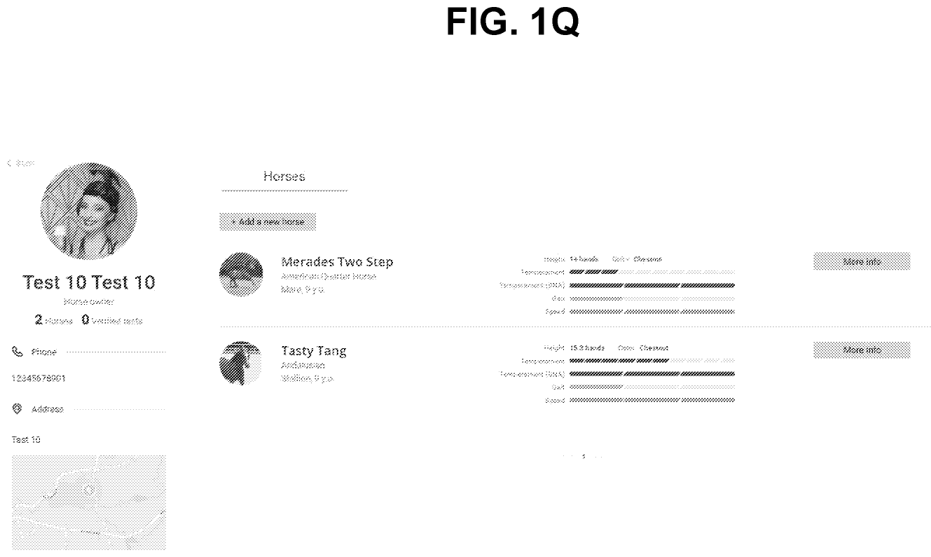

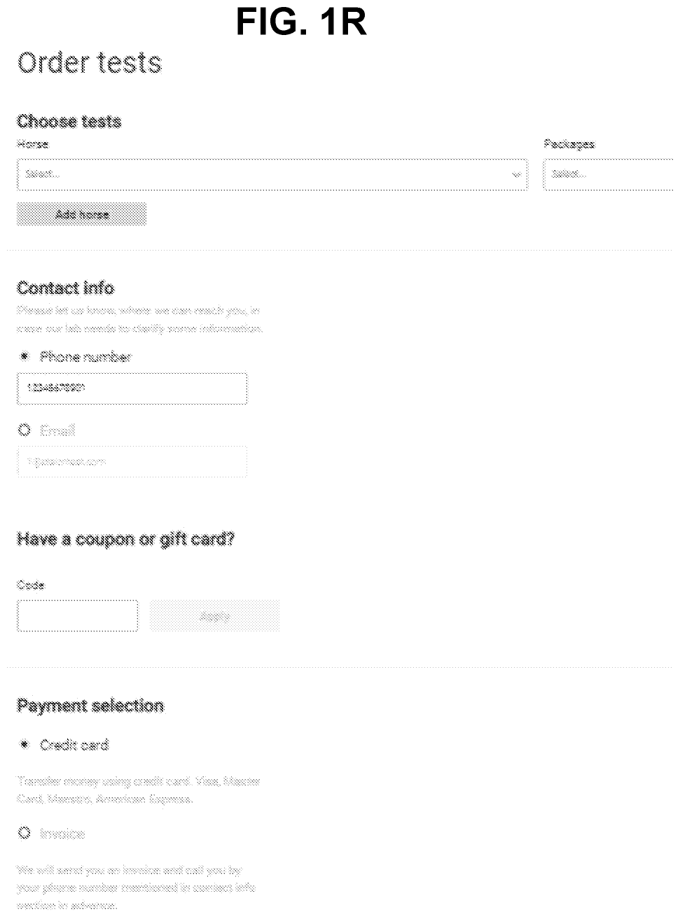

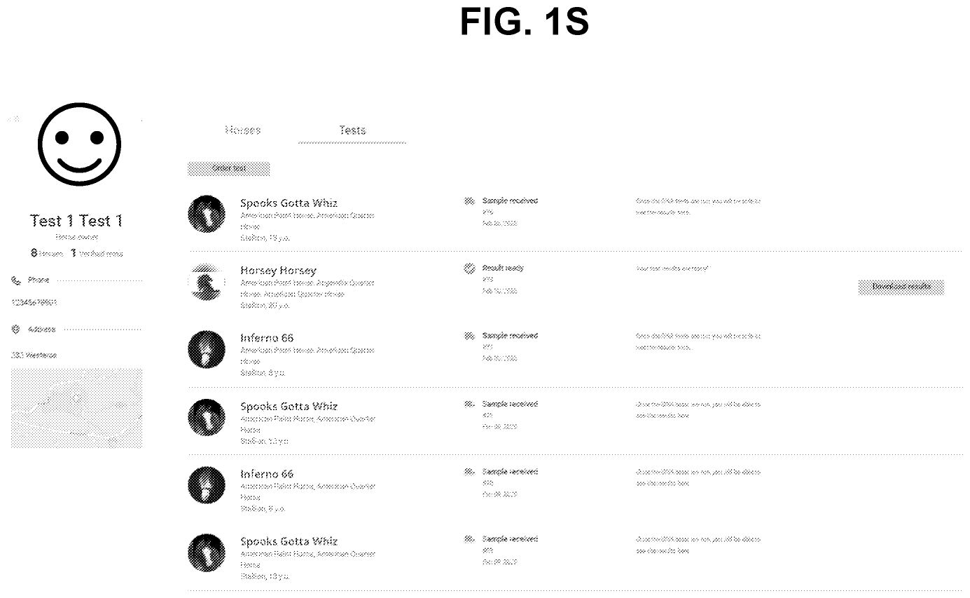

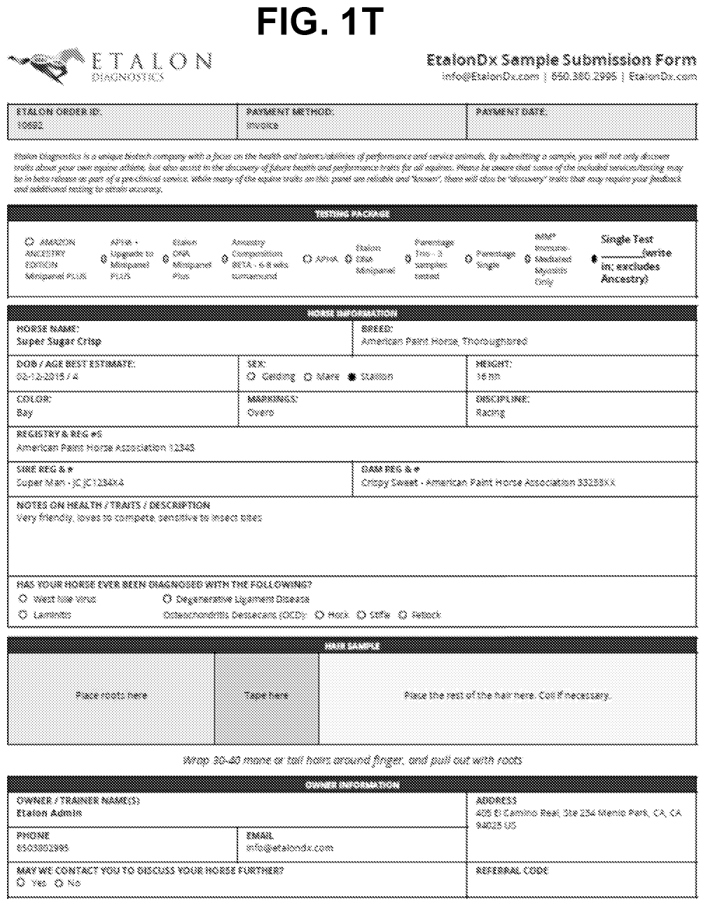

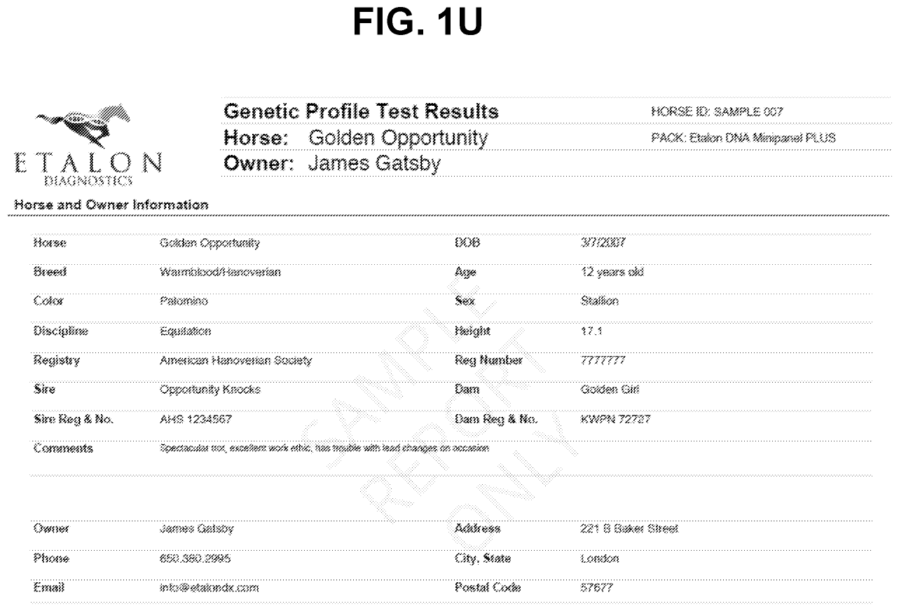

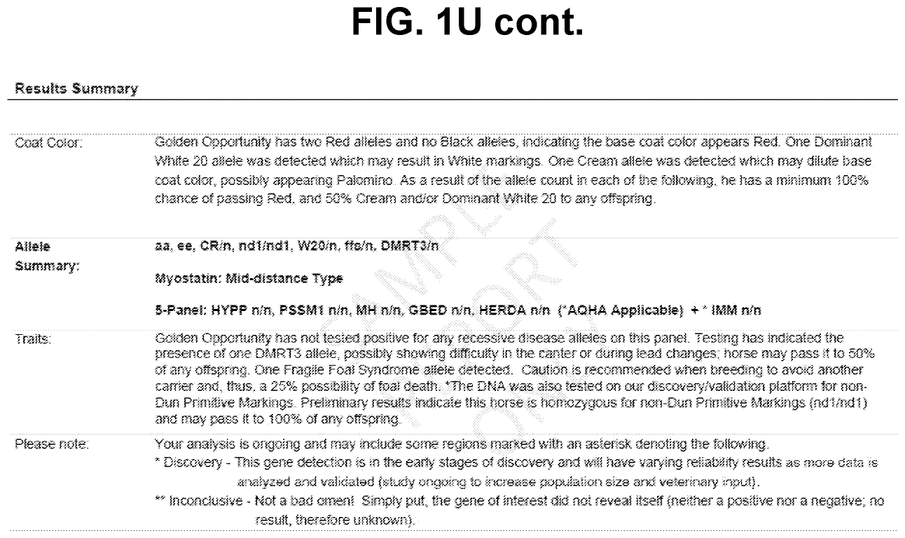

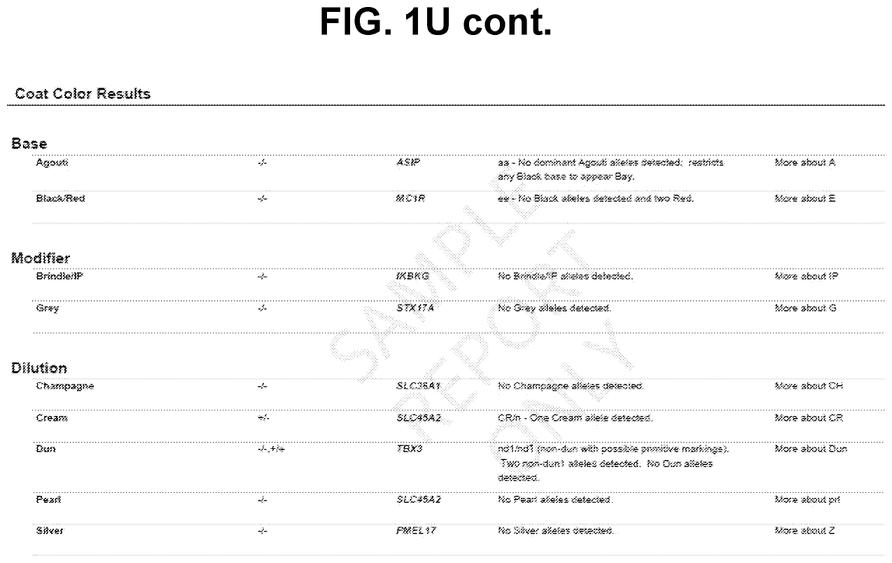

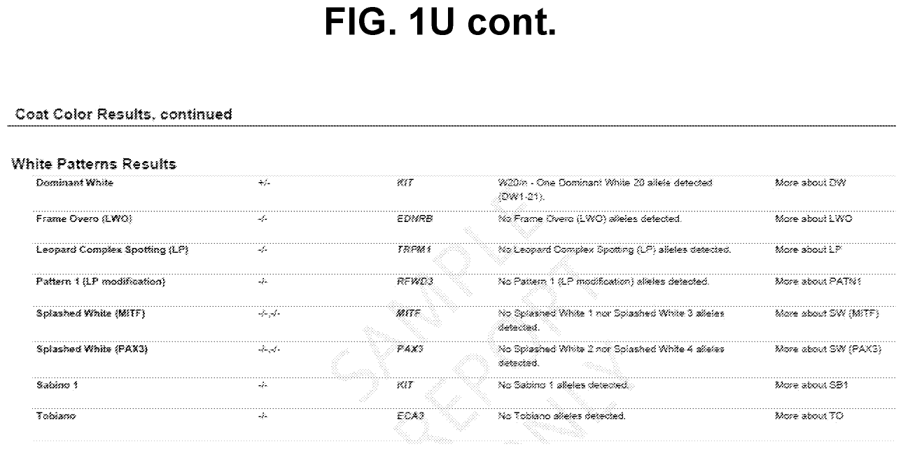

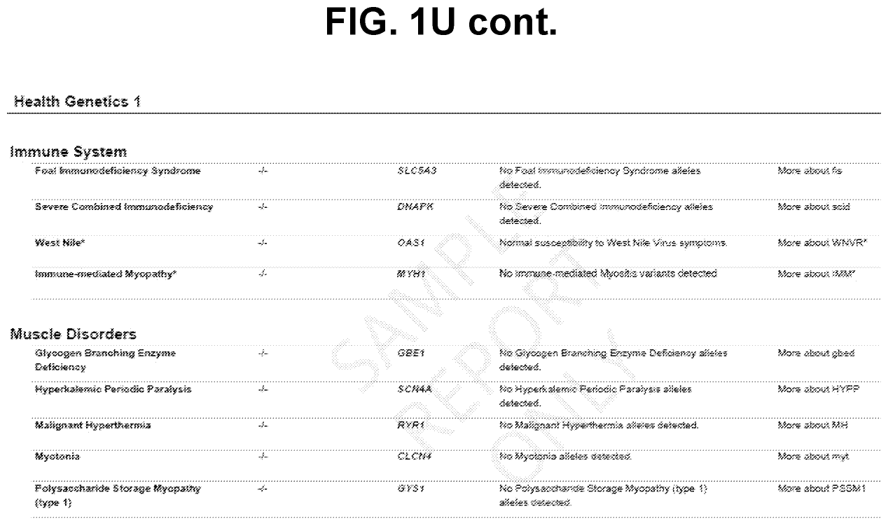

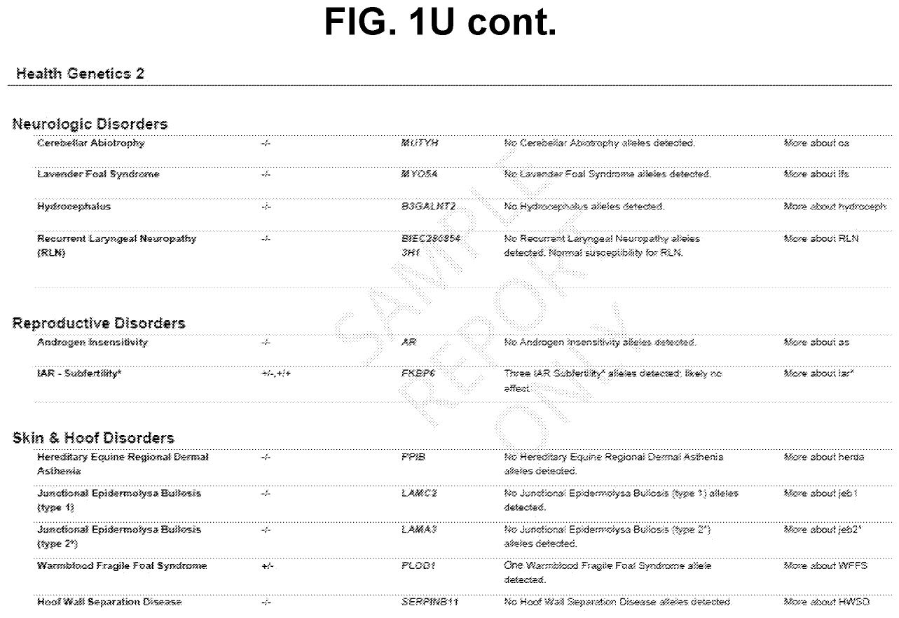

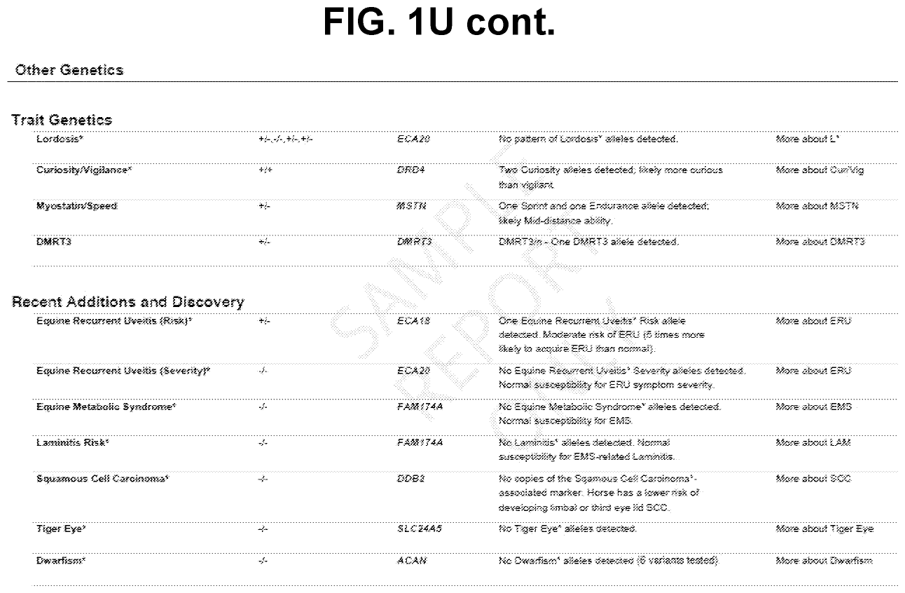

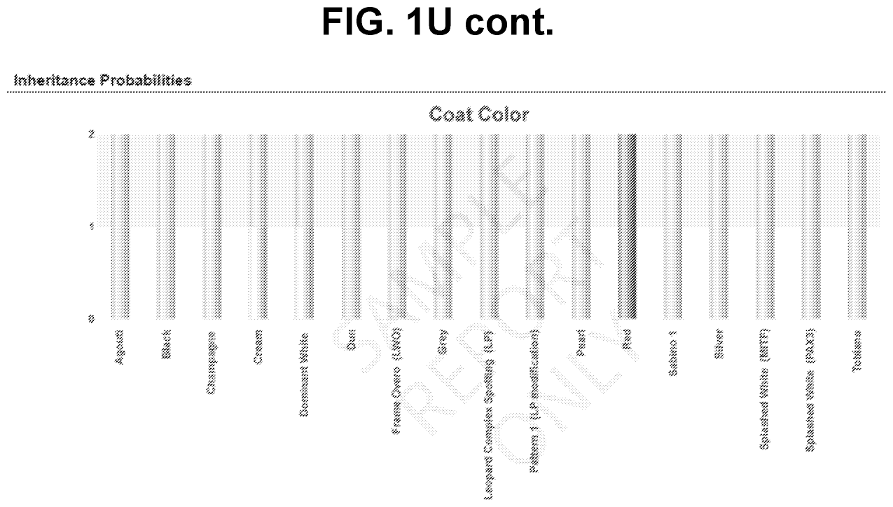

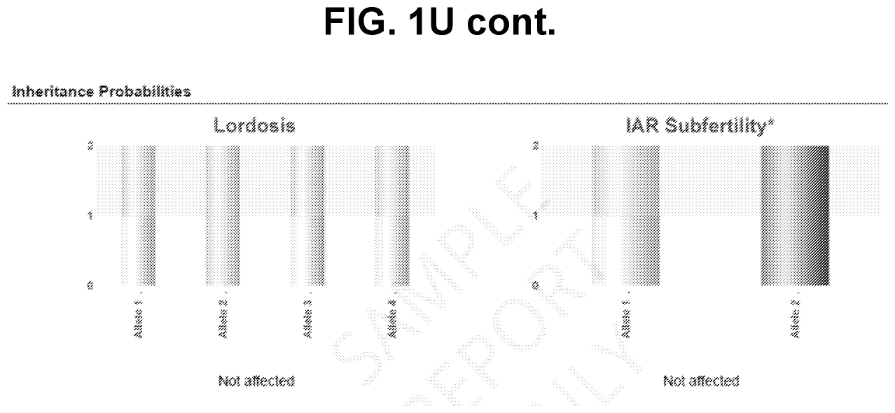

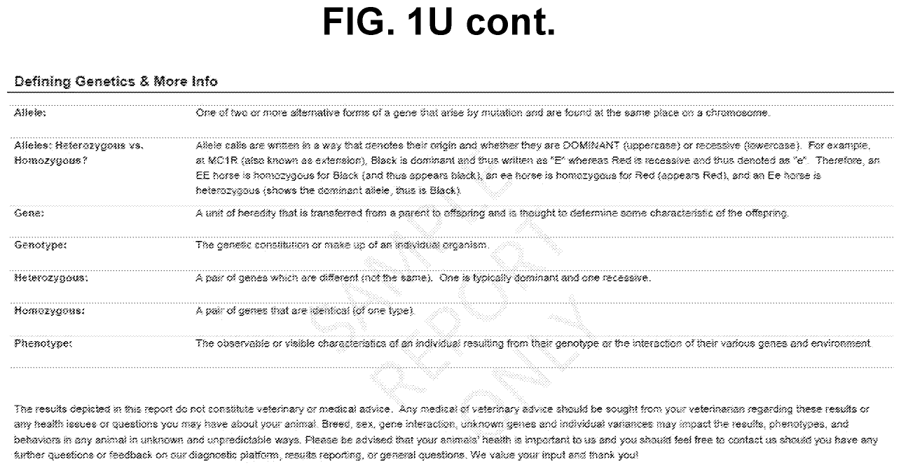

The present disclosure further relates to a method of producing a gRNA for use with a CRISPR-Cas-based gene editing system in conjunction with the methods and systems disclosed herein to produce an offspring animal having one or more predetermined traits. For example, a user of the database system of the disclosure may desire to produce a horse having a specific set of predetermined traits. Upon selecting said traits using the computer-implemented user interface system, the database system of the disclosure may report that the specific set of selected traits is likely to produce a potentially deleterious disease or non-disease condition in the foal (e.g., GBED; see FIG. 1O). The user interface may provide the user with an option to genetically modify the embryo of the foal in order avoid the disease or non-disease condition or to modify any other trait in the animal. Once the user opts to genetically modify the subject foal using the database system, the database system will utilize the genetic information of the foal as it relates to GBED to produce one or more gRNAs specifically targeting the affected region of the target GBE1 gene responsible for GBED. The one or more gRNAs may then be introduced into the embryo of the subject foal using methods described herein to produce a horse having one or more predetermined traits selected by the user and lacking the potentially deleterious risk of having GBED. Similar steps may be applied to achieve a desired phenotype in a non-human animal with regard to other traits disclosed herein.

### Computer-Implemented Graphical User Interface

The present disclosure features a computer-implemented, remote (e.g., web-based), graphical user interface that allows a user to execute a variety of functions including, but not limited to creating, viewing, and modifying a user profile, a non-human animal (e.g., a horse) profile, and/or a farm profile, configuring user settings, ordering and viewing diagnostic test results (e.g., genetic test results pertaining to one or more predetermined traits), searching for animals (e.g., horses) having one or more predetermined traits, selecting gene pools from which one or more candidate animals of a breeding pair can be selected, selecting one or more predetermined traits of a non-human animal to be produced, bought, or leased, selecting one or more predetermined traits of at least one member of a breeding pair capable of producing a non-human having one or more predetermined traits, viewing and selecting potential breeding pair matches capable of producing an animal having one or more predetermined traits with a high probability (e.g., 6.25%, 12.5%, 25%, 50%, 60%, 70%, 80%, 90%, 95%, 99%, or 100%.), saving, viewing, and selecting among trending presets containing discovered animals matching criteria based on previously selected trait combinations of a non-human animal, selecting one or more traits of a non-human animal that do not occur naturally and which may be achieved through the production of a transgenic non-human animal having those traits, performing ancestry analysis on a biological sample obtained from the non-human animal, providing payment information and making payments pertaining to services rendered by the platform of the disclosure, signing up for membership to the user platform, uploading and downloading data files (e.g., test results, images of user, animal, or farm), among others. The sections that follow describe the various uses and components of the computer-implemented user interface in detail.

### Use of a Computer-Implemented User Interface to Produce an Animal Having a Desired Phenotype

The computer-implemented user interface may be used to assist a user in producing, procuring, and/or identifying one or more non-human animals having one or more predetermined traits. The interface can be configured to allow a user to interrogate the computer-implemented database system described herein. Interrogation of the database may include selecting one or more predetermined traits of interest for the one or more non-human animals (see FIGS. 1E, 1F, and 1H-1I). The interrogation may further include identifying one or more of the non-human animals catalogued in the database system and generating a probability matrix for each of the one or more non-human animals identified using the database system (see, e.g., FIG. 1K). The probability matrix may include an array of probability values pertaining to the likelihood that the non-human animal has the one or more predetermined traits or the likelihood that the non-human animal, when used as a breeder animal in a breeding pair, will produce an offspring animal having the one or more predetermined traits. The system can then display an overall assessment of each of the non-human animals (e.g., one or more parent or offspring animals) having the one or more predetermined traits (see, e.g., FIG. 1O).

The user interface can include an option for the user to produce, purchase, or lease one or more of the non-human animals having the one or more predetermined traits, if desired, by, e.g., interrogating the database system of the disclosure to identify one or more non-human animals having the one or more predetermined traits. In the case that a user elects to identify a breeding pair capable of producing an offspring animal having one or more predetermined traits, the user may utilize the user interface to select one or more predetermined traits of at least one parent animal in a breeding pair (e.g., breed and/or discipline; see, e.g., FIG. 1I). A user may additionally utilize the user-interface to select a gene pool from which the one or more of the non-human animals (e.g., one or more of the parent animals in a breeding pair) are obtained (e.g., the user's own animals, animals owned by other users, an animal registry, and/or animals located within a specified geographical area (see, e.g., FIG. 1O)).

### User Input

A. Custom Selection of One or More Predetermined Traits

The computer-implemented user interface of the disclosure allows a user to select traits from a vast catalog of physical, behavioral, and genetic traits in order to produce, procure, and/or identify a non-human animal having a particular phenotype, such as a phenotype suitable for a specific discipline of the animal (e.g., any one of the disciplines disclosed herein). For example, the disclosed methods and systems are suitable for producing a non-human offspring animal having one or more predetermined traits. A non-human offspring animal having one or more predetermined traits may be produced by, e.g., interrogating a database system of the disclosure to identify one or more breeding pairs capable of producing a desired offspring animal. Production of the offspring animal may in some instances require genetically modifying the offspring animal or one or more parent animals to increase the likelihood of producing the desired phenotype in the offspring animal. A user may also employ the methods and systems disclosed herein to genetically modify the procured animal to promote the expression of the one or more predetermined traits in the animal.

The computer-implemented user interface described herein allows a user to select among one or more (e.g., 1, 2, 3, 4, 5, 6, 7, 8, 9, 10, or more) traits desired in a non-human animal from a panel of traits that may include, for example, coat color, coat color modifier, coat texture, coat thickness, facial marking, leg marking, eye color, skin color, mane color, tail color, temperament, speed, gait, and health.

Non-limiting examples of coat, mane, and/or tail color selections for a desired non-human animal (e.g., a horse) may include amber champagne, amber champagne dun, amber cream, amber dun, amber dun pearl, apricot dun, apricot pearl, agouti, bay, bay cream pearl, bay double cream, bay pearl, black, black bay, black cream pearl, black double cream, black dun, black pearl, black/red, blanket appaloosa, blood bay, blue roan, brindle, brown, buckskin, buttermilk buckskin, champagne, champagne amber pearl, champagne classic pearl, champagne dun, champagne dun pearl, champagne gold pearl, champagne pearl, chesetnut, chestnut cream pearl, chocolate, classic champagne, classic champagne dun, classic cream, classic dun, cream, cream champagne, cream grullo, cream pearl, cremello, dapple grey, dark bay, dark brown, dark chestnut, dominant white, dun, dun bay cream, dun bay double cream, dun black cream, dun black double cream, dun chestnut cream, dun chestnut double cream, dun pearl, dun pearl bay, dun pearl black, dunalino, dunolino, dunskin, dunskin splash, flaxen chestnut, fleabitten grey, frame overo, frosted appaloosa, grey, gold champagne, gold cream, gold dun, gold dun pearl, grey, grullo, half blanket, leopard appaloosa, light grey, liver chestnut, marbled appaloosa, overo, palomino, pearl, perlino, perlino dun, pseudocream classic, red, red chestnut, red dun, red roan, rose grey, sabino, seal brown, silver, silver amber champagne, silver amber dun, silver bay cream, silver bay cream pearl, silver bay double cream, silver black, silver black cream pearl, silver black double cream, silver champagne, silver champagne dun, silver champagne pearl, silver classic champagne, silver classic champagne pearl, silver cream pearl, silver dapple, silver dapple bay, silver dapple buckskin, silver dapple cream grullo, silver dapple dun, silver dapple dunskin, silver dapple grullo, silver dapple pearl, silver dapple perlino, silver dapple perlino dun, silver dun, silver dun bay cream, silver dun bay double cream, silver dun black, silver dun black double cream, silver dun pearl, silver dun pearl bay, silver dun pearl black, silver pearl bay, silver pearl black, silver red, silver smoky cream, skewbald, smoky black, smoky cream, smoky grullo, snowflake appaloosa, sorrel, splashed white, steel grey, sun, tobiano, tovero, tri-color, white, and white spotting.

According to the methods and systems disclosed herein, the user may choose to create a custom coat color of the non-human animal using a custom color palette that enables the selection of naturally-occurring or non-naturally-occurring coat colors of the animal. For example, the user may select a coat color modifier which may also include notation describing the animal genotype. Specifically, the genotype is represented by two letters or symbols separated by a slash, e.g., Z/n, wherein a dominant allele of a gene is represented by an uppercase letter and the recessive allele is represented by a lowercase letter. The wild-type allele may be represented using a “+” or an “n.” Exemplary coat color modifiers that can be selected by a user include agouti (e.g., NA or A/a), black (e.g., E/E or E/e), champagne (e.g., CH/CH or CH/n), cream (e.g., CR/n), curly coat, D/nd1, D/nd2, dominant white (e.g., W13, W13/W15, W13/W19, W13/W20, W13/W22, W15, W15/W15, W15/W19, W15/W20, W15/W22, W19, W19/W20, W19/W22, W20, W20/W20, W20/W22, W22, W22/W22, W5, W5/W13, W5/W15, W5/W19, W5/W20, W5/W22, W5/W5, W6, W6/W13, W6/W15, W6/W19, W6/W20, or W6/W22), dun (e.g., D/D), grey, leopard spotting complex (e.g., LP/n), overo (e.g., LWO/n), pattern 1, pearl (e.g., prl/n or prl/prl), red (e.g., e/e), sabino, silver (e.g., Z/Z or Z/n), splashed white (e.g., SW1, SW1/SW1, SW1/SW3, SW1/SW5, SW2, SW2/SW2, SW2/SW4, SW3, SW3/SW3, SW3/SW5, SW4, SW4/SW4, SW5, or SW5/SW5), apron, barring on body, barring on shoulder, belly spots (large or small), belly stripe, belted, bend or spots on body, bend or spots on head, birdcatcher spots, black spots, blagdon, blanket with roaning, blanket with spots, blaze, blaze with freckling, body spots, body white, brindle, brow spots, calico, coon/skunk tail, dapples, dilute, dorsal stripe, double dilute, ermine markings, few white hairs on body, fewspot, flaxen mane/tail, flea-bitten, fleshmark, frosted, grullo, heart marking, highlights in mane/tail, lacing pattern, leopard spotted, lightning marks, line back, frosting mane/tail, maximum tobiano, maximum overo, maximum white, maximum white sabino, medicine hat, minimal overo, minimal sabino, minimal tobiano, mixed mane/tail, mottled, no marking, overo, pangare, paw prints, pearl, piebald, pinto markings, rabicano, roan, roaning on body, rose, rump spots, sabino, skewbald, smudge marks, smutty, snip, snowcap, sooty, splash markings, star, stripe, tobiano, tovero, umbilical spots, white head, white mane/tail, white throat latch, white tipped ears, white with fading edges, white with ragged edges, white with smooth edges, wither spot, no markings, solid, white blaze, and white star.

The user may also select from a panel of facial marking selections desired in the non-human animal (e.g., a horse), including apron face, badger face, bald face, blaze, interrupted stripe, face, snip, star, stripe markings, both eyes amber, both eyes blue, both eyes brown, both eyes green, both eyes tiger, eyebrows, face mask, few white hairs on forehead, left eye amber, left eye blue, left eye brown, left eye green, left eye partial blue, left eye tiger, partial bald face, pigment around eye, right eye amber, right eye blue, right eye brown, right eye green, right eye partial blue, right eye tiger, white around eye, white chin, white jaw, white lip, white nose, and white sclera.

The user interface further allows a user to make selections pertaining to leg marking in a non-human animal (e.g., a horse), including, but not limited to two back white stockings, two white front socks, two white front stockings, two white hind socks, two white hind stockings, four white socks, four white stockings, back left above hock, back left cannon (¾ stocking), back left coronet, back left ermine spots, back left fetlock, back left half pastern, back left heel, back left partial heel, back left pastern, back left sock, back left stocking, back left stripes, back right above hock, back right cannon, back right coronet, back right ermine spots, back right fetlock, back right half pastern, back right heel, back right partial heel, back right pastern, back right sock, back right stocking, back right stripes, barring on legs, bend or spots on legs, front left above knee, front left cannon, front left coronet, front left ermine spots, front left fetlock, front left half pastern, front left heel, front left partial heel, front left pastern, front left sock, front left stocking, front left stripes, front right above knee, front right cannon, front right coronet, fright right ermine spots, front right fetlock, front right half pastern, front right heel, front right partial heel, front right pastern, front right sock, front right stocking, front right stripes, left front hoof stripes, left front white foot, left hind hoof stripes, left hind white foot, right front hoof stripes, right front white foot, right hind hoof stripes, right hind white foot, and roaning on white socks/stockings.

Selection of coat texture traits in the non-human animal may be made by a user from a list including smooth, rough, curly, straight, downy, spiky, or brindle. Additionally, coat thickness may also be selected according to the disclosed methods, wherein the coat thickness may be thin, medium, or thick.

Furthermore, the user interface of the disclosure allows a user to select an eye color, such as, e.g., blue, amber, yellow, orange, hazel, green, or brown, and skin color, such as, e.g., black, brown, pink, yellow, and white, for the desired offspring animal.

Additionally, the user interface allows a user to select an input for each of coat color, coat color modifier, coat texture, coat thickness, facial marking, eye color, skin color, mane color, tail color, and leg marking traits, such as those listed above, in alone or in combination with each of the above-listed traits. The aforementioned lists of coat colors, facial markings, and leg markings selections are not exhaustive and others known in the art may be incorporated into the database for selection by a user.

A user may employ the user interface of the disclosure to input a selection for a temperament trait of the desired non-human animal. Non-limiting examples of temperament traits that can be selected by a user include vigilant, curious, curious/vigilant, spooky, non-spooky, hot, cold, and medium. Other temperament traits known in the art could also be catalogued in the database of the present disclosure and interrogated by a user using the user interface of the system described herein.

Other examples of trait selections that can be made by a user utilizing the user interface include a speed trait for the desired non-human animal. The speed trait may be selected from sprint, mid-distance, and endurance, among others. Other speed trait selections can be catalogued in the database of the present disclosure and interrogated using the user interface of the system described herein.

The user interface described herein also facilitates the selection of a gait trait of the non-human animal. Accordingly, the user may select a gait trait in the animal from a list that includes, but is not limited to gaited, non-gaited, and gait carrier. Other gait options of the animal may also be incorporated into the database for selection by a user. Selection of a gaited animal may further include selection between the gaits including, but not limited to walk, trot, canter, gallop, pacing, fox trot, racking, country pleasure, Indian shuffle, jogging, paso fino, paso corto, paso largo, paso llano, sobrandando, marcha picada, rack, running walk, stepping pace, singlefoot, tölt, and ravaal.

According to the present disclosure, a user may desire to select one or more health conditions (e.g., diseases, such as, e.g., genetic diseases) to be avoided when creating a non-animal having the desired phenotype. Therefore, the present disclosure provides for selection of health traits that includes a selection of one or more disease conditions to be excluded or permitted in the animal of interest (e.g., a horse). Disease conditions that may not be desirable in the non-animal include but are not limited to those disclosed in Table 10, below, as well as anhidrosis, degenerative suspensory ligament disease, equine asthma, equine herpes virus risk, epistaxis risk, kissing spines, tendinopathy, wobbler syndrome, and behavioral abnormality (e.g., cribbing, weaving, anxiety, depression, extreme aggression, extreme fearfulness, bucking, striking, biting, self-mutilating, or head shaking). Other disease conditions may be selected to be excluded or permitted in the non-human animal of interest.

TABLE 10
Genetic basis for disease and non-disease conditions in horses

| SEQ ID | | |
|---|---|---|
| NO: | Gene/chromosomal location | Conditions |
| 1 | Androgen receptor (AR) | Androgen insensitivity syndrome |
| 2 | MutY DNA glycosylase (MUTYH) | Cerebellar abiotrophy |
| 3 | Teashirt Zinc finger homeobox 1 (TSHZ1) | Curved ear tips |
| 4 | Aggrecan (ACAN) | Dwarfism |
| 5 | Family with sequence similarity 174 member A | Equine metabolic syndrome and |
| | (FAM174A) | laminitis |
| 6 & 39 | Interleukin 17A (IL17A) and interleukin 17B | Equine recurrent uveitis |
| | (IL17B) | |
| 7-11 | Chemokine (C-X-C motif) ligand 16 (CXCL16) | Equine viral arteritis susceptibility |
| 12 | Solute carrier family 5 member 3 (SLC5A3) | Foal immunodeficiency syndrome |
| 13 | Doublesex and Mab-3 related transcription factor 3 | Gait transition (loss of canter) |
| | (DMRT3) | |
| 14 | Glycogen branching enzyme (GBE1) | Glycogen branching enzyme |
| | | disorder/deficiency |
| 15 | Peptidylprolyl isomerase B (PPIB) | Hereditary equine regional dermal |
| | | asthenia |
| 16 | Serpin family B member 11 (SERPINB11) | Hoof wall separation disease |
| 17 | Beta-1,3-N-acetylgalactosaminyltransferase 2 | Hydrocephalus |
| | (B3GALNT2) | |
| 18 | Sodium voltage-gated channel alpha subunit 4 | Hyperkalemic periodic paralysis |
| | (SCN4A) | |
| 19 | Myosin heavy chain 1 (MYH1) | Immune-mediated myositis |
| 20-22 | FKBP prolyl isomerase 6 (FKBP6) | Impaired acrosomal reaction |
| | | (subfertility) |
| 23 | Laminin subunit gamma 2 (LAMC2) | Junctional epidermolysis bullosa |
| 24 | Myosin 5A (MYO5A) | Lavender foal syndrome |
| 25-28 | Equine chromosome 20 (ECA20) | Lordosis |
| 29 | Ryanodine receptor 1 (RYR1) | Malignant hyperthermia |
| 30 | Chloride voltage-gated channel 4 (CLCN4) | Myotonia |
| 31 | Glycogen synthase 1 (GYS1) | Polysaccharide storage myopathy |
| 32 | Equine chromosome 3 (ECA3) | Recurrent laryngeal neuropathy |
| | | (roaring) |
| 33 | Protein kinase, DNA-activated, catalytic subunit | Severe combined immunodeficiency |
| | (DNAPK) | |
| 34 | Damage specific DNA binding protein 2 (DDB2) | Squamous cell carcinoma |
| 35 | Procollagen-lysine, 2-oxoglutarate 5-dioxygenase | Warmblood fragile foal syndrome |
| | 1 (PLOD1) | |
| 36 | 2′-5′-oligoadenylate synthetase 1 (OAS1) | West Nile Virus susceptibility |

The user interface disclosed herein also allows for the selection of one or more predetermined traits in one or more parents of a non-human offspring animal. For example, a user may select a filter that limits the selection of one or more of the parents of the offspring animal to exhibit one or more desired traits. Non-limiting examples of parent traits include breed, discipline, and height, among others. The breed of one or more of the parent animals may be selected from a list that includes, for example, Abyssianian/Ethiopian/Oromo/Gala horse, Abyssinian, Akhal Teke, Albanian, Albanian Horse, Alt-Olderburger, Altai Horse, American Cream Draft, American Cream and White, American Curly, American Gaited Mountain Horse, American Gaited Pony, American Paint Horse, American Quarter Horse, American Saddlebred, American Walking Pony, American Warmblood, Andalusian, Andravida, Andravida/Eleia Horse, Anglo European Warmblood, Anglo-Arab, Anglo-Kabarda, Appaloosa, Appendix Quarter Horse, AraAppaloosa, Arabian, Arabian Sporthorse, Ardennes, Ardennes Horse, Argentine Criollo, Asturcon, Asturian, Australian Brumby, Australian Stock Horse, Austrian Warmblood, Azteca, Baise Horse, Balearic, Baluchi, Baluchi Horse, Ban-ei, Banker, Banker Horse, Barb, Barb/Berber Horse, Bashkir, Bashkir Curly, Bashkir Curly Horse, Bashkir Horse, Basotho Pony, Basuto Pony, Belgian, Belgian Draft, Belgian Warmblood, Bhirum Pony, Bhirum/Nigerian Pony, Bhotia Pony, Bhutia Pony, Black Forest, Black Forest Pony, Blue Eyed Horse Association, Boer, Boerperd, Brandenburger, Breton, Breton Horse, British Riding Pony, British Warmblood, Buckskin, Budyonny, Budyonny Horse, Byelorussian Harness, Byelorussian Harness Horse, Camargue, Camargue Horse, Campolina, Canadian Horse, Canadian Sport Horse, Canadian Suffield Mustang, Canadian Warmblood, Carolina Marsh Tacky, Carthusian, Caspian, Caspian Horse, Cayuse, Celtic Warmblood, Cheju, Chicoteague Pony, Chilean Corralero, Chilean Corralero Horse, Chincoteague Pony, Cleveland Bay, Clydesdale, Colorado Ranger Horse, Connemara, Cream Draft, Cremello, Criollo, Crioulo, Curly Horse, Dales Pony, Danish Warmblood, Danube, Danube Delta Horse, Dartmoor Pony, Deliboz, Deliboz/Delibozskaya, Djerma, Dole, Dole Gudbrandsdal/Dolahest/Døle, Dongola, Donkey, Draft, Draft Cross, Draft Horse, Drum Horse, Dutch Draft, Dutch Harness, Dutch Harness Horse, Dutch Warmblood, East Bulgarian, East Friesian, Egyptian, Egyptian Arabian, Eriskay Pony, Estonian Horse/Estonian Native/Estonian Klepper/Eisti Hobune, Estonian Native, Exmoor Pony, Faeroe Pony, Faeroes Pony, Falabella, Falabella Miniature Horse, Fell Pony, Ferghana, Finnhorse, Finnhorse/Finnish Horse, Fjord, Fjord Horse, Fleuve, Florida Cracker, Florida Cracker Horse, Foundation Quarter Horse, Fouta, Foutanke/Fouta, Frederiksborg, Frederiksborg Horse, French Saddlebred, French Saddlebred Horse, French Trotter, Friesian, Friesian Cross, Galiceño, Galician Pony, Galician/Galician Mountain Horse, Gelderlander, Georgian Grande, German Bred, German Riding Pony, Gidran, Gidran/Hungarian Anglo-Arab, Golden American Saddlebred, Gotland, Gotland Pony, Groningen, Groningen Horse, Guangxi, Gypsy Cob, Gypsy Vanner, Hackney, Hackney Horse, Hackney Pony, Haflinger, Half-Arabian, Hanoverian, Hanoverian Warmblood, Hequ, Highland Pony, Hokkaido, Hokkaido Horse, Holsteiner, Holsteiner Warmblood, Hucul, Hucul Pony, Hungarian Warmblood, Iberian Warmblood, Icelandic, Icelandic Horse, lomud, lomud/Yomud/Yamud/Yomut, Irish Cob, Irish Draft, Irish Draught, Irish Draught Horse, Irish Sport Horse, Jeju Horse, Jinzhou, Jutland, Kabarda, Kabarda/Kabardin, Karabair, Karabakh, Kazakh, Kazakh Horse, Kentucky Mountain horse, Kentucky Mountain Saddle Horse, Kerry Bog Pony, KigeR Mustang, Kirdi Pony, Kisber Felver, Kiso, Kiso Horse, Kladbury, Kladbury Horse, Knabstrup, Knabstrupper Warmblood, Kushum, Kushum Horse, Kustanai, Kustanai Horse, Kyrgyz/Kirgiiz Horse, Latvian, Latvian Horse, Lipizzan, Lithuanian Heavy Draft, Lokai, Losino, Losino Horse, Lusitano, M Bayar, Mallorquin, Malopolski, Mangalarga, Mangalarga Marchador, Marsh Tacky, Marwari, Marwari Horse, Wrens Pony, Messara, Messara Horse, Miniature, Miniature Horse, Misaki, Misaki Horse, Missouri Fox Trotter, Missouri Fox Trotting Horse, Miyako, Miyako Pony, Mongolian, Mongolian Horse, Morab, Morgan, Mountain Pleasure Horse, Moyle, Mule, Murgese, Mustang, National Show horse, National Spotted horse, New Forest Pony, New Kirgiz, Newfoundland Pony, Nokota, Nokota Horse, Noma, Noma Horse, Nooitgedacht Pony, Nordland, Nordlandshest/Lyngshest/Nordland Pony, Norfolk Trotter, Noric, North Swedish horse, Northeastern, Ob, Oldenburg, Oldenburg Warmblood, Orlov Trotter, Paint (Overo), Paint (Solid), Paint (Tobiano), Paint (Tovero), Paints (All), Palomino, Pantaneiro, Pantaneiro horse, Paso Fino, Paulista Marchador, Peneia Pony, Percheron, Peruvian Paso, Pindos Pony, Pinia, Pintabian, Pinto, POA, Polish Horse, Ponies (All), Pony, Pony of America, Pottok, Priob/Ob, Przewalski, Przewalski Horse, Pura Raza Espanola (PRE), Pyrenean Tarpan, Qatgani, Qatgani/Qataghani Horse, Quarab, Quarter Horse, Quarter Horse Cross, Quarter Pony, Racking Horse, Rheinland Pfalz Saar, Rhenish Warmblood, Rhinelander horse, Rocky Mountain, Rocky Mountain Horse, Russian Don, Russian Heavy Draft, Russian Trakhener, Russian Trotter, Sanhe, Sanhe Horse, Schleswiger Heavy Draft, Scharzwälder Fuchs, Selle Francais, Shagya, Shagya Arabian, Shetland Pony, Shire, Single-Footing Horse, Skyros Pony, Somali Pony, Sorraia, Soviet Heavy Draft, Spanish Mustang, Spanish-Barb, Spanish-Norman, Spanish-Normal Horse, Sport Horse of Color, Spotted Draft, Spotted Draft Horse, Spotted Saddle, Spotted Saddle Horse, Standardbred, Sudan Country-Bred, Sudan Country-Bred Horse, Suffolk, Suffolk/Suffolk Punch/Suffolk Sorrel, Swedish Warmblood, Taishu/Tsushima, Taishuh, Tarpan, Tawleed, Tennessee Walker, Tennessee Walking Horse/Tennessee Walker, Tersk, Tersk Horse, Thessalian, Thessalian/Thessaly Horse, Thoroughbred, Thoroughbred Cross, Tibetan Pony, Tiger horse, Tokara, Tori, Tori Horse, Trakehner, Ukrainian Riding Horse/Ukrainian Saddle Horse, Ukrainian Saddle, Virginia Highlander, Vlaamperd, Vladimir Heavy Draft, Vyatka, Vyatka Horse, Walkaloosa, Warmblood, Warmblood Cross, Warmblood (All), Warmbloods of Color, Welara Pony, Welsh Cob, Welsh Pony, Weser-Em Pony, West African Barb, Western Sudan Pony, Westphalian, Wielkopolski, Wurttemberger Warmblood, Xilingol, Yakut, Yanqi, Yili, Yonaguni, Zangersheide, Zaniskari Pony, Zhemaichu, and Zorse.

The user may further select the discipline of one or more of the parents of the non-human offspring animal. Non-limiting examples of discipline include barrel racing, beginner/family, breeding, brood mare, calf roping, companion only, competitive trail competitions, country pleasure, cowboy mounted shooting, cutting, draft, dressage, drill team, driving, endurance riding, English pleasure, equitation, eventing, field hunter, gaited, halter, harness, horsemanship, hunter, hunter under saddle, judged pleasure rides, jumper, lesson horse, longe-line, pleasure driving, pole bending, polo, racing, ranch horse, ranch sorting, reined cow horse, reining, rodeo, roping, showmanship, saddle seat, sidesaddle, steer wrestling, team penning, team roping, team sorting, trail horse, vaulting, Western pleasure, Western pleasure (show), Western riding, working cattle, youth/4-H horse, all around, 4-in hand driving, agility, breed shows, breeding stallion, broodmare, bull fighting, camping, cart, combined driving, cowboy dressage, cowboy racing, doma vaquero, field trials, gymkhana, harness racing, hazing, horse ball, marathon driving, mounted archery, mounted games, mounted patrol, mounted police, native costume, natural horsemanship, nurse mare, pacing, pack horse, parades, park, playday, polocrosse, ranch versatility, recipient broodmare, roadster, rodeo bronc, rodeo pickup horse, rodeo queen horse, roping, saddleseat, search and rescue, show hack, ski joring, steer roping, stock horse, sulky racing, team driving, team roping head horse, team roping heel horse, therapeutic riding, trial class competition, recreational trail riding, turnback horse, Western dressage, working equitation, hunt seat, show jumper, riding, pleasure, and bucking horse, among others. Other breeds, disciplines, and height traits not disclosed herein may be incorporated into the database for selection by a user.

Moreover, a user may employ the user interface of the disclosure to filter through animals of interest on the basis of specific parameters pertaining to ancestry and genetic relatedness. As is described herein, the user may rely on one or more of shared cM, kinship coefficient, heterozygosity score, inbreeding coefficient (F), breed composition, and identity-by-descent metrics as inclusion or exclusion criteria for the selection of non-animals having a desired degree of inbreeding, genetic relatedness, or a specific breed composition.

B. Selection of One or More Predetermined Traits from Trending Presets

As an alternative to selecting among a catalog of multiple distinct traits in order to produce a non-human animal having a desired phenotype (i.e., a custom build), the user may instead select an animal having a predetermined set of traits from a catalog of trending presets that are frequently queried by existing users of the platform of the present disclosure. The trending presets may include animals having one or more predetermined traits (e.g., breed, speed, discipline, gait, temperament, health, and the gene pool from which an animal is obtained) commonly sought out by users of the platform. Exemplary presets may include “Most popular searches for June 2019,” “What are the locals searching for,” “Tobiano is trending now,” “Child friendly,” “Breeding favorites,” “Buy them all,” “Champagne,” “5 Star Show Jumpers,” and “Clean Testing Sale Horses.”

C. Gene Pool Selection

The present disclosure allows the user to select the gene pool from which a non-human animal or its parents are obtained (produced, purchased, or procured). As used herein, the term gene pool corresponds to the physical source from which an animal or its parents are obtained, such as the user's own animals, (e.g., “My Horses” and “My Farms” gene pools) and/or other users' animals (e.g., “My Favorites, “Exclusions,” “Location Radius,” “Available for Lease,” “Available for Sale,” “Open to Offers,” “Stallion Standing,” “All Public Profiles,” “Registries.” The “My Horses” gene pool corresponds to animals added by the user onto the user platform, which may be viewed and modified by the user and viewed other users. “My Farms” gene pool represents animals that presently reside on one or more of the user's farms (e.g., an animal farm or a ranch). “My Favorites” gene pool includes animals found on the user platform of the present disclosure that have been designated as favorites by the user. Relatedly, an “Exclusions” gene pool may be included in the user interface of the disclosure as a selection criterion for a desired non-human animal. For example, a user may select identified horses to be categorized under the “Exclusions” group to exclude the selected animals from being identified as an animal having the one or more pre-determined traits. The animals designated as favorites may be the user's own animals or animals owned by other users. The “Location Radius” gene pool corresponds to a gene pool defined by a distance threshold relative to the user's location, which may be custom set by the user. For example, the user may select the selection radius to be no more than 50 km, 500 km, or more than 500 km away from the current location of the user. “Available for Lease” gene pool corresponds to animals owned by other users that are available for lease under terms and conditions set by the animal owner. Relatedly, the “Available for Sale” gene pool corresponds to animals owned by other users that are available for sale under terms and conditions set by the animal owner. The “Open to Offers” gene pool corresponds to animals that an owner, trainer, or veterinarian may be willing to sell, trade, lease, or otherwise consider allowing another interested party to acquire or use the animal. The “Stallion Standing” gene pool corresponds to male animals that are available or offered to breed with other animals from other farms, programs, organizations, registries, or within their own breeding program. “All Public Profiles” gene pool represents all animals that have searchable profiles on the platform of the instant disclosure. “Registries” gene pool corresponds to animals available through a registry (e.g., a horse registry).

The user interface allows the user to select more one or more gene pool filter to refine the search results. For example, the user may desire to view all animals available for sale within a given radius with respect to the user's location. To this end, the user may select the “Available for Sale” and “Location Radius” gene pool filters, wherein the user may specify the desired radius (e.g., miles or kilometers) within which the user may purchase an animal having the desired traits.

A. Ancestry Report

The computer-implemented user interface described herein may also be used to produce an Ancestry Report for one or more non-human animals of interest, as is described herein. The Ancestry Report provides information regarding the breed composition of a particular animal(s) that is based on principal components analysis (PCA) or ADMIXTURE method described in Alexander et al., Genome Res. 19:1655-64 (2009). The user may submit a biological sample obtained from the animal using the user interface system of the disclosure (see FIG. 7), which is subsequently processed to perform various analyses described herein in order to ascertain the breed composition and genetic profile of the animal. Non-limiting examples of analyses include PCA- and ADMIXTURE-based breed decomposition, heterozygosity analysis, inbreeding analysis, and “more like me” analysis, as is described in detail herein. Once the ancestry analysis is completed, the information generated by this analysis can be incorporated into the computer-implemented database system of the disclosure to further characterize a particular non-human animal or the information from the analysis can be used to inform purchasing and breeding decisions.

E. “Most Like Me” Function

The present disclosure provides a computer-implemented user interface that can allow a user to identify or produce non-human animals having features similar to an existing non-human animal having a known and defined genotype and phenotype. As is described herein, the user may search the database for animals having a similar or identical profile to a known animal by providing a biological sample obtained from the known animal and using the “Most like me” function in the interface to find one or more matches having similar phenotypic and/or genotypic characteristics. Alternatively, the user may specify a custom set of characteristics in the desired animal that match the known animal, according to the methods described herein, to identify or produce an animal with the desired phenotype.

### Interface Output—Breeding Cross-Match Evaluation and Potential Outcomes

Following the selection of one or more predetermined traits of a non-human animal (e.g., a horse), the user interface of the disclosure provides a visual output of results that may match the query selected by the user. The results may include, for example, an animal available for sale or lease that matches the phenotype set specified by the user or a breeding cross-match evaluation that provides information about a potential breeding pair match (e.g., a stallion and a mare) capable of producing a non-human offspring animal having one or more predetermined traits with a high probability (e.g., 6.25%, 12.5%, 25%, 50%, 60%, 70%, 80%, 90%, 95%, 99%, or 100%). The breeding cross-match evaluation may include a probability matrix that includes an array of probability values pertaining to the likelihood that a breeding pair produces an offspring animal having one or more predetermined traits. The breeding cross-match evaluation may further provide detailed information about the potential breeding pair match such as, for example, the names, photographs, and location of the stallion and mare as well as means for accessing the animal profile of each stallion and mare, information pertaining to one or more predetermined traits in each of the stallion and mare (e.g., speed, temperament, gait, and genotype), means of contacting the owner of each stallion and mare, as well as information about one or more traits of each stallion and mare.

The breeding cross-match evaluation further provides a summary report pertaining to the non-human animal that may be produced from the potential breeding pair (e.g., a foal). The summary report may include several different categories of information pertaining to the non-human animal having the desired characteristics set by the user. For example, the summary report may include information about animal (e.g., foal) probabilities, which include but are not limited to a quantitative readout of a probability of producing a particular coat color (e.g., 100% probability of having bay color; 25% of having overo color; and 25% of having sabino color), probability of having or developing a lethal genetic condition (e.g., 25% of having a lethal frame overo genotype), and a probability of having or developing a genetic disorder, such as any one of the genetic disorders listed in Table 10, among others (e.g., 50% of having or developing impaired acrosomal reaction; 50% chance of having or developing lordosis, and 50% chance of having or developing polysaccharide storage myopathy type 1). The summary report may further include information pertaining to one or more behavioral characteristics of the animal, such as, for example, speed (e.g., sprint/endurance), temperament (e.g., curiosity/vigilance), and gait (e.g., gaited/non-gaited). The summary report pertaining to one or more behavioral characteristics may be provided as a visual readout represented on an ordinal scale (e.g., sprint represented on the left end of the scale and endurance represented on the right end of the scale, with intermediate points on the scale corresponding to animals with speed characteristics somewhere between sprint and endurance).

The summary report may be provided in full or abbreviated form. The abbreviated form may contain information regarding the probability of the offspring animal having a selected speed, temperament, gait, color, disease or condition, and information about the potential breeding pair capable of producing the offspring animal such as names, ages, and images of the potential breeding pair. The full report may include information about the speed, temperament, gait, and genotype of the breeding pair, probabilities of the offspring animal having specific coat color speed, temperament, and gait, as well as probabilities that specific diseases or conditions could afflict the offspring animal. The full report may also provide a graphical representation of a probability matrix containing an array of probability values pertaining to the likelihood of the offspring animal having one or more predetermined traits.

The output generated by the database system related to non-human animals having a desired phenotype (e.g., Find-A-Horse) or potential breeding pairs capable of producing a non-human animal having the desired phenotype (e.g., Build-A-Horse) may be rank ordered on the basis of several factors, including but not limited to: (1) the magnitude of the probability of the target animal having a desirable trait (e.g., a breeding pair capable of producing a foal having a higher probability of a desired sprint phenotype, such as a CT mid-distance myostatin variant, may be ranked higher in the output results as compared to those breeding pairs/foals that would produce a lower probability of having the phenotype); (2) the magnitude of a probability or the binary presence of an adverse health condition that results from the selection of a desired trait set may be ranked lower as compared to trait sets that produce a lower or absent risk of adverse health conditions (e.g., selection of traits that produces Lethal White Overo or Fragile Foal Syndrome would reduce the rank of a particular breeding pair match); and (3) user's preference for a particular trait over another (e.g., user preference for dunskin coat color over bay coat color). Thus, the rank ordering of the output matches is determined by a combination of priorities set by the user (e.g., one or more predetermined traits) and risk(s) of producing adverse health consequences. This rank ordering of the output matches set by the database system may be overridden or suspended by the user in the case that the user prioritizes a phenotype set that is capable of producing a potentially deleterious or fatal phenotype. For example, the database system may produce an output match that identifies a breeding pair in which both sire and dam carry a risk allele for Fragile Foal Syndrome. The database system, in this case, would issue a warning indicating a high risk of the resulting offspring animal carrying the disease. Nevertheless, the user may still prioritize this match and ignore the high risk of the disease if the other traits possessed by the breeding pair are highly desirable.

### Diagnostic Testing

The computer-implemented user interface disclosed herein allows for a user to order clinical/veterinary tests (e.g., genetic testing pertaining to pedigree determination, health status, and predispositions towards genetic conditions) to be performed on a biological sample obtained from one or more animals owned by the user (e.g., a horse). The diagnostic testing may also be used to ascertain the ancestries of an animal of interest, e.g., by producing a report containing information on one or more (or all) of the shared cM metric, kinship coefficient, heterozygosity score, inbreeding coefficient, identity-by-descent, or breed composition metrics related to the animal, as is described herein. The user may order a test for a given animal by providing information such as the name of the horse, the type of test desired to be performed, contact information of the user (e.g., telephone number), payment information (e.g., credit card information), as well as any relevant gift card or coupon code information (see FIG. 1R). The user may input personal information to the platform such as, for example, salutation, full name, e-mail (and visibility thereof to other users), telephone number (and visibility thereof to other users), date of birth, user photograph (and visibility thereof to other users), and address (and visibility thereof to other users). The user interface of the disclosure also allows the user to update user settings, such as changing the user password.

### User, Animal, and Farm Profiles

A. User Profile

The computer-implemented user interface of the present disclosure allows the user to create a personal profile page which may be viewed and modified by the user and may be viewed by other users of the platform. The user profile may include information about the user including name, address or general location (e.g., city, state, country), and a profile picture, list of animals (e.g., horses) owned by the user as well as visual displays of physical or behavioral trait metrics of the animal, a “Favorites” list including animals designated as Favorites on the user platform, means for adding animals of the user into a “Favorites” list, means for contacting the user (e.g., by e-mail), means for obtaining a submission form related to genetic testing of an animal, and a means for downloading test results (e.g., genetic tests) pertaining to one or more traits of the animal (see, e.g., FIG. 1Q).

B. Non-Human Animal Profile

The computer-implemented user interface disclosed herein allows the user to create and to modify a profile page of a non-human animal. The animal profile may include information about the animal including name, breed, age, profile pictures, physical traits including height, colors, and markings, behavioral traits including rideability and temperament, discipline, genetic information about the animal (e.g., presence of particular gene variants in the animal), information pertaining to membership in an animal registry, owner information (e.g., name, address, profile picture), information pertaining to other horses owned by the same owner (e.g., name, breed, age, and picture), order status information pertaining to tests performed on tissue obtained from the animal (e.g., order identification number, date of order, status such as ready or pending), means for downloading test results (e.g., genetic test results) onto the user's device, and means for editing the animal profile (see, e.g., FIG. 1L). The aforementioned list is not exhaustive, and other information and features may be included in the animal profile page.

C. Farm Profile

The computer-implemented user interface disclosed herein allows the user to create and modify a profile page of a farm (e.g., an animal farm or ranch). The farm profile may include information about the farm including name, farm type (e.g., horse farm), farm location, farm profile picture or logo, information pertaining to farm members (e.g., member name, picture, number of horses owned), information pertaining to animals situated at the farm (e.g., name, breed, age, picture, and visual displays of physical or behavioral trait metrics of the animal), means for contacting the farm (e.g., by e-mail). These features and information are not exhaustive and other options may for the farm profile page may be included.

### Hardware Implementation

The user interface described herein can be implemented in digital electronic circuitry, or in computer hardware, firmware, software, or in combinations of them. Apparatus of the disclosure can be implemented in a computer program product tangibly embodied in an information carrier, e.g., in a machine-readable storage device or in a propagated signal, for execution by a programmable processor; and method steps of the disclosure can be performed by a programmable processor executing a program of instructions to perform functions of the disclosure by operating on input data and generating output. The disclosure can be implemented in one or more computer programs that are executable on a programmable system including at least one programmable processor coupled to receive data and instructions from, and to transmit data and instructions to, a data storage system (e.g., a database), at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the disclosure can be implemented on a computer having a display device such as a CRT (cathode ray tube), LCD (liquid crystal display), or LED (light-emitting diode) monitor for displaying information to the user and a keyboard and a pointing device such as a mouse or a trackball by which the user can provide input to the computer.

The disclosure can be implemented in a computer system that includes a back-end component, such as a data server, or that includes a middleware component, such as an application server or an Internet server, or that preferably includes a front-end component, such as a client computer having a user interface (e.g., such as the user interface of the present disclosure) and/or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network. The relationship of client and server arises by virtue of computer programs running on the respective computers and having a client-server relationship to each other. The computer system may further include portable communications devices such as a mobile telephone that also contains functions such as an Internet browser. Other portable electronic devices, such as laptops or tablet computers may also be used with the present disclosure. It should also be understood that the computer system is not a portable communications device, but may be, e.g., a desktop computer.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1: Use of a Computer-Implemented Graphical User Interface for Producing, Procuring, and/or Identifying a Non-Human Animal Having One or More Predetermined Traits

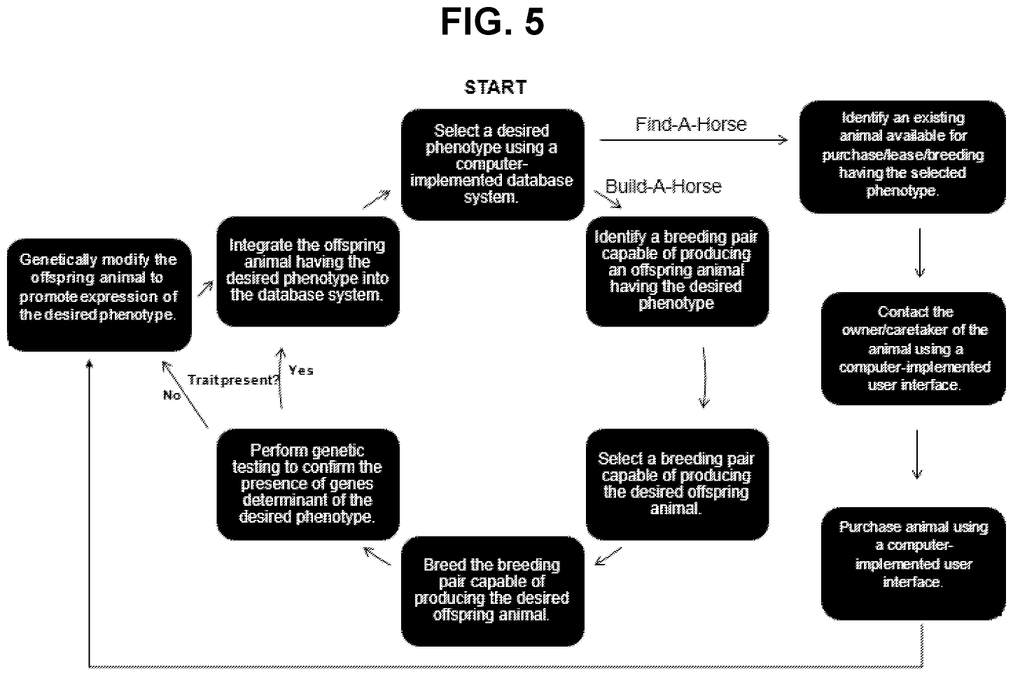

In order to produce a non-human animal (e.g., a horse, such as, e.g., a foal) having one or more predetermined traits, a computer-implemented user interface is provided to assist a user in selecting traits that are desired in the horse, identifying an existing horse having said pre-determined traits or a breeding pair capable of producing said horse, and producing or procuring said horse. As shown in FIGS. 1A-1U, the user interface can be implemented as a webpage-instantiated graphical user interface on a general purpose computer or hand-held device, allowing a user to create a unique and custom build for a foal or identifying an existing horse having the desired build by selecting the gene pool from which the foal or the breeding pair is obtained, the traits desired in the foal or breeding pair (e.g., speed, temperament, gait, color, color modifier, health; FIGS. 1E, 1F, 1H-1J, and FIG. 5), an option to buy or lease the foal or one or more animals of a breeding pair (FIG. 1D).

The user interface further allows a user to view profiles of existing horses having a phenotype selected by the user or a horse that can be used as a breeder to produce the foal having a predetermined phenotype. The horse profile provides information pertaining to the horses name, age, breed, sex, genotype, temperament, gait, speed, height, discipline, color, markings, parents, owner, location, as well as other horses owned by the owner (FIG. 1L). Furthermore, the user interface allows a user to view one or more breeding pairs capable of producing a foal having a desired phenotype. The list of breeding pairs also includes a summary of a probability matrix that displays an array of probability values pertaining to the likelihood that the foal will have one or more desired traits (FIG. 1M). Once a user has identified a preferred breeding pair, the user can view information about the breeding pair in more detail, including information about the speed, temperament, gait, genotype, appearance, name, location, and owner of each of the horses in the breeding pair (FIG. 1N). Upon selecting a preferred breeding pair, the user can then view a detailed summary report about a potential foal that could be produced by breeding the breeding pair. The summary report includes a graphical representation of a probability matrix describing the probabilities of the potential foal having one or more desired traits, such as, e.g., a particular coat color, speed, temperament, gait, and/or health issues (FIG. 1O). The user interface also permits the user to identify popular builds queried by other users of the database system based on frequency of searches (FIG. 1P).

The disclosed user interface further allows a user to create their own user profile (FIG. 1Q), including information about the user, such as, e.g., name, photo, username, location, contact information, and animals owned by the user. The user may also employ the interface to order genetic diagnostic tests for one or more animals owned or leased by the user by providing a sample (e.g., a tissue sample obtained by hair sample or biopsy, blood, serum, plasma, urine, sputum, nail/hoof clippings, cell-free fetal DNA, mitochondria, a gamete (e.g., oocyte or spermatozoon), or an embryo of the offspring animal implanted as part of an in vitro fertilization (IVF) procedure) from the animal by mail (FIG. 1R). The user may then keep track of ordered and performed tests by viewing their status and results on the user profile (FIG. 1S). An example of a Sample Submission Form for genetic testing is provided in FIG. 1T. The results of the genetic testing produced using the database system of the disclosure are described in FIG. 1U.

### Example 2: Use of a Computer-Implemented Database System for Producing a Non-Human Offspring Animal Having One or More Predetermined Traits

Using the systems and methods disclosed herein, a user (e.g., animal breeder, caretaker, owner, or seller) can interrogate the disclosed computer-implemented database system to produce a non-human offspring animal (horse, cow, sheep, dog, cat, camel, pig, goat, alpaca, donkey, llama, red fox, mouse, rat, ferret, non-human primate, rabbit, gerbil, hamster, chinchilla, or guinea pig) having one or more (e.g., 1, 2, 3, 4, 5, 6, 7, 8, 9, 10, or more) predetermined traits. A computer-implemented graphical user interface (e.g., as is described in Example 1) is used to query and interrogate the database system. As a first step, the user selects one or more predetermined traits (e.g., coat color, coat color modifier, coat texture, coat thickness, facial marking, leg marking, eye color, skin color, mane color, tail color, speed, gait, temperament, or health) that are desired in the offspring animal by way of the user interface. The interface queries the database system to allow the user to identify one or more (1, 2, 3, 4, 5, 6, 7, 8, 9, 10, 12, 14, 16, 18, 20, or more) breeding pairs (e.g., a stallion and a mare) capable of producing the offspring animal. For each identified breeding pair, the database system generates a probability matrix containing an array of probability values pertaining to the likelihood that the offspring animal produced by each of the breeding pairs will have the one or more predetermined traits. As a next step, the user selects, based on the array of probability values of the probability matrix, one or more of the breeding pairs that are likely to produce the offspring animal having the one or more predetermined traits. Subsequent to the selection of one or more candidate breeding pairs, the user can elect to breed one or more of the selected breeding pairs to produce the offspring animal having one or more predetermined traits. In order to detect the presence of the one or more predetermined traits, the user can perform genetic diagnostic testing on the offspring animal during prenatal (e.g., embryonic) development using methods well-known in the art (e.g., Sanger sequencing, Next Generation Sequencing, microarray, AmpliSeq, genomic sequencing, or real-time quantitative polymerase chain reaction (RT-qPCR)) on a sample obtained from the offspring animal (e.g., blood, tissue, hair, cell-free fetal DNA, mitochondria, or a gamete). In the case of a horse, the prenatal genetic testing can be performed during embryonic development (e.g., 3-60 days after conception). In the case that the genetic testing detects the presence of the one or more predetermined traits, the user may continue with the birthing of the offspring animal. Subsequent to the birthing of the offspring animal (e.g., one day to four years after birth), the user can again perform further genetic testing, if desired, to ascertain the presence of the one or more predetermined traits. The user may further confirm the expression of the one or more traits in the offspring animal using non-genetic assessment, such as, e.g., observing the phenotypic expression of the one or more traits; obtaining or having obtained information from someone knowledgeable about the offspring animal pertaining to the one or more predetermined traits; performing or having performed one or more clinical assays on biological tissue obtained from the offspring animal pertaining to the one or more predetermined traits; performing or having performed one or more diagnostic tests on the offspring animal pertaining to the one or more predetermined traits; and/or obtaining scores assigned to the offspring animal during a competition pertaining to the one or more predetermined traits. Upon confirmation of the presence of the one or more predetermined traits in the offspring animal, the user may choose to incorporate information about the offspring animal having the one or more predetermined traits into the database system for use by the user, or a subsequent user, in the production of another offspring animal.

### Example 3: Generation of a Computer-Implemented Database System for Producing, Procuring, or Identifying a Non-Human Animal Having One or More Predetermined Traits

Using the systems and methods disclosed herein, a user can produce or improve the robustness of a computer-implemented database system by integrating information about one or more predetermined traits from a plurality (e.g., 2, 3, 4, 5, 10, 15, 20, 30, 40, 50, or more) of non-human animals. The provided information may include results of previously performed genetic testing and/or non-genetic assessment of the non-human animal that identify the animal as having the one or more predetermined traits and/or confirm the presence of one or more of the predetermined traits in the animal. The user may catalogue the information about the non-human animal pertaining to the one or more predetermined traits into the database system. The user may register the non-human animal in the database system as a breeder animal. Once the non-human animal is integrated into the database system, a user can interrogate the database system to produce a probability matrix containing an array of probability values pertaining to the likelihood of one or more predetermined traits being expressed in a parent or offspring animal, which may include results associated with the newly input breeding animal as a “parental” animal. In particular, the probability matrix may be produced by a series of conditional genotypic or phenotypic associations that may confer a high or low likelihood of producing the one or more predetermined traits. Such associations are stored within the database system and updated as new associations become known. For example, in a situation in which a breeder desires a horse with blue eyes and white facial markings, the database system of the disclosure may identify an association between blue eyes and facial markings with deafness, thereby producing a probability matrix showing a high probability (e.g., 6.25%, 12.5%, 25%, 50%, 60%, 70%, 80%, 90%, 95%, 99%, or 100%) of the horse having a hearing impairment along with the other two traits. Such an association has been shown, e.g., in horses harboring a heterozygous 63-kb deletion spanning exons 6-9 of the MITF gene (Henkel et al. Animal Genetics 50(2):172-4, 2019). Other associations can be input into the database system and would be identified in the probability matrix output produced based on the input criteria selected by a user.

### Example 4: Use of a Computer-Implemented Database System to Produce a Set of Guidelines for Breeding of a Non-Human Animal Having One or More Predetermined Traits

Using the systems and methods disclosed herein, a user can obtain a set of guidelines for breeding a non-human animal from the computer-implemented database system. First, the user can query the database system to identify a non-human animal and assess the animal for the presence of one or more predetermined traits. The identification and assessment steps can be performed by generating a probability matrix for the non-human animal using the database system and displaying the probability matrix on a graphical user interface in a user-friendly format. The probability matrix can contain an array of probability values pertaining to the likelihood that the non-human animal has the one or more predetermined traits. The user may then determine, based on the identification and assessment steps above, one or more conditions under which the non-human animal will develop or is at risk of developing a disease or non-disease condition (e.g., one or more disease or non-disease conditions listed in Table 10). Following the risk determination step, the database system may generate for the user a set of guidelines for breeding the non-human animal to avoid manifestation of the disease or non-disease conditions. The set of guidelines may include, e.g., one or more recommendations for mitigating or avoiding the one or more conditions that would result in manifestation of one or more symptoms of the disease or non-disease condition and/or reducing the likelihood of producing an effect in the offspring animal resulting from development of the disease or non-disease condition. Additionally, when the non-human animal is a male or female parent that is part of a breeding pair used for producing an offspring animal, the set of guidelines can provide one or more recommendations that would increase the likelihood of producing an offspring animal having one or more predetermined traits without manifestation of an undesired phenotype or disease condition. The one or more recommendations may include, e.g., guidance pertaining to diet, exercise, regime, discipline, environmental exposure, medication, supplementary treatments, training, conditioning, use, and/or limitation of the non-human animal.

For example, as is discussed above, horses harboring genetic deletions in the MITF gene locus are known to exhibit striking blue eyes and coat depigmentation (Henkel et al. Animal Genetics 50(2):172-4, 2019). This mutation is also accompanied by deafness. Therefore, upon learning from the probability matrix described above that the desired blue eyes and coat depigmentation carries a high risk of the horse being hearing impaired, the database system may produce a set of guidelines that can be used by the user for training or acclimating a hearing-impaired animal.

In another example, a horse produced by a user using the methods and systems disclosed herein may have exceptional jumping ability at maturity. The database system of the disclosure may then be used to produce a set of guidelines that can assist the user in the care of an animal selected to be an excellent jumper, such as, e.g., recommendations to feed a low-protein diet to avoid excessive growth and injections of Adequan joint supplement to ensure proper joint lubrication and preparations.

In another example, a female parent animal may be identified as a potential breeder animal using the disclosed methods and systems to produce a foal having a desirable variant of a bay coat color (e.g., light bay versus dark bay color). It is well-known in the art of animal breeding that specific nutrients are known to be involved in the synthesis of the protein found in hair (e.g., copper, zinc, biotin, fatty acids, and amino acids such as, e.g., methionine). Copper and zinc are known to be essential in the production of melanocytes (i.e., pigment cells) that confer specific coat colors in horses and other animals. Relatedly, high fat diets are known to lubricate the hair of the coat, thereby rendering its appearance as shiny and dark. Thus, if the user prefers to produce an offspring animal having a darker bay coat, the database system of the disclosure can provide a recommendation to the user to provide a high-fat diet to a female parent animal during prenatal development of the offspring animal. Relatedly, if the user prefers to produce an offspring animal having a red(-ish) bay coat, the database system of the disclosure can provide a recommendation to the user to provide the female parent a diet rich in oats, which are known to confer a red coloration to a bay coat in offspring animals.

In another example, a breeder may use the database system of the disclosure to select one or more desired traits that, in combination, dramatically increase the likelihood that a horse will have kissing spine, a condition characterized by positioning of two or more spinous processes (e.g., flanges of bone sticking up from the vertebra of the spine) in a way that they rub against each other, often producing back pain, bone cysts, arthritic changes, and other problems in affected horses. Thus, the database system of the disclosure can generate a set of guidelines for the breeder pertaining to the husbandry of a horse with kissing spines, including, e.g., avoiding training regimens that require jumping.

In an additional example, a breeder may use the database system of the disclosure to select one or more desired traits that, in combination, increase the susceptibility of a horse to, e.g., infection by the West Nile virus. Accordingly, the database system of the disclosure can generate a set of guidelines for the breeder that recommend timely vaccination of the susceptible horse.

In another example, a breeder may use the database system of the disclosure to select one or more desired traits that, in combination, increase the susceptibility of a horse to, e.g., Polysaccharide Storage Myopathy (PSSM). Accordingly, the database system of the disclosure can generate a set of guidelines for the breeder to provide a specific feed required to avoid the impending symptoms of PSSM, such as “tie-up,” muscle cramps, lameness, or pain. Furthermore, the guidelines may instruct to provide a horse with PSSM with regular exercise and low-sugar diets to avoid disease symptoms.

Additionally, a breeder may use the database system of the disclosure to select one or more desired traits that, in combination, increase the susceptibility of a horse to, e.g., equine asthma or other respiratory irritations (e.g., “bleeders”). Accordingly, the database system of the disclosure can generate a set of guidelines for the breeder to steam or pre-treat any grass, hay, or plant-based feeds that may contain allergenic particulates, airway irritants, or dust to reduce inflammation in the horse.

In yet another example, a breeder may use the database system of the disclosure to select one or more desired traits that, in combination, increase the susceptibility of a horse to, e.g., great size (e.g., “tall” type variants). Accordingly, the database system of the disclosure can generate a set of guidelines for the breeder to feed the horse a low-protein diet or other veterinarian-recommended or -supervised diets that mitigate accelerated growth and the known medical sequalae associated with such growth, such as, e.g., joint problems or Equine Osteochondritis Dissecans (OCD).

### Example 5: Use of a Computer-Implemented Database System to Produce a Genetically-Modified Non-Human Offspring Animal Having One or More Predetermined Traits

Using the systems and methods disclosed herein, a user (e.g., animal breeder, caretaker, owner, or seller) can interrogate the disclosed computer-implemented database system to produce a non-human offspring animal having one or more (e.g., 1, 2, 3, 4, 5, 6, 7, 8, 9, 10, or more) predetermined traits. A computer-implemented graphical user interface is used to query and interrogate the database system. As a first step, the user selects one or more predetermined traits that are desired in the offspring animal by way of the user interface, wherein the interface queries the database system to allow the user to identify one or more breeding pairs capable of producing the offspring animal.

In some cases, the database system of the disclosure may unsuccessfully identify an existing breeding pair capable of producing an offspring animal having one or more predetermined traits. To remedy this scenario, a user may genetically modify one or more (1, 2, 3, 4, or more) non-human animals of a breeding a pair to increase (e.g., by 5%, 10%, 15%, 20%, 30%, 40%, 50%, 60%, 70%, 80%, 90%, 100% or more) the likelihood of producing an offspring animal having the one or more predetermined traits using methods well-known in the art (e.g., transcription activator-like effector nuclease (TALEN), zinc finger nuclease (ZFN), or clustered regularly interspaced short palindromic repeats (CRISPR)). The user may interrogate the database system of the disclosure to identify one or more genotype-phenotype correlations to produce a genetic modification that would promote the expression of the desired phenotype in the parent and/or the offspring animal. The genotype-phenotype correlation may include a monogenic correlation (i.e., single gene controls the expression of a single phenotype) or a polygenic correlation (e.g., two or more genes control the expression of a single phenotype). This genotype-phenotype correlation may be used to determine which specific genes should be genetically modified in the one or more parent animals to increase the likelihood of producing an offspring animal having one or more predetermined traits. The user may further interrogate the database system of the disclosure to identify any deleterious or desirable genotype-genotype and/or phenotype-phenotype correlations to guide decision-making about trait selection prior to the genetic modification steps. Genotype-genotype correlations may be useful to apprise the user about any genetic associations between expression of a particular gene and a potentially deleterious genetic polymorphism or mutation in a region of the genome within or adjacent to the gene of interest. Phenotype-phenotype correlations may be particularly useful to a user by informing about a potential association between two or more phenotypes in a non-human animal. For example, if the user learns from the database system of the present disclosure that a particular desired phenotype may be associated with another undesired phenotype, the user may use this information to guide their decision about how to breed parental animals to obtain the desired offspring animal or about how to produce the offspring animal using genetic manipulation.

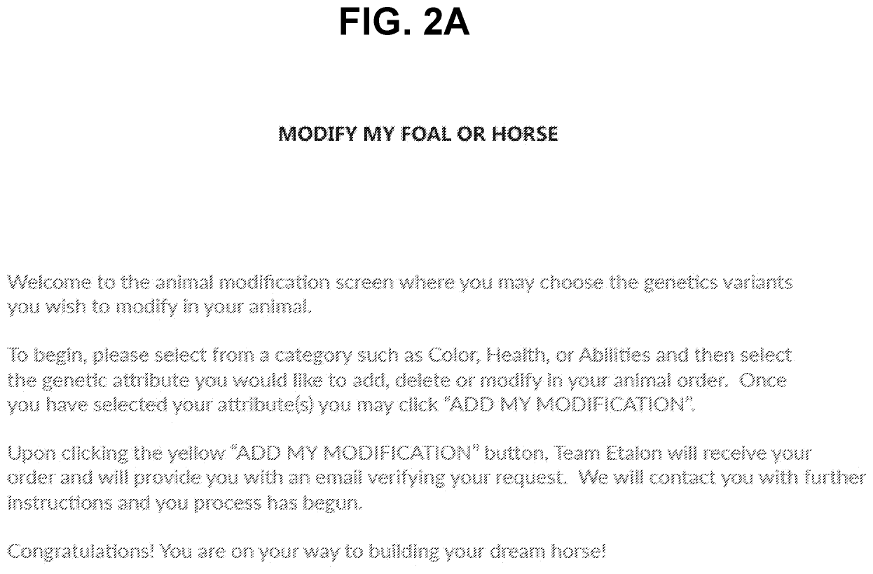

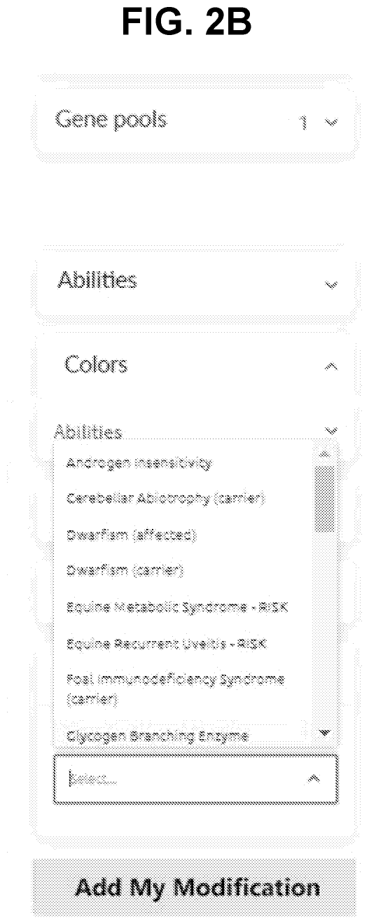

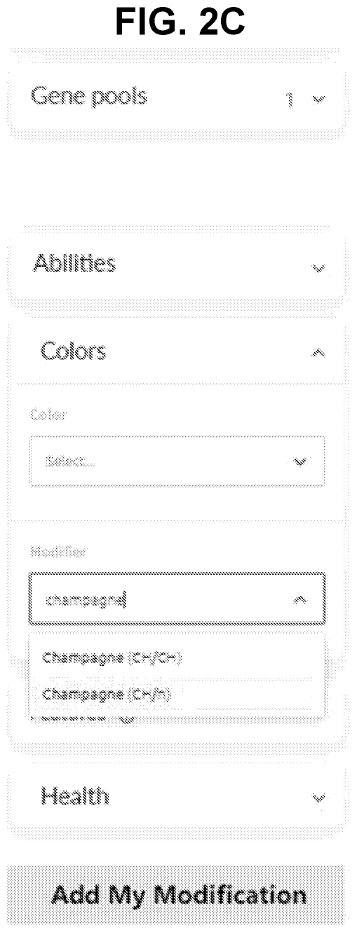

In other cases, the user may be unsuccessful in producing an offspring animal having one or more predetermined traits after breeding a breeding pair identified by the database system of the disclosure. In such instances, a user may decide to genetically modify the offspring animal to increase the likelihood that the offspring animal will have the one or more predetermined traits. For example, the user may use the database system of the disclosure to produce an offspring animal (e.g., according to the methods described in Example 1 above; see FIGS. 2A-2C). Genetic testing may be performed on a sample obtained from the animal during prenatal (e.g., embryonic) development (e.g., 3-60 days after conception) or after birth (e.g., one day to four years after birth) to determine the presence of the one or more predetermined traits. Non-genetic assessment may also be performed on the animal after birth (e.g., one day to four years after birth) to assist in the determination of the presence of the one or more predetermined traits in the offspring animal. If the genetic assessment during prenatal development or the genetic and/or the non-genetic assessment after birth indicates that the offspring lacks the one or more predetermine traits, the user may genetically modify the offspring animal to promote the expression of the one or more predetermined traits.

Additional cases in which genetic modification of a non-human offspring animal may be required include cases in which some, but not all desired traits are expressed in an offspring animal produced using the disclosed methods and systems. For example, in the case of a horse, a breeder may want to produce a gaited horse having a bay coat and white facial markings; however, upon producing the offspring animal using the disclosed methods, the breeder discovers (e.g., by way of genetic or non-genetic assessment of the offspring animal) that the offspring animal is gaited and has a bay coat, but lacks the white facial markings. In such a case, the breeder may employ the methods and systems of the disclosure to genetically modify the animal to also produce white facial markings in addition to the bay coat and the gaited phenotype.

Other cases that may require genetic modification of a non-human offspring animals include cases in which a particular trait of interest is partially, but not fully expressed to a level suitable for the breeder. For example, in the case of a horse, a breeder may wish to produce a horse that excels at jumping. After producing the offspring animal selected by the breeder to be a good jumper using the database system of the disclosure, the breeder may discover that the horse is a good jumper, but still underperforms relative to the breeder's expectation. In such a case, the breeder may use the disclosed methods and systems to genetically modify the offspring animal to ensure complete expression of the “excellent jumper” trait.

The genetic modification may be performed on one or more parent animals of a breeding pair or an offspring animal to modify at least one (e.g., at least 1, 2, 3, 4, 5, 6, 7, 8, 9, 10, or more) genes in the animal(s). In the case of an offspring animal, the genetic modification can be performed prenatally (e.g., during embryonic development, such as, e.g., 3-60 days after conception) or postnatally (e.g., after birth, such as, e.g., one day to four years after birth). The genetic modification may include editing of an endogenous gene in the one or more parent or offspring animals, such as, e.g., an endogenous gene having a genetic polymorphism (e.g., a single nucleotide polymorphism (SNP)) or a genetic mutation (e.g., insertion, deletion (e.g., knockout), translocation, inversion, single point mutation, or other mutation). The editing of the endogenous gene may include modifying, deleting, or replacing at least one (e.g., at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or more) nucleotides of a DNA or an RNA encoding the endogenous gene in the parent animal. Alternatively, editing of an endogenous gene may include inserting a donor polynucleotide into a region of the endogenous gene for incorporation of the donor polynucleotide into the endogenous gene locus (e.g., by way of homologous recombination or non-homologous end-joining mechanisms). The donor polynucleotide may include a segment of an endogenous gene that lacks the genetic polymorphism or mutation. As another alternative, editing of an endogenous gene may include introducing a heterologous transgene into the genome of the one or more parent or offspring animals. As a further alternative, editing of an endogenous gene may include modulating (e.g., increasing or decreasing, e.g., by 1%, 5%, 10%, 15%, 20%, 25%, 30%, 45%, 50%, 55%, 60%, 65%, 70%, 75%, 80%, 85%, 90%, 95%, 100%, or more) the expression of an endogenous gene in the one or more parent or offspring animals. Modulation of endogenous gene expression may be performed, e.g., using a nuclease-inactivated CRISPR-Cas9 (dCas9) system containing a transcriptional regulator (e.g., activator or repressor). For increasing expression of an endogenous gene, a dCas9 system coupled to a transcriptional activator (e.g., VP16, VP64, p65, RTA, VPR, SAM, or SunTag) may be used. If the goal is to reduce expression of an endogenous gene, a dCas9 system coupled to a transcriptional repressor (e.g., KRAB) may be employed. Subsequent to the genetic modification, genetic and/or non-genetic assessment of the one or more parent or offspring animals may be performed to confirm the expression of the one or more predetermined traits. Genetic diagnostic testing may be performed using methods well-known in the art (Sanger sequencing, Next Generation Sequencing, microarray, AmpliSeq, genomic sequencing, or RT-qPCR). Non-genetic assessment may be performed by, e.g., observing the phenotypic expression of the one or more traits; obtaining or having obtained information from someone knowledgeable about the one or more parent or offspring animals pertaining to the one or more predetermined traits; performing or having performed one or more clinical assays on biological tissue obtained from the one or more parent or offspring animals pertaining to the one or more predetermined traits; performing or having performed one or more diagnostic tests on the one or more parent or offspring animals pertaining to the one or more predetermined traits; and/or obtaining scores assigned to the one or more parent or offspring animals during a competition pertaining to the one or more predetermined traits

Upon confirmation of the presence of the one or more predetermined traits in the offspring animal, the user may incorporate information about the offspring animal having the one or more predetermined traits into the database system.

### Example 6: Ancestry Determination in Horses Using a Computer-Implemented Database System

A user may interrogate the database of the present disclosure in order to determine or verify the pedigree of a non-human animal. As is discussed herein, horse enthusiasts have traditionally selected for specific traits in their foals based on information relating to the pedigree of the stallion and mare. The breeder optimized the likelihood of achieving the desired phenotype (e.g., one or more predetermined traits) in the foal by selecting a breeding pair capable of producing the desired phenotype. This strategy is encumbered by various sources of error that introduce uncertainty of achieving the target phenotype. For example, breeding decisions based on pedigree have long been made on the basis of self-reported testimony of the horse seller and/or based on an assumption that the foal inherits 50% of its genetic information from each parent, thereby relying on Mendelian genetics to determine the horse's pedigree. There are at least two problems with this approach, namely that (1) pedigree assignments based on presumed genetic inheritance often underestimate the degree of genetic relatedness (i.e., inbreeding) between the stallion and the mare; and (2) small changes in genotype can produce profound changes in phenotype, thus potentially confounding the initial judgment of the breed of the stallion and/or mare.

As an example, an ancestry analysis was performed for a particular horse (named “Cruising”) using the database system of the disclosure. A tissue sample was obtained from Cruising and submitted by a user via the user interface system of the disclosure, requesting genetic testing to be performed on the sample to ascertain the pedigree of the horse. The reference sample set was analyzed using genome-wide single nucleotide polymorphism (SNP) equine 70 K SNP chip (Illumina) using Admixture, PLINK, and Illumina's software. Breeds were sampled globally and the analysis on the 70,000 SNPs for all horses were compared to reference sets in the literature. After filtering for relevance, a subset of the SNPs were determined to be relevant for regional determination and comparison of referenced, known and reported genetic variation by global region. Each subset of SNPs corresponds to a region of reported “breed” or “type” form that global region, creating a new “reference” genetics set against which new horse samples are compared. Such comparison may be used to analyze similarities and/or differences between the subject horse and the reference set. Such analysis can then be used to determine the likelihood of likeness of “breed” or genetic inheritance similarity. Principal component analysis (PCA) was subsequently performed on the results of the genetic tests to determine the presence or absence of an overlap in genetic markers between a plurality of known breeds, groups, or registries of horses. Horses having similar genotypes appear as clusters of the same group color on the PCA plot (FIG. 3D). PCA analysis identified Cruising as being predominately a Thoroughbred (66%) and partly European Heavy Horse (Belgian, Clydesdale, and Franches Montagnes; 20.6%), Carriage Horse (French Trotter, Saddlebred, and Standardbred; 6.1%), Nordic Sea (Icelandic, North Swedish, and Shetland; 3.7%), British Isles Native/Exmoor (Exmoor and Fell Pony; 2.7%), and Iberian (Andalusian, Lusitano, Mangalara Paulista, and Pura Raza Espaniola; 0.7%; FIG. 3A-3D).

In another example, an ancestry analysis was performed for another horse named “Spooks Gotta Whiz” (“Spooks”). A tissue sample was obtained from Spooks and submitted by a user via the user interface system of the disclosure, requesting genetic testing to be performed on the sample to ascertain Spooks's breed composition. In addition to the PCA analysis described above for Cruising, an ADMIXTURE-based breed composition analysis (Alexander et al. Genome Res. 19:1655-64, 2009; incorporated by reference herein) was performed to assess the contribution of various known breeds to Spooks's genetic profile. The PCA-based analysis identified Spooks as being predominately Thoroughbred (34%) and partially Carriage Horse (24.3%), European Heavy Horse (15.5%), Near East (12.3), Iberian (11.3%), Exmoor (1.3%), and North Sea (1.3%) (FIGS. 8A, 8B, and 8E). The ADMIXTURE-based analysis provided a further view into Spooks's breed composition, further showing that the Near East component includes an Arabian breed, the Iberian component includes 8% Puerto Rican Paso and 3% Pura Raza Espanola, the Carriage Horse component includes 14% Saddlebred, 4% Standardbred, and 6% French Trotter, the European Heavy Horse component includes 2% Clydesdale, 9% Franches Montagnes, and 4% Belgian, and the North Sea component includes 1% Norwegian Fjord (FIGS. 8A-8D).

Furthermore, the analysis revealed a Heterozygosity score of 31.5%—this metric is a measure of genetic diversity (e.g., the number of genetic variants within an individual) and generally correlates with overall health of resilience to illness of the animal. The analysis further revealed Spooks as having an inbreeding score (F value) of 6.532%—this measure is derived from the genetic diversity within an individual and is based on information obtained from genetic testing, as is described herein.

These findings indicate that the computer-implemented database system of the disclosure can be used in combination with the user interface system described herein to ascertain ancestral relationships within and between particular animals and is, therefore, useful for animal breeders in identifying or producing animals having an advantageous phenotypic profile. In particular, the ancestry/breed information for a given animal(s) generated using the database system of the disclosure can be iteratively incorporated into the database system to facilitate the selection or production of animals having one or more predetermined traits, to facilitate trait discovery, to identify specific predispositions or health conditions within the animal, and to identify new correlations between genetic and non-genetic traits of the animal.

### Example 7: Parentage Determination in a Horse Using the Database System of the Disclosure

A user may interrogate the database of the present disclosure in order to determine or verify the parentage of a non-human offspring animal (e.g., horse) whose parentage is fully or partially unknown (i.e., both parents are unknown, or a single parent is unknown). For example, results of genetic testing of a non-human animal (e.g., horse) can be analyzed and compared to results of genetic testing of a plurality of horses catalogued in the database system of the disclosure. A variety of genetic markers (e.g., SNPs, such as, e.g., exclusion SNPs or microsatellites) can be used to test across a large number of carefully selected genes (e.g., any one of the genes disclosed herein, among others) to test for parentage between horses. An exclusion SNP can be used to rule out the likelihood that an offspring animal and a candidate parent animal are related. Generally, SNPs can be used to define an exclusion probability, which is the average capability of any genetic marker to exclude a group of unrelated individuals from a family. Typically, an individual is excluded as the parent of an offspring if the offspring's genotype at a particular genetic locus cannot be produced from the genotype of the candidate parent animal based on Mendelian inheritance and genetic mutations.

The database system may employ a comparison engine that assesses the degree of overlap between the genotype of a subject offspring horse whose parentage is fully or partially unknown and a plurality of candidate parent animals. The database system may then produce a probability matrix containing an array of exclusion probability values for one or more candidate parent animals. A low exclusion probability value (e.g., 12% or less, such as, e.g., 12%, 11%, 10%, 9%, 8%, 7%, 6%, 5%, 4%, 3%, 2%, 1%, or less) would indicate a high likelihood that the offspring animal and the candidate parent animal are related. Similarly, the database system may produce the absolute quantities for the number of exclusion SNPs identified in the one or more candidate parent animals (e.g., see FIG. 4A). A low number (e.g., less than 5, such as, e.g., 4, 3, 2, 1, or 0) of exclusion SNPs identified between the subject offspring animal and a candidate parent animal would also indicate a high probability that the two animals are related.

In a particular example, parentage analysis was performed for an offspring animal (“Mystery Foal”) using the database system of the disclosure. A tissue sample was obtained from Mystery foal and submitted for genetic testing using the database system. The genetic profile of Mystery Foal was assessed across a panel of select biomarkers and compared to the genetic profiles of a plurality (98) of horses catalogued in the database system to identify candidate parents. The database system identified a particular horse (“GUAPE: 033920_032”) as a likely parent of Mystery Foal based on the fact that no exclusion SNPs were identified between the two horses, suggesting a high probability of relatedness. Other horses (e.g., TEQUE: 033920_033) were ruled out as a candidate parent based on the presence of a large number (9) of exclusion SNPs observed between the pair (FIG. 4).

### SPECIFIC EMBODIMENTS

Some specific embodiments are listed below. The below enumerated embodiments should not be construed to limit the scope of the invention, rather, the below are presented as some examples of the utility of the invention.
E1. A method of producing a non-human offspring animal having one or more predetermined traits, the method comprising:
   (a) selecting one more predetermined traits desired in the offspring animal using a computer-implemented database system;
   (b) interrogating the database system to identify one or more breeding pairs capable of producing the offspring animal;
   (c) generating a probability matrix for each of the one or more breeding pairs using the database system, wherein the probability matrix comprises an array of probability values pertaining to the likelihood that the offspring animal produced by each said breeding pair will have the one or more predetermined traits;
   (d) selecting, based on the array of probability values, one or more of the breeding pairs likely to produce the offspring animal having the one or more predetermined traits;
   (e) breeding one or more of the selected breeding pairs to produce the offspring animal;
   (f) performing genetic testing of the offspring animal to detect the presence of the one or more predetermined traits; and
   (g) genetically modifying the offspring animal lacking the one or more predetermined traits to promote or improve expression of the one or more predetermined traits.
E2. The method of E1, wherein the genetic testing of step (f) and/or the genetic modification of step (g) is performed in the offspring animal during embryonic development.
E3. The method of E1, wherein the genetic testing of step (f) and/or the genetic modification of step (g) is performed in the offspring animal 3-60 days after conception.
E4. The method of any one of E1-E3, further comprising birthing the offspring animal.
E5. The method of E1 or E4, wherein the genetic testing of step (f) and/or the genetic modification of step (g) is performed in the offspring animal one day to four years after birth.
E6. The method of any one of E1-E5, further comprising confirming the presence of one or more predetermined traits in the offspring animal after birth.
E7. The method of any one of E1-E6, wherein the genetic testing is performed on a sample obtained from the offspring animal.
E8. The method of E7, wherein the sample comprises blood, tissue, hair, an embryo of the offspring animal implanted as part of an in vitro fertilization procedure, cell-free fetal DNA, mitochondria, or a gamete.
E9. The method of E8, wherein the gamete is an oocyte.
E10. The method of E8, wherein the gamete is a spermatozoon.
E11. The method of any one of E8-E10, wherein the gamete comprises a cytoplasm, a nucleus, a mitochondrion.
E12. The method of any one of E1-E11, wherein the genetic testing is performed using Sanger sequencing, Next Generation Sequencing, microarray, AmpliSeq, genomic sequencing, or real-time quantitative polymerase chain reaction (RT-qPCR).
E13. The method of any one of E4-E12, further comprising genetically modifying the offspring animal lacking the one or more predetermined traits after birth.
E14. The method of any one of E1-E13, wherein the expression of the one or more predetermined traits results from expression of one or more genes.
E15. The method of any one of E1-E14, wherein genetically modifying the offspring animal comprises modifying at least 1, at least 2, at least 3, at least 4, at least 5, at least 6, at least 7, at least 8, at least 9, or at least 10 genes in the offspring animal.
E16. The method of any one of E1-E15, wherein genetically modifying the offspring animal comprises editing of an endogenous gene in the offspring animal.
E17. The method of E16, wherein the endogenous gene comprises a polymorphism.
E18. The method of E16, wherein the endogenous gene comprises a genetic mutation.
E19. The method of any one of E16-E18, wherein the editing of the endogenous gene comprises modifying, deleting, or replacing at least one nucleotide of a DNA or an RNA encoding the endogenous gene in the offspring animal.
E20. The method of any one of E16-E19, wherein the editing of an endogenous gene comprises inserting a donor polynucleotide into a region of the endogenous gene in the offspring animal.
E21. The method of E20, wherein the donor polynucleotide comprises a segment of the endogenous gene that lacks the polymorphism.
E22. The method of E20, wherein the donor polynucleotide comprises a segment of the endogenous gene that lacks the genetic mutation.
E23. The method of any one of E16-E18, wherein the editing of an endogenous gene comprises administering to the offspring animal a polynucleotide encoding one or more copies of a wild type variant of the endogenous gene.
E24. The method of any one of E16-E18, wherein editing of an endogenous gene comprises introducing a heterologous transgene into the genome of the offspring animal.
E25. The method of any one of E16-E24, wherein editing of an endogenous gene comprises modulating expression of an endogenous gene in the offspring animal.
E26. The method of any one of E16-E25, wherein genetically modifying the offspring animal is performed using a clustered regularly interspaced short palindromic repeats (CRISPR)-Cas system, a transcription activator-like effector nuclease (TALEN), or a zinc finger nuclease (ZFN).
E27. The method of E26, wherein the CRISPR-Cas system comprises at least one guide RNA (gRNA) and at least one Cas9 endonuclease.
E28. The method of E27, wherein the Cas9 endonuclease comprises a nuclease-competent Cas9 endonuclease or a nuclease-inactivated Cas9 (dCas9) endonuclease.
E29. The method of E28, wherein the dCas9 endonuclease is fused to a transcriptional regulator domain.
E30. The method of E29, wherein the transcriptional regulator domain is selected from a group consisting of VP16, VP64, p65, RTA, VPR, SAM, SunTag, and KRAB.
E31. The method of any one of E1-E30, wherein the breeding pair capable of producing the offspring animal is integrated into the database system by:
   (a) providing information about one or more predetermined traits in the breeding pair, wherein the information comprises:
      i. results of previously performed genetic testing and/or non-genetic assessment of the breeding pair, wherein the results of the previously performed genetic testing or non-genetic assessment identify the breeding pair as having one or more of the predetermined traits; and/or
      ii. results of genetic testing and/or non-genetic assessment of the breeding pair confirm the presence of one or more of the predetermined traits the breeding pair;
   (b) cataloguing the information of step (a) into the database system;
wherein the database system is configured for interrogation by a user and is configured to produce the probability matrix of step (c) of E1 following said interrogation.
E32. The method of any one of E1 to E31, further comprising registering the offspring animal having the one or more predetermined traits in the database system as a breeder animal.
E33. A method for interrogating a computer-implemented database system to identify one or more breeding pairs capable of producing a non-human offspring animal having one or more predetermined traits, the method comprising the steps of:
   (a) selecting one or more predetermined traits desired in the offspring animal using the database system;
   (b) identifying the one or more breeding pairs capable of producing the offspring animal using the database system;
   (c) generating a probability matrix for each of the one or more breeding pairs using the database system, wherein the probability matrix comprises an array of probability values pertaining to the likelihood that the offspring animal produced by each said breeding pair will have the one or more predetermined traits and displaying the probability matrix on a computer-implemented interface;
   (d) selecting, based on the array of probability values, one or more of the breeding pairs likely to produce the offspring animal having the one or more predetermined traits; and
   (e) breeding the one or more breeding pairs to produce the offspring animal;
wherein the database comprises a panel of at least 2 predetermined traits.
E34. The method of E33, wherein the database comprises a panel of 2-40 predetermined traits.
E35. The method of E34, wherein the database comprises a panel of at least 4 predetermined traits.
E36. The method of E35, wherein the database comprises a panel of at least 6 predetermined traits.
E37. The method of E36, wherein the database comprises a panel of at least 8 predetermined traits.
E38. The method of E37, wherein the database comprises a panel of at least 10 predetermined traits.
E39. The method of E33, wherein the method comprises selecting a panel of 2-40 predetermined traits.
E40. The method of E39, wherein the method comprises selecting a panel of 2 or more predetermined traits.
E41. The method of E40, wherein the method comprises selecting a panel of 4 or more predetermined traits.
E42. The method of E41, wherein the method comprises selecting a panel of 6 or more predetermined traits.
E43. The method of E42, wherein the method comprises selecting a panel of 8 or more predetermined traits.
E44. The method of E43, wherein the method comprises selecting a panel of 10 or more predetermined traits.
E45. The method of E33, further comprising confirming the presence of one or more of the predetermined traits in the offspring animal.
E46. The method of E45, wherein the method comprises confirming the presence of 2-40 predetermined traits.
E47. The method of E46, wherein the method comprises confirming the presence of 2 or more of the predetermined traits.
E48. The method of E47, wherein the method comprises confirming the presence of 4 or more of the predetermined traits.
E49. The method of E48, wherein the method comprises confirming the presence of 6 or more of the predetermined traits.
E50. The method of E49, wherein the method comprises confirming the presence of 8 or more of the predetermined traits.
E51. The method of E50, wherein the method comprises confirming the presence of 10 or more of the predetermined traits.
E52. The method of any one of E33 to E51, wherein genetic testing was previously performed on the breeding pair.
E53. The method of any one of E33 to E52 further comprising registering the offspring animal having the one or more predetermined traits in the database system as a breeder animal.
E54. A method of producing a set of guidelines for breeding of a non-human animal, comprising:
   (a) identifying the non-human animal in a computer-implemented database and assessing the presence of one or more predetermined traits in the non-human animal using the computer-implemented database system, comprising:
   (b) generating a probability matrix for the non-human animal using the database system, wherein the probability matrix comprises an array of probability values pertaining to the likelihood that the non-human animal has the one or more predetermined traits; and
   (c) displaying the probability matrix on a graphical user interface;
   (d) determining, based on the assessment of step (a), one or more conditions under which the non-human animal will develop or is at risk of developing a disease or non-disease condition; and
   (e) generating the set of guidelines for breeding of the non-human animal to avoid manifestation of the disease or non-disease condition.
E55. The method of E54, wherein the non-human animal is used in a breeding pair to produce an offspring animal.
E56. The method of E54 or E55, wherein the set of guidelines comprises one or more recommendations for mitigating or avoiding the one or more conditions that would result in manifestation of one or more symptoms of the disease or non-disease condition and/or for reducing the likelihood of producing an effect in the offspring animal resulting from development of the disease or non-disease condition.
E57. The method of any one of E54-E56, wherein the non-human animal is a female parent animal.
E58. The method of any one of E54-E56, wherein the non-human animal is a male parent animal.
E59. The method of any one of E54-E58, wherein the set of guidelines provides one or more recommendations that increases the likelihood of producing a non-human offspring animal having one or more predetermined traits.
E60. The method of E59, wherein the one or more recommendations pertain to a diet, exercise regime, discipline, environmental exposure, medication, supplementary treatments, training, conditioning, use, or and/or limitation the non-human animal.
E61. A method of generating a computer-implemented database system for producing a non-human offspring animal having one or more predetermined traits, comprising:
   (a) providing information about the one or more predetermined traits from a plurality of non-human animals of a same type as the offspring animal, wherein the information comprises:
      (a) results of previously performed genetic testing and/or non-genetic assessment of the plurality of non-human animals, wherein the results of the previously performed genetic testing or non-genetic assessment identify the plurality of non-human animals as having one or more of the predetermined traits; and/or
      (b) results of genetic testing and/or non-genetic assessment of the plurality of non-human animals that confirm the presence of one or more of the predetermined traits in the plurality of non-human animals;
   (b) cataloguing the information of step (a) into the database system;
wherein the database system is configured for interrogation by a user and is configured to produce a probability matrix based on the information catalogued in step (b) following said interrogation.
E62. The method of E61, wherein the interrogation comprises selecting one or more predetermined traits desired in the offspring animal and identifying from the plurality of non-human animals a breeding pair capable of producing said offspring animal.
E63. The method of E61 or E62, further comprising selecting a breeding pair capable of producing the offspring having the one or more predetermined traits.
E64. The method of E63, wherein the probability matrix comprises an array of probability values pertaining to the likelihood of the selected breeding pair producing a non-human offspring animal having the one or more predetermined traits.
E65. The method of any one of E61-E63, wherein the probability matrix comprises an array of probability values pertaining to the likelihood of the plurality of non-human animals having the one or more predetermined traits.
E66. The method of any one of E61-E65, wherein the non-genetic assessment comprises:
   (a) obtaining or having obtained information from someone knowledgeable about the non-human animal pertaining to the one or more predetermined traits;
   (b) performing or having performed one or more clinical assays on biological tissue obtained from the non-human animal pertaining to the one or more predetermined traits;
   (c) performing or having performed one or more diagnostic tests on the non-human animal pertaining to the one or more predetermined traits; and/or
   (d) obtaining scores assigned to the non-human animal during a competition pertaining to the one or more predetermined traits.
E67. A computer-implemented interface for assisting a user in producing, procuring, and/or identifying one or more non-human animals having one or more predetermined traits, wherein the interface is configured to allow a user to interrogate a computer-implemented database system, the interrogation comprising:
   (a) selecting the one or more predetermined traits of interest for the one or more non-human animals;
   (b) identifying one or more of the non-human animals catalogued in the database system and generating a probability matrix for each of the one or more non-human animals identified using the database system;
   (c) based on the probability matrix of step (d), displaying an overall assessment of each of the non-human animals having the one or more predetermined traits; and
   (d) providing an option for the user to produce, purchase, or lease one or more of the non-human animals having the one or more predetermined traits;
wherein the computer-implemented interface is in communication with the database system.
E68. The method of E67, wherein step (a) further comprises selecting one or more predetermined traits of at least one parent of the one or more non-human animals.
E69. The interface of E67 or E68, wherein step (a) further comprises selecting a gene pool from which the one or more non-human animals or the at least one parent of the non-human animal are obtained.
E70. The method of E69, wherein the gene pool is selected from a group consisting of the user's own animals, animals owned by other users, an animal registry, and animals located within a specified geographical area.
E71. The method of any one of E67-E70, wherein the probability matrix comprises an array of probability values pertaining to the likelihood of the non-human animal having one or more predetermined traits.
E72. A method using a computer-implemented database system to identify a non-human animal as having one or more predetermined traits, comprising:
   (a) providing information about the non-human animal;
   (b) cataloguing the information of step (a) into the database system;
   (c) comparing the information of step (b) to information about a plurality of non-human animals having the one or more predetermined traits, wherein the information about the plurality of non-human animals has been previously catalogued in the database system;
   (d) generating a probability matrix for the non-human animal based on the comparison of step (c) using the database system, wherein the probability matrix comprises an array of probability values pertaining to the likelihood that the non-human animal has the one or more predetermined traits;
   (e) identifying the non-human animal as having the one or more predetermined traits based on the probability matrix of step (d); and
   (f) optionally, genetically modifying the non-human animal to promote or suppress an expression of the one or more predetermined traits in the non-human animal;
wherein the information comprises results of previously performed genetic testing and/or non-genetic assessment of the non-human animal or the plurality of non-human animals.
E73. The method of any one of E1-E72, wherein the one or more predetermined traits comprise coat color, coat color modifier, coat texture, coat thickness, facial marking, leg marking, eye color, skin color, mane color, tail color, speed, gait, temperament, and health.
E74. The method of E73, wherein the coat color, mane color and/or tail color is selected from a group consisting of amber champagne, amber champagne dun, amber cream, amber dun, amber dun pearl, apricot dun, apricot pearl, agouti, bay, bay cream pearl, bay double cream, bay pearl, black, black bay, black cream pearl, black double cream, black dun, black pearl, black/red, blanket appaloosa, blood bay, blue roan, brindle, brown, buckskin, buttermilk buckskin, champagne, champagne amber pearl, champagne classic pearl, champagne dun, champagne dun pearl, champagne gold pearl, champagne pearl, chesetnut, chestnut cream pearl, chocolate, classic champagne, classic champagne dun, classic cream, classic dun, cream, cream champagne, cream grullo, cream pearl, cremello, dapple grey, dark bay, dark brown, dark chestnut, dominant white, dun, dun bay cream, dun bay double cream, dun black cream, dun black double cream, dun chestnut cream, dun chestnut double cream, dun pearl, dun pearl bay, dun pearl black, dunalino, dunolino, dunskin, dunskin splash, flaxen chestnut, fleabitten grey, frame overo, frosted appaloosa, grey, gold champagne, gold cream, gold dun, gold dun pearl, grey, grullo, half blanket, leopard appaloosa, light grey, liver chestnut, marbled appaloosa, overo, palomino, pearl, perlino, perlino dun, pseudocream classic, red, red chestnut, red dun, red roan, rose grey, sabino, seal brown, silver, silver amber champagne, silver amber dun, silver bay cream, silver bay cream pearl, silver bay double cream, silver black, silver black cream pearl, silver black double cream, silver champagne, silver champagne dun, silver champagne pearl, silver classic champagne, silver classic champagne pearl, silver cream pearl, silver dapple, silver dapple bay, silver dapple buckskin, silver dapple cream grullo, silver dapple dun, silver dapple dunskin, silver dapple grullo, silver dapple pearl, silver dapple perlino, silver dapple perlino dun, silver dun, silver dun bay cream, silver dun bay double cream, silver dun black, silver dun black double cream, silver dun pearl, silver dun pearl bay, silver dun pearl black, silver pearl bay, silver pearl black, silver red, silver smoky cream, skewbald, smoky black, smoky cream, smoky grullo, snowflake appaloosa, sorrel, splashed white, steel grey, sun, tobiano, tovero, tri-color, white, and white spotting.
E75. The method of E73, wherein the coat color modifier is selected from a group consisting of apron, barring on body, barring on shoulder, belly spots (large or small), belly stripe, belted, bend or spots on body, bend or spots on head, birdcatcher spots, black spots, blagdon, blanket with roaning, blanket with spots, blaze, blaze with freckling, body spots, body white, brindle, brow spots, calico, coon/skunk tail, dapples, dilute, dorsal stripe, double dilute, ermine markings, few white hairs on body, fewspot, flaxen mane/tail, flea-bitten, fleshmark, frosted, grullo, heart marking, highlights in mane/tail, lacing pattern, leopard spotted, lightning marks, line back, frosting mane/tail, maximum tobiano, maximum overo, maximum white, maximum white sabino, medicine hat, minimal overo, minimal sabino, minimal tobiano, mixed mane/tail, mottled, no marking, overo, pangare, paw prints, pearl, piebald, pinto markings, rabicano, roan, roaning on body, rose, rump spots, sabino, skewbald, smudge marks, smutty, snip, snowcap, sooty, splash markings, star, stripe, tobiano, umbilical spots, white head, white mane/tail, white throat latch, white tipped ears, white with fading edges, white with ragged edges, white with smooth edges, wither spot, no markings, solid, white blaze, and white star.
E76. The method of E73, wherein the facial marking is selected from a group consisting of apron face, badger face, bald face, blaze, interrupted stripe, face, snip, star, stripe markings, both eyes amber, both eyes blue, both eyes brown, both eyes green, both eyes tiger, eyebrows, face mask, few white hairs on forehead, left eye amber, left eye blue, left eye brown, left eye green, left eye partial blue, left eye tiger, partial bald face, pigment around eye, right eye amber, right eye blue, right eye brown, right eye green, right eye partial blue, right eye tiger, white around eye, white chin, white jaw, white lip, white nose, and white sclera.
E77. The method of E73, wherein the leg marking is selected from a group consisting of two back white stockings, two white front socks, two white front stockings, two white hind socks, two white hind stockings, four white socks, four white stockings, back left above hock, back left cannon (¾ stocking), back left coronet, back left ermine spots, back left fetlock, back left half pastern, back left heel, back left partial heel, back left pastern, back left sock, back left stocking, back left stripes, back right above hock, back right cannon, back right coronet, back right ermine spots, back right fetlock, back right half pastern, back right heel, back right partial heel, back right pastern, back right sock, back right stocking, back right stripes, barring on legs, bend or spots on legs, front left above knee, front left cannon, front left coronet, front left ermine spots, front left fetlock, front left half pastern, front left heel, front left partial heel, front left pastern, front left sock, front left stocking, front left stripes, front right above knee, front right cannon, front right coronet, fright right ermine spots, front right fetlock, front right half pastern, front right heel, front right partial heel, front right pastern, front right sock, front right stocking, front right stripes, left front hoof stripes, left front white foot, left hind hoof stripes, left hind white foot, right front hoof stripes, right front white foot, right hind hoof stripes, right hind white foot, and roaning on white socks/stockings.
E78. The method of E73, wherein the coat texture comprises smooth, rough, curly, straight, downy, spiky, or brindle.
E79. The method of E73, wherein the coat thickness comprises thick, medium, or thin.
E80. The method of E73, wherein the eye color comprises blue, amber, yellow, orange, hazel, green, or brown.
E81. The method of E73, wherein the skin color comprises pink, black, brown, yellow, and white.
E82. The method of E73, wherein the speed is selected from the group consisting of endurance, mid-distance, and sprint.
E83. The method of E73, wherein the gait is selected from the group consisting of non-gaited, gait carrier, and gaited.
E84. The method of E83, wherein gaited comprises walk, trot, canter, gallop, pacing, fox trot, racking, country pleasure, Indian shuffle, jogging, paso fino, paso corto, paso largo, paso llano, sobrandando, marcha picada, rack, running walk, stepping pace, singlefoot, tölt, and ravaal.
E85. The method of E73, wherein the temperament is selected from the group consisting of vigilant, curious/vigilant, curious, spooky, non-spooky, hot, cold, and medium.
E86. The method of E73, wherein the health comprises variants of one or more genes associated with one or more disease or non-disease conditions that are undesired in the non-human animal.
E87. The method of any one of E1-E86, wherein each of the one or more breeding pairs comprise at least a first non-human parent animal and a second non-human parent animal.
E88. The method of any one of E1-E87, wherein the method further comprises selecting the sex, breed, and/or discipline of the offspring animal, the first parent animal, and/or the second parent animal.
E89. The method of E88, wherein the breed is selected from the group consisting of Abyssianian/Ethiopian/Oromo/Gala horse, Abyssinian, Akhal Teke, Albanian, Albanian Horse, Alt-Olderburger, Altai Horse, American Cream Draft, American Cream and White, American Curly, American Gaited Mountain Horse, American Gaited Pony, American Paint Horse, American Quarter Horse, American Saddlebred, American Walking Pony, American Warmblood, Andalusian, Andravida, Andravida/Eleia Horse, Anglo European Warmblood, Anglo-Arab, Anglo-Kabarda, Appaloosa, Appendix Quarter Horse, AraAppaloosa, Arabian, Arabian Sporthorse, Ardennes, Ardennes Horse, Argentine Criollo, Asturcon, Asturian, Australian Brumby, Australian Stock Horse, Austrian Warmblood, Azteca, Baise Horse, Balearic, Baluchi, Baluchi Horse, Ban-ei, Banker, Banker Horse, Barb, Barb/Berber Horse, Bashkir, Bashkir Curly, Bashkir Curly Horse, Bashkir Horse, Basotho Pony, Basuto Pony, Belgian, Belgian Draft, Belgian Warmblood, Bhirum Pony, Bhirum/Nigerian Pony, Bhotia Pony, Bhutia Pony, Black Forest, Black Forest Pony, Blue Eyed Horse Association, Boer, Boerperd, Brandenburger, Breton, Breton Horse, British Riding Pony, British Warmblood, Buckskin, Budyonny, Budyonny Horse, Byelorussian Harness, Byelorussian Harness Horse, Camargue, Camargue Horse, Campolina, Canadian Horse, Canadian Sport Horse, Canadian Suffield Mustang, Canadian Warmblood, Carolina Marsh Tacky, Carthusian, Caspian, Caspian Horse, Cayuse, Celtic Warmblood, Cheju, Chicoteague Pony, Chilean Corralero, Chilean Corralero Horse, Chincoteague Pony, Cleveland Bay, Clydesdale, Colorado Ranger Horse, Connemara, Cream Draft, Cremello, Criollo, Crioulo, Curly Horse, Dales Pony, Danish Warmblood, Danube, Danube Delta Horse, Dartmoor Pony, Deliboz, Deliboz/Delibozskaya, Djerma, Dole, Dole Gudbrandsdal/Dolahest/Døle, Dongola, Donkey, Draft, Draft Cross, Draft Horse, Drum Horse, Dutch Draft, Dutch Harness, Dutch Harness Horse, Dutch Warmblood, East Bulgarian, East Friesian, Egyptian, Egyptian Arabian, Eriskay Pony, Estonian Horse/Estonian Native/Estonian Klepper/Eisti Hobune, Estonian Native, Exmoor Pony, Faeroe Pony, Faeroes Pony, Falabella, Falabella Miniature Horse, Fell Pony, Ferghana, Finnhorse, Finnhorse/Finnish Horse, Fjord, Fjord Horse, Fleuve, Florida Cracker, Florida Cracker Horse, Foundation Quarter Horse, Fouta, Foutanke/Fouta, Frederiksborg, Frederiksborg Horse, French Saddlebred, French Saddlebred Horse, French Trotter, Friesian, Friesian Cross, Galiceño, Galician Pony, Galician/Galician Mountain Horse, Gelderlander, Georgian Grande, German Bred, German Riding Pony, Gidran, Gidran/Hungarian Anglo-Arab, Golden American Saddlebred, Gotland, Gotland Pony, Groningen, Groningen Horse, Guangxi, Gypsy Cob, Gypsy Vanner, Hackney, Hackney Horse, Hackney Pony, Haflinger, Half-Arabian, Hanoverian, Hanoverian Warmblood, Hequ, Highland Pony, Hokkaido, Hokkaido Horse, Holsteiner, Holsteiner Warmblood, Hucul, Hucul Pony, Hungarian Warmblood, Iberian Warmblood, Icelandic, Icelandic Horse, lomud, lomud/Yomud/Yamud/Yomut, Irish Cob, Irish Draft, Irish Draught, Irish Draught Horse, Irish Sport Horse, Jeju Horse, Jinzhou, Jutland, Kabarda, Kabarda/Kabardin, Karabair, Karabakh, Kazakh, Kazakh Horse, Kentucky Mountain horse, Kentucky Mountain Saddle Horse, Kerry Bog Pony, KigeR Mustang, Kirdi Pony, Kisber Felver, Kiso, Kiso Horse, Kladbury, Kladbury Horse, Knabstrup, Knabstrupper Warmblood, Kushum, Kushum Horse, Kustanai, Kustanai Horse, Kyrgyz/Kirgiiz Horse, Latvian, Latvian Horse, Lipizzan, Lithuanian Heavy Draft, Lokai, Losino, Losino Horse, Lusitano, M Bayar, Mallorquin, Malopolski, Mangalarga, Mangalarga Marchador, Marsh Tacky, Marwari, Marwari Horse, Wrens Pony, Messara, Messara Horse, Miniature, Miniature Horse, Misaki, Misaki Horse, Missouri Fox Trotter, Missouri Fox Trotting Horse, Miyako, Miyako Pony, Mongolian, Mongolian Horse, Morab, Morgan, Mountain Pleasure Horse, Moyle, Mule, Murgese, Mustang, National Show horse, National Spotted horse, New Forest Pony, New Kirgiz, Newfoundland Pony, Nokota, Nokota Horse, Noma, Noma Horse, Nooitgedacht Pony, Nordland, Nordlandshest/Lyngshest/Nordland Pony, Norfolk Trotter, Noric, North Swedish horse, Northeastern, Ob, Oldenburg, Oldenburg Warmblood, Orlov Trotter, Paint (Overo), Paint (Solid), Paint (Tobiano), Paint (Tovero), Paints (All), Palomino, Pantaneiro, Pantaneiro horse, Paso Fino, Paulista Marchador, Peneia Pony, Percheron, Peruvian Paso, Pindos Pony, Pinia, Pintabian, Pinto, POA, Polish Horse, Ponies (All), Pony, Pony of America, Pottok, Priob/Ob, Przewalski, Przewalski Horse, Pura Raza Espanola (PRE), Pyrenean Tarpan, Qatgani, Qatgani/Qataghani Horse, Quarab, Quarter Horse, Quarter Horse Cross, Quarter Pony, Racking Horse, Rheinland Pfalz Saar, Rhenish Warmblood, Rhinelander horse, Rocky Mountain, Rocky Mountain Horse, Russian Don, Russian Heavy Draft, Russian Trakhener, Russian Trotter, Sanhe, Sanhe Horse, Schleswiger Heavy Draft, Scharzwälder Fuchs, Selle Francais, Shagya, Shagya Arabian, Shetland Pony, Shire, Single-Footing Horse, Skyros Pony, Somali Pony, Sorraia, Soviet Heavy Draft, Spanish Mustang, Spanish-Barb, Spanish-Norman, Spanish-Normal Horse, Sport Horse of Color, Spotted Draft, Spotted Draft Horse, Spotted Saddle, Spotted Saddle Horse, Standardbred, Sudan Country-Bred, Sudan Country-Bred Horse, Suffolk, Suffolk/Suffolk Punch/Suffolk Sorrel, Swedish Warmblood, Taishu/Tsushima, Taishuh, Tarpan, Tawleed, Tennessee Walker, Tennessee Walking Horse/Tennessee Walker, Tersk, Tersk Horse, Thessalian, Thessalian/Thessaly Horse, Thoroughbred, Thoroughbred Cross, Tibetan Pony, Tiger horse, Tokara, Tori, Tori Horse, Trakehner, Ukrainian Riding Horse/Ukrainian Saddle Horse, Ukrainian Saddle, Virginia Highlander, Vlaamperd, Vladimir Heavy Draft, Vyatka, Vyatka Horse, Walkaloosa, Warmblood, Warmblood Cross, Warmblood (All), Warmbloods of Color, Welara Pony, Welsh Cob, Welsh Pony, Weser-Em Pony, West African Barb, Western Sudan Pony, Westphalian, Wielkopolski, Wurttemberger Warmblood, Xilingol, Yakut, Yanqi, Yili, Yonaguni, Zangersheide, Zaniskari Pony, Zhemaichu, and Zorse. E90. The method of E89, wherein the discipline is selected from the group consisting of barrel racing, beginner/family, breeding, brood mare, calf roping, companion only, competitive trail competitions, country pleasure, cowboy mounted shooting, cutting, draft, dressage, drill team, driving, endurance riding, English pleasure, equitation, eventing, field hunter, gaited, halter, harness, horsemanship, hunter, hunter under saddle, judged pleasure rides, jumper, lesson horse, longe-line, pleasure driving, pole bending, polo, racing, ranch horse, ranch sorting, reined cow horse, reining, rodeo, roping, showmanship, saddle seat, sidesaddle, steer wrestling, team penning, team roping, team sorting, trail horse, vaulting, Western pleasure, Western pleasure (show), Western riding, working cattle, youth/4-H horse, all around, 4-in hand driving, agility, breed shows, breeding stallion, broodmare, bull fighting, camping, cart, combined driving, cowboy dressage, cowboy racing, doma vaquero, field trials, gymkhana, harness racing, hazing, horse ball, marathon driving, mounted archery, mounted games, mounted patrol, mounted police, native costume, natural horsemanship, nurse mare, pacing, pack horse, parades, park, playday, polocrosse, ranch versatility, recipient broodmare, roadster, rodeo bronc, rodeo pickup horse, rodeo queen horse, roping, saddleseat, search and rescue, show hack, ski joring, steer roping, stock horse, sulky racing, team driving, team roping head horse, team roping heel horse, therapeutic riding, trial class competition, recreational trail riding, turnback horse, Western dressage, working equitation, hunt seat, show jumper, riding, pleasure, and bucking horse.
E91. The method of any one of E1-E90, wherein the likelihood that the offspring animal produced by each said breeding pair will have the one or more predetermined traits is at least 25%, 15%, 25%, 50%, 60%, 70%, 80%, 90%, 95%, 99%, or 0%.
E92. The method of any one of E54-E91, wherein the disease or non-disease condition is selected from a group consisting of anhidrosis, androgen insensitivity syndrome, cerebellar abiotrophy, curved ear tips, dwarfism, degenerative suspensory ligament disease, equine asthma, equine herpes virus risk, epistaxis risk, equine metabolic syndrome, laminitis, equine recurrent uveitis, equine viral arteritis susceptibility, foal immunodeficiency syndrome, gait transition, glycogen branching enzyme disorder, hereditary equine regional dermal asthenia, hoof wall separation disease, hydrocephalus, hyperkalemic periodic paralysis, immune-mediated myositis, impaired acrosomal reaction, junctional epidermolysis bullosa, kissing spines, lavender foal syndrome, lordosis, malignant hyperthermia, myotonia, navicular disease, osteochondritis dissecans, polysaccharide storage myopathy, recurrent laryngeal neuropathy, severe combined immunodeficiency, squamous cell carcinoma, tendinopathy, warmblood fragile foal syndrome, West Nile Virus susceptibility, wobbler syndrome, and behavioral abnormality.
E93. The method of E92, wherein the behavioral abnormality is selected from the group consisting of cribbing, teeth grinding, weaving, anxiety, depression, extreme aggression, extreme fearfulness, bucking, striking, biting, self-mutilating, and head shaking.
E94. The method of any one of E1-E93, wherein the one or more predetermined traits comprise at least 2 or more predetermined traits.
E95. The method of E94, wherein the one or more predetermined traits comprise at least 4 or more predetermined traits.
E96. The method of E95, wherein the one or more predetermined traits comprise at least 6 or more predetermined traits.
E97. The method of E96, wherein the one or more predetermined traits comprise at least 8 or more predetermined traits.
E98. The method of E97, wherein the one or more predetermined traits comprise at least 10 or more predetermined traits.
E99. The method of any one of E1-E98, wherein the computer-implemented database system comprises genetic and non-genetic information about the non-human animal, non-human parent animal, and/or non-human offspring animal.
E100. The method of any one of E1-E99, wherein the genetic testing comprises genotyping the non-human animal for any one of the following genetic markers:
   (a) agouti, black/red, brindle, champagne, cream, dominant, white, frame overo, grey, pearl, sabino, silver, splashed white, tobiano, and white spotting;
   (b) a curiosity gene that is a dopamine D4 receptor (DRD4) gene;
   (c) a speed gene that is a myostatin (MSTN) gene;
   (d) a gait gene that is a doublesex and mab-3-related transcription factor 3 (DMRT3) gene; and/or
   (e) a health gene selected from the group consisting of androgen receptor (AR), mutY DNA glycosylase (MUTYH), teashirt zinc finger homeobox 1 (TSHZ1), aggrecan (ACAN), family with sequence similarity 174 member A (FAM174A), interleukin 17A (IL17A), interleukin 17B (IL17B), chemokine (C-X-C motif) ligand 16 (CXCL16), solute carrier family 5 member 3
   (SLC5A3), glycogen branching enzyme (GBE1), peptidylprolyl isomerase B (PPIB), serpin family B member 11 (SERPINB11), beta-1,3-N-acetylgalactosaminyltransferase 2 (B3GALNT2), sodium voltage-gated channel alpha subunit 4 (SCN4A), myosin heavy chain 1 (MYH1), FKBP prolyl isomerase 6 (FKBP6), laminin subunit gamma 2 (LAMC2), myosin 5A (MYOSA), equine chromosome 20 (ECA20), ryanodine receptor 1 (RYR1), chloride voltage-gated channel 4 (CLCN4), glycogen synthase 1 (GYS1), equine chromosome 3 (ECA3), protein kinase DNA-activated catalytic subunit (DNAPK), damage specific DNA binding protein 2 (DDB2), procollagen-lysine, 2-oxoglutarate 5-dioxygenase 1 (PLOD1), 2′-5′-oligoadenylate synthetase 1 (OAS1), melanocyte inducing transcription factor (MITF), paired box (PAX), proto-oncogene receptor tyrosine kinase (KIT), high mobility group AT-hook 2 (HMGA2), zinc finger and AT-hook domain containing (ZFAT), ligand dependent nuclear receptor corepressor like (LCORL), non-SMC condensing I complex subunit G (NACPG), and LIM and SH3 protein 1 (LASP1).
E101. The method of any one of E1-E100, wherein the genetic testing comprises performing analysis of an entire genome of the non-human animal.
E102. The method of E101, wherein the analysis of an entire genome comprises genome wide association study (GWAS).
E103. The method of any one of E1-E102, wherein the non-human animal, non-human parent animal, or non-human offspring animal is selected from the group consisting of horse, cattle, sheep, dog, cat, camel, pig, goat, alpaca, donkey, llama, red fox, mouse, rat, ferret, non-human primate, rabbit, gerbil, hamster, chinchilla, or guinea pig.
E104. The method of any one of E1-E103, wherein the method further comprises generating a heterozygosity score for the non-human animal or non-human offspring animal based on a sample obtained from the non-human animal or non-human offspring animal, respectively.
E105. The method of any one of E1-E104, wherein the method further comprises generating an inbreeding coefficient (F) for the non-human animal or non-human offspring animal based on a sample obtained from the non-human animal or non-human offspring animal, respectively.
E106. The method of any one of E1-E105, wherein the method further comprises performing a breed composition analysis on a sample from the non-human animal or the non-human offspring animal.
E107. The method of any one of E1-E106, wherein the method further comprises performing an identity-by-descent analysis on a sample from the non-human animal or the non-human offspring animal.
E108. The method of any one of E1-E107, wherein the method further comprises generating a shared centimorgan (cM) value for the non-human animal or non-human offspring animal based on a sample obtained from the non-human animal or non-human offspring animal, respectively.
E109. The method of any one of E1-E108, wherein the method further comprises generating a kinship coefficient for the non-human animal or non-human offspring animal based on a sample obtained from the non-human animal or non-human offspring animal, respectively.
E110. The method of any one of E104-E109, wherein the sample is a biological sample
E111. The method of E110, wherein the biological sample is a hair sample, such as a hair sample comprising one or more hair follicles.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations for use in the compositions and methods of the invention following, in general, the principles for use in the compositions and methods of the invention and including such departures from the present disclosure that come within known or customary practice within the art to which the invention pertains and may be applied to the essential features hereinbefore set forth, and follows in the scope of the claims.

1. A method of producing a non-human offspring animal having one or more predetermined traits, the method comprising:
(a) selecting one more predetermined traits desired in the offspring animal using a computer-implemented database system;
(b) interrogating the database system to identify one or more breeding pairs capable of producing the offspring animal;
(c) generating a probability matrix for each of the one or more breeding pairs using the database system, wherein the probability matrix comprises an array of probability values pertaining to the likelihood that the offspring animal produced by each said breeding pair will have the one or more predetermined traits;
(d) selecting, based on the array of probability values, one or more of the breeding pairs likely to produce the offspring animal having the one or more predetermined traits;
(e) breeding one or more of the selected breeding pairs to produce the offspring animal;
(f) performing genetic testing of the offspring animal to detect the presence of the one or more predetermined traits; and
(g) genetically modifying the offspring animal lacking the one or more predetermined traits to promote or improve expression of the one or more predetermined traits.

2. The method of claim 1, wherein the genetic testing of step (f) and/or the genetic modification of step (g) is performed in the offspring animal during embryonic development.

3. The method of claim 1, wherein the genetic testing of step (f) and/or the genetic modification of step (g) is performed in the offspring animal 3-60 days after conception.

4. The method of claim 1, further comprising birthing the offspring animal.

5. The method of claim 1, wherein the genetic testing of step (f) and/or the genetic modification of step (g) is performed in the offspring animal one day to four years after birth.

6. The method of claim 1, further comprising confirming the presence of one or more predetermined traits in the offspring animal after birth.

7. The method of claim 1, wherein the genetic testing is performed on a sample obtained from the offspring animal.

8. The method of claim 7, wherein the sample comprises blood, tissue, hair, an embryo of the offspring animal implanted as part of an in vitro fertilization procedure, cell-free fetal DNA, mitochondria, or a gamete.

9. The method of claim 8, wherein the gamete is an oocyte.

10. The method of claim 8, wherein the gamete is a spermatozoon.

11. The method of claim 8, wherein the gamete comprises a cytoplasm, a nucleus, a mitochondrion.

12. The method of claim 1, wherein the genetic testing is performed using Sanger sequencing, Next Generation Sequencing, microarray, AmpliSeq, genomic sequencing, or real-time quantitative polymerase chain reaction (RT-qPCR).

13. The method of claim 4, further comprising genetically modifying the offspring animal lacking the one or more predetermined traits after birth.

14. The method of claim 1, wherein the expression of the one or more predetermined traits results from expression of one or more genes.

15. The method of claim 1, wherein genetically modifying the offspring animal comprises modifying at least 1, at least 2, at least 3, at least 4, at least 5, at least 6, at least 7, at least 8, at least 9, or at least 10 genes in the offspring animal.

16. The method of claim 1, wherein genetically modifying the offspring animal comprises editing of an endogenous gene in the offspring animal.

17. The method of claim 16, wherein the endogenous gene comprises a polymorphism.

18. The method of claim 16, wherein the endogenous gene comprises a genetic mutation.

19. The method of claim 16, wherein the editing of the endogenous gene comprises modifying, deleting, or replacing at least one nucleotide of a DNA or an RNA encoding the endogenous gene in the offspring animal.

20. The method of claim 16, wherein the editing of an endogenous gene comprises inserting a donor polynucleotide into a region of the endogenous gene in the offspring animal.

21. The method of claim 20, wherein the donor polynucleotide comprises a segment of the endogenous gene that lacks the polymorphism.

22. The method of claim 20, wherein the donor polynucleotide comprises a segment of the endogenous gene that lacks the genetic mutation.

23. The method of claim 16, wherein the editing of an endogenous gene comprises administering to the offspring animal a polynucleotide encoding one or more copies of a wild type variant of the endogenous gene.

24. The method of claim 16, wherein editing of an endogenous gene comprises introducing a heterologous transgene into the genome of the offspring animal.

25. The method of claim 16, wherein editing of an endogenous gene comprises modulating expression of an endogenous gene in the offspring animal.

26. The method of claim 16, wherein genetically modifying the offspring animal is performed using a clustered regularly interspaced short palindromic repeats (CRISPR)-Cas system, a transcription activator-like effector nuclease (TALEN), or a zinc finger nuclease (ZFN).

27. The method of claim 26, wherein the CRISPR-Cas system comprises at least one guide RNA (gRNA) and at least one Cas9 endonuclease.

28. The method of claim 27, wherein the Cas9 endonuclease comprises a nuclease-competent Cas9 endonuclease or a nuclease-inactivated Cas9 (dCas9) endonuclease.

29. The method of claim 28, wherein the dCas9 endonuclease is fused to a transcriptional regulator domain.

30. The method of claim 29, wherein the transcriptional regulator domain is selected from a group consisting of VP16, VP64, p65, RTA, VPR, SAM, SunTag, and KRAB.

31. The method of claim 1, wherein the breeding pair capable of producing the offspring animal is integrated into the database system by:
(a) providing information about one or more predetermined traits in the breeding pair, wherein the information comprises:
(i) results of previously performed genetic testing and/or non-genetic assessment of the breeding pair, wherein the results of the previously performed genetic testing or non-genetic assessment identify the breeding pair as having one or more of the predetermined traits; and/or
(ii) results of genetic testing and/or non-genetic assessment of the breeding pair confirm the presence of one or more of the predetermined traits the breeding pair;
(b) cataloguing the information of step (a) into the database system;
wherein the database system is configured for interrogation by a user and is configured to produce the probability matrix of step (c) of claim 1 following said interrogation.

32. The method of claim 1, further comprising registering the offspring animal having the one or more predetermined traits in the database system as a breeder animal.

33. A method for interrogating a computer-implemented database system to identify one or more breeding pairs capable of producing a non-human offspring animal having one or more predetermined traits, the method comprising the steps of:
(a) selecting one or more predetermined traits desired in the offspring animal using the database system;
(b) identifying the one or more breeding pairs capable of producing the offspring animal using the database system;
(c) generating a probability matrix for each of the one or more breeding pairs using the database system, wherein the probability matrix comprises an array of probability values pertaining to the likelihood that the offspring animal produced by each said breeding pair will have the one or more predetermined traits and displaying the probability matrix on a computer-implemented interface;
(d) selecting, based on the array of probability values, one or more of the breeding pairs likely to produce the offspring animal having the one or more predetermined traits; and
(e) breeding the one or more breeding pairs to produce the offspring animal;
wherein the database comprises a panel of at least 2 predetermined traits.

34. The method of claim 33, wherein the database comprises a panel of 2-40 predetermined traits.

35. The method of claim 34, wherein the database comprises a panel of at least 4 predetermined traits.

36. The method of claim 35, wherein the database comprises a panel of at least 6 predetermined traits.

37. The method of claim 36, wherein the database comprises a panel of at least 8 predetermined traits.

38. The method of claim 37, wherein the database comprises a panel of at least 10 predetermined traits.

39. The method of claim 33, wherein the method comprises selecting a panel of 2-40 predetermined traits.

40. The method of claim 39, wherein the method comprises selecting a panel of 2 or more predetermined traits.

41. The method of claim 40, wherein the method comprises selecting a panel of 4 or more predetermined traits.

42. The method of claim 41, wherein the method comprises selecting a panel of 6 or more predetermined traits.

43. The method of claim 42, wherein the method comprises selecting a panel of 8 or more predetermined traits.

44. The method of claim 43, wherein the method comprises selecting a panel of 10 or more predetermined traits.

45. The method of claim 33, further comprising confirming the presence of one or more of the predetermined traits in the offspring animal.

46. The method of claim 45, wherein the method comprises confirming the presence of 2-40 predetermined traits.

47. The method of claim 46, wherein the method comprises confirming the presence of 2 or more of the predetermined traits.

48. The method of claim 47, wherein the method comprises confirming the presence of 4 or more of the predetermined traits.

49. The method of claim 48, wherein the method comprises confirming the presence of 6 or more of the predetermined traits.

50. The method of claim 49, wherein the method comprises confirming the presence of 8 or more of the predetermined traits.

51. The method of claim 50, wherein the method comprises confirming the presence of 10 or more of the predetermined traits.

52. The method of claim 33, wherein genetic testing was previously performed on the breeding pair.

53. The method of claim 33, further comprising registering the offspring animal having the one or more predetermined traits in the database system as a breeder animal.

54. A method of producing a set of guidelines for breeding of a non-human animal, comprising:
(a) identifying the non-human animal in a computer-implemented database and assessing the presence of one or more predetermined traits in the non-human animal using the computer-implemented database system, comprising:
(i) generating a probability matrix for the non-human animal using the database system, wherein the probability matrix comprises an array of probability values pertaining to the likelihood that the non-human animal has the one or more predetermined traits; and
(ii) displaying the probability matrix on a graphical user interface;
(b) determining, based on the assessment of step (a), one or more conditions under which the non-human animal will develop or is at risk of developing a disease or non-disease condition; and
(c) generating the set of guidelines for breeding of the non-human animal to avoid manifestation of the disease or non-disease condition.

55. The method of claim 54, wherein the non-human animal is used in a breeding pair to produce an offspring animal.

56. The method of claim 54, wherein the set of guidelines comprises one or more recommendations for mitigating or avoiding the one or more conditions that would result in manifestation of one or more symptoms of the disease or non-disease condition and/or for reducing the likelihood of producing an effect in the offspring animal resulting from development of the disease or non-disease condition.

57. The method of claim 54, wherein the non-human animal is a female parent animal.

58. The method of claim 54, wherein the non-human animal is a male parent animal.

59. The method of claim 54, wherein the set of guidelines provides one or more recommendations that increases the likelihood of producing a non-human offspring animal having one or more predetermined traits.

60. The method of claim 59, wherein the one or more recommendations pertain to a diet, exercise regime, discipline, environmental exposure, medication, supplementary treatments, training, conditioning, use, or and/or limitation the non-human animal.

61. A method of generating a computer-implemented database system for producing a non-human offspring animal having one or more predetermined traits, comprising:
(a) providing information about the one or more predetermined traits from a plurality of non-human animals of a same type as the offspring animal, wherein the information comprises:
(i) results of previously performed genetic testing and/or non-genetic assessment of the plurality of non-human animals, wherein the results of the previously performed genetic testing or non-genetic assessment identify the plurality of non-human animals as having one or more of the predetermined traits; and/or
(ii) results of genetic testing and/or non-genetic assessment of the plurality of non-human animals that confirm the presence of one or more of the predetermined traits in the plurality of non-human animals;
(b) cataloguing the information of step (a) into the database system;
wherein the database system is configured for interrogation by a user and is configured to produce a probability matrix based on the information catalogued in step (b) following said interrogation.

62. The method of claim 61, wherein the interrogation comprises selecting one or more predetermined traits desired in the offspring animal and identifying from the plurality of non-human animals a breeding pair capable of producing said offspring animal.

63. The method of claim 61, further comprising selecting a breeding pair capable of producing the offspring having the one or more predetermined traits.

64. The method of claim 63, wherein the probability matrix comprises an array of probability values pertaining to the likelihood of the selected breeding pair producing a non-human offspring animal having the one or more predetermined traits.

65. The method of claim 61, wherein the probability matrix comprises an array of probability values pertaining to the likelihood of the plurality of non-human animals having the one or more predetermined traits.

66. The method of claim 61, wherein the non-genetic assessment comprises:
(a) obtaining or having obtained information from someone knowledgeable about the non-human animal pertaining to the one or more predetermined traits;
(b) performing or having performed one or more clinical assays on biological tissue obtained from the non-human animal pertaining to the one or more predetermined traits;
(c) performing or having performed one or more diagnostic tests on the non-human animal pertaining to the one or more predetermined traits; and/or
(d) obtaining scores assigned to the non-human animal during a competition pertaining to the one or more predetermined traits.

67. A computer-implemented interface for assisting a user in producing, procuring, and/or identifying one or more non-human animals having one or more predetermined traits, wherein the interface is configured to allow a user to interrogate a computer-implemented database system, the interrogation comprising:
(a) selecting the one or more predetermined traits of interest for the one or more non-human animals;
(b) identifying one or more of the non-human animals catalogued in the database system and generating a probability matrix for each of the one or more non-human animals identified using the database system;
(c) based on the probability matrix of step (d), displaying an overall assessment of each of the non-human animals having the one or more predetermined traits; and
(d) providing an option for the user to produce, purchase, or lease one or more of the non-human animals having the one or more predetermined traits;
wherein the computer-implemented interface is in communication with the database system.

68. The interface of claim 67, wherein step (a) further comprises selecting one or more predetermined traits of at least one parent of the one or more non-human animals.

69. The interface of claim 67, wherein step (a) further comprises selecting a gene pool from which the one or more non-human animals or the at least one parent of the non-human animal are obtained.

70. The interface of claim 69, wherein the gene pool is selected from a group consisting of the user's own animals, animals owned by other users, an animal registry, and animals located within a specified geographical area.

71. The interface of claim 67, wherein the probability matrix comprises an array of probability values pertaining to the likelihood of the non-human animal having one or more predetermined traits.

72. A method using a computer-implemented database system to identify a non-human animal as having one or more predetermined traits, comprising:
(a) providing information about the non-human animal;
(b) cataloguing the information of step (a) into the database system;
(c) comparing the information of step (b) to information about a plurality of non-human animals having the one or more predetermined traits, wherein the information about the plurality of non-human animals has been previously catalogued in the database system;
(d) generating a probability matrix for the non-human animal based on the comparison of step (c) using the database system, wherein the probability matrix comprises an array of probability values pertaining to the likelihood that the non-human animal has the one or more predetermined traits;
(e) identifying the non-human animal as having the one or more predetermined traits based on the probability matrix of step (d); and
(f) optionally, genetically modifying the non-human animal to promote or suppress an expression of the one or more predetermined traits in the non-human animal;
wherein the information comprises results of previously performed genetic testing and/or non-genetic assessment of the non-human animal or the plurality of non-human animals.

73. The method of any one of claims 1, 33, 54, 61, and 72, wherein the one or more predetermined traits comprise coat color, coat color modifier, coat texture, coat thickness, facial marking, leg marking, eye color, skin color, mane color, tail color, speed, gait, temperament, and health.

74. The method of claim 73, wherein the coat color, mane color and/or tail color is selected from a group consisting of amber champagne, amber champagne dun, amber cream, amber dun, amber dun pearl, apricot dun, apricot pearl, agouti, bay, bay cream pearl, bay double cream, bay pearl, black, black bay, black cream pearl, black double cream, black dun, black pearl, black/red, blanket appaloosa, blood bay, blue roan, brindle, brown, buckskin, buttermilk buckskin, champagne, champagne amber pearl, champagne classic pearl, champagne dun, champagne dun pearl, champagne gold pearl, champagne pearl, chesetnut, chestnut cream pearl, chocolate, classic champagne, classic champagne dun, classic cream, classic dun, cream, cream champagne, cream grullo, cream pearl, cremello, dapple grey, dark bay, dark brown, dark chestnut, dominant white, dun, dun bay cream, dun bay double cream, dun black cream, dun black double cream, dun chestnut cream, dun chestnut double cream, dun pearl, dun pearl bay, dun pearl black, dunalino, dunolino, dunskin, dunskin splash, flaxen chestnut, fleabitten grey, frame overo, frosted appaloosa, grey, gold champagne, gold cream, gold dun, gold dun pearl, grey, grullo, half blanket, leopard appaloosa, light grey, liver chestnut, marbled appaloosa, overo, palomino, pearl, perlino, perlino dun, pseudocream classic, red, red chestnut, red dun, red roan, rose grey, sabino, seal brown, silver, silver amber champagne, silver amber dun, silver bay cream, silver bay cream pearl, silver bay double cream, silver black, silver black cream pearl, silver black double cream, silver champagne, silver champagne dun, silver champagne pearl, silver classic champagne, silver classic champagne pearl, silver cream pearl, silver dapple, silver dapple bay, silver dapple buckskin, silver dapple cream grullo, silver dapple dun, silver dapple dunskin, silver dapple grullo, silver dapple pearl, silver dapple perlino, silver dapple perlino dun, silver dun, silver dun bay cream, silver dun bay double cream, silver dun black, silver dun black double cream, silver dun pearl, silver dun pearl bay, silver dun pearl black, silver pearl bay, silver pearl black, silver red, silver smoky cream, skewbald, smoky black, smoky cream, smoky grullo, snowflake appaloosa, sorrel, splashed white, steel grey, sun, tobiano, tovero, tri-color, white, and white spotting.

75. The method of claim 73, wherein the coat color modifier is selected from a group consisting of apron, barring on body, barring on shoulder, belly spots (large or small), belly stripe, belted, bend or spots on body, bend or spots on head, birdcatcher spots, black spots, blagdon, blanket with roaning, blanket with spots, blaze, blaze with freckling, body spots, body white, brindle, brow spots, calico, coon/skunk tail, dapples, dilute, dorsal stripe, double dilute, ermine markings, few white hairs on body, fewspot, flaxen mane/tail, flea-bitten, fleshmark, frosted, grullo, heart marking, highlights in mane/tail, lacing pattern, leopard spotted, lightning marks, line back, frosting mane/tail, maximum tobiano, maximum overo, maximum white, maximum white sabino, medicine hat, minimal overo, minimal sabino, minimal tobiano, mixed mane/tail, mottled, no marking, overo, pangare, paw prints, pearl, piebald, pinto markings, rabicano, roan, roaning on body, rose, rump spots, sabino, skewbald, smudge marks, smutty, snip, snowcap, sooty, splash markings, star, stripe, tobiano, umbilical spots, white head, white mane/tail, white throat latch, white tipped ears, white with fading edges, white with ragged edges, white with smooth edges, wither spot, no markings, solid, white blaze, and white star.

76. The method of claim 73, wherein the facial marking is selected from a group consisting of apron face, badger face, bald face, blaze, interrupted stripe, face, snip, star, stripe markings, both eyes amber, both eyes blue, both eyes brown, both eyes green, both eyes tiger, eyebrows, face mask, few white hairs on forehead, left eye amber, left eye blue, left eye brown, left eye green, left eye partial blue, left eye tiger, partial bald face, pigment around eye, right eye amber, right eye blue, right eye brown, right eye green, right eye partial blue, right eye tiger, white around eye, white chin, white jaw, white lip, white nose, and white sclera.

77. The method of claim 73, wherein the leg marking is selected from a group consisting of two back white stockings, two white front socks, two white front stockings, two white hind socks, two white hind stockings, four white socks, four white stockings, back left above hock, back left cannon (¾ stocking), back left coronet, back left ermine spots, back left fetlock, back left half pastern, back left heel, back left partial heel, back left pastern, back left sock, back left stocking, back left stripes, back right above hock, back right cannon, back right coronet, back right ermine spots, back right fetlock, back right half pastern, back right heel, back right partial heel, back right pastern, back right sock, back right stocking, back right stripes, barring on legs, bend or spots on legs, front left above knee, front left cannon, front left coronet, front left ermine spots, front left fetlock, front left half pastern, front left heel, front left partial heel, front left pastern, front left sock, front left stocking, front left stripes, front right above knee, front right cannon, front right coronet, fright right ermine spots, front right fetlock, front right half pastern, front right heel, front right partial heel, front right pastern, front right sock, front right stocking, front right stripes, left front hoof stripes, left front white foot, left hind hoof stripes, left hind white foot, right front hoof stripes, right front white foot, right hind hoof stripes, right hind white foot, and roaning on white socks/stockings.

78. The method of claim 73, wherein the coat texture comprises smooth, rough, curly, straight, downy, spiky, or brindle.

79. The method of claim 73, wherein the coat thickness comprises thick, medium, or thin.

80. The method of claim 73, wherein the eye color comprises blue, amber, yellow, orange, hazel, green, or brown.

81. The method of claim 73, wherein the skin color comprises pink, black, brown, yellow, and white.

82. The method of claim 73, wherein the speed is selected from the group consisting of endurance, mid-distance, and sprint.

83. The method of claim 73, wherein the gait is selected from the group consisting of non-gaited, gait carrier, and gaited.

84. The method of claim 83, wherein gaited comprises walk, trot, canter, gallop, pacing, fox trot, racking, country pleasure, Indian shuffle, jogging, paso fino, paso corto, paso largo, paso llano, sobrandando, marcha picada, rack, running walk, stepping pace, singlefoot, tölt, and ravaal.

85. The method of claim 73, wherein the temperament is selected from the group consisting of vigilant, curious/vigilant, curious, spooky, non-spooky, hot, cold, and medium.

86. The method of claim 73, wherein the health comprises variants of one or more genes associated with one or more disease or non-disease conditions that are undesired in the non-human animal.

87. The method of any one of claims 1, 33, 55, and 62, wherein each of the one or more breeding pairs comprise at least a first non-human parent animal and a second non-human parent animal.

88. The method of claim 87, wherein the method further comprises selecting the sex, breed, and/or discipline of the offspring animal, the first parent animal, and/or the second parent animal.

89. The method of claim 88, wherein the breed is selected from the group consisting of Abyssianian/Ethiopian/Oromo/Gala horse, Abyssinian, Akhal Teke, Albanian, Albanian Horse, Alt-Olderburger, Altai Horse, American Cream Draft, American Cream and White, American Curly, American Gaited Mountain Horse, American Gaited Pony, American Paint Horse, American Quarter Horse, American Saddlebred, American Walking Pony, American Warmblood, Andalusian, Andravida, Andravida/Eleia Horse, Anglo European Warmblood, Anglo-Arab, Anglo-Kabarda, Appaloosa, Appendix Quarter Horse, AraAppaloosa, Arabian, Arabian Sporthorse, Ardennes, Ardennes Horse, Argentine Criollo, Asturcon, Asturian, Australian Brumby, Australian Stock Horse, Austrian Warmblood, Azteca, Baise Horse, Balearic, Baluchi, Baluchi Horse, Ban-ei, Banker, Banker Horse, Barb, Barb/Berber Horse, Bashkir, Bashkir Curly, Bashkir Curly Horse, Bashkir Horse, Basotho Pony, Basuto Pony, Belgian, Belgian Draft, Belgian Warmblood, Bhirum Pony, Bhirum/Nigerian Pony, Bhotia Pony, Bhutia Pony, Black Forest, Black Forest Pony, Blue Eyed Horse Association, Boer, Boerperd, Brandenburger, Breton, Breton Horse, British Riding Pony, British Warmblood, Buckskin, Budyonny, Budyonny Horse, Byelorussian Harness, Byelorussian Harness Horse, Camargue, Camargue Horse, Campolina, Canadian Horse, Canadian Sport Horse, Canadian Suffield Mustang, Canadian Warmblood, Carolina Marsh Tacky, Carthusian, Caspian, Caspian Horse, Cayuse, Celtic Warmblood, Cheju, Chicoteague Pony, Chilean Corralero, Chilean Corralero Horse, Chincoteague Pony, Cleveland Bay, Clydesdale, Colorado Ranger Horse, Connemara, Cream Draft, Cremello, Criollo, Crioulo, Curly Horse, Dales Pony, Danish Warmblood, Danube, Danube Delta Horse, Dartmoor Pony, Deliboz, Deliboz/Delibozskaya, Djerma, Dole, Dole Gudbrandsdal/Dolahest/Døle, Dongola, Donkey, Draft, Draft Cross, Draft Horse, Drum Horse, Dutch Draft, Dutch Harness, Dutch Harness Horse, Dutch Warmblood, East Bulgarian, East Friesian, Egyptian, Egyptian Arabian, Eriskay Pony, Estonian Horse/Estonian Native/Estonian Klepper/Eisti Hobune, Estonian Native, Exmoor Pony, Faeroe Pony, Faeroes Pony, Falabella, Falabella Miniature Horse, Fell Pony, Ferghana, Finnhorse, Finnhorse/Finnish Horse, Fjord, Fjord Horse, Fleuve, Florida Cracker, Florida Cracker Horse, Foundation Quarter Horse, Fouta, Foutanke/Fouta, Frederiksborg, Frederiksborg Horse, French Saddlebred, French Saddlebred Horse, French Trotter, Friesian, Friesian Cross, Galiceño, Galician Pony, Galician/Galician Mountain Horse, Gelderlander, Georgian Grande, German Bred, German Riding Pony, Gidran, Gidran/Hungarian Anglo-Arab, Golden American Saddlebred, Gotland, Gotland Pony, Groningen, Groningen Horse, Guangxi, Gypsy Cob, Gypsy Vanner, Hackney, Hackney Horse, Hackney Pony, Haflinger, Half-Arabian, Hanoverian, Hanoverian Warmblood, Hequ, Highland Pony, Hokkaido, Hokkaido Horse, Holsteiner, Holsteiner Warmblood, Hucul, Hucul Pony, Hungarian Warmblood, Iberian Warmblood, Icelandic, Icelandic Horse, lomud, lomud/Yomud/Yamud/Yomut, Irish Cob, Irish Draft, Irish Draught, Irish Draught Horse, Irish Sport Horse, Jeju Horse, Jinzhou, Jutland, Kabarda, Kabarda/Kabardin, Karabair, Karabakh, Kazakh, Kazakh Horse, Kentucky Mountain horse, Kentucky Mountain Saddle Horse, Kerry Bog Pony, KigeR Mustang, Kirdi Pony, Kisber Felver, Kiso, Kiso Horse, Kladbury, Kladbury Horse, Knabstrup, Knabstrupper Warmblood, Kushum, Kushum Horse, Kustanai, Kustanai Horse, Kyrgyz/Kirgiiz Horse, Latvian, Latvian Horse, Lipizzan, Lithuanian Heavy Draft, Lokai, Losino, Losino Horse, Lusitano, M Bayar, Mallorquin, Malopolski, Mangalarga, Mangalarga Marchador, Marsh Tacky, Marwari, Marwari Horse, Wrens Pony, Messara, Messara Horse, Miniature, Miniature Horse, Misaki, Misaki Horse, Missouri Fox Trotter, Missouri Fox Trotting Horse, Miyako, Miyako Pony, Mongolian, Mongolian Horse, Morab, Morgan, Mountain Pleasure Horse, Moyle, Mule, Murgese, Mustang, National Show horse, National Spotted horse, New Forest Pony, New Kirgiz, Newfoundland Pony, Nokota, Nokota Horse, Noma, Noma Horse, Nooitgedacht Pony, Nordland, Nordlandshest/Lyngshest/Nordland Pony, Norfolk Trotter, Noric, North Swedish horse, Northeastern, Ob, Oldenburg, Oldenburg Warmblood, Orlov Trotter, Paint (Overo), Paint (Solid), Paint (Tobiano), Paint (Tovero), Paints (All), Palomino, Pantaneiro, Pantaneiro horse, Paso Fino, Paulista Marchador, Peneia Pony, Percheron, Peruvian Paso, Pindos Pony, Pinia, Pintabian, Pinto, POA, Polish Horse, Ponies (All), Pony, Pony of America, Pottok, Priob/Ob, Przewalski, Przewalski Horse, Pura Raza Espanola (PRE), Pyrenean Tarpan, Qatgani, Qatgani/Qataghani Horse, Quarab, Quarter Horse, Quarter Horse Cross, Quarter Pony, Racking Horse, Rheinland Pfalz Saar, Rhenish Warmblood, Rhinelander horse, Rocky Mountain, Rocky Mountain Horse, Russian Don, Russian Heavy Draft, Russian Trakhener, Russian Trotter, Sanhe, Sanhe Horse, Schleswiger Heavy Draft, Scharzwälder Fuchs, Selle Francais, Shagya, Shagya Arabian, Shetland Pony, Shire, Single-Footing Horse, Skyros Pony, Somali Pony, Sorraia, Soviet Heavy Draft, Spanish Mustang, Spanish-Barb, Spanish-Norman, Spanish-Normal Horse, Sport Horse of Color, Spotted Draft, Spotted Draft Horse, Spotted Saddle, Spotted Saddle Horse, Standardbred, Sudan Country-Bred, Sudan Country-Bred Horse, Suffolk, Suffolk/Suffolk Punch/Suffolk Sorrel, Swedish Warmblood, Taishu/Tsushima, Taishuh, Tarpan, Tawleed, Tennessee Walker, Tennessee Walking Horse/Tennessee Walker, Tersk, Tersk Horse, Thessalian, Thessalian/Thessaly Horse, Thoroughbred, Thoroughbred Cross, Tibetan Pony, Tiger horse, Tokara, Tori, Tori Horse, Trakehner, Ukrainian Riding Horse/Ukrainian Saddle Horse, Ukrainian Saddle, Virginia Highlander, Vlaamperd, Vladimir Heavy Draft, Vyatka, Vyatka Horse, Walkaloosa, Warmblood, Warmblood Cross, Warmblood (All), Warmbloods of Color, Welara Pony, Welsh Cob, Welsh Pony, Weser-Em Pony, West African Barb, Western Sudan Pony, Westphalian, Wielkopolski, Wurttemberger Warmblood, Xilingol, Yakut, Yanqi, Yili, Yonaguni, Zangersheide, Zaniskari Pony, Zhemaichu, and Zorse.

90. The method of claim 89, wherein the discipline is selected from the group consisting of barrel racing, beginner/family, breeding, brood mare, calf roping, companion only, competitive trail competitions, country pleasure, cowboy mounted shooting, cutting, draft, dressage, drill team, driving, endurance riding, English pleasure, equitation, eventing, field hunter, gaited, halter, harness, horsemanship, hunter, hunter under saddle, judged pleasure rides, jumper, lesson horse, longe-line, pleasure driving, pole bending, polo, racing, ranch horse, ranch sorting, reined cow horse, reining, rodeo, roping, showmanship, saddle seat, sidesaddle, steer wrestling, team penning, team roping, team sorting, trail horse, vaulting, Western pleasure, Western pleasure (show), Western riding, working cattle, youth/4-H horse, all around, 4-in hand driving, agility, breed shows, breeding stallion, broodmare, bull fighting, camping, cart, combined driving, cowboy dressage, cowboy racing, doma vaquero, field trials, gymkhana, harness racing, hazing, horse ball, marathon driving, mounted archery, mounted games, mounted patrol, mounted police, native costume, natural horsemanship, nurse mare, pacing, pack horse, parades, park, playday, polocrosse, ranch versatility, recipient broodmare, roadster, rodeo bronc, rodeo pickup horse, rodeo queen horse, roping, saddleseat, search and rescue, show hack, ski joring, steer roping, stock horse, sulky racing, team driving, team roping head horse, team roping heel horse, therapeutic riding, trial class competition, recreational trail riding, turnback horse, Western dressage, working equitation, hunt seat, show jumper, riding, pleasure, and bucking horse.

91. The method of any one of claims 1, 33, 55, and 62, wherein the likelihood that the offspring animal produced by each said breeding pair will have the one or more predetermined traits is at least 6.25%, 12.5%, 25%, 50%, 60%, 70%, 80%, 90%, 95%, 99%, or 100%.

92. The method of claim 54, wherein the disease or non-disease condition is selected from a group consisting of anhidrosis, androgen insensitivity syndrome, cerebellar abiotrophy, curved ear tips, dwarfism, degenerative suspensory ligament disease, equine asthma, equine herpes virus risk, epistaxis risk, equine metabolic syndrome, laminitis, equine recurrent uveitis, equine viral arteritis susceptibility, foal immunodeficiency syndrome, gait transition, glycogen branching enzyme disorder, hereditary equine regional dermal asthenia, hoof wall separation disease, hydrocephalus, hyperkalemic periodic paralysis, immune-mediated myositis, impaired acrosomal reaction, junctional epidermolysis bullosa, kissing spines, lavender foal syndrome, lordosis, malignant hyperthermia, myotonia, navicular disease, osteochondritis dissecans, polysaccharide storage myopathy, recurrent laryngeal neuropathy, severe combined immunodeficiency, squamous cell carcinoma, tendinopathy, warmblood fragile foal syndrome, West Nile Virus susceptibility, wobbler syndrome, and behavioral abnormality.

93. The method of claim 92, wherein the behavioral abnormality is selected from the group consisting of cribbing, teeth grinding, weaving, anxiety, depression, extreme aggression, extreme fearfulness, bucking, striking, biting, self-mutilating, and head shaking.

94. The method of any one of claims 1, 33, 54, 61, and 72, wherein the one or more predetermined traits comprise at least 2 or more predetermined traits.

95. The method of claim 94, wherein the one or more predetermined traits comprise at least 4 or more predetermined traits.

96. The method of claim 95, wherein the one or more predetermined traits comprise at least 6 or more predetermined traits.

97. The method of claim 96, wherein the one or more predetermined traits comprise at least 8 or more predetermined traits.

98. The method of claim 97, wherein the one or more predetermined traits comprise at least 10 or more predetermined traits.

99. The method of any one of claims 1, 33, 54, 61 72, and 87, wherein the computer-implemented database system comprises genetic and non-genetic information about the non-human animal, non-human parent animal, and/or non-human offspring animal.

100. The method of any one of claims 1, 52, 61, and 72, wherein the genetic testing comprises genotyping the non-human animal for any one of the following genetic markers:
(a) agouti, black/red, brindle, champagne, cream, dominant, white, frame overo, grey, pearl, sabino, silver, splashed white, tobiano, and white spotting;
(b) a curiosity gene that is a dopamine D4 receptor (DRD4) gene;
(c) a speed gene that is a myostatin (MSTN) gene;
(d) a gait gene that is a doublesex and mab-3-related transcription factor 3 (DMRT3) gene; and/or
(e) a health gene selected from the group consisting of androgen receptor (AR), mutY DNA glycosylase (MUTYH), teashirt zinc finger homeobox 1 (TSHZ1), aggrecan (ACAN), family with sequence similarity 174 member A (FAM174A), interleukin 17A (IL17A), interleukin 17B (IL17B), chemokine (C-X-C motif) ligand 16 (CXCL16), solute carrier family 5 member 3 (SLC5A3), glycogen branching enzyme (GBE1), peptidylprolyl isomerase B (PPIB), serpin family B member 11 (SERPINB11), beta-1,3-N-acetylgalactosaminyltransferase 2 (B3GALNT2), sodium voltage-gated channel alpha subunit 4 (SCN4A), myosin heavy chain 1 (MYH1), FKBP prolyl isomerase 6 (FKBP6), laminin subunit gamma 2 (LAMC2), myosin 5A (MYOSA), equine chromosome 20 (ECA20), ryanodine receptor 1 (RYR1), chloride voltage-gated channel 4 (CLCN4), glycogen synthase 1 (GYS1), equine chromosome 3 (ECA3), protein kinase DNA-activated catalytic subunit (DNAPK), damage specific DNA binding protein 2 (DDB2), procollagen-lysine, 2-oxoglutarate 5-dioxygenase 1 (PLOD1), 2′-5′-oligoadenylate synthetase 1 (OAS1), melanocyte inducing transcription factor (MITF), paired box (PAX), proto-oncogene receptor tyrosine kinase (KIT), high mobility group AT-hook 2 (HMGA2), zinc finger and AT-hook domain containing (ZFAT), ligand dependent nuclear receptor corepressor like (LCORL), non-SMC condensing I complex subunit G (NACPG), and LIM and SH3 protein 1 (LASP1).

101. The method of claim 100, wherein the genetic testing comprises performing analysis of an entire genome of the non-human animal.

102. The method of claim 101, wherein the analysis of an entire genome comprises genome wide association study (GWAS).

103. The method of any one of claims 1, 54, and 87, wherein the non-human animal, non-human parent animal, or non-human offspring animal is selected from the group consisting of horse, cattle, sheep, dog, cat, camel, pig, goat, alpaca, donkey, llama, red fox, mouse, rat, ferret, non-human primate, rabbit, gerbil, hamster, chinchilla, or guinea pig.

104. The method of any one of claims 1, 33, 54, 61, and 72, wherein the method further comprises generating a shared centimorgan (cM) value for the non-human animal or non-human offspring animal based on a sample obtained from the non-human animal or non-human offspring animal, respectively.

105. The method of any one of claims 1, 33, 54, 61, and 72, wherein the method further comprises generating a kinship coefficient for the non-human animal or non-human offspring animal based on a sample obtained from the non-human animal or non-human offspring animal, respectively.

106. The method of any one of claims 1, 33, 54, 61, and 72, wherein the method further comprises generating a heterozygosity score for the non-human animal or non-human offspring animal based on a sample obtained from the non-human animal or non-human offspring animal, respectively.

107. The method of any one of claims 1, 33, 54, 61, and 72, wherein the method further comprises generating an inbreeding coefficient (F) for the non-human animal or non-human offspring animal based on a sample obtained from the non-human animal or non-human offspring animal, respectively.

108. The method of any one of claims 1, 33, 54, 61, and 72, wherein the method further comprises performing a breed composition analysis on a sample from the non-human animal or the non-human offspring animal.

109. The method of any one of claims 1, 33, 54, 61, and 72, wherein the method further comprises performing an identity-by-descent analysis on a sample from the non-human animal or the non-human offspring animal.

110. The method of claim 104, wherein the sample is a biological sample

111. The method of claim 110, wherein the biological sample is a hair sample, such as a hair sample comprising one or more hair follicles.
